(19) United States
(12) Patent Application Publication
(10) Pub. No.: US 2024/0384274 A1
(43) Pub. Date: Nov. 21, 2024

(54) COMPOSITIONS FOR AND METHODS OF TREATING AND/OR PREVENTING GLUTARIC ACIDURIA TYPE-1

(71) Applicants: Duke University, Durham, NC (US); Baylor College of Medicine, Houston, TX (US)

(72) Inventors: Collin G. Johnson, Houston, TX (US); Maria de las Mercedes Barzi Dieguez, Durham, NC (US); Karl-Dimiter Bissig, Durham, NC (US)

(21) Appl. No.: 18/694,292

(22) PCT Filed: Sep. 23, 2022
(86) PCT No.: PCT/US2022/076982

§ 371 Date: Mar. 21, 2024

Related U.S. Application Data
(60) Provisional application No. 63/247,900, filed on Sep. 24, 2021.
(60) Provisional application No. 63/247,888, filed on Sep. 24, 2021.

(51) Int. Cl. C12N 15/113; A61K 31/436; A61K 31/519; A61K 31/573; A61K 31/69; A61K 38/46; A61K 39/395; A61K 48/00; A61P 1/16; A61P 3/00; C12N 9/02; C12N 9/22; C12N 15/11; C12N 15/86

(52) U.S. Cl. CPC C12N 15/1137; A61K 31/436; A61K 31/519; A61K 31/573; A61K 31/69; A61K 38/465; A61K 39/39516; A61K 39/3955; A61K 48/0033; A61P 1/16; A61P 3/00; C12N 9/001; C12N 9/22; C12N 15/11; C12N 15/86; C12N 2310/14; C12N 2310/20; C12N 2750/14143

(57) ABSTRACT

Disclosed herein are compositions for use in methods of treating and/or preventing Glutaric Aciduria Type 1 and in methods of reprogramming a metabolic pathway.

### I. CROSS-REFERENCE TO RELATED APPLICATIONS

This application claims priority to U.S. Provisional Application No. 63/247,900 filed 24 Sep. 2021 and U.S. Provisional Application No. 63/247,888 filed 24 Sep. 2021, each of which is incorporated herein in its entirety.

### II. REFERENCE TO THE SEQUENCE LISTING

The Sequence Listing submitted 23 Sep. 2022 as a .xml file named “22_2055_WO_Sequence_Listing”, created on 23 Sep. 2022 and having a size of 229 kilobytes is hereby incorporated by reference pursuant to 37 C.F.R. § 1.52(e)(5).

### III. BACKGROUND

Glutaric aciduria type I (GA-1), is a rare neurometabolic organic aciduria caused by glutaryl-CoA dehydrogenase (GCDH) deficiency. It is an autosomal recessive inborn error of lysine (primarily) and tryptophan catabolism with an estimated worldwide prevalence of 1:30,000 to 1:100,000 live births (Lindner M, et al. (2006) J Inherit Metab Dis. 29:378-382; Kolker S, et al. (2006) Pediatr Res. 59:840-847). Due to founder gene mutations, the incidence of GA-1 is higher in the old order Amish population of Pennsylvania (Strauss K A, et al. (2003) Am J Med Genet C Semin Med Genet. 121C(1):38-52) and the Lumbee Native Indian Tribe population of North Carolina (Basinger A A, et al. (2006) Mol Genet Metab. 88:90-92). Even though GCDH expression is mainly hepatic, loss of its enzymatic activity leads to accumulation of toxic intermediates with predominantly neurological sequelae. Symptomatic patients present with neonatal macrocephaly, subdural hematomas and acute retinal hemorrhage. Infants are at risk of acute encephalopathic crises triggered by recurrent febrile illness, or poor intake, damaging the brain striatum. Infantile acute striatal necrosis is the hallmark of the disease and the primary cause of morbidity and mortality. Putamin injury is associated with behavioral regression. (Strauss K A, et al. (2003) Am J Med Genet C Semin Med Genet. 121C(1):38-52). Some patients have insidious onset disease with late-onset neurologic sequelae. (Strauss K A, et al. (2003) Am J Med Genet C Semin Med Genet. 121C(1):38-52).

Standard of care therapy is dietary restriction of lysine and tryptophan, carnitine supplementation, symptomatic treatment of neurological manifestations, and high calorie glucose infusion during physiologic stress to prevent metabolic crises and strokes. Despite early diagnosis made possible with newborn screening (NBS) and improved management of patients with GA-1, 25-33% of these patients continue to develop acute and long-term neurological complications. (Strauss K A, et al. (2003) Am J Med Genet C Semin Med Genet. 121C: 53-70; Sauer S W, et al. (2006) J Neurochem. 97:899-910). This indicates that dietary treatment is imperfect and reveals an urgent need to develop alternative, more effective therapies.

Consequently, the present disclosure provides compositions for and methods of treating and/or preventing glutaric aciduria type-I (GA-I) and methods of reprogramming a metabolic pathway.

### IV. BRIEF DESCRIPTION OF THE FIGURES

FIG. 1A-FIG. 1F show knockout mice and transplantation experiments. FIG. 1A shows lysine catabolism pathway scheme in peroxisomes and mitochondria. FIG. 1B shows Kaplan-Meier survival curves of Gcdh^{-/-} knockout mice transplanted with wild type hepatocytes (Gcdh^{+/+}) on high protein diet (casein). FIG. 1C shows Glutaric acid (GA) and 3-hydroxy GA (3-OH-GA) of groups after 10 days (non-transplanted) or 160 days (transplanted). FIG. 1D shows RFP immunohistochemistry only detecting healthy hepatocytes (Gcdh^{+/+}, transgenic mTmG) while FIG. 1E shows Western blot (GCDH and beta actin) with liver lysates of low and high repopulated transplanted animals (4 each). FIG. 1F shows Kaplan-Meier survival curves of single (Gcdh^{-/-}) knockout and double knockout (Gcdh^{-/-}/Aass^{-/-}) mice on high protein diet. FIG. 1G shows GA and 3-OH-GA of single (Gcdh^{-/-}) knockout and double knockout (Gcdh^{-/-}/Aass^{-/-}) mice after 5 days on high protein diet. FIG. 1H shows Kaplan-Meier survival curves of double (Gcdh^{-/-}/Aass^{-/-}) knockout mice transplanted with Gcdh^{-/-} hepatocytes. FIG. 1I shows GA and 3-OH-GA of double (Gcdh^{-/-}/Aass^{-/-}) knockout mice transplanted with Gcdh^{-/-} hepatocytes. FIG. 1J shows AASS immunostaining from transplanted double (Gcdh^{-/-}/Aass^{-/-}) knockout mice. FIG. 1K shows Western blot of livers from transplanted double (Gcdh^{-/-}/Aass^{-/-}) knockout mice. FIG. 1L shows a summary of transplantation models and their outcome. Significance was validated with t test (*p≤0.05, **p<0.01 ***p<0.005 and ****p<0.0001).

FIG. 2A-FIG. 2C show the neuropathological evaluation of Gcdh^{-/-} mice transplanted with healthy (Gcdh^{-/-}) hepatocytes. FIG. 2A shows H&E staining of hippocampal brain sections. Hippocampal vacuolation (arrows) and meningeal hemorrhage (arrowheads). FIG. 2B shows quantification of vacuolation and meningeal hemorrhage. FIG. 2C shows C57B6, transplanted and non-transplanted Gcdh^{-/-} knockout mice repopulated with wild type (Gcdh^{-/-}) hepatocytes. Significance was validated with t test (*p<0.05, and **p<0.01).

FIG. 3A-FIG. 3G show the motor performance of Gcdh^{-/-} mice transplanted with healthy (Gcdh^{-/-}) hepatocytes. FIG. 3A shows latency to fall from the rotarod across trials. FIG. 3B shows grip strength for the fore paws and FIG. 3C shows grip strength for the hind paws. FIG. 3D open field cumulative locomotor activity, FIG. 3E shows cumulative rearing activity, FIG. 3F shows cumulative distance traveled in the center zone, and FIG. 3G shows the velocity of locomotion. Data are presented as means±SEM. Significance was validated with t test, (*p<0.05 and **p<0.01).

FIG. 4A-FIG. 4B show the generation of knockout mice. FIG. 4A shows a schematic representation of murine Aass and Gcdh genes and the sgRNAs used to generate the single (Gcdh^{+/+}) and double (Gcdh^{+/+}/Aass^{-/-}) knockout strains. Exonic sgRNA target sites are marked. FIG. 4B shows an image of a DNA gel electrophoresis showing both the wild type and deleted bands of Aass and Gcdh amplified by PCR using genomic DNA from knockout mice generated by CRISPR/Cas9.

FIG. 5A-FIG. 5C show neuropathological evaluation of double (Gcdh^{+/+}/Aass^{-/-}) knockout mice transplanted with healthy (Gcdh^{-/-}) hepatocytes. FIG. 5A shows H&E staining of hippocampal brain sections. Hippocampal vacuolation (arrows). FIG. 5B shows quantification of vacuolation of C57BL, Gcdh^{-/-}Aass^{-/-} double knockout repopulated with Gcdh^{-/-} hepatocytes and Gcdh^{-/-}Aass^{-/-} double knockout control (non-transplanted) mice. FIG. 5C shows quantification of meningeal hemorrhage of C57BL, Gcdh^{-/-}Aass^{-/-} double knockout repopulated with Gcdh^{-/-} hepatocytes and Gcdh^{-/-}Aass^{-/-} double knockout control (non-transplanted) mice. Data are presented as means±SEM. Significance was validated with t test (****p<0.0001).

FIG. 6A shows a schematic representation of AAV virus expressing murine Gcdh cDNA sequence. FIG. 6B shows a pAAV-Gcdh plasmid sequence with annotations extracted from SnapGene.

FIG. 7A-FIG. 7G show liver directed gene therapy in Gcdh^{-/-} mice. FIG. 7A-7E show 5-week-old Gcdh^{-/-} mice treated with AAV-Gcdh, AAV-GFP (1.5×10¹² vg/mouse at 3 weeks of age) or no injection. FIG. 7A shows Kaplan Meier survival curves of Gcdh^{-/-} mice on high protein. FIG. 7B shows Western blot of liver and brain lysates from AAV treated mice after harvesting or expiration (controls). FIG. 7C shows C5-DC metabolite levels in blood (before and 4 days after high protein diet) and FIG. 7D shows Glutaric Acid levels in blood and 3-OH-Glutaric Acid levels in liver and brain of Gcdh^{-/-} mice at 140 days (AAV-Gcdh) and upon expiration (AAV-GFP and untreated). FIG. 7E-FIG. 7F show neonatal Gcdh^{-/-} pups treated with low (3×10¹¹ vg/mouse), intermediate (7.5×10¹¹ vg/mouse) and high (1.5×10¹² vg/mouse) dose of AAV. FIG. 7F shows Kaplan Meier survival curves of treated Gcdh^{-/-} mice on high protein after weaning. FIG. 7G shows Western blot for GCDH of treated Gcdh^{-/-} mice after expiration. Significance was validated with t test (*p≤0.05, **p≤0.01 ***p≤0.005 and ****p≤0.0001).

FIG. 8A-FIG. 8C show neuropathological evaluation of Gcdh^{-/-} mice treated with AAV-Gcdh or AAV-GFP control. FIG. 8A shows H&E staining of hippocampal brain sections. Hippocampal vacuolation (arrows). FIG. 8B shows quantification of vacuolation of C57BL, Gcdh^{-/-} single knockout controls (AAV-GFP injected) and Gcdh^{-/-} single knockout mice injected with AAV-mGcdh. FIG. 8C shows quantification of meningeal hemorrhage of C57BL, Gcdh^{-/-} single knockout controls (AAV-GFP injected) and Gcdh^{-/-} single knockout mice injected with AAV-mGcdh. Data are presented as means±SEM. Significance was validated with t test (*p<0.05 and **p<0.01).

FIG. 9A-FIG. 9G shows motor performance of Gcdh^{-/-} mice treated with AAV-Gcdh FIG. 9A shows latency to fall from the rotarod across trials. FIG. 9B shows grip strength for the fore paws. FIG. 9C shows grip strength for the hind paws. FIG. 9D shows open field cumulative locomotor activity. FIG. 9E shows cumulative rearing activity. FIG. 9F shows cumulative distance traveled in the center zone. FIG. 9G shows velocity of locomotion. Data are presented as means±SEM. Significance was validated with t test (*p<0.05 and ***p<0.005).

FIG. 10A shows schematic representation of the murine Aass gene and sgRNAs used to delete the gene. FIG. 10B shows a schematic representation of the AAV-CRISPR-Aass viruses used to knock out the gene targeting exons 6 and exon 7 of Aass. FIG. 10C shows a pAAV-CRISPR-Aass-Exon6-sgRNA plasmid map. FIG. 10D shows a pAAV-CRISPR-Aass-Exon7-sgRNA plasmid map.

FIG. 11A-FIG. 11D shows liver specific deletion of Aass in Gcdh^{-/-} mice using AAV-CRISPR. Neonatal Gcdh^{-/-} mice were injected with a low (2.4×10¹¹ vg/mouse), intermediate (6×10¹¹ vg/mouse) and high (1×10¹² vg/mouse) dose of AAV expressing Cas9 under a liver specific promoter and sgRNA targeting the Aass gene. FIG. 11A shows Kaplan Meier survival curves of experimental groups after exposure to high protein diet. FIG. 11B shows Glutaric Acid and 3-OH-Glutaric Acid levels in liver and brain of wild-type C57B6J mice and treated (AAV-CRISP, high dose) Gcdh^{-/-} mice after 60 days high protein diet and upon expiration (day 4, AAV-GFP). FIG. 11C shows AASS immunostaining of livers of depict groups. FIG. 11D shows representative sections of the hippocampus of mice injected with AAV-CRISPR or AAV-GFP or showing vacuolation (arrows) and hemorrhage (arrowheads). The bottom of the drawing provides the quantification of hippocampal vacuolation and meningeal hemorrhage by blinded veterinarian pathologist. Data are presented as means±SEM Significance was validated with t test (**p<0.01, ***p 0.005 and ****p 0.0001.

FIG. 12A-FIG. 12G show motor performance of Gcdh^{-/-} mice treated with AAV-CRISPR-Aass virus. FIG. 12A shows latency to fall from the rotarod across trials. FIG. 12B shows grip strength for the fore paws. FIG. 12C shows grip strength for the hind paws. FIG. 12D shows open field cumulative locomotor activity, FIG. 12E shows cumulative rearing activity, FIG. 12F shows cumulative distance traveled in the center zone, and FIG. 12G shows the velocity of locomotion. Data are presented as means±SEM. Significance was validated with t test (**p≤0.01 and ****p<0.0001).

FIG. 13A shows C5-DC metabolite in blood from wild type mice (C57BL) and Gcdh^{-/-}-AAV-Aass-CRISPR mice before and 4 days after high protein exposure. FIG. 13B shows lysine in blood from wild type mice (C57BL) and Gcdh^{-/-}-AAV-Aass-CRISPR mice before and 4 days after high protein exposure. FIG. 13C shows tryptophan levels in blood from wild type mice (C57BL) and Gcdh^{-/-}-AAV-Aass-CRISPR mice before and 4 days after high protein exposure. Data are presented as means±SEM. Significance was validated with t test (*p≤0.05, **p≤0.01).

FIG. 14A-FIG. 14B show intravenous injection with siRNA against AASS in Gcdh^{-/-} mice. FIG. 14A shows Kaplan Meier survival curves of treated (siRNA against Aass, at day −1) and control (siRNA non targeting mouse genome, at day −1) Gcdh^{-/-} mice on high protein diet (Days: days after high protein diet). FIG. 14B shows immunostaining for AASS of livers postmortem of the treatment group (siRNA against Aass). *p<0.05 for survival curves using Log-rank (Mantel-Cox) test.

### V. BRIEF SUMMARY

Disclosed herein is an isolated nucleic acid molecule comprising a nucleic acid sequence encoding glutaryl-CoA dehydrogenase.

Disclosed herein is an expression cassette comprising a disclosed isolated nucleic acid molecule comprising a nucleic acid sequence encoding a glutaryl-CoA dehydrogenase, wherein the nucleic acid sequence comprises the sequence set forth in SEQ ID NO:01 or SEQ ID NO:02, and is operably linked a promoter. Disclosed herein is an expression cassette comprising a disclosed isolated nucleic acid molecule comprising a nucleic acid sequence encoding a glutaryl-CoA dehydrogenase, wherein the nucleic acid sequence comprises the sequence set forth in SEQ ID NO:17 or SEQ ID NO:18, and is operably linked a promoter.

Disclosed herein is an isolated nucleic acid molecule comprising a nucleic acid sequence encoding one or more element of a gene editing system. Disclosed herein is an isolated nucleic acid molecule comprising a nucleic acid sequence encoding one or more element of a gene editing system, wherein the one or more elements comprises an endonuclease and a sgRNA directed at a target sequence in a target gene of interest. Disclosed herein is an isolated nucleic acid molecule comprising a nucleic acid sequence encoding one or more element of a gene editing system, wherein the one or more elements comprises an Cas9 endonuclease and a sgRNA directed a target sequence in the aminoadipate-semialdehyde synthase gene.

Disclosed herein is a viral vector comprising a disclosed isolated nucleic acid molecule. Disclosed herein is a viral vector comprising a disclosed isolated nucleic acid molecule comprising a nucleic acid sequence encoding glutaryl-CoA dehydrogenase. Disclosed herein is a viral vector comprising a disclosed isolated nucleic acid molecule comprising a nucleic acid sequence encoding aminoadipate-semialdehyde synthase.

Disclosed herein is a viral vector comprising a nucleic acid sequence encoding a glutaryl-CoA dehydrogenase, wherein the nucleic acid sequence comprise the sequence set forth in SEQ ID NO:01 or a fragment thereof. Disclosed herein is a viral vector comprising a nucleic acid sequence encoding a glutaryl-CoA dehydrogenase, wherein the nucleic acid sequence comprise the sequence set forth in SEQ ID NO:02 or a fragment thereof. Disclosed herein is a viral vector comprising an isolated nucleic acid molecule comprising a nucleic acid sequence encoding one or more element of a gene editing system, wherein the one or more elements comprises an Cas9 endonuclease and a sgRNA directed at a target sequence in the glutaryl-CoA dehydrogenase gene. Disclosed herein is a viral vector comprising an isolated nucleic acid molecule comprising a nucleic acid sequence encoding one or more element of a gene editing system, wherein the one or more elements comprises an Cas9 endonuclease and a sgRNA directed at a target sequence in the aminoadipate-semialdehyde synthase gene.

Disclosed herein is a recombinant AAV vector comprising an isolated nucleic acid molecule comprising a nucleic acid sequence encoding one or more element of a gene editing system, wherein the one or more elements comprises an Cas9 endonuclease and a sgRNA directed at a target sequence in the glutaryl-CoA dehydrogenase gene. Disclosed herein is a recombinant AAV vector comprising an isolated nucleic acid molecule comprising a nucleic acid sequence encoding one or more element of a gene editing system, wherein the one or more elements comprises an Cas9 endonuclease and a sgRNA directed at a target sequence in the glutaryl-CoA dehydrogenase gene.

Disclosed herein is a viral vector comprising the sequence set forth in SEQ ID NO:19. Disclosed herein is a viral vector comprising the sequence set forth in SEQ ID NO:20. Disclosed herein is a viral vector comprising the sequence set forth in SEQ ID NO:21.

Disclosed herein is a method of restoring the expression of glutaryl-CoA dehydrogenase, the method comprising administering to a subject in need thereof a therapeutically effective amount of a vector viral vector comprising a nucleic acid sequence encoding glutaryl-CoA dehydrogenase, wherein expression of the nucleic acid sequence generates a functional glutaryl-CoA dehydrogenase. Disclosed herein is a method of treating and/or preventing GA-1 disease progression, the method comprising administering to a subject in need thereof a therapeutically effective amount of a vector viral vector comprising a nucleic acid sequence encoding glutaryl-CoA dehydrogenase, wherein expression of glutaryl-CoA dehydrogenase is restored.

Disclosed herein is a method of treating and/or preventing GA-1 disease progression, the method comprising administering to a subject in need thereof a therapeutically effective amount of a vector viral vector comprising the nucleic acid sequence set forth in SEQ ID NO:01, wherein expression of glutaryl-CoA dehydrogenase is restored. Disclosed herein is a method of treating and/or preventing GA-1 disease progression, the method comprising administering to a subject in need thereof a therapeutically effective amount of a vector viral vector comprising the nucleic acid sequence set forth in SEQ ID NO:02, wherein expression of glutaryl-CoA dehydrogenase is restored.

Disclosed herein is a method of treating and/or preventing GA-1 disease progression, the method comprising treating a subject in need thereof by administering to the subject a therapeutically effective amount of a vector viral vector comprising a nucleic acid sequence encoding glutaryl-CoA dehydrogenase, wherein expression of glutaryl-CoA dehydrogenase is restored.

Disclosed herein is a method of reprogramming a metabolic pathway, the method comprising administering to a subject in need thereof a therapeutically effective amount of a viral vector comprising an isolated nucleic acid molecule comprising a nucleic acid sequence encoding one or more element of a gene editing system, wherein the expression of the nucleic acid molecule disrupts the expression and/or function of the target gene.

Disclosed herein is a method of reprogramming a metabolic pathway, the method comprising administering to a subject in need thereof a therapeutically effective amount of a viral vector comprising a nucleic acid sequence encoding an endonuclease and a sgRNA directed at a target sequence in a target gene of interest, wherein the expression of the nucleic acid molecule disrupts the expression and/or function of the target gene.

Disclosed herein is a method of reprogramming a metabolic pathway, the method comprising administering to a subject in need thereof a therapeutically effective amount of a first viral vector comprising an isolated nucleic acid molecule comprising a nucleic acid sequence encoding one or more element of a gene editing system, and administering a therapeutically effective amount of a second viral vector comprising an isolated nucleic acid molecule comprising a nucleic acid sequence encoding one or more element of a gene editing system.

Disclosed herein is a method of reprogramming a metabolic pathway, the method comprising administering to a subject in need thereof a therapeutically effective amount of a first viral vector comprising a nucleic acid sequence encoding an endonuclease and a sgRNA directed at a target gene of interest, and administering a therapeutically effective amount of a second viral vector comprising a nucleic acid sequence encoding an endonuclease and a sgRNA directed at a target sequence in the target gene of interest. Disclosed herein is a method of reprogramming a metabolic pathway, the method comprising administering to a subject in need thereof a therapeutically effective amount of a viral vector comprising a nucleic acid sequence encoding a Cas9 endonuclease and a sgRNA directed at a target sequence in the aminoadipate-semialdehyde synthase gene; and administering to the subject a therapeutically effective amount of a second viral vector comprising a nucleic acid sequence encoding a Cas9 endonuclease and a sgRNA directed at a target sequence in the aminoadipate-semialdehyde synthase gene.

Disclosed herein is a method of reprogramming a metabolic pathway, the method comprising administering to a subject in need thereof a therapeutically effective amount of a viral vector comprising a nucleic acid sequence encoding an endonuclease and a sgRNA directed at a target sequence in a target gene of interest, wherein the target gene comprises the aminoadipate-semialdehyde synthase gene, α-aminoadipic semialdehyde gene, the kynurenine aminotransferase 2 gene, the dehydrogenase E1 and transketolase domain-containing protein 1 gene, the L-lysine alpha-oxidase gene, the ketimine reductase mu-crystallin protein gene, the peroxisomal sarcosine oxidase gene, or the pyrroline-5-carboxylate reductase gene, and wherein the expression of the nucleic acid molecule disrupts the expression and/or function of the target gene.

### VI. DETAILED DESCRIPTION

The present disclosure describes formulations, compounded compositions, kits, capsules, containers, and/or methods thereof. It is to be understood that the inventive aspects of which are not limited to specific synthetic methods unless otherwise specified, or to particular reagents unless otherwise specified, as such may, of course, vary. It is also to be understood that the terminology used herein is for the purpose of describing particular aspects only and is not intended to be limiting. Although any methods and materials similar or equivalent to those described herein can be used in the practice or testing of the present invention, example methods and materials are now described.

All publications mentioned herein are incorporated herein by reference to disclose and describe the methods and/or materials in connection with which the publications are cited. The publications discussed herein are provided solely for their disclosure prior to the filing date of the present application. Nothing herein is to be construed as an admission that the present invention is not entitled to antedate such publication by virtue of prior invention.

### A. Abbreviations

SaCas9: Staphylococcus Aureus Cas9; Syn PolyA: Synthetic Poly Adenylation Signal; ITR: Inverted Terminal Repeat; HLP: Hybrid Liver Promoter; GCDH: Glutaryl-Co-A Dehydrogenase; ORF: Open Reading Frame; Syn PolyA: Synthetic Poly Adenylation Signal; AASS: Alpha Aminoadipate-Semialdehyde Synthase; C5-DC: glutarylcarnitine.

### B. Definitions

Before the present compounds, compositions, articles, systems, devices, and/or methods are disclosed and described, it is to be understood that they are not limited to specific synthetic methods unless otherwise specified, or to particular reagents unless otherwise specified, as such may, of course, vary. It is also to be understood that the terminology used herein is for the purpose of describing particular aspects only and is not intended to be limiting. Although any methods and materials similar or equivalent to those described herein can be used in the practice or testing of the present invention, example methods and materials are now described.

This disclosure describes inventive concepts with reference to specific examples. However, the intent is to cover all modifications, equivalents, and alternatives of the inventive concepts that are consistent with this disclosure.

As used in the specification and the appended claims, the singular forms “a”, “an”, and “the” include plural referents unless the context clearly dictates otherwise.

The phrase “consisting essentially of” limits the scope of a claim to the recited components in a composition or the recited steps in a method as well as those that do not materially affect the basic and novel characteristic or characteristics of the claimed composition or claimed method. The phrase “consisting of” excludes any component, step, or element that is not recited in the claim. The phrase “comprising” is synonymous with “including”, “containing”, or “characterized by”, and is inclusive or open-ended. “Comprising” does not exclude additional, unrecited components or steps.

As used herein, when referring to any numerical value, the term “about” means a value falling within a range that is ±10% of the stated value.

Ranges can be expressed herein as from “about” one particular value, and/or to “about” another particular value. When such a range is expressed, a further aspect includes from the one particular value and/or to the other particular value. Similarly, when values are expressed as approximations, by use of the antecedent “about,” it will be understood that the particular value forms a further aspect. It will be further understood that the endpoints of each of the ranges are significant both in relation to the other endpoint and independently of the other endpoint. It is also understood that there are a number of values disclosed herein, and that each value is also herein disclosed as “about” that particular value in addition to the value itself. For example, if the value “10” is disclosed, then “about 10” is also disclosed. It is also understood that each unit between two particular units are also disclosed. For example, if 10 and 15 are disclosed, then 11, 12, 13, and 14 are also disclosed.

References in the specification and concluding claims to parts by weight of a particular element or component in a composition denotes the weight relationship between the element or component and any other elements or components in the composition or article for which a part by weight is expressed. Thus, in a compound containing 2 parts by weight component X and 5 parts by weight component Y, X and Y are present at a weight ratio of 2:5 and are present in such ratio regardless of whether additional components are contained in the compound.

As used herein, the terms “optional” or “optionally” means that the subsequently described event or circumstance can or cannot occur, and that the description includes instances where said event or circumstance occurs and instances where it does not. In an aspect, a disclosed method can optionally comprise one or more additional steps, such as, for example, repeating an administering step or altering an administering step.

As used herein, the term “subject” refers to the target of administration, e.g., a human being. The term “subject” also includes domesticated animals (e.g., cats, dogs, etc.), livestock (e.g., cattle, horses, pigs, sheep, goats, etc.), and laboratory animals (e.g., mouse, rabbit, rat, guinea pig, fruit fly, etc.). Thus, the subject of the herein disclosed methods can be a vertebrate, such as a mammal, a fish, a bird, a reptile, or an amphibian. Alternatively, the subject of the herein disclosed methods can be a human, non-human primate, horse, pig, rabbit, dog, sheep, goat, cow, cat, guinea pig, or rodent. The term does not denote a particular age or sex, and thus, geriatric, adult, adolescent, and child subjects, as well as fetuses, whether male or female, are intended to be covered. In an aspect, a subject can be a human subject. In an aspect, a subject can have a disease or disorder characterized by lysine catabolism dysfunction.

As used herein, the term “diagnosed” means having been subjected to an examination by a person of skill, for example, a physician, and found to have a condition (such as GA-1) that can be diagnosed or treated by one or more of the disclosed compositions or by one or more of the disclosed methods. For example, “diagnosed with a disease or disorder characterized by lysine catabolism dysfunction” means having been subjected to an examination by a person of skill, for example, a physician, and found to have a condition (GA-1) that can be treated by one or more of the disclosed compositions or by one or more of the disclosed methods. For example, “suspected of having a disease or disorder characterized by lysine catabolism dysfunction” can mean having been subjected to an examination by a person of skill, for example, a physician, and found to have a condition (such as GA-1) that can likely be treated by one or more of the disclosed compositions or by one or more of the disclosed methods. In an aspect, an examination can be physical, can involve various tests (e.g., blood tests, genotyping, biopsies, etc.), diagnostic evaluations (e.g., X-ray, CT scan, etc.), and assays (e.g., enzymatic assay), or a combination thereof. In an aspect, an examination can be objective and/or subjective.

As used herein, “isolated” means altered or removed from the natural state through human intervention. For example, naturally occurring siRNAs in living animals are not “isolated”, but synthetic siRNAs or siRNAs that are partially or completely separated from coexisting materials in their natural state are “isolated”. An isolated siRNA can be in substantially purified form or in a non-native environment, such as a cell into which the siRNA has been introduced.

A “patient” refers can refer to a subject afflicted with a disease or disorder such as GA-1. In an aspect, a patient can refer to a subject that has been diagnosed with or is suspected of having GA-1. In an aspect, a patient can refer to a subject that has been diagnosed with or is suspected of having GA-1 and is seeking treatment or receiving treatment for GA-1. In an aspect, a “patient” can refer to a subject afflicted with a disease or disorder characterized by lysine catabolism dysfunction and/or malfunction. In an aspect, a patient can refer to a subject that has been diagnosed with or is suspected of having a disease or disorder a disease or disorder characterized by lysine catabolism dysfunction and/or malfunction. In an aspect, a patient can refer to a subject that has been diagnosed with or is suspected of having a disease or disorder and is seeking treatment or receiving treatment for a disease or disorder (such GA-1).

As used herein, “codon optimization” can refer to a process of modifying a nucleic acid sequence for enhanced expression in the host cells of interest by replacing one or more codons or more of the native sequence with codons that are more frequently or most frequently used in the genes of that host cell while maintaining the native amino acid sequence. Various species exhibit particular bias for certain codons of a particular amino acid. As contemplated herein, genes can be tailored for optimal gene expression in a given organism based on codon optimization. Codon usage tables are readily available, for example, at the “Codon Usage Database.” Many methods and software tools for codon optimization have been reported previously. (See, for example, genomes.urv.es/OPTIMIZER/).

As used herein, the phrase “identified to be in need of treatment,” or the like, refers to selection of a subject based upon need for treatment of a disease or disorder characterized by lysine catabolism dysfunction and/or malfunction (such as GA-1). For example, a subject can be identified as having a need for treatment based upon an earlier diagnosis by a person of skill and thereafter subjected to treatment for a disease or disorder characterized by lysine catabolism dysfunction and/or malfunction (such as GA-1). In an aspect, the identification can be performed by a person different from the person making the diagnosis. In an aspect, the administration can be performed by one who performed the diagnosis.

As used herein, “oligonucleotides” and similar terms relate to short oligos composed of naturally occurring nucleotides as well as to oligos composed of synthetic or modified nucleotides, as described in the preceding section on RNAi and siRNA. The terms “polynucleotide” and “oligonucleotide” are used synonymously.

As used herein, “inhibit,” “inhibiting”, and “inhibition” mean to diminish or decrease an activity, level, response, condition, severity, disease, or other biological parameter. In an aspect, “inhibiting” can refer to diminishing the intensity, the duration, the amount, or a combination thereof of symptoms, complications, issues due to a subject's lysine catabolism dysfunction and/or malfunction (such as GA-1). This can include, but is not limited to, the complete ablation of the activity, level, response, condition, severity, disease, or other biological parameter. This can also include, for example, a 10% inhibition or reduction in the activity, level, response, condition, severity, disease, or other biological parameter as compared to the native or control level (e.g., a subject not having a disease or disorder characterized by lysine catabolism dysfunction and/or malfunction (such as GA-1)) or to the level prior to the onset of a disease or disorder characterized by lysine catabolism dysfunction and/or malfunction (such as GA-1). Thus, in an aspect, the inhibition or reduction can be a 10%, 20%, 30%, 40%, 50%, 60%, 70%, 80%, 90%, 100%, or any amount of reduction in between as compared to native or control levels or to the subject's level prior to the onset of lysine catabolism dysfunction and/or malfunction. In an aspect, the inhibition or reduction can be 10-20%, 20-30%, 30-40%, 40-50%, 50-60%, 60-70%, 70-80%, 80-90%, or 90-100% as compared to native or control levels or to the subject's level prior to the onset of lysine catabolism dysfunction and/or malfunction (such as GA-1). In an aspect, the inhibition or reduction can be 0-25%, 25-50%, 50-75%, or 75-100% as compared to native or control levels or to the subject's level prior to the onset of lysine catabolism dysfunction and/or malfunction (such as GA-1).

The words “treat” or “treating” or “treatment” include palliative treatment, that is, treatment designed for the relief of symptoms rather than the curing of a disease or disorder characterized by lysine catabolism dysfunction and/or malfunction (such as GA-1); preventative treatment, that is, treatment directed to minimizing or partially or completely inhibiting the development of a disease or disorder characterized by lysine catabolism dysfunction and/or malfunction (such as GA-1); and supportive treatment, that is, treatment employed to supplement another specific therapy directed toward the improvement of a disease or disorder characterized by lysine catabolism dysfunction and/or malfunction (such as GA-1). In an aspect, the terms cover any treatment of a subject, including a mammal (e.g., a human), and includes: (i) preventing the undesired physiological change and/or pathological condition from occurring in a subject that can be predisposed to a disease or disorder characterized by lysine catabolism dysfunction and/or malfunction (such as GA-1) but has not yet been diagnosed as having it; (ii) inhibiting the physiological change and/or pathological condition (a disease or disorder characterized by lysine catabolism dysfunction and/or malfunction (such as GA-1)); or (iii) relieving the physiological change and/or pathological condition, i.e., causing regression of a disease or disorder characterized by lysine catabolism dysfunction and/or malfunction (such as GA-1). For example, in an aspect, treating a disease or disorder can reduce the severity of an established a disease or disorder in a subject by 1%-100% as compared to a control (such as, for example, an individual not having a disease or disorder characterized by lysine catabolism dysfunction and/or malfunction (such as GA-1)). In an aspect, treating can refer to a 1%, 2%, 3%, 4%, 5%, 6%, 7%, 8%, 9%, 10%, 20%, 30%, 40%, 50%, 60%, 70%, 80%, 90%, or 100% reduction in the severity of a disease or a disorder or a condition (such as a disease or disorder characterized by lysine catabolism dysfunction and/or malfunction (such as GA-1)). For example, treating a disease or a disorder can reduce one or more symptoms of a disease or disorder in a subject by 1%-100% as compared to a control (such as, for example, an individual not having a disease or disorder characterized by lysine catabolism dysfunction and/or malfunction (such as GA-1)). In an aspect, treating can refer to 1%, 2%, 3%, 4%, 5%, 6%, 7%, 8%, 9%, 10%, 20%, 30%, 40%, 50%, 60%, 70%, 80%, 90%, 100% reduction of one or more symptoms of an established a disease or a disorder or a condition (e.g., lysine catabolism dysfunction and/or malfunction (such as GA-1)). It is understood that treatment does not necessarily refer to a cure or complete ablation or eradication of a disease or disorder characterized by lysine catabolism dysfunction and/or malfunction (such as GA-1). However, in an aspect, treatment can refer to a cure or complete ablation or eradication of a disease or a disorder or a condition (such as lysine catabolism dysfunction and/or malfunction (such as GA-1)).

As used herein, the term “prevent” or “preventing” or “prevention” refers to precluding, averting, obviating, forestalling, stopping, or hindering something from happening, especially by advance action. It is understood that where reduce, inhibit, or prevent are used herein, unless specifically indicated otherwise, the use of the other two words is also expressly disclosed. In an aspect, preventing lysine catabolism dysfunction and/or malfunction (such as GA-1) or the worsening of lysine catabolism dysfunction and/or malfunction (such as GA-1) is intended. The words “prevent” and “preventing” and “prevention” also refer to prophylactic or preventative measures for protecting or precluding a subject (e.g., an individual) not having lysine catabolism dysfunction and/or malfunction (such as GA-1) related complication from progressing to that complication.

As used herein, “operably linked” means that expression of a gene or a transgene is under the control of a promoter with which it is spatially connected. A promoter can be positioned 5′ (upstream) or 3′ (downstream) of a gene under its control. The distance between the promoter and a gene can be approximately the same as the distance between that promoter and the gene it controls in the gene from which the promoter is derived. As is known in the art, variation in this distance can be accommodated without loss of promoter function.

As used herein, an “enhancer” such as a transcription or transcriptional enhancer refers to regulatory DNA segment that is typically found in multicellular eukaryotes. An enhancer can strongly stimulate (“enhance”) the transcription of a linked transcription unit, i.e., it acts in cis. An enhancer can activate transcription over very long distances of many thousand base pairs, and from a position upstream or downstream of the site of transcription initiation. An enhancer can have a modular structure by being composed of multiple binding sites for transcriptional activator proteins. Many enhancers control gene expression in a cell type-specific fashion. Several remote enhancers can control the expression of a singular gene while a singular enhance can stimulate the transcription of one or more genes.

As used herein, “expression cassette” or “transgene cassette” can refer to a distinct component of vector DNA comprising a transgene and one or more regulatory sequences to be expressed by a transfected cell. Generally, an expression cassette or transgene cassette can comprise a promoter sequence, an open reading frame (i.e., the transgene), and a 3′ untranslated region (e.g., in eukaryotes a polyadenylation site).

As used herein, “promoter” or “promoters” are known to the art. Depending on the level and tissue-specific expression desired, a variety of promoter elements can be used. A promoter can be tissue-specific or ubiquitous and can be constitutive or inducible, depending on the pattern of the gene expression desired. A promoter can be native (endogenous) or foreign (exogenous) and can be a natural or a synthetic sequence. By foreign or exogenous, it is intended that the transcriptional initiation region is not found in the wild-type host into which the transcriptional initiation region is introduced.

“Tissue-specific promoters” are known to the art and include, but are not limited to, neuron-specific promoters, muscle-specific promoters, liver-specific promoters, skeletal muscle-specific promoters, and heart-specific promoters.

“Liver-specific promoters” are known to the art and include, but are not limited to, the thyroxin binding globulin (TBG) promoter, the al-microglobulin/bikunin enhancer/thyroid hormone-binding globulin promoter, the human albumin (hALB) promoter, the thyroid hormone-binding globulin promoter, the α-1-anti-trypsin promoter, the bovine albumin (bAlb) promoter, the murine albumin (mAlb) promoter, the human al-antitrypsin (hAAT) promoter, the ApoEhAAT promoter comprising the ApoE enhancer and the hAAT promoter, the transthyretin (TTR) promoter, the liver fatty acid binding protein promoter, the hepatitis B virus (HBV) promoter, the DC172 promoter comprising the hAAT promoter and the al-microglobulin enhancer, the DC190 promoter comprising the human albumin promoter and the prothrombin enhancer, or any other natural or synthetic liver-specific promoter.

In an aspect, a disclosed liver-specific promoter can comprise any liver-specific promoter known to the art. In an aspect, a liver specific promoter can comprise about 845-bp and comprise the thyroid hormone-binding globulin promoter sequences (2382 to 13), two copies of al-microglobulinybikunin enhancer sequences (22,804 through 22,704), and a 71-bp leader sequence (Ill C R, et al. (1997) Blood Coagul Fibrinolysis. 8 Suppl 2:S23-S30).

As used herein, the terms “administering” and “administration” refer to any method of providing one or more of the disclosed compositions (such as, for example, a disclosed viral vector). Such methods are well-known to those skilled in the art and include, but are not limited to, the following: oral administration, transdermal administration, administration by inhalation, nasal administration, topical administration, in utero administration, intrahepatic administration, intravaginal administration, epidural administration (such as epidural injection), intracerebroventricular (ICV) administration, ophthalmic administration, intraaural administration, depot administration, topical (skin) administration, otic administration, intra-articular (such as joint or vertebrate injection), intracerebral administration, rectal administration, sublingual administration, buccal administration, and parenteral administration, including injectable such as intravenous administration, intra-CSF administration, intra-cistern magna (ICM) administration, intra-arterial administration, intrathecal (ITH) administration, intramuscular administration, and subcutaneous administration. Administration of a disclosed composition, a disclosed viral vector, a disclosed pharmaceutical formulation, a disclosed therapeutic agent, a disclosed immune modulator, a disclosed proteasome inhibitor, a disclosed small molecule, a disclosed endonuclease, a disclosed oligonucleotide, and/or a disclosed RNA therapeutic can comprise administration directly into the CNS or the PNS. Administration can be continuous or intermittent. Administration can comprise a combination of one or more route. In an aspect, a disclosed composition, a disclosed viral vector, a disclosed pharmaceutical formulation, or any combination thereof can be concurrently and/or serially administered to a subject via multiple routes of administration. For example, in an aspect, administering a disclosed composition, a disclosed viral vector, a disclosed pharmaceutical formulation, or any combination thereof can comprise intravenous administration and intra-cistern magna (ICM) administration. In an aspect, administering a disclosed composition, a disclosed viral vector, a disclosed pharmaceutical formulation, or any combination thereof can comprise IV administration and intrathecal (ITH) administration. Various combinations of administration are known to the art.

As used herein, “immune modulator” refers to an agent that is capable of adjusting a given immune response to a desired level (e.g., as in immunopotentiation, immunosuppression, or induction of immunologic tolerance). Examples of immune modulators include but are not limited to, a disclosed immune modulator can comprise aspirin, azathioprine, belimumab, betamethasone dipropionate, betamethasone valerate, bortezomib, bredinin, cyazathioprine, cyclophosphamide, cyclosporine, deoxyspergualin, didemnin B, fluocinolone acetonide, folinic acid, ibuprofen, IL6 inhibitors (such as sarilumab) indomethacin, inebilizumab, intravenous gamma globulin (IVIG), methotrexate, methylprednisolone, mycophenolate mofetil, naproxen, prednisolone, prednisone, prednisolone indomethacin, rapamycin, rituximab, sirolimus, sulindac, synthetic vaccine particles containing rapamycin (SVP-Rapamycin or ImmTOR), thalidomide, tocilizumab, tolmetin, triamcinolone acetonide, anti-CD3 antibodies, anti-CD4 antibodies, anti-CD19 antibodies, anti-CD20 antibodies, anti-CD22 antibodies, anti-CD40 antibodies, anti-FcRN antibodies, anti-IL6 antibodies, anti-IGF1R antibodies, an IL2 mutein, a BTK inhibitor, or a combination thereof. In an aspect, a disclosed immune modulator can comprise one or more Treg (regulatory T cells) infusions (e.g., antigen specific Treg cells to AAV).

By “determining the amount” is meant both an absolute quantification of a particular analyte (e.g., a toxic catabolite) or a determination of the relative abundance of a particular analyte (e.g., a toxic catabolite). The phrase includes both direct or indirect measurements of abundance or both. In an aspect, determining the amount can refer to measuring the expression of GCDH.

As used herein, “modifying the method” can comprise modifying or changing one or more features or aspects of one or more steps of a disclosed method. In an aspect, a method can be altered by changing the amount of one or more of the disclosed compositions (e.g., a disclosed viral vector) used in a disclosed method, or by changing the frequency of administration of one or more disclosed compositions (e.g., a disclosed viral vector) in a disclosed method, by changing the duration of time that one or more disclosed compositions (e.g., a disclosed viral vector) is administered in a disclosed method, or by substituting for one or more of the disclosed components and/or reagents with a similar or equivalent component and/or reagent.

As used herein, “concurrently” means (1) simultaneously in time, or (2) at different times during a common treatment schedule.

In an aspect, “CpG-free” can mean completely free of CpGs or partially free of CpGs. In an aspect, “CpG-free” can mean “CpG-depleted”. In an aspect, “CpG-depleted” can mean “CpG-free”. In an aspect, “CpG-depleted” can mean completely depleted of CpGs or partially depleted of CpGs. In an aspect, “CpG-free” can mean “CpG-optimized” for a desired and/or ideal expression level. CpG depletion and/or optimization is known to the skilled person in the art.

The term “contacting” as used herein refers to bringing one or more of the disclosed compositions (e.g., a disclosed viral vector) together with a target area or intended target area (e.g., a population of cells) in such a manner that the disclosed compositions can exert an effect on the intended target or targeted area either directly or indirectly. A target area or intended target area can be one or more cells (e.g., brain cells, liver cells, or both) and/or one or more tissues having toxic catabolite build-up (e.g., the brain, the liver, or both), or any combination thereof. In an aspect, a target area or intended target area can be any cell or any organ infected by a disease or disorder (such as GA-1). In an aspect, a target area or intended target area can be any organ, tissue, or cells that are affected by a disease or disorder characterized by lysine metabolic dysfunction.

As used herein, “determining” can refer to measuring or ascertaining the presence and severity of a disease or disorder, such as, for example, characterized by lysine catabolism dysfunction and/or malfunction (such as GA-1). “Determining” can refer to measuring or ascertaining an expression level of a protein or gene of interest. “Determining” can refer to measuring or ascertaining the reprogramming of a metabolic pathway. “Determining” can refer to ascertaining or measuring some type of neurologic, physiologic, and/or metabolic function and/or response.

Methods and techniques used to determine the presence and/or severity of a disease or disorder characterized by lysine catabolism dysfunction and/or malfunction (such as GA-1) are typically known to the medical arts. For example, the art is familiar with the ways to identify and/or diagnose the presence, severity, or both of a disease or disorder characterized by lysine catabolism dysfunction and/or malfunction (such as GA-1). Methods can be based on objective and/or subjective means.

As used herein, “effective amount” and “amount effective” can refer to an amount that is sufficient to achieve the desired result such as, for example, the treatment and/or prevention of a disease or disorder characterized by lysine catabolism dysfunction and/or malfunction (such as GA-1). As used herein, the terms “effective amount” and “amount effective” can refer to an amount that is sufficient to achieve the desired an effect on an undesired condition (e.g., a disease or disorder characterized by lysine catabolism dysfunction and/or malfunction (such as GA-1)). For example, a “therapeutically effective amount” refers to an amount that is sufficient to achieve the desired therapeutic result or to have an effect on undesired symptoms, but is generally insufficient to cause adverse side effects.

In an aspect, “therapeutically effective amount” means an amount of the disclosed composition that (i) treats a disease or disorder characterized by lysine catabolism dysfunction and/or malfunction (such as GA-1), (ii) attenuates, ameliorates, or eliminates one or more symptoms associated with a disease or disorder characterized by lysine catabolism dysfunction and/or malfunction (such as GA-1), or (iii) delays the onset of one or more symptoms of a disease or disorder characterized by lysine catabolism dysfunction and/or malfunction (such as GA-1). The specific therapeutically effective dose level for any particular patient will depend upon a variety of factors including the disease or disorder characterized by lysine catabolism dysfunction and/or malfunction (such as GA-1) being treated; the disclosed compositions employed; the disclosed methods employed; the age, body weight, general health, sex and diet of the patient; the time of administration; the route of administration; the rate of excretion of the disclosed compositions employed; the duration of the treatment; drugs used in combination or coincidental with the disclosed compositions employed, and other like factors well known in the medical arts. For example, it is well within the skill of the art to start doses of the disclosed compositions at levels lower than those required to achieve the desired therapeutic effect and to gradually increase the dosage until the desired effect is achieved. If desired, then the effective daily dose can be divided into multiple doses for purposes of administration. Consequently, a single dose of the disclosed compositions, disclosed viral vectors, disclosed pharmaceutical formulations, disclosed therapeutic agents, or a combination thereof can contain such amounts or submultiples thereof to make up the daily dose. The dosage can be adjusted by the individual physician in the event of any contraindications. Dosage can vary, and can be administered in one or more dose administrations daily, for one or several days. Guidance can be found in the literature for appropriate dosages for given classes of pharmaceutical products. In further various aspects, a preparation can be administered in a “prophylactically effective amount”; that is, an amount effective for prevention of a sign or symptom associated with a disease or disorder characterized by lysine catabolism dysfunction and/or malfunction (such as GA-1).

As used herein, the term “package insert” is used to refer to instructions customarily included in commercial packages of therapeutic products, that contain information about the indications, usage, dosage, administration, contraindications and/or warnings concerning the use of such therapeutic products.

Disclosed are the components to be used to prepare the disclosed compositions, disclosed viral vectors, disclosed pharmaceutical formulations, disclosed therapeutic agents, or a combination thereof used within the methods disclosed herein. These and other materials are disclosed herein, and it is understood that when combinations, subsets, interactions, groups, etc. of these materials are disclosed that while specific reference of each various individual and collective combinations and permutation of these compounds cannot be explicitly disclosed, each is specifically contemplated and described herein. For example, if a particular compound is disclosed and discussed and a number of modifications that can be made to a number of molecules including the compounds are discussed, specifically contemplated is each and every combination and permutation of the compound and the modifications that are possible unless specifically indicated to the contrary. Thus, if a class of molecules A, B, and C are disclosed as well as a class of molecules D, E, and F and an example of a combination molecule, A-D is disclosed, then even if each is not individually recited each is individually and collectively contemplated meaning combinations, A-E, A-F, B-D, B-E, B-F, C-D, C-E, and C-F are considered disclosed. Likewise, any subset or combination of these is also disclosed. Thus, for example, the sub-group of A-E, B-F, and C-E would be considered disclosed. This concept applies to all aspects of this application including, but not limited to, steps in methods of making and using the compositions of the invention. Thus, if there are a variety of additional steps that can be performed it is understood that each of these additional steps can be performed with any specific embodiment or combination of embodiments of the methods of the invention.

### C. Glutaric Aciduria Type-1 (GA-1)

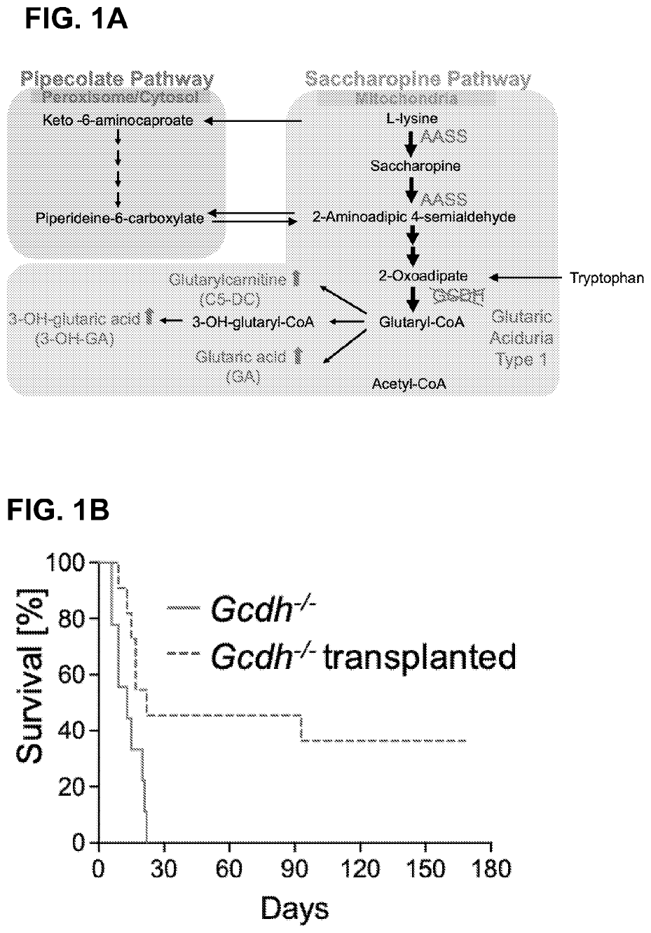

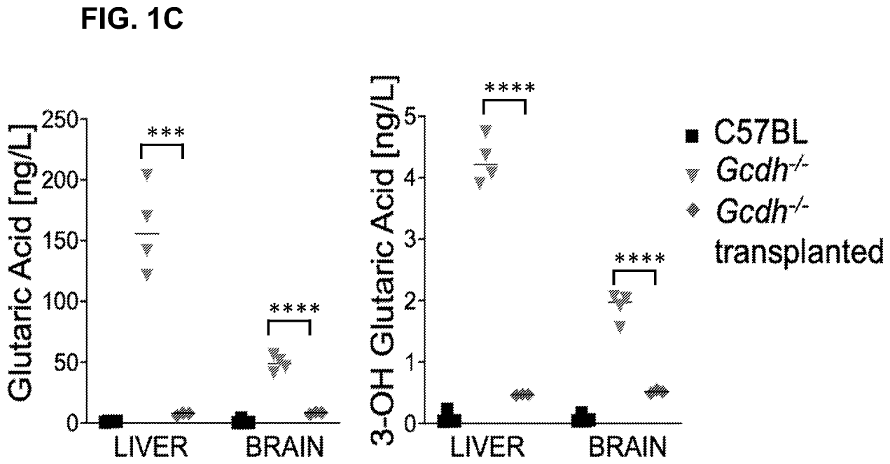

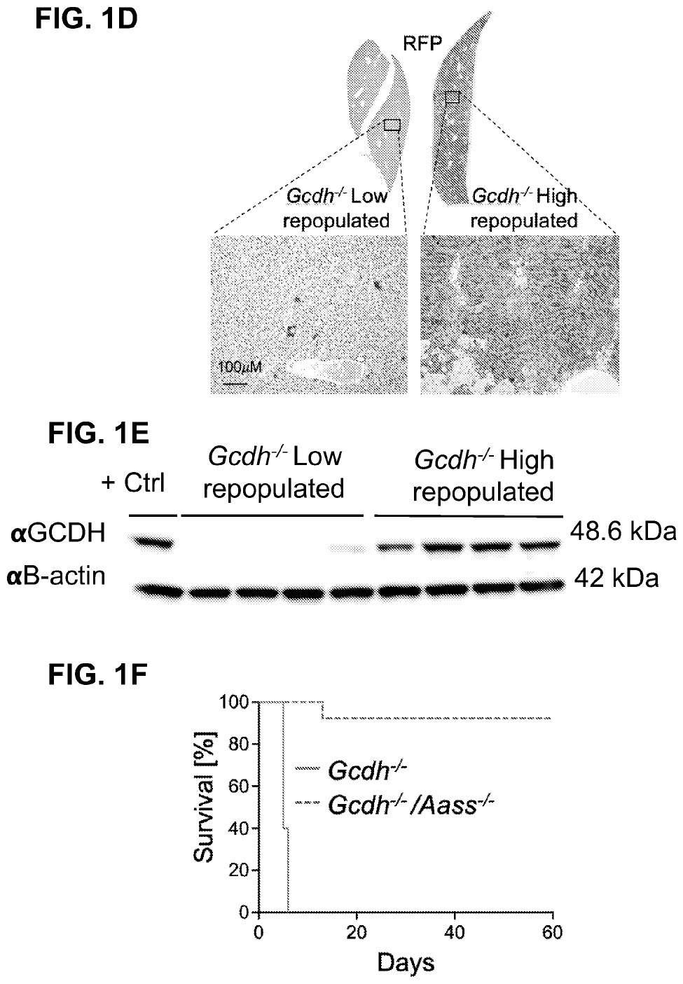

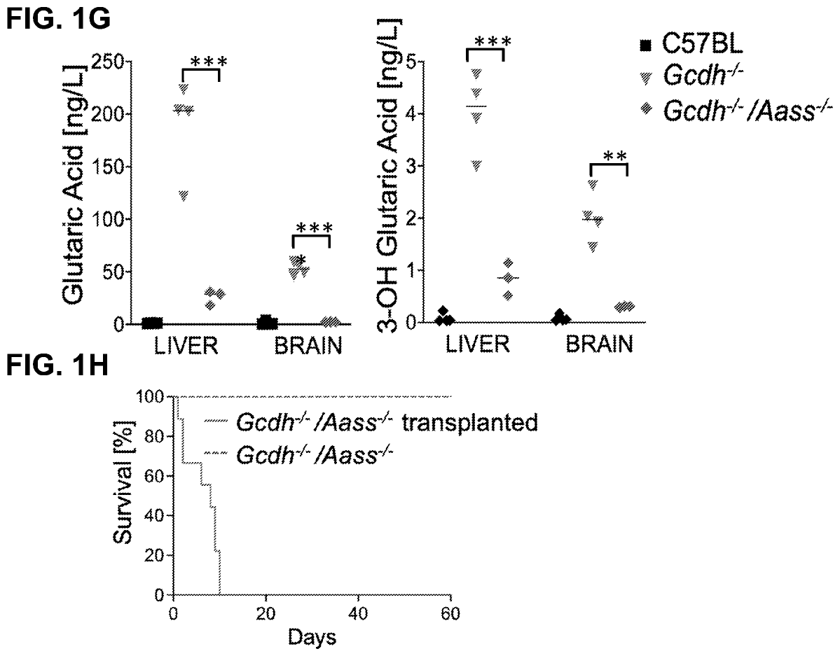

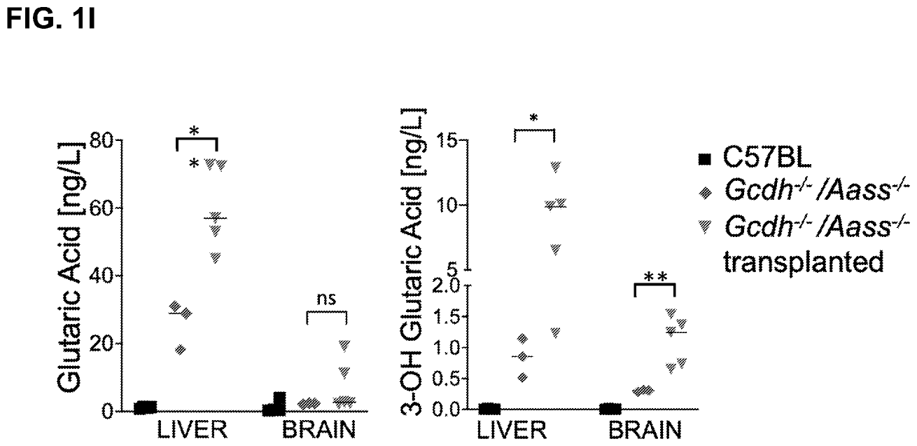

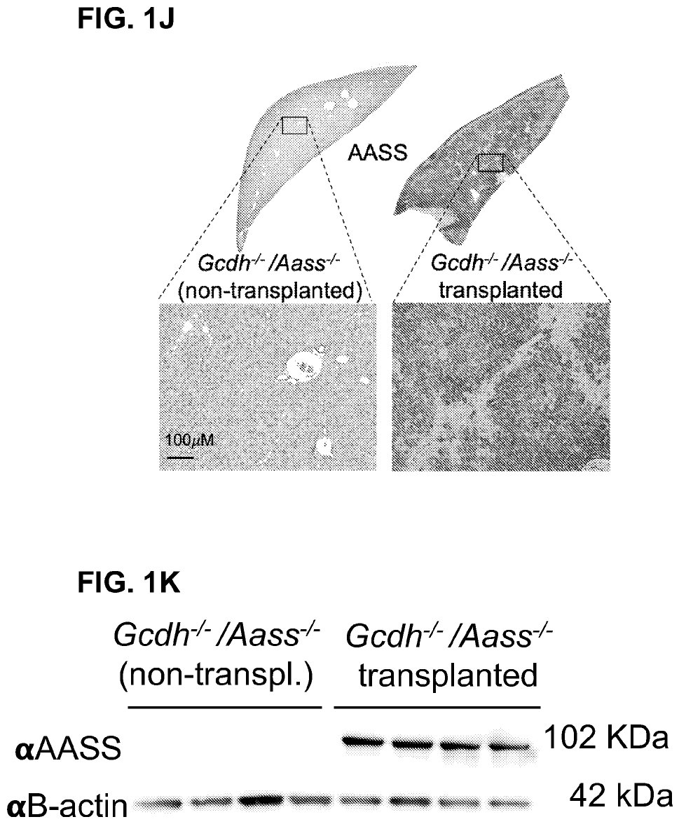

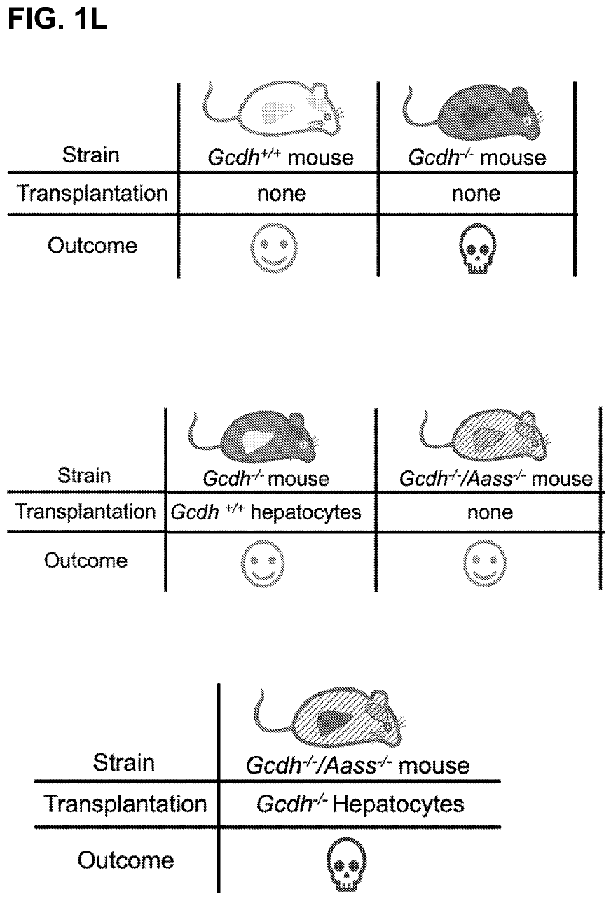

Glutaric aciduria type-1 (GA-1) is a cerebral organic aciduria with neurometabolic features due to Glutaryl-CoA dehydrogenase (GCDH) deficiency. It is an autosomal recessive inborn error of lysine (primarily) and tryptophan metabolism with a worldwide prevalence estimated to be between 1:30,000 to 1:100,000 live births. (Lindner M, et al. (2006) J Inherit Metab Dis. 29:378-382; Kolker S, et al. (2006) Pediatr Res. 59:840-847). GCDH catalyzes mitochondrial oxidative decarboxylation of glutaryl-CoA into crotonyl CoA and C02 in the L-lysine (FIG. 1), L-hydroxylysine and L-tryptophan catabolic pathway. Mutations in the GCDH gene cause characteristic clinical and biochemical phenotypes. The clinical phenotype is neurologic, characterized by macrocephaly at birth, subdural hematomas and acute retinal hemorrhage. During infancy and early childhood, patients with GA-1 are at risk of acute encephalopathic crises triggered by intercurrent illness, fever or fasting; damaging the brain striatum. Infantile acute striatal necrosis is the hallmark of GA-1 and the primary cause of morbidity and mortality leading to dystonia, and multisystem complications. Putamin injury is associated with motor and behavioral regression. Brain imaging shows selective regional (basal ganglia) findings that are symmetric and irreversible. (Strauss K A, et al. (2003) Am J Med Genet C Semin Med Genet. 121C(1):38-52). Some patients have an insidious onset disease presentation not associated with acute crises. These patients usually present later in life with late-onset neurologic disease (Kolker S, et al. (2006) Pediatr Res. 59:840-847). As an extracerebral manifestation an increased frequency of chronic renal failure in adults has been reported. (Boy N, et al. (2017) J Inherit Metab Dis 40: 75-101).

The biochemical phenotype is diagnostic in high GA-1 excretors showing elevated 3-hydroxyglutaric acid (neurotoxic), and glutaric acid in urine organic acids and elevated glutarylcarnitine (C5DC) in blood, the latter being the biomarker detected on newborn screening (NBS). These catabolites are also elevated in the blood, urine, CSF, and in liver, kidney, and brain tissues. In patients characterized as low excretors, biochemical studies may not be diagnostic, and in that case, GCDH mutation testing may help provide the diagnosis. (Lindner M, et al. (2006) J Inherit Metab Dis. 29:378-382). Early diagnosis and treatment following NBS often has led to more favorable outcomes when compliant with treatment plans. The standard of care treatment in GA-1, aims at reducing lysine; the main offending substrate that feeds into the pathway. This is achieved by restricting protein, mainly exogenous lysine and tryptophan; carnitine supplementation; and intensified emergency therapy with a high caloric glucose infusion to promote anabolism during illness. After the age of 6 years, dietary treatment is less protein restricted with continued metabolic supervision and follow up. There is no specific targeted therapy for GA-1 and untreated or poorly managed individuals with GA-1 develop acute encephalopathic crises during the first 6 years of life leading to poor outcomes and limited response to therapy. (Kolker S, et al. (2006) Pediatr Res. 59:840-847).

### 1. Lysine Catabolism.

Lysine is an essential amino acid necessary for protein synthesis. When lysine is not needed for protein synthesis, it proceeds to degradation via two catabolic pathways. Through saccharopine formation by ε-deamination, or pipecolic acid (PA) formation by α-deamination or transamination. Both pathways lead to formation of Δ1-piperideine-6-carboxylate (P6C) and its open form α-aminoadipic semialdehyde (AASA), which is then converted to α-aminoadipic acid (AAA) by the AASA dehydrogenase (ALDH7A1).

### Saccharopine Pathway.

The saccharopine pathway in liver mitochondria is the major pathway for degradation of L-lysine into acetyl-CoA. Lysine is first converted into saccharopine, which is subsequently oxidized to AASA. This ultimately leads to generation of acetyl-CoA that enters the tricarboxylic acid cycle. This pathway is key for irreversible catabolism of extra-cerebral lysine. While the liver is the major organ for lysine catabolism, the kidney is involved, as well as the brain to a lesser degree.

### The Pipecolate Pathway.

The pipecolate Pathway in brain peroxisomes is used for breakdown of the fraction of D-lysine that is catabolized in brain peroxisomes. There the α-amino group of lysine is converted to an α-keto function and further metabolized to P6C. During fetal development, the saccharopine pathway prevails in both fetal brain and extracerebral tissues, whereas after birth it maintains a pivotal role in extracerebral lysine catabolism through the liver (mainly), and kidney. Conversely, in adult life, the pipecolate pathway emerges to play the predominant role in brain lysine catabolism with only a minor role in extracerebral tissues.

2. Clinical Disorders Associated with Lysine Degradation.

Hyperlysinemia Type 1 is an autosomal recessive condition due to an isolated mutation in the LKR subdomain of the AASS gene or mutations causing loss of function of both the LKR and saccharopine dehydrogenase (SDH) domains of the AASS gene. The condition is usually asymptomatic, with benign hyperlysinemia without neurological sequelae.

Hyperlysinemia Type 2 is also known as saccharopinuria and is due to a deficiency of the SDH domain of AAS. This is a rare recessive inborn error of lysine metabolism associated with mutation of the AASS-SDH subdomain (with preserved LKR function). Clinically, patients with this disorder show signs of developmental delay, cognitive impairment, and spastic diplegia; biochemically, the condition is characterized by both hyperlysinemia and saccharopinuria. Saccharopinuria is due to mutation of the AASS-SDH subdomain and is associated with mitochondrial toxicity.

Pyridoxine dependent epilepsy is an autosomal recessive disorder caused by mutations in ALDH7A1 an enzyme central to the lysine degradation pathways. Accumulation of high levels of the ALDH7A1 substrate AASA, and its cyclic form P6C is considered diagnostic markers in blood, urine, and CSF. Pipecolate elevations in body fluids can be observed but is not a reliable biomarker. Accumulation of AASA leads to depletion of pyridoxal phosphate, an essential coenzyme derived from vitamin B6. Patients with PDE present early in life with neonatal intractable seizures which are responsive to high doses of pyridoxine.

### D. Gene Editing Components

Generally, the CRISPR-Cas system relies on two main components: a guide RNA (gRNA) and CRISPR-associated (Cas) nuclease. The guide RNA is a specific RNA sequence that recognizes the target DNA region of interest and directs the Cas nuclease there for editing. The gRNA is made up of two parts: crispr RNA (crRNA), a 17-20 nucleotide sequence complementary to the target DNA, and a tracr RNA, which serves as a binding scaffold for the Cas nuclease. The CRISPR-associated protein is a non-specific endonuclease. It is directed to the specific DNA locus by a gRNA, where it makes a double-strand break.

CRISPR systems are adaptive defense systems originally discovered in bacteria and archaea. CRISPR systems use RNA-guided nucleases termed CRISPR-associated or “Cas” endonucleases (e.g., Cas9 or Cpf1) to cleave foreign DNA. In a typical CRISPR/Cas system, an endonuclease is directed to a target nucleotide sequence (e.g., a site in the genome that is to be sequence-edited) by sequence-specific, non-coding “guide RNAs” that target single- or double-stranded DNA sequences. Three classes (I-III) of CRISPR systems have been identified. The class II CRISPR systems use a single Cas endonuclease (rather than multiple Cas proteins). One class II CRISPR system includes a type II Cas endonuclease such as Cas9, a CRISPR RNA (“crRNA”), and a trans-activating crRNA (“tracrRNA”). The crRNA contains a “guide RNA”, typically about 20-nucleotide RNA sequence that corresponds to a target DNA sequence. The crRNA also contains a region that binds to the tracrRNA to form a partially double-stranded structure which is cleaved by RNase III, resulting in a crRNA/tracrRNA hybrid. The crRNA/tracrRNA hybrid then directs the Cas9 endonuclease to recognize and cleave the target DNA sequence. The target DNA sequence must generally be adjacent to a “protospacer adjacent motif” (“PAM”) that is specific for a given Cas endonuclease; however, PAM sequences appear throughout a given genome. For example, some CRISPR endonucleases identified from various prokaryotic species have unique PAM sequence requirements; examples of PAM sequences include 5′-NGG (Streptococcus pyogenes), 5′-NNAGAA (Streptococcus thermophilus CRISPRI), 5′-NGGNG (Streptococcus thermophilus CRISPR3), and 5′-NNNGATT (Neisseria meningiditis). In an aspect, a SpCas9 (3′NGG—PAM sequence) can comprise SpCas9 VQR (3′NGAN or 3′NGNG), SpCas9 EQR (3′NGAG), or SpCas9 VRER (3′NGCG).

As used herein, “CRISPR or clustered regularly interspaced short palindromic repeat” is an ideal tool for correction of genetic abnormalities as the system can be designed to target genomic DNA directly. A CRISPR system involves two main components—a Cas9 enzyme and a guide (gRNA). The gRNA contains a targeting sequence for DNA binding and a scaffold sequence for Cas9 binding. Cas9 nuclease is often used to “knockout” target genes hence it can be applied for deletion or suppression of oncogenes that are essential for cancer initiation or progression. Similar to ASOs and siRNAs, CRISPR offers a great flexibility in targeting any gene of interest hence, potential CRISPR based therapies can be designed based on the genetic mutation in individual patients. An advantage of CRISPR is its ability to completely ablate the expression of disease genes which can only be suppressed partially by RNA interference methods with ASOs or siRNAs. Furthermore, multiple gRNAs can be employed to suppress or activate multiple genes simultaneously, hence increasing the treatment efficacy and reducing resistance potentially caused by new mutations in the target genes. In an aspect, a disclosed sgRNA can be directed at any functional domain of a target sequence.

As used herein, “CRISPR-based endonucleases” include RNA-guided endonucleases that comprise at least one nuclease domain and at least one domain that interacts with a guide RNA. As known to the art, a guide RNA directs the CRISPR-based endonucleases to a targeted site in a nucleic acid at which site the CRISPR-based endonucleases cleaves at least one strand of the targeted nucleic acid sequence. As the guide RNA provides the specificity for the targeted cleavage, the CRISPR-based endonuclease is universal and can be used with different guide RNAs to cleave different target nucleic acid sequences. CRISPR-based endonucleases are RNA-guided endonucleases derived from CRISPR/Cas systems. Bacteria and archaea have evolved an RNA-based adaptive immune system that uses CRISPR (clustered regularly interspersed short palindromic repeat) and Cas (CRISPR-associated) proteins to detect and destroy invading viruses or plasmids. CRISPR/Cas endonucleases can be programmed to introduce targeted site-specific double-strand breaks by providing target-specific synthetic guide RNAs (Jinek et al. (2012) Science. 337:816-821).

In an aspect, a disclosed CRISPR-based endonuclease can be derived from a CRISPR/Cas type I, type II, or type III system. Non-limiting examples of suitable CRISPR/Cas proteins include Cas3, Cas4, Cas5, Cas5e (or CasD), Cas6, Cas6e, Cas6f, Cas7, Cas8a1, Cas8a2, Cas8b, Cas8c, Cas9, Cas10, Cas10d, CasF, CasG, CasH, Csy1, Csy2, Csy3, Cse1 (or CasA), Cse2 (or CasB), Cse3 (or CasE), Cse4 (or CasC), Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csz1, Csx15, Csf1, Csf2, Csf3, Csf4, and Cu1966.

In an aspect, a disclosed CRISPR-based endonuclease can be derived from a type II CRISPR/Cas system. For example, in an aspect, a CRISPR-based endonuclease can be derived from a Cas9 protein. The Cas9 protein can be from Streptococcus pyogenes, Streptococcus thermophilus, Streptococcus sp, Nocardiopsis dassonvillei, Streptomyces pristinaespiralis, Streptomyces viridochromogenes, Streptomyces viridochromogenes, Streptosporangium roseum, Streptosporangium roseum, Alicyclobacillus acidocaldarius, Bacillus pseudomycoides, Bacillus selenitireducens, Exiguobacterium sibiricum, Lactobacillus delbrueckii, Lactobacillus salivarius, Microscilla marina, Burkholderiales bacterium, Polaromonas naphthalenivorans, Polaromonas sp., Crocosphaera watsonii, Cyanothece sp., Microcystis aeruginosa, Synechococcus sp., Acetohalobium arabaticum, Ammonifex degensii, Caldicelulosiruptor becscii, Candidatus Desulforudis, Clostridium botulinum, Clostridium difficile, Finegoldia magna, Natranaerobius thermophilus, Pelotomaculum thermopropionicum, Acidithiobacillus caldus, Acidithiobacillus ferrooxidans, Allochromatium vinosum, Marinobacter sp., Nitrosococcus halophilus, Nitrosococcus watsoni, Pseudoalteromonas haloplanktis, Ktedonobacter racemifer, Methanohalobium evestigatum, Anabaena variabilis, Nodularia spumigena, Nostoc sp., Arthrospira maxima, Arthrospira platensis, Arthrospira sp., Lyngbya sp., Microcoleus chthonoplastes, Oscillatoria sp., Petrotoga mobilis, Thermosipho africanus, or Acaryochloris marina. In an aspect, the CRISPR-based nuclease can be derived from a Cas9 protein from Staphylococcus Aureus or Streptococcus pyogenes.

In general, CRISPR/Cas proteins can comprise at least one RNA recognition and/or RNA binding domain. RNA recognition and/or RNA binding domains can interact with the guide RNA such that the CRISPR/Cas protein is directed to a specific genomic or genomic sequence. CRISPR/Cas proteins can also comprise nuclease domains (i.e., DNase or RNase domains), DNA binding domains, helicase domains, protein-protein interaction domains, dimerization domains, as well as other domains.

The CRISPR-based endonuclease can be a wild type CRISPR/Cas protein, a modified CRISPR/Cas protein, or a fragment of a wild type or modified CRISPR/Cas protein. The CRISPR/Cas protein can be modified to increase nucleic acid binding affinity and/or specificity, alter an enzymatic activity, and/or change another property of the protein. For example, in an aspect, nuclease (i.e., DNase, RNase) domains of the CRISPR/Cas protein can be modified, deleted, or inactivated. A CRISPR/Cas protein can be truncated to remove domains that are not essential for the function of the protein. A CRISPR/Cas protein also can be truncated or modified to optimize the activity of the protein or an effector domain fused with a CRISPR/Cas protein.

In an aspect, a disclosed CRISPR-based endonuclease can be derived from a wild type Cas9 protein or fragment thereof. In an aspect, a disclosed CRISPR-based endonuclease can be derived from a modified Cas9 protein. For example, the amino acid sequence of a disclosed Cas9 protein can be modified to alter one or more properties (e.g., nuclease activity, affinity, stability, etc.) of the protein. Alternatively, domains of the Cas9 protein not involved in RNA-guided cleavage can be eliminated from the protein such that the modified Cas9 protein is smaller than the wild type Cas9 protein.

As known to the art, once the target gene and Cas nuclease have been selected, the next step is to design the specific guide RNA sequence. Several software tools exist for designing an optimal guide with minimum off-target effects and maximum on-target efficiency. Commercially available software programs include, but are not limited to, Synthego Design Tool, Broad Institute GPP sgRNA Designer, CRISPOR, CHOPCHOP, Off-Spotter, Cas-OFFinder, CRISPR-Era, Benchling CRISPR Guide RNA Design tool, and E-CRISP. The skilled person can use these programs without undue experimentation.

As used herein, CRISPR-mediated insertion of exon (CRISPIE) is a technique that allows for the nearly error-free insertion of coding sequences with high efficiency. Instead of targeting gene exons, CRISPIE targets introns and inserts a designer donor module, which includes an exon encoding the desired protein sequence and the surrounding intronic sequences. INDELs occurring at the insertion junction within the intronic region of DNA will be spliced out, resulting in very low error rates at the mRNA level (>98% correct). CRISPIE is flexible and broadly compatible with: (1) both N- and C-terminal labeling, (2) proteins with diverse structures and functions, including pre- and post-synaptic proteins and cytoskeletal proteins, (3) all major transfection methods, (4) FPs with diverse colors, and (5) multiple animal species. In part because introns offer ample editing sites to choose from, and because INDELs at the DNA level do not affect the success of editing, a high labeling efficiency (up to 43%) was achieved in cortical neurons of living mice. Importantly, CRISPIE-mediated DNA insertions are erasable. By flanking the donor module with additional designer CRISPR editing sites in the intronic region, the inserted DNA fragment can be erased at a later time. CRISPIE may allow for the routine labeling of proteins at endogenous levels and can be expanded to the insertion of other genetically encoded functional sequences to manipulate protein function. (See Zhong H, et al. (2021) eLife.10:e64911, which is incorporated by reference for its teaching of CRISPIE).

### E. RNA Interference (RNAi) and Small Interfering RNA (siRNA)

RNA interference (RNAi) is a sequence-specific RNA degradation process that provides a relatively easy and direct way to knock down, or silence, theoretically any gene. In naturally occurring RNAi, a double-stranded RNA (dsRNA) is cleaved by an RNase III/helicase protein, Dicer, into small interfering RNA (siRNA) molecules, a dsRNA of 19-27 nucleotides (nt) with 2-nt overhangs at the 3′ ends. These siRNAs are incorporated into a multicomponent-ribonuclease called RNA-induced silencing complex (RISC). One strand of siRNA remains associated with RISC and guides the complex toward a cognate RNA that has sequence complementary to the guider ss-siRNA in RISC. This siRNA-directed endonuclease digests the RNA, thereby inactivating it. Recent studies have revealed that chemically synthesized 21-27-nt siRNAs exhibit RNAi effects in mammalian cells, and the thermodynamic stability of siRNA hybridization (at terminals or in the middle) plays a central role in determining the molecule's function. These and other characteristics of RISC, siRNA molecules, and RNAi have been described.

Applications of RNAi in mammalian cells in the laboratory or, potentially, in therapeutic settings, use either chemically synthesized siRNAs or endogenously expressed molecules. The endogenous siRNA is first expressed as small hairpin RNAs (shRNAs) by an expression vector (plasmid or virus vector) and is then processed by Dicer into siRNAs.

### F. Compositions for Use in the Disclosed Methods

### 1. GCDH Nucleic Acid Molecules

Disclosed herein is an isolated nucleic acid molecule comprising a nucleic acid sequence encoding glutaryl-CoA dehydrogenase. Disclosed herein is an isolated nucleic acid molecule comprising a nucleic acid sequence encoding the one or more functional domains of glutaryl-CoA dehydrogenase. In an aspect, a disclosed nucleic acid sequence encoding a glutaryl-CoA dehydrogenase can be derived from a non-mammalian species or from a mammalian species. In an aspect, a disclosed nucleic acid sequence encoding a glutaryl-CoA dehydrogenase can be that of a non-mammalian species or that of a mammalian species.

In an aspect, a disclosed nucleic acid sequence encoding a glutaryl-CoA dehydrogenase can comprise the sequence set forth in SEQ ID NO:01 or in SEQ ID NO:02. In an aspect, a disclosed nucleic acid sequence encoding a glutaryl-CoA dehydrogenase can comprise the functional domains of the sequence set forth in SEQ ID NO:01 or in SEQ ID NO:02. In an aspect, a disclosed nucleic acid sequence encoding a glutaryl-CoA dehydrogenase can comprise a sequence having about 50%, about 55%, about 60%, about 65%, about 70%, about 75%, about 80%, about 85%, about 90%, about 95%, or more identity to the sequence set forth in SEQ ID NO:01 or in SEQ ID NO:02.

In an aspect, a disclosed nucleic acid sequence encoding a glutaryl-CoA dehydrogenase can comprise the sequence set forth in SEQ ID NO:17 or in SEQ ID NO:18. In an aspect, a disclosed nucleic acid sequence encoding a glutaryl-CoA dehydrogenase can comprise a sequence having about 50%, about 55%, about 60%, about 65%, about 70%, about 75%, about 80%, about 85%, about 90%, about 95%, or more identity to the sequence set forth in SEQ ID NO:17 or in SEQ ID NO:18.

In an aspect, a disclosed nucleic acid sequence encoding a glutaryl-CoA dehydrogenase can comprise the sequence of Gene ID 2639 or of Gene ID 270076. In an aspect, a disclosed nucleic acid sequence encoding a glutaryl-CoA dehydrogenase can comprise a sequence having about 50%, about 55%, about 60%, about 65%, about 70%, about 75%, about 80%, about 85%, about 90%, about 95%, or more identity to the sequence of Gene ID 2639 or the sequence of Gene ID 270076.

In an aspect, a disclosed nucleic acid sequence encoding a glutaryl-CoA dehydrogenase can comprise the sequence of Gene ID 2639 or of Gene ID 270076 or one or more functional domains thereof. In an aspect, a disclosed nucleic acid sequence encoding a glutaryl-CoA dehydrogenase can comprise a sequence having about 50%, about 55%, about 60%, about 65%, about 70%, about 75%, about 80%, about 85%, about 90%, about 95%, or more identity to the sequence of Gene ID 2639 or the sequence of Gene ID 270076 or one or more functional domains thereof.

In an aspect, a disclosed nucleic acid sequence encoding a glutaryl-CoA dehydrogenase can comprise the sequence set forth in NCBI Reference Sequence NG_009292 from bases 5001 to 13840 or the sequence set forth in NCBI Reference Sequence NC_000074.7 from bases 85629378 to 85613016. In an aspect, a disclosed nucleic acid sequence encoding a glutaryl-CoA dehydrogenase can comprise a sequence having about 50%, about 55%, about 60%, about 65%, about 70%, about 75%, about 80%, about 85%, about 90%, about 95%, or more identity to the sequence set forth in NCBI Reference Sequence NG_009292 from bases 5001 to 13840 or the sequence set forth in NCBI Reference Sequence NC_000074.7 from bases 85629378 to 85613016.

In an aspect, a disclosed nucleic acid sequence encoding a glutaryl-CoA dehydrogenase can comprise one or more nucleotide substitutions, insertions, deletions, modifications, or any combination thereof. The techniques to introduce one or more substitutions, insertions, deletions, modifications, or any combination thereof are known to the skilled person.

In an aspect, a disclosed isolated nucleic acid molecule can be codon-optimized for expression in a mammalian cell or a human cell. In an aspect, a disclosed isolated nucleic acid molecule can be CpG-free or CpG-depleted. In an aspect, a disclosed nucleic acid sequence encoding a glutaryl-CoA dehydrogenase can be codon-optimized for expression in a mammalian cell or a human cell. In an aspect, a disclosed nucleic acid sequence encoding a glutaryl-CoA dehydrogenase can be CpG-free or CpG-depleted.

In an aspect, a disclosed isolated nucleic acid molecule can further comprise a nucleic acid sequence encoding a carboxy-terminal fluorescent label and/or fluorescent tag, an amino-terminal fluorescent label and/or fluorescent tag, or a combination thereof.

In an aspect, a disclosed fluorescent label and/or fluorescent tag can comprise green fluorescent protein (EGFP), mEmerald, enhanced yellow fluorescent protein (EYFP), mApple, TdTomato, mCherry, miRFP670, any known fluorescent label or tag, or any combination thereof. Fluorophores and fluorescent labels are known in the art.

In an aspect, a disclosed nucleic acid sequence encoding a glutaryl-CoA dehydrogenase can restore the functionality of a missing, dysfunctional, and/or mutated glutaryl-CoA dehydrogenase in a cell or a subject. In an aspect, a disclosed nucleic acid sequence encoding a glutaryl-CoA dehydrogenase (i) can restore liver-specific modulation of lysine catabolism, (ii) restore one or more aspects of lysine homeostasis, (iii) can reduce or decrease the level of toxic catabolites in the liver and/or brain of a subject, (iv) can restore the metabolic flux from glutaryl-CoA to crotonyl-CoA, (v) can improve motor performance (e.g., strength, gait, balance, coordination, and combinations thereof) of a subject, (vi) can improve memory function of a subject, (vii) can reduce anxiety in a subject, (viii) can reduce and/or prevent neurological sequelae (e.g., neonatal macrocephaly, subdural hematomas, acute retinal hemorrhage, encephalopathy, striatal necrosis, and combinations thereof), (ix) can improve and/or reduce and/or eliminate vascular dysfunction in a subject, (x) can improve a subject's quality of life, (xi) can increase and/or prolong a subject's life span, (xii) can increase a subject's survivability, or (xiii) any combination thereof.

In an aspect, a disclosed nucleic acid sequence encoding a glutaryl-CoA dehydrogenase (i) can restore liver-specific modulation of lysine catabolism, (ii) restore one or more aspects of lysine homeostasis in the subject's liver, (iii) can reduce or decrease the level of toxic catabolites in the liver and/or brain of a subject, (iv) can restore the metabolic flux from glutaryl-CoA to crotonyl-CoA in the subject's liver, or (v) any combination thereof.

In an aspect, a disclosed isolated nucleic acid sequence encoding a glutaryl-CoA dehydrogenase can treat and/or prevent Glutaric Aciduria Type-1 in a subject. In an aspect, a disclosed isolated nucleic acid sequence encoding a glutaryl-CoA dehydrogenase can improve and/or diminish and/or ameliorate one or more symptoms associated Glutaric Aciduria Type-1 in a subject. In an aspect, a disclosed isolated nucleic acid sequence encoding a glutaryl-CoA dehydrogenase can improve and/or diminish and/or ameliorate one or more pathologies associated with Glutaric Aciduria Type-1 in a subject.

In an aspect, a disclosed isolated nucleic acid molecule can comprise the nucleic acid sequence for one or more regulatory elements. In an aspect, a disclosed regulatory element can comprise a promoter, an enhancer, an internal ribosomal entry sites (IRES), and other expression control elements (e.g., transcription termination signals, such as polyadenylation signals and poly-U sequences), or any combination thereof. Regulatory elements can include those that direct constitutive expression of a nucleotide sequence in many types of host cells and those that direct expression of the nucleotide sequence only in certain host cells (e.g., tissue-specific regulatory sequences such as brain cells or neurons).

In an aspect, a disclosed isolated nucleic acid molecule can comprise a promoter operably linked to a nucleic acid sequence encoding a glutaryl-CoA dehydrogenase. In an aspect, a disclosed promoter can comprise a tissue specific promoter. In an aspect, a disclosed tissue specific promoter can comprise a neuron-specific promoter, a muscle-specific promoter, a liver-specific promoter, a skeletal muscle-specific promoter, and heart-specific promoter. In an aspect, a disclosed tissue-specific promoter can comprise a brain cell specific promoter. Brain cell specific promoter are known to the art and can comprise a synapsin 1 (Syn1) promoter, a calmodulin/calcium dependent kinase II (CAMKII) promoter, a glial fibrillary acidic protein (GFAP) promoter, a Rgs5 promoter, a S100 beta promoter, a neuron-specific enolase (NSE) promoter, a Thy1 promoter, or any combination thereof. In an aspect, a disclosed promoter can comprise a promoter/enhancer.

In an aspect, a disclosed promoter can comprise a liver-specific promoter. Liver specific promoters are known to the art. In an aspect, a disclosed liver promoter can comprise the sequence set forth in SEQ ID NO:26. In an aspect, a disclosed promoter can comprise a type III RNA polymerase III promoter. Type III RNA polymerase III promoters are known to the art. In an aspect, a disclosed type III RNA polymerase III promoter can comprise a U6 promoter. In an aspect, a disclosed U6 promoter can comprise the sequence set forth in SEQ ID NO:27.

In an aspect, a disclosed isolated nucleic acid molecule can comprise one or more OLLAS tag. In an aspect, a disclosed OLLAS tag can comprise the sequence set forth in SEQ ID NO:31.

In an aspect, a disclosed isolated nucleic acid molecule can comprise a nuclear localization signal (NLS). In an aspect, a disclosed NLS can comprise the sequence set forth in SEQ ID NO:30 or SEQ ID NO:32. NLS are known to the skilled person in the art. In an aspect, a disclosed isolated nucleic acid molecule can comprise one or more inverted terminal repeats (ITRs). In an aspect, the one or more disclosed ITRs can be derived from AAV2 or AAV9. In an aspect, a disclosed ITR can comprise the sequence set forth in any one of SEQ ID NO:22 or SEQ ID NO:23. In an aspect, a disclosed isolated nucleic acid molecule can comprise a polyA sequence. In an aspect, a disclosed polyA sequence can comprise the sequence set forth in SEQ ID NO:24 or SEQ ID NO:25. In an aspect, a disclosed isolated nucleic acid molecule can comprise one or more hemagglutinin (HA) tags. In an aspect, a disclosed HA tag can comprise the sequence set forth in SEQ ID NO:29. In an aspect, a disclosed isolated nucleic acid molecule can comprise a TracrRNA sequence. In an aspect, a TracrRNA sequence can comprise the sequence set forth in SEQ ID NO:11 or SEQ ID NO:12.

Disclosed herein is an isolated nucleic acid sequence comprising the sequence set forth in SEQ ID NO:19 or a fragment thereof. Disclosed herein is an isolated nucleic acid sequence comprising a sequence having at least 50%, at least 55%, at least 60%, at least 65%, at least 70%, at least 75%, at least 80%, at least 85%, at least 90%, at least 95%, or more to the sequence set forth in SEQ ID NO:19 or a fragment thereof.

In an aspect, a disclosed isolated nucleic acid molecule encoding a disclosed isolated nucleic acid sequence encoding a glutaryl-CoA dehydrogenase can be packaged in a viral vector (as discussed infra) or a non-viral vector. In an aspect, a disclosed non-viral vector can be a polymer-based vector, a peptide-based vector, a lipid nanoparticle, a solid lipid nanoparticle, or a cationic lipid-based vector. In an aspect, a disclosed isolated nucleic acid molecule comprising a nucleic acid sequence encoding a glutaryl-CoA dehydrogenase can be packaged in a recombinant AAV viral vector (e.g., AAV8 or AAVcc47).

In an aspect, a disclosed encoded glutaryl-CoA dehydrogenase can be derived from a non-mammalian species or from a mammalian species. In an aspect, a disclosed encoded glutaryl-CoA dehydrogenase can be that of a non-mammalian species or that of a mammalian species.

In an aspect, a disclosed encoded glutaryl-CoA dehydrogenase can comprise the sequence set forth in SEQ ID NO:03 or in SEQ ID NO:04. In an aspect, a disclosed encoded glutaryl-CoA dehydrogenase can comprise a sequence having about 50%, about 55%, about 60%, about 65%, about 70%, about 75%, about 80%, about 85%, about 90%, about 95%, or more identity to the sequence set forth in SEQ ID NO:03 or in SEQ ID NO:04.

In an aspect, a disclosed encoded glutaryl-CoA dehydrogenase can comprise the sequence of NCBI Reference Sequence NP_000150.1 or the sequence of NCBI Reference Sequence XP_036009971.1. In an aspect, a disclosed encoded glutaryl-CoA dehydrogenase can comprise one or more amino acid substitutions, insertions, deletions, modifications, or any combination thereof. The techniques to introduce one or more substitutions, insertions, deletions, modifications, or any combination thereof are known to the skilled person.

In an aspect, a disclosed encoded glutaryl-CoA dehydrogenase (i) can restore liver-specific modulation of lysine catabolism, (ii) restore one or more aspects of lysine homeostasis, (iii) can reduce or decrease the level of toxic catabolites in the liver and/or brain of a subject, (iv) can restore the metabolic flux from glutaryl-CoA to crotonyl-CoA, (v) can improve motor performance (e.g., strength, gait, balance, coordination, and combinations thereof) of a subject, (vi) can improve memory function of a subject, (vii) can reduce anxiety in a subject, (viii) can reduce and/or prevent neurological sequelae (e.g., neonatal macrocephaly, subdural hematomas, acute retinal hemorrhage, encephalopathy, striatal necrosis, and combinations thereof), (ix) can improve and/or reduce and/or eliminate vascular dysfunction in a subject, (x) can improve a subject's quality of life, (xi) can increase and/or prolong a subject's life span, (xii) can increase a subject's survivability, or (xiii) any combination thereof.

In an aspect, a disclosed encoded glutaryl-CoA dehydrogenase can treat and/or prevent Glutaric Aciduria Type-1 in a subject. In an aspect, a disclosed encoded glutaryl-CoA dehydrogenase can improve and/or diminish and/or ameliorate one or more symptoms associated Glutaric Aciduria Type-1 in a subject. In an aspect, a disclosed encoded glutaryl-CoA dehydrogenase encoding a glutaryl-CoA dehydrogenase can improve and/or diminish and/or ameliorate one or more pathologies associated with Glutaric Aciduria Type-1 in a subject.

Disclosed herein is an isolated nucleic acid molecule comprising a nucleic acid sequence encoding green fluorescent protein (GFP).

Disclosed herein is an expression cassette comprising a disclosed isolated nucleic acid molecule comprising a nucleic acid sequence encoding a glutaryl-CoA dehydrogenase operably linked a promoter. Disclosed herein is an expression cassette comprising a disclosed isolated nucleic acid molecule comprising a nucleic acid sequence encoding a glutaryl-CoA dehydrogenase, wherein the nucleic acid sequence comprises the sequence set forth in SEQ ID NO:01 or in SEQ ID NO:02, and is operably linked a promoter.

Disclosed herein is an expression cassette comprising a disclosed isolated nucleic acid molecule comprising a nucleic acid sequence encoding a glutaryl-CoA dehydrogenase, wherein the nucleic acid sequence comprises the sequence set forth in SEQ ID NO:17 or in SEQ ID NO:18, and is operably linked a promoter.

### 2. CRISPR Based Nucleic Acid Molecules

Disclosed herein is an isolated nucleic acid molecule comprising a nucleic acid sequence encoding one or more element of a gene editing system. Disclosed herein is an isolated nucleic acid molecule comprising a nucleic acid sequence encoding one or more element of a gene editing system, wherein the one or more elements comprises an endonuclease and a sgRNA directed at a target sequence in a target gene of interest. Disclosed herein is an isolated nucleic acid molecule comprising a nucleic acid sequence encoding one or more element of a gene editing system, wherein the one or more elements comprises an Cas9 endonuclease and a sgRNA directed at a target sequence in a target in the aminoadipate-semialdehyde synthase gene.

Disclosed herein is an isolated nucleic acid molecule comprising a nucleic acid sequence encoding one or more element of a gene editing system, wherein the one or more elements comprises an Cas9 endonuclease and a sgRNA directed at a target sequence in the α-aminoadipic semialdehyde gene. Disclosed herein is an isolated nucleic acid molecule comprising a nucleic acid sequence encoding one or more element of a gene editing system, wherein the one or more elements comprises an Cas9 endonuclease and a sgRNA directed at a target sequence in the α-aminoadipic semialdehyde gene. Disclosed herein is an isolated nucleic acid molecule comprising a nucleic acid sequence encoding one or more element of a gene editing system, wherein the one or more elements comprises an Cas9 endonuclease and a sgRNA directed at a target sequence in the kynurenine aminotransferase 2 gene. Disclosed herein is an isolated nucleic acid molecule comprising a nucleic acid sequence encoding one or more element of a gene editing system, wherein the one or more elements comprises an Cas9 endonuclease and a sgRNA directed at a target sequence in the dehydrogenase E1 and transketolase domain-containing protein 1 gene. Disclosed herein is an isolated nucleic acid molecule comprising a nucleic acid sequence encoding one or more element of a gene editing system, wherein the one or more elements comprises an Cas9 endonuclease and a sgRNA directed at a target sequence in the L-lysine alpha-oxidase gene. Disclosed herein is an isolated nucleic acid molecule comprising a nucleic acid sequence encoding one or more element of a gene editing system, wherein the one or more elements comprises an Cas9 endonuclease and a sgRNA directed at a target sequence in the ketimine reductase mu-crystallin protein gene. Disclosed herein is an isolated nucleic acid molecule comprising a nucleic acid sequence encoding one or more element of a gene editing system, wherein the one or more elements comprises an Cas9 endonuclease and a sgRNA directed at a target sequence in the peroxisomal sarcosine oxidase gene. Disclosed herein is an isolated nucleic acid molecule comprising a nucleic acid sequence encoding one or more element of a gene editing system, wherein the one or more elements comprises an Cas9 endonuclease and a sgRNA directed at a target sequence in the pyrroline-5-carboxylate reductase gene.

In an aspect, a disclosed element of a gene editing system can a CRISPR-based endonuclease. In an aspect, a disclosed endonuclease can be Cas9. In an aspect, a disclosed Cas9 can be that of Staphylococcus aureus or Streptococcus pyogenes. In an aspect, a disclosed Cas9 can be derived from Staphylococcus aureus or Streptococcus pyogenes. In an aspect, a disclosed Cas9 can be that of or derived from a species other than S. aureus or S. Pyogenes. In an aspect, a disclosed Cas9 can be any known Cas9 (see, e.g., those discussed supra). In an aspect, a disclosed Cas9 can be any Cas9 analog. Cas9 is well known to the art and the skilled person can identify and employ a Cas9 from one or more species without undue experimentation.

In an aspect, a disclosed Cas9 can have a sequence having at least 75%, at least 80%, at least 85%, at least 90%, or at least 95% identity to the set forth in SEQ ID NO:28 or a fragment thereof. In an aspect, a disclosed Cas9 can comprise one or more amino acid substitutions, insertions, deletions, modifications, or any combination thereof. The techniques to introduce one or more substitutions, insertions, deletions, modifications, or any combination thereof into a sequence encoding Cas9 are known to the skilled person.

In an aspect, a disclosed element of a gene editing system can comprise a sgRNA. The art is familiar with sgRNAs and the skilled person can identify and employ a sgRNA without undue experimentation. In an aspect, a disclosed sgRNA can be directed at any functional domain of a target sequence. In an aspect, a disclosed sgRNA can be directed at a target sequence in the glutaryl-CoA dehydrogenase gene. In an aspect, a disclosed sgRNA can comprise an sgRNA directed at a target sequence in a disclosed mouse glutaryl-CoA dehydrogenase (mGcdh) gene or at a disclosed human glutaryl-CoA dehydrogenase gene. In an aspect, a disclosed mGcdh gene can comprise the sequence set forth in SEQ ID NO:01. In an aspect, a disclosed hGCDH gene can comprise the sequence set forth in SEQ ID NO:02. In an aspect, a disclosed hGCDH gene can comprise the sequence set forth in SEQ ID NO: 17. In an aspect, a disclosed hGCDH gene can comprise the sequence set forth in SEQ ID NO:18. In an aspect, a disclosed sgRNA can comprise an sgRNA directed at a target sequence in exon 3 of mouse glutaryl-CoA dehydrogenase (mGcdh) gene. In an aspect, a disclosed sgRNA directed at a target sequence in exon 3 of mGcdh gene can comprise the sequence set forth in SEQ ID NO:05. In an aspect, a disclosed sgRNA can comprise an sgRNA directed at a target sequence in exon 5 of mouse glutaryl-CoA dehydrogenase (mGcdh) gene. In an aspect, a disclosed sgRNA directed at a target sequence in exon 5 of mGcdh can comprise the sequence set forth in SEQ ID NO:06.

In an aspect, a disclosed sgRNA can be directed at aminoadipate-semialdehyde synthase gene. For example, in an aspect, a disclosed aminoadipate-semialdehyde synthase can comprise a human or a mouse aminoadipate-semialdehyde synthase. In an aspect, a disclosed sgRNA can comprise an sgRNA directed at a target sequence in exon 6 of mouse aminoadipate-semialdehyde synthase (mAass) gene. In an aspect, a disclosed sgRNA directed at a target sequence in exon 6 of mAass gene can comprise the sequence set forth in SEQ ID NO:07 or SEQ ID NO:08. In an aspect, a disclosed sgRNA can comprise an sgRNA directed at a target sequence in exon 7 of mouse aminoadipate-semialdehyde synthase (mAass) gene. In an aspect, a disclosed sgRNA directed at a target sequence in exon 7 of mAass gene can comprise the sequence set forth in SEQ ID NO:09 or SEQ ID NO:10. In an aspect, a disclosed sgRNA can be directed at any functional domain of a target sequence.

In an aspect, a disclosed nucleic acid sequence encoding an aminoadipate-semialdehyde synthase can be derived from a non-mammalian species or from a mammalian species. In an aspect, a disclosed nucleic acid sequence encoding an aminoadipate-semialdehyde synthase can be that of a non-mammalian species or that of a mammalian species.

In an aspect, a disclosed nucleic acid sequence encoding an aminoadipate-semialdehyde synthase can comprise the sequence set forth in SEQ ID NO:01 or in SEQ ID NO:02. In an aspect, a disclosed nucleic acid sequence encoding an aminoadipate-semialdehyde synthase can comprise a sequence having about 50%, about 55%, about 60%, about 65%, about 70%, about 75%, about 80%, about 85%, about 90%, about 95%, or more identity to the sequence set forth in SEQ ID NO:01 or in SEQ ID NO:02.

In an aspect, a disclosed nucleic acid sequence encoding an aminoadipate-semialdehyde synthase can comprise the sequence set forth in SEQ ID NO:17 or in SEQ ID NO:18. In an aspect, a disclosed nucleic acid sequence encoding an aminoadipate-semialdehyde synthase can comprise a sequence having about 50%, about 55%, about 60%, about 65%, about 70%, about 75%, about 80%, about 85%, about 90%, about 95%, or more identity to the sequence set forth in SEQ ID NO:17 or in SEQ ID NO:18.

In an aspect, a disclosed encoded aminoadipate-semialdehyde synthase can comprise the sequence of NCBI Reference Sequence NP_000150.1 or the sequence of NCBI Reference Sequence XP_036009971.1.

In an aspect, a disclosed encoded aminoadipate-semialdehyde synthase can comprise one or more amino acid substitutions, insertions, deletions, modifications, or any combination thereof. The techniques to introduce one or more substitutions, insertions, deletions, modifications, or any combination thereof are known to the skilled person.

In an aspect, a disclosed element of a gene editing system can comprise a TracrRNA. In an aspect, a TracrRNA can be directed a target gene of interest. For example, a TracrRNA can be directed at at a target sequence in human or mouse aminoadipate-semialdehyde synthase gene or at a human or mouse glutaryl-CoA dehydrogenase gene. In an aspect, a disclosed TracrRNA can be directed at a target sequence in exon 6 of Aaas or exon 7 of Aass. In an aspect, a disclosed TracrRNA can comprise the sequence set forth in SEQ ID NO:11 or in SEQ ID NO:12.

In an aspect, a disclosed an isolated nucleic acid molecule comprising a nucleic acid sequence encoding one or more element of a gene editing system can comprise a nucleic acid sequence encoding two ITRs, a first promoter, a sgRNA, a TracrRNA, a second promoter, and a Cas9. In an aspect, a disclosed first promoter is operably linked to a disclosed sgRNA. In an aspect, a disclosed second promoter is operably linked to a disclosed Cas9.

In an aspect, a disclosed an isolated nucleic acid molecule comprising a nucleic acid sequence encoding one or more element of a gene editing system can comprise a nucleic acid sequence encoding two ITRs, a promoter, a sgRNA, a TracrRNA, a second promoter, and a Cas9. In an aspect, a disclosed promoter is operably linked to a disclosed sgRNA. In an aspect, a disclosed promoter is operably linked to a disclosed Cas9.

In an aspect, a disclosed isolated nucleic acid molecule comprising a nucleic acid sequence encoding one or more element of a gene editing system can comprise one or more nucleotide substitutions, insertions, deletions, modifications, or any combination thereof. The techniques to introduce one or more substitutions, insertions, deletions, modifications, or any combination thereof are known to the skilled person. In an aspect, a disclosed isolated nucleic acid molecule comprising a nucleic acid sequence encoding one or more element of a gene editing system can be codon-optimized for expression in a mammalian cell or a human cell. In an aspect, a disclosed isolated nucleic acid molecule can be CpG-free or CpG-depleted.

In an aspect, a disclosed isolated nucleic acid molecule can further comprise a nucleic acid sequence encoding a carboxy-terminal fluorescent label and/or fluorescent tag, an amino-terminal fluorescent label and/or fluorescent tag, or a combination thereof. In an aspect, a disclosed fluorescent label and/or fluorescent tag can comprise green fluorescent protein (EGFP), mEmerald, enhanced yellow fluorescent protein (EYFP), mApple, TdTomato, mCherry, miRFP670, any known fluorescent label or tag, or any combination thereof. Fluorophores and fluorescent labels are known in the art.

In an aspect, a disclosed isolated nucleic acid molecule comprising a nucleic acid sequence encoding one or more element of a gene editing system (i) can restore liver-specific modulation of lysine catabolism, (ii) restore one or more aspects of lysine homeostasis, (iii) can reduce or decrease the level of toxic catabolites in the liver and/or brain of a subject, (iv) can restore the metabolic flux from glutaryl-CoA to crotonyl-CoA, (v) can improve motor performance (e.g., strength, gait, balance, coordination, and combinations thereof) of a subject, (vi) can improve memory function of a subject, (vii) can reduce anxiety in a subject, (viii) can reduce and/or prevent neurological sequelae (e.g., neonatal macrocephaly, subdural hematomas, acute retinal hemorrhage, encephalopathy, striatal necrosis, and combinations thereof), (ix) can improve and/or reduce and/or eliminate vascular dysfunction in a subject, (x) can improve a subject's quality of life, (xi) can increase and/or prolong a subject's life span, (xii) can increase a subject's survivability, or (xiii) any combination thereof.

In an aspect, a disclosed isolated nucleic acid molecule comprising a nucleic acid sequence encoding one or more element of a gene editing system can treat and/or prevent Glutaric Aciduria Type-1 in a subject. In an aspect, a disclosed isolated nucleic acid molecule comprising a nucleic acid sequence encoding one or more element of a gene editing system can improve and/or diminish and/or ameliorate one or more symptoms associated Glutaric Aciduria Type-1 in a subject.

In an aspect, a disclosed isolated nucleic acid molecule comprising a nucleic acid sequence encoding one or more element of a gene editing system can comprise the nucleic acid sequence for one or more regulatory elements. In an aspect, a disclosed regulatory element can comprise a promoter, an enhancer, an internal ribosomal entry sites (IRES), and other expression control elements (e.g., transcription termination signals, such as polyadenylation signals and poly-U sequences), or any combination thereof. Regulatory elements can include those that direct constitutive expression of a nucleotide sequence in many types of host cells and those that direct expression of the nucleotide sequence only in certain host cells (e.g., tissue-specific regulatory sequences such as brain cells or neurons).

In an aspect, a disclosed isolated nucleic acid molecule comprising a nucleic acid sequence encoding one or more element of a gene editing system can improve and/or diminish and/or ameliorate one or more pathologies associated with Glutaric Aciduria Type-1 in a subject.

In an aspect, a first disclosed promoter can comprise a type III RNA polymerase III promoter. Type III RNA polymerase III promoters are known to the art. In an aspect, a disclosed type III RNA polymerase III promoter can comprise a U6 promoter. In an aspect, a disclosed U6 promoter can comprise the sequence set forth in SEQ ID NO:27. In an aspect, a first disclosed promoter can comprise the sequence set forth in SEQ ID NO:27.

In an aspect, a first disclosed promoter (e.g., a disclosed U6 promoter) can be operably linked to a disclosed sgRNA (such as, for example, a disclosed sgRNA for mouse or human aminoadipate-semialdehyde synthase). In an aspect, a second disclosed promoter can comprise a liver-specific promoter. Liver specific promoters are known to the art. In an aspect, a disclosed liver promoter can comprise the sequence set forth in SEQ ID NO:26. In an aspect, a second disclosed promoter (e.g., a liver-specific promoter) can be operably linked to a disclosed Cas9 (such as, for example, a disclosed SaCas9).

In an aspect, a disclosed isolated nucleic acid molecule comprising a nucleic acid sequence encoding one or more element of a gene editing system can comprise one or more inverted terminal repeats (ITRs). In an aspect, the one or more disclosed ITRs can be derived from AAV2 or AAV9. In an aspect, a disclosed ITR can comprise the sequence set forth in any one of SEQ ID NO:22 or SEQ ID NO:23. In an aspect, a disclosed isolated nucleic acid molecule comprising a nucleic acid sequence encoding one or more element of a gene editing system can comprise a first 5′ ITR and a second 3′ ITR.

In an aspect, a disclosed isolated nucleic acid molecule comprising a nucleic acid sequence encoding one or more element of a gene editing system can comprise one or more OLLAS tag. In an aspect, a disclosed OLLAS tag can comprise the sequence set forth in SEQ ID NO:31.

In an aspect, a disclosed isolated nucleic acid molecule comprising a nucleic acid sequence encoding one or more element of a gene editing system can comprise a nuclear localization signal (NLS). In an aspect, a disclosed NLS can comprise the sequence set forth in SEQ ID NO:30 or SEQ ID NO:32. NLS are known to the skilled person in the art. In an aspect, a disclosed isolated nucleic acid molecule comprising a nucleic acid sequence encoding one or more element of a gene editing system can comprise a polyA sequence. In an aspect, a disclosed polyA sequence can comprise the sequence set forth in SEQ ID NO:24 or SEQ ID NO:25. In an aspect, a disclosed isolated nucleic acid molecule comprising a nucleic acid sequence encoding one or more element of a gene editing system can comprise one or more hemagglutinin (HA) tags. In an aspect, a disclosed HA tag can comprise the sequence set forth in SEQ ID NO:29.

In an aspect, a disclosed isolated nucleic acid molecule comprising a nucleic acid sequence encoding one or more element of a gene editing system can be packaged in a viral vector (as discussed infra) or a non-viral vector. In an aspect, a disclosed non-viral vector can be a polymer-based vector, a peptide-based vector, a lipid nanoparticle, a solid lipid nanoparticle, or a cationic lipid-based vector. In an aspect, a disclosed isolated nucleic acid molecule comprising a nucleic acid sequence encoding one or more element of a gene editing system can be packaged in a recombinant AAV viral vector (e.g., AAV8 or AAVcc47).

Disclosed herein is an isolated nucleic acid molecule comprising a nucleic acid sequence encoding one or more element of a gene editing system, wherein the nucleic acid sequence comprises the sequence set forth in SEQ ID NO:20 or SEQ ID NO:21, or a fragment thereof. Disclosed herein is an isolated nucleic acid molecule comprising a nucleic acid sequence encoding one or more element of a gene editing system, wherein the nucleic acid sequence comprises a sequence having at least 50%, at least 55%, at least 60%, at least 65%, at least 70%, at least 75%, at least 80%, at least 85%, at least 90%, at least 95%, or more to the sequence set forth in SEQ ID NO:20 or SEQ ID NO:21, or a fragment thereof.

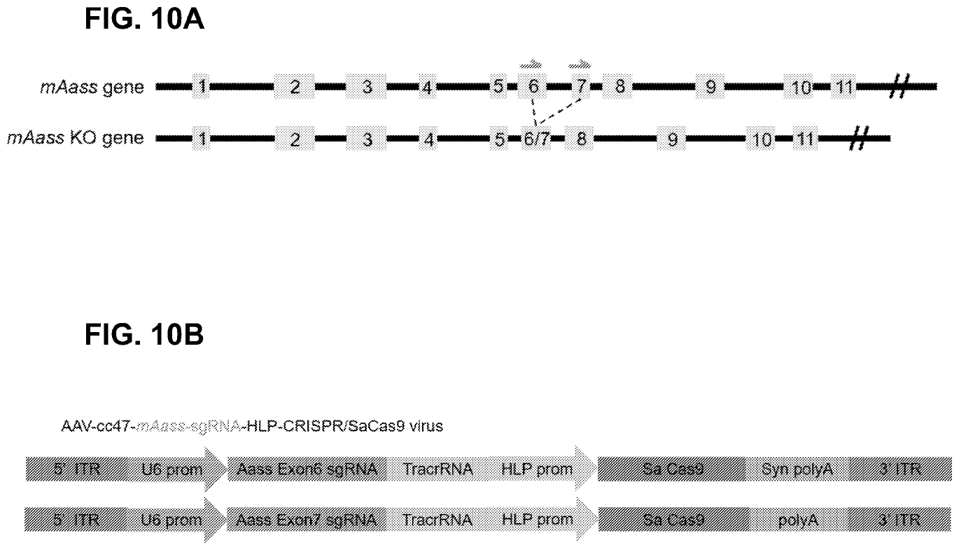

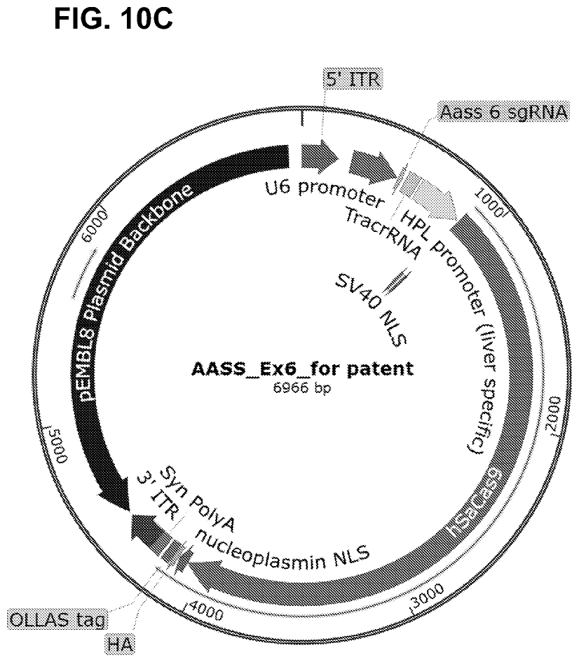

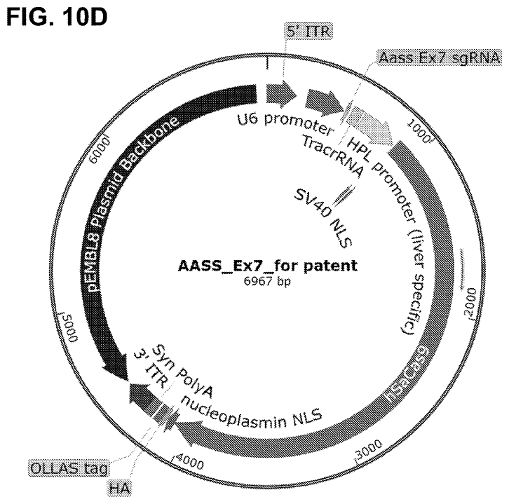

Disclosed herein is an isolated nucleic acid molecule comprising a nucleic acid sequence encoding one or more element of a gene editing system, wherein the nucleic acid molecule is represented by FIG. 10C. Disclosed herein is an isolated nucleic acid molecule comprising a nucleic acid sequence encoding one or more element of a gene editing system, wherein the nucleic acid molecule is represented by FIG. 10D.

Disclosed herein is an expression cassette comprising a disclosed isolated nucleic acid molecule comprising a nucleic acid sequence encoding one or more element of a gene editing system operably linked to one or more promoters.

Disclosed herein is an expression cassette comprising a disclosed isolated nucleic acid molecule comprising a nucleic acid sequence encoding one or more element of a gene editing system, wherein the one or more elements comprises an endonuclease and a sgRNA directed at a target sequence in a target gene of interest, wherein the endonuclease is operably linked a promoter and wherein the sgRNA is operably linked to a promoter.

Disclosed herein is an expression cassette comprising a disclosed isolated nucleic acid molecule comprising a nucleic acid sequence encoding one or more element of a gene editing system, wherein the one or more elements comprises an Cas9 endonuclease and a sgRNA directed at a target sequence in aminoadipate-semialdehyde synthase gene, wherein the endonuclease is operably linked a promoter and wherein the sgRNA is operably linked to a promoter.

### 3. Other Nucleic Acid Molecules

Generally, as known to the art, siRNA can be used to silence genes. In an aspect, a gene to be silenced or the silence gene is AASS. In an aspect, the process is as follows: (i) double-stranded RNA is cleaved by the Dicer enzyme, which forms siRNA, (ii) double-stranded siRNA then enters the cell and forms the RNA-induced silencing complex (RISC) with other proteins, (iii) this is unwound, which forms the single-stranded siRNA, (iv) the strand of RNA with the 5′ end base pairing that is thermodynamically less stable remains part of the RISC complex, which strand can now scan for complementary mRNA, (v) once this anti-sense strand binds to the target mRNA, mRNA cleavage is induced, and (vi) the foreign mRNA is recognized by the host cell as abnormal and is degraded. Now, translation is not possible, and the gene is silenced. Disclosed herein is an siRNA that can target any part of an aminoadipate-semialdehyde synthase gene. Disclosed herein is an siRNA that can target any part of the aminoadipate-semialdehyde synthase gene comprising the sequence set forth in SEQ ID NO:35 or SEQ ID NO:36. Disclosed herein is an siRNA that can target any part of an aminoadipate-semialdehyde synthase gene comprising the sequence set forth in SEQ ID NO:38 or SEQ ID NO:39. In an aspect, a targeted part of an AASS sequence can comprise about 15 to about 35 base pairs. In an aspect, a targeted part of an AASS sequence can comprise about 20 to about 30 base pairs. In an aspect, a targeted part of an AASS sequence can comprise about 20 to about 24 base pairs. In an aspect, a targeted part of an AASS sequence can comprise about 21 to about 22 base pairs. In an aspect, a disclosed siRNA effects the complete silencing of the aminoadipate-semialdehyde synthase gene. Disclosed herein is an siRNA that can target any part of an the aminoadipate-semialdehyde synthase sequence comprising the sequence set forth in SEQ ID NO:35 or SEQ ID NO:36. Disclosed herein is an siRNA that can target any part of the aminoadipate-semialdehyde synthase sequence comprising the sequence set forth in SEQ ID NO:38 or SEQ ID NO:39.

In an aspect, a disclosed siRNA can effect the partial silencing of the aminoadipate-semialdehyde synthase gene. In an aspect, a disclosed siRNA can effect the complete silencing of the aminoadipate-semialdehyde synthase gene. In an aspect, a disclosed siRNA can effect the partial silencing of the aminoadipate-semialdehyde synthase gene in a subject's liver. In an aspect, a disclosed siRNA can effect the complete silencing of the aminoadipate-semialdehyde synthase gene in a subject's liver.

Disclosed herein is an siRNA that can target any part of the aminoadipate-semialdehyde synthase gene, α-aminoadipic semialdehyde gene, the kynurenine aminotransferase 2 gene, the dehydrogenase E1 and transketolase domain-containing protein 1 gene, the L-lysine alpha-oxidase gene, the ketimine reductase mu-crystallin protein gene, the peroxisomal sarcosine oxidase gene, or the pyrroline-5-carboxylate reductase gene.

In an aspect, a targeted part of the α-aminoadipic semialdehyde gene, the kynurenine aminotransferase 2 gene, the dehydrogenase E1 and transketolase domain-containing protein 1 gene, the L-lysine alpha-oxidase gene, the ketimine reductase mu-crystallin protein gene, the peroxisomal sarcosine oxidase gene, or the pyrroline-5-carboxylate reductase gene can comprise about 15 to about 35 base pairs. In an aspect, a targeted part of the α-aminoadipic semialdehyde gene, the kynurenine aminotransferase 2 gene, the dehydrogenase E1 and transketolase domain-containing protein 1 gene, the L-lysine alpha-oxidase gene, the ketimine reductase mu-crystallin protein gene, the peroxisomal sarcosine oxidase gene, or the pyrroline-5-carboxylate reductase gene can comprise about 20 to about 30 base pairs. In an aspect, a targeted part of the α-aminoadipic semialdehyde gene, the kynurenine aminotransferase 2 gene, the dehydrogenase E1 and transketolase domain-containing protein 1 gene, the L-lysine alpha-oxidase gene, the ketimine reductase mu-crystallin protein gene, the peroxisomal sarcosine oxidase gene, or the pyrroline-5-carboxylate reductase gene can comprise about 20 to about 24 base pairs. In an aspect, a targeted part of the α-aminoadipic semialdehyde gene, the kynurenine aminotransferase 2 gene, the dehydrogenase E1 and transketolase domain-containing protein 1 gene, the L-lysine alpha-oxidase gene, the ketimine reductase mu-crystallin protein gene, the peroxisomal sarcosine oxidase gene, or the pyrroline-5-carboxylate reductase gene can comprise about 21 to about 22 base pairs.

In an aspect, a disclosed siRNA can effect the partial silencing of the α-aminoadipic semialdehyde gene, the kynurenine aminotransferase 2 gene, the dehydrogenase E1 and transketolase domain-containing protein 1 gene, the L-lysine alpha-oxidase gene, the ketimine reductase mu-crystallin protein gene, the peroxisomal sarcosine oxidase gene, or the pyrroline-5-carboxylate reductase gene. In an aspect, a disclosed siRNA can effect the complete silencing of the α-aminoadipic semialdehyde gene, the kynurenine aminotransferase 2 gene, the dehydrogenase E1 and transketolase domain-containing protein 1 gene, the L-lysine alpha-oxidase gene, the ketimine reductase mu-crystallin protein gene, the peroxisomal sarcosine oxidase gene, or the pyrroline-5-carboxylate reductase gene. In an aspect, a disclosed siRNA can effect the partial silencing of the α-aminoadipic semialdehyde gene, the kynurenine aminotransferase 2 gene, the dehydrogenase E1 and transketolase domain-containing protein 1 gene, the L-lysine alpha-oxidase gene, the ketimine reductase mu-crystallin protein gene, the peroxisomal sarcosine oxidase gene, or the pyrroline-5-carboxylate reductase gene in a subject's liver. In an aspect, a disclosed siRNA can effect the complete silencing of the α-aminoadipic semialdehyde gene, the kynurenine aminotransferase 2 gene, the dehydrogenase E1 and transketolase domain-containing protein 1 gene, the L-lysine alpha-oxidase gene, the ketimine reductase mu-crystallin protein gene, the peroxisomal sarcosine oxidase gene, or the pyrroline-5-carboxylate reductase gene in a subject's liver.

Disclosed herein is mRNA therapy that can target or can be directed one or more enzymes in the pipecolate pathway, the saccharopine pathway, or both.

In an aspect, a disclosed mRNA molecule can further comprise a polyA tail. In an aspect, a disclosed mRNA molecule can comprise a 5′ cap. In an aspect, a disclosed mRNA molecule can comprise at least one nonstandard nucleobase, such as, for example, 5-methyl-cytidine, pseudouridine, and 2-thio-uridine.

In an aspect, a disclosed mRNA molecule can be used to induce functional GCDH expression in a mammal or a mammalian cell. In an aspect, the disclosed functional GCDH can be induced in a subject's liver or brain or in cells found in the liver or in the brain.

In an aspect, a disclosed mRNA molecule can be used to induce functional α-aminoadipic semialdehyde, the kynurenine aminotransferase 2, the dehydrogenase E1 and transketolase domain-containing protein 1, the L-lysine alpha-oxidase, the ketimine reductase mu-crystallin protein, the peroxisomal sarcosine oxidase, or the pyrroline-5-carboxylate reductase expression in a mammal or a mammalian cell. In an aspect, the disclosed functional α-aminoadipic semialdehyde, the kynurenine aminotransferase 2, the dehydrogenase E1 and transketolase domain-containing protein 1, the L-lysine alpha-oxidase, the ketimine reductase mu-crystallin protein, the peroxisomal sarcosine oxidase, or the pyrroline-5-carboxylate reductase can be induced in a subject's liver or brain or in cells found in the liver or in the brain.

In an aspect, a disclosed mRNA molecule can be encapsulated within a nanoparticle. For example, in an aspect, a disclosed nanoparticle can be a liposome. In an aspect, a disclosed liposome can comprise one or more cationic lipids, one or more non-cationic lipids, and one or more PEG-modified lipids. In an aspect, a disclosed liposome can comprise one or more cholesterol-based lipids. In an aspect, a disclosed liposome comprises one or more cationic lipids, one or more non-cationic lipids, one or more cholesterol-based lipids and one or more PEG-modified lipids. In an aspect, a disclosed liposome can comprise no more than three distinct lipid components (such as, for example, a sterol-based cationic lipid). In an aspect, a disclosed sterol-based cationic lipid can be imidazole cholesterol ester (ICE), GL-TES-SA-DME-E18-2, TL1-01D-DMA, SY-3-E14-DMAPr, TL1-10D-DMA, Guan-SS-Chol, GL-TES-SA-DMP-E18-2, HEP-E4-E10, HEP-E3-E10, TL1-04D-DMA, GL-TES-SA-DME-E18-2, TL1-O1D-DMA, SY-3-E14-DMAPr, TL1-10D-DMA, or a combination thereof.

In an aspect, a disclosed liposome can have a size of less than about 200 nm, or less than about 150 nm, or less than about 120 nm, or less than about 110 nm, or less than about 100 nm, or less than about 80 nm, or less than about 60 nm, or less than about 50 nm, or less than about 40 nm, or less than about 30 nm.

### 4. Viral Vectors

Disclosed herein is a viral vector comprising a disclosed isolated nucleic acid molecule. Disclosed herein is a viral vector comprising a disclosed isolated nucleic acid molecule comprising a nucleic acid sequence encoding glutaryl-CoA dehydrogenase. Disclosed herein is a viral vector comprising a disclosed isolated nucleic acid molecule comprising a nucleic acid sequence encoding aminoadipate-semialdehyde synthase. Disclosed herein is viral vector comprising an isolated nucleic acid molecule comprising a nucleic acid sequence encoding green fluorescent protein (GFP). Disclosed herein is a viral vector comprising a disclosed isolated nucleic acid molecule comprising a nucleic acid sequence encoding glutaryl-CoA dehydrogenase, aminoadipate-semialdehyde synthase, or green fluorescent protein.

Disclosed herein is a viral vector comprising a nucleic acid sequence encoding a glutaryl-CoA dehydrogenase, wherein the nucleic acid sequence comprise the sequence set forth in SEQ ID NO:01 or a fragment thereof. Disclosed herein is a viral vector comprising a nucleic acid sequence encoding a glutaryl-CoA dehydrogenase, wherein the nucleic acid sequence comprise the sequence set forth in SEQ ID NO:02 or a fragment thereof. Disclosed herein is a viral vector comprising a nucleic acid sequence encoding a glutaryl-CoA dehydrogenase, wherein the nucleic acid sequence comprise the sequence set forth in SEQ ID NO:17 or a fragment thereof. Disclosed herein is a viral vector comprising a nucleic acid sequence encoding a glutaryl-CoA dehydrogenase, wherein the nucleic acid sequence comprise the sequence set forth in SEQ ID NO:18 or a fragment thereof.

Disclosed herein is a viral vector comprising a nucleic acid sequence encoding a glutaryl-CoA dehydrogenase, wherein the nucleic acid sequence comprises the sequence set forth in SEQ ID NO:01, wherein the sequence comprises one or more nucleotide substitutions, insertions, deletions, modifications, or any combination thereof. Disclosed herein is a viral vector comprising a nucleic acid sequence encoding a glutaryl-CoA dehydrogenase, wherein the nucleic acid sequence comprises the sequence set forth in SEQ ID NO:02, wherein the sequence comprises one or more nucleotide substitutions, insertions, deletions, modifications, or any combination thereof.

Disclosed herein is a viral vector comprising a nucleic acid sequence encoding a glutaryl-CoA dehydrogenase, wherein the nucleic acid sequence comprises the sequence set forth in SEQ ID NO:17, wherein the sequence comprises one or more nucleotide substitutions, insertions, deletions, modifications, or any combination thereof. Disclosed herein is a viral vector comprising a nucleic acid sequence encoding a glutaryl-CoA dehydrogenase, wherein the nucleic acid sequence comprises the sequence set forth in SEQ ID NO:18, wherein the sequence comprises one or more nucleotide substitutions, insertions, deletions, modifications, or any combination thereof.

Disclosed herein is a viral vector comprising an isolated nucleic acid molecule comprising a nucleic acid sequence encoding one or more element of a gene editing system.

Disclosed herein is a viral vector comprising an isolated nucleic acid molecule comprising a nucleic acid sequence encoding one or more element of a gene editing system, wherein the one or more elements comprises an endonuclease and a sgRNA directed at a target sequence in a target gene of interest.

Disclosed herein is a viral vector comprising an isolated nucleic acid molecule comprising a nucleic acid sequence encoding one or more element of a gene editing system, wherein the one or more elements comprises an Cas9 endonuclease and a sgRNA directed at a target sequence in glutaryl-CoA dehydrogenase gene.

Disclosed herein is a viral vector comprising an isolated nucleic acid molecule comprising a nucleic acid sequence encoding one or more element of a gene editing system, wherein the one or more elements comprises an Cas9 endonuclease and a sgRNA directed at a target sequence in aminoadipate-semialdehyde synthase gene

Disclosed herein is a recombinant AAV vector comprising an isolated nucleic acid molecule comprising a nucleic acid sequence encoding one or more element of a gene editing system. Disclosed herein is a recombinant AAV vector comprising an isolated nucleic acid molecule comprising a nucleic acid sequence encoding one or more element of a gene editing system, wherein the one or more elements comprises an endonuclease and a sgRNA directed at a target sequence in a target gene of interest. Disclosed herein is a recombinant AAV vector comprising an isolated nucleic acid molecule comprising a nucleic acid sequence encoding one or more element of a gene editing system, wherein the one or more elements comprises an Cas9 endonuclease and a sgRNA directed at a target sequence in the glutaryl-CoA dehydrogenase gene.

Disclosed herein is a recombinant AAV vector comprising an isolated nucleic acid molecule comprising a nucleic acid sequence encoding one or more element of a gene editing system, wherein the one or more elements comprises an Cas9 endonuclease and a sgRNA directed at a target sequence in the aminoadipate-semialdehyde synthase gene.

Disclosed herein is a recombinant AAVcc47 vector comprising an isolated nucleic acid molecule comprising a nucleic acid sequence encoding one or more element of a gene editing system. Disclosed herein is a recombinant AAVcc47 vector comprising an isolated nucleic acid molecule comprising a nucleic acid sequence encoding one or more element of a gene editing system, wherein the one or more elements comprises an endonuclease and a sgRNA directed at a target sequence in a target gene of interest. Disclosed herein is a recombinant AAVcc47 vector comprising an isolated nucleic acid molecule comprising a nucleic acid sequence encoding one or more element of a gene editing system, wherein the one or more elements comprises an Cas9 endonuclease and a sgRNA directed at a target sequence in the glutaryl-CoA dehydrogenase gene. Disclosed herein is a recombinant AAVcc47 vector comprising an isolated nucleic acid molecule comprising a nucleic acid sequence encoding one or more element of a gene editing system, wherein the one or more elements comprises an Cas9 endonuclease and a sgRNA directed at a target sequence in the aminoadipate-semialdehyde synthase gene.

Disclosed herein is a viral vector comprising the sequence set forth in SEQ ID NO:19. Disclosed herein is a viral vector comprising the sequence set forth in SEQ ID NO:20. Disclosed herein is a viral vector comprising the sequence set forth in SEQ ID NO:21.

In an aspect, a disclosed viral vector can comprise a nucleic acid sequence encoding a carboxy-terminal fluorescent label and/or fluorescent tag, an amino-terminal fluorescent label and/or fluorescent tag, or a combination thereof. In an aspect, a disclosed fluorescent label and/or fluorescent tag can comprise green fluorescent protein (EGFP), mEmerald, enhanced yellow fluorescent protein (EYFP), mApple, TdTomato, mCherry, miRFP670, any known fluorescent label or tag, or any combination thereof. Fluorophores and fluorescent labels are known.

In an aspect, a disclosed viral vector can be an adenovirus vector, an AAV vector, a herpes simplex virus vector, a retrovirus vector, a lentivirus vector, and alphavirus vector, a flavivirus vector, a rhabdovirus vector, a measles virus vector, a Newcastle disease viral vector, a poxvirus vector, or a picornavirus vector. In an aspect, a disclosed viral vector can be an adenovirus vector, an adenovirus-associated (AAV) vector, or a lentivirus vector. In an aspect, a disclosed AAV vector can be a recombinant AAV (rAAV) vector.

In an aspect, a disclosed AAV vector can include naturally isolated serotypes including, but not limited to, AAV1, AAV2, AAV3 (including 3a and 3b), AAV4, AAV5, AAV6, AAV7, AAV8, AAVrh8, AAV9, AAV10, AAVrh10, AAV11, AAV12, AAV13, AAVrh39, AAVrh43, AAVcy.7 as well as bovine AAV, caprine AAV, canine AAV, equine AAV, ovine AAV, avian AAV, primate AAV, non-primate AAV, and any other virus classified by the International Committee on Taxonomy of Viruses (ICTV) as an AAV. In an aspect, an AAV capsid can be a chimera either created by capsid evolution or by rational capsid engineering from a naturally isolated AAV variants to capture desirable serotype features such as enhanced or specific tissue tropism and/or a host immune response escape. Naturally isolated AAV variants include, but not limited to, AAV-DJ, AAV-HAE1, AAV-HAE2, AAVM41, AAV-1829, AAV2 Y/F, AAV2 T/V, AAV2i8, AAV2.5, AAV9.45, AAV9.61, AAV-B1, AAV-AS, AAV9.45A-String (e.g., AAV9.45-AS), AAV9.45Angiopep, AAV9.47-Angiopep, and AAV9.47-AS, AAV-PHP.B, AAV-PHP.eB, AAV-PHP.S, AAV-F, AAVcc.47, and AAVcc.81. In an aspect, a disclosed AAV vector can be AAV-Rh74 or a related variant (e.g., capsid variants like RHM4-1). In an aspect, a disclosed AAV vector can comprise AAVcc.47.

In an aspect of a disclosed AAV vector, a disclosed nucleic acid sequence can have a coding sequence that is less than about 4.5 kilobases.

In an aspect, a disclosed vector can comprise the nucleic acid sequence for one or more regulatory elements. In an aspect, a disclosed regulatory element can comprise a promoter, an enhancer, an internal ribosomal entry sites (IRES), and other expression control elements (e.g., transcription termination signals, such as polyadenylation signals and poly-U sequences), or any combination thereof. Regulatory elements can include those that direct constitutive expression of a nucleotide sequence in many types of host cells and those that direct expression of the nucleotide sequence only in certain host cells (e.g., tissue-specific regulatory sequences such as brain cells or neurons).

In an aspect, a disclosed vector can comprise a promoter operably linked to a nucleic acid sequence encoding a glutaryl-CoA dehydrogenase. In an aspect of a disclosed vector, a disclosed promoter can comprise a tissue specific promoter. In an aspect of a disclosed vector, a disclosed tissue specific promoter can comprise a neuron-specific promoter, a muscle-specific promoter, a liver-specific promoter, a skeletal muscle-specific promoter, and heart-specific promoter. In an aspect of a disclosed vector, a disclosed tissue-specific promoter can comprise a brain cell specific promoter. Brain cell specific promoter are known to the art and can comprise a synapsin 1 (Syn1) promoter, a calmodulin/calcium dependent kinase II (CAMKII) promoter, a glial fibrillary acidic protein (GFAP) promoter, a Rgs5 promoter, a S100 beta promoter, a neuron-specific enolase (NSE) promoter, a Thy1 promoter, or any combination thereof.

In an aspect of a disclosed vector, a disclosed promoter can comprise a liver-specific promoter. Liver specific promoters are known to the art. In an aspect of a disclosed vector, a disclosed liver promoter can comprise the sequence set forth in SEQ ID NO:26. In an aspect of a disclosed vector, a disclosed promoter can comprise a type III RNA polymerase III promoter. Type III RNA polymerase III promoters are known to the art. In an aspect, a disclosed type III RNA polymerase III promoter can comprise a U6 promoter. In an aspect of a disclosed vector, a disclosed U6 promoter can comprise the sequence set forth in SEQ ID NO:27.

In an aspect, a disclosed vector can comprise one or more OLLAS tag. In an aspect, a disclosed OLLAS tag can comprise the sequence set forth in SEQ ID NO:31. In an aspect, a disclosed vector can comprise a nuclear localization signal (NLS). In an aspect, a disclosed NLS can comprise the sequence set forth in SEQ ID NO:30 or SEQ ID NO:32. NLS are known to the skilled person in the art. In an aspect, a disclosed vector can comprise one or more inverted terminal repeats (ITRs). In an aspect, the one or more disclosed ITRs can be derived from AAV2 or AAV9. In an aspect, a disclosed ITR can comprise the sequence set forth in any one of SEQ ID NO:22 or SEQ ID NO:23. In an aspect, a disclosed vector can comprise a polyA sequence. In an aspect, a disclosed polyA sequence can comprise the sequence set forth in SEQ ID NO:24 or SEQ ID NO:25. In an aspect, a disclosed vector can comprise one or more hemagglutinin (HA) tags. In an aspect, a disclosed HA tag can comprise the sequence set forth in SEQ ID NO:29. In an aspect, a disclosed vector can comprise a TracrRNA sequence. In an aspect, a TracrRNA sequence can comprise the sequence set forth in SEQ ID NO:11 or SEQ ID NO:12.

In an aspect, a therapeutically effective amount of disclosed vector can comprise a range of about 1×10¹⁰ vg/kg to about 2×10¹⁴·vg/kg. In an aspect, for example, a disclosed vector can be administered at a dose of about 1×10¹¹ to about 8×10¹³ vg/kg or about 1×10¹² to about 8×10¹³ vg/kg. In an aspect, a disclosed vector can be administered at a dose of about 1×10¹³ to about 6×10¹³ vg/kg. In an aspect, a disclosed vector can be administered at a dose of at least about 1×10¹⁰, at least about 5×10¹⁰, at least about 1×10¹¹, at least about 5×10¹¹, at least about 1×10¹², at least about 5×10¹², at least about 1×10¹³, at least about 5×10¹³, or at least about 1×10¹⁴ vg/kg. In an aspect, a disclosed vector can be administered at a dose of no more than about 1×10¹⁰, no more than about 5×10¹⁰, no more than about 1×10¹¹, no more than about 5×10¹¹, no more than about 1×10¹², no more than about 5×10¹², no more than about 1×10¹³, no more than about 5×10¹³, or no more than about 1×10¹⁴ vg/kg. In an aspect, a disclosed vector can be administered at a dose of about 1×10¹² vg/kg. In an aspect, a disclosed vector can be administered at a dose of about 1×10¹¹ vg/kg. In an aspect, a disclosed vector can be administered in a single dose, or in multiple doses (such as 2, 3, 4, 5, 6, 7, 8, 9 or 10 doses) as needed for the desired therapeutic results (such as for example, reprogramming a metabolic pathway).

In an aspect, a disclosed viral vector can be validated and/or characterized using an animal model such as mice and/or C. elegans.

In an aspect, a disclosed vector can restore the functionality of a missing, dysfunctional, and/or mutated glutaryl-CoA dehydrogenase in a cell or a subject. In an aspect, a disclosed vector (i) can restore liver-specific modulation of lysine catabolism, (ii) restore one or more aspects of lysine homeostasis, (iii) can reduce or decrease the level of toxic catabolites in the liver and/or brain of a subject, (iv) can restore the metabolic flux from glutaryl-CoA to crotonyl-CoA, (v) can improve motor performance (e.g., strength, gait, balance, coordination, and combinations thereof) of a subject, (vi) can improve memory function of a subject, (vii) can reduce anxiety in a subject, (viii) can reduce and/or prevent neurological sequelae (e.g., neonatal macrocephaly, subdural hematomas, acute retinal hemorrhage, encephalopathy, striatal necrosis, and combinations thereof), (ix) can improve and/or reduce and/or eliminate vascular dysfunction in a subject, (x) can improve a subject's quality of life, (xi) can increase and/or prolong a subject's life span, (xii) can increase a subject's survivability, or (xiii) any combination thereof.

In an aspect, a disclosed vector can treat and/or prevent Glutaric Aciduria Type-1 in a subject. In an aspect, a disclosed vector can improve and/or diminish and/or ameliorate one or more symptoms associated Glutaric Aciduria Type-1 in a subject. In an aspect, a disclosed vector can improve and/or diminish and/or ameliorate one or more pathologies associated with Glutaric Aciduria Type-1 in a subject.

### 5. Formulations

Disclosed herein is a pharmaceutical formulation comprising one or more disclosed GCDH nucleic acid molecules, disclosed CRISPR based nucleic acid molecules, disclosed viral vectors, disclosed cells, disclosed plasmids, or any combination thereof, and at least one pharmaceutically acceptable carrier.

Disclosed herein is a pharmaceutical formulation comprising an isolated nucleic acid molecule comprising a nucleic acid sequence encoding glutaryl-CoA dehydrogenase, and at least one pharmaceutically acceptable carrier.

Disclosed herein is a pharmaceutical formulation comprising an isolated nucleic acid molecule comprising a nucleic acid sequence encoding one or more element of a gene editing system, and at least one pharmaceutically acceptable carrier. Disclosed herein is a pharmaceutical formulation comprising an isolated nucleic acid molecule comprising a nucleic acid sequence encoding one or more element of a gene editing system, wherein the one or more elements comprises an endonuclease and a sgRNA directed at a target sequence in a target gene of interest, and at least one pharmaceutically acceptable carrier. Disclosed herein is a pharmaceutical formulation comprising an isolated nucleic acid molecule comprising a nucleic acid sequence encoding one or more element of a gene editing system, wherein the one or more elements comprises an Cas9 endonuclease and a sgRNA directed at a target sequence in the aminoadipate-semialdehyde synthase gene, and at least one pharmaceutically acceptable carrier. Disclosed herein is a pharmaceutical formulation comprising a viral vector comprising a disclosed isolated nucleic acid molecule, and at least one pharmaceutically acceptable carrier. Disclosed herein is a pharmaceutical formulation comprising a viral vector comprising a disclosed isolated nucleic acid molecule comprising a nucleic acid sequence encoding glutaryl-CoA dehydrogenase, and at least one pharmaceutically acceptable carrier. Disclosed herein is a pharmaceutical formulation comprising a viral vector comprising a disclosed isolated nucleic acid molecule comprising a nucleic acid sequence encoding aminoadipate-semialdehyde synthase, and at least one pharmaceutically acceptable carrier.

Disclosed herein is a pharmaceutical formulation comprising a viral vector comprising an isolated nucleic acid molecule comprising a nucleic acid sequence encoding green fluorescent protein (GFP), and at least one pharmaceutically acceptable carrier.

Disclosed herein is a pharmaceutical formulation comprising a viral vector comprising a disclosed isolated nucleic acid molecule comprising a nucleic acid sequence encoding glutaryl-CoA dehydrogenase, aminoadipate-semialdehyde synthase, or green fluorescent protein, and at least one pharmaceutically acceptable carrier.

Disclosed herein is a pharmaceutical formulation comprising a viral vector comprising a nucleic acid sequence encoding a glutaryl-CoA dehydrogenase, wherein the nucleic acid sequence comprise the sequence set forth in SEQ ID NO:01 or a fragment thereof, and at least one pharmaceutically acceptable carrier. Disclosed herein is a pharmaceutical formulation comprising a viral vector comprising a nucleic acid sequence encoding a glutaryl-CoA dehydrogenase, wherein the nucleic acid sequence comprise the sequence set forth in SEQ ID NO:02 or a fragment thereof, and at least one pharmaceutically acceptable carrier. Disclosed herein is a pharmaceutical formulation comprising a viral vector comprising a nucleic acid sequence encoding a glutaryl-CoA dehydrogenase, wherein the nucleic acid sequence comprises the sequence set forth in SEQ ID NO:01 or SEQ ID NO:02, wherein the sequence comprises one or more nucleotide substitutions, insertions, deletions, modifications, or any combination thereof, and at least one pharmaceutically acceptable carrier. Disclosed herein is a pharmaceutical formulation comprising a viral vector comprising a nucleic acid sequence encoding a glutaryl-CoA dehydrogenase, wherein the nucleic acid sequence comprises the sequence set forth in SEQ ID NO:17 or SEQ ID NO:18, wherein the sequence comprises one or more nucleotide substitutions, insertions, deletions, modifications, or any combination thereof, and at least one pharmaceutically acceptable carrier.

Disclosed herein is a pharmaceutical formulation comprising a viral vector comprising an isolated nucleic acid molecule comprising a nucleic acid sequence encoding one or more element of a gene editing system, and at least one pharmaceutically acceptable carrier. Disclosed herein is a pharmaceutical formulation comprising a viral vector comprising an isolated nucleic acid molecule comprising a nucleic acid sequence encoding one or more element of a gene editing system, wherein the one or more elements comprises an endonuclease and a sgRNA directed at a target sequence in a target gene of interest, and at least one pharmaceutically acceptable carrier. Disclosed herein is a pharmaceutical formulation comprising a viral vector comprising an isolated nucleic acid molecule comprising a nucleic acid sequence encoding one or more element of a gene editing system, wherein the one or more elements comprises an Cas9 endonuclease and a sgRNA directed at a target sequence in the glutaryl-CoA dehydrogenase gene, and at least one pharmaceutically acceptable carrier. Disclosed herein is a pharmaceutical formulation comprising a viral vector comprising an isolated nucleic acid molecule comprising a nucleic acid sequence encoding one or more element of a gene editing system, wherein the one or more elements comprises an Cas9 endonuclease and a sgRNA directed at a target sequence in the aminoadipate-semialdehyde synthase gene, and at least one pharmaceutically acceptable carrier.

Disclosed herein is a pharmaceutical formulation comprising a recombinant AAV vector comprising an isolated nucleic acid molecule comprising a nucleic acid sequence encoding one or more element of a gene editing system, and at least one pharmaceutically acceptable carrier. Disclosed herein is a pharmaceutical formulation comprising a recombinant AAV vector comprising an isolated nucleic acid molecule comprising a nucleic acid sequence encoding one or more element of a gene editing system, wherein the one or more elements comprises an endonuclease and a sgRNA directed at a target sequence in a target gene of interest, and at least one pharmaceutically acceptable carrier.

Disclosed herein is a pharmaceutical formulation comprising a recombinant AAV vector comprising an isolated nucleic acid molecule comprising a nucleic acid sequence encoding one or more element of a gene editing system, wherein the one or more elements comprises an Cas9 endonuclease and a sgRNA directed at glutaryl-CoA dehydrogenase, and at least one pharmaceutically acceptable carrier. Disclosed herein is a pharmaceutical formulation comprising a recombinant AAV vector comprising an isolated nucleic acid molecule comprising a nucleic acid sequence encoding one or more element of a gene editing system, wherein the one or more elements comprises an Cas9 endonuclease and a sgRNA directed at a target sequence in the aminoadipate-semialdehyde synthase gene, and at least one pharmaceutically acceptable carrier. Disclosed herein is a pharmaceutical formulation comprising a recombinant AAVcc47 vector comprising an isolated nucleic acid molecule comprising a nucleic acid sequence encoding one or more element of a gene editing system, and at least one pharmaceutically acceptable carrier.

Disclosed herein is a pharmaceutical formulation comprising a recombinant AAVcc47 vector comprising an isolated nucleic acid molecule comprising a nucleic acid sequence encoding one or more element of a gene editing system, wherein the one or more elements comprises an endonuclease and a sgRNA directed at a target sequence in the target gene of interest, and at least one pharmaceutically acceptable carrier.

Disclosed herein is a pharmaceutical formulation comprising a recombinant AAVcc47 vector comprising an isolated nucleic acid molecule comprising a nucleic acid sequence encoding one or more element of a gene editing system, wherein the one or more elements comprises an Cas9 endonuclease and a sgRNA directed at a target sequence in the glutaryl-CoA dehydrogenase gene, and at least one pharmaceutically acceptable carrier. Disclosed herein is a pharmaceutical formulation comprising a recombinant AAVcc47 vector comprising an isolated nucleic acid molecule comprising a nucleic acid sequence encoding one or more element of a gene editing system, wherein the one or more elements comprises an Cas9 endonuclease and a sgRNA directed at a target sequence in the aminoadipate-semialdehyde synthase gene, and at least one pharmaceutically acceptable carrier. Disclosed herein is a pharmaceutical formulation comprising a viral vector comprising the sequence set forth in SEQ ID NO:19, SEQ ID NO:20, or SEQ ID NO:21, and at least one pharmaceutically acceptable carrier.

In an aspect, a disclosed pharmaceutical formulation can comprise at least one lyoprotectant. In an aspect, a disclosed lyoprotectant can comprise peptone, glycerol, lactose, gelatin, glucose, sucrose, trehalose, dextran, maltodextrin, adonitol, sodium glutamate, or any combination thereof. Lyoprotectants are known to those skilled in the art.

In an aspect, a disclosed pharmaceutical formulation can comprise at least one gelling agent, preferably a pharmaceutically acceptable gelling agent. In an aspect, a disclosed pharmaceutical formulation can comprise at least preservative such as, for example, benzyl alcohol, cresols, benzoic acid, phenol, parabens, or sorbic acid. In an aspect, a disclosed pharmaceutical formulation can comprise at least one stabilizer such as, for example, a surfactant, a polymer, a polyol, a poloxamer, an albumin, a gelatin, a trehalose, a protein, a sugar, a polyvinylpyrrolidone, a N-acetyl-tryptophan (NAT), a caprylate (e.g., sodium caprylate), a polysorbate (e.g., P80), an amino acid, and a divalent metal cation (e.g., zinc). 6. Cells

Disclosed herein is a cell comprising a disclosed isolated nucleic acid molecule or a disclosed plasmid. Disclosed herein are cells transfected by one or more disclosed nucleic acid molecules. Disclosed herein are cells transduced by one or more disclosed vectors.

Disclosed herein are cells having a GCDH^{-/-} genotype. Disclosed herein are cells having a AASS⁻/⁻ genotype. Disclosed herein are cells having a GCDH⁻/⁻ and AASS⁻/⁻ genotype.

Disclosed herein are cells having a Gcdh^{-/-} genotype. Disclosed herein are cells having a Aass^{-/-} genotype. Disclosed herein are cells having a Gcdh⁻/⁻ and Aass⁻/⁻ genotype.

Disclosed herein are cells demonstrating a GCDH^{-/-} genotype following transduction with a disclosed viral vector. Disclosed herein are cells demonstrating a AASS⁻/⁻ genotype following transduction with a disclosed viral vector. Disclosed herein are cells demonstrating a GCDH⁻/⁻ and AASS⁻/⁻ genotype following transduction with a disclosed viral vector.

Disclosed herein are cells demonstrating a Gcdh⁻/⁻ genotype following transduction with a disclosed viral vector. Disclosed herein are cells demonstrating a Aass⁻/⁻ genotype following transduction with a disclosed viral vector. Disclosed herein are cells demonstrating a Gcdh⁻/⁻ and Aass⁻/⁻ genotype following transduction with a disclosed viral vector.

In an aspect, disclosed transduced cells can comprise any central nervous system cells. CNS cells include but are not limited to neurons, glial cells, vascular cells, and combinations thereof. As known to the art, neurons include sensory neurons, motor neurons, interneurons, brain neurons, and combinations thereof. Neurons includes multipolar neurons, unipolar neurons, bipolar neurons, pseudo-unipolar neurons, and combinations thereof. In an aspect, disclosed transduced cells can comprise hepatocytes. In an aspect, disclosed transduced cells can comprise mammalian brain cells or mammalian hepatocytes.

Disclosed herein are cells transfected by a disclosed plasmid. Disclosed herein are cells transduced by a vector comprising the sequence set forth in SEQ ID NO:19. Disclosed herein are cells transduced by a vector comprising the sequence set forth in SEQ ID NO:20. Disclosed herein are cells transduced by a vector comprising the sequence set forth in SEQ ID NO:21. Disclosed herein are cells transfected by an isolated nucleic acid molecule comprising the sequence set forth in SEQ ID NO:01 or SEQ ID NO:02. Disclosed herein are cells transfected by an isolated nucleic acid molecule comprising the sequence set forth in SEQ ID NO:17 or in SEQ ID NO:18. Disclosed herein are cells transfected by an isolated nucleic acid molecule comprising the sequence set forth in SEQ ID NO:35 or in SEQ ID NO:36. Disclosed herein are cells transfected by an isolated nucleic acid molecule comprising the sequence set forth in SEQ ID NO:37 or in SEQ ID NO:38.

In an aspect, disclosed cells can comprise cells harvested and/or obtained from a subject. In an aspect, disclosed cells can comprise cells harvested and/or obtained from a subject suspected of having or diagnosed with GA-1. Techniques to achieve transfection are known to the art and using transfected cells are known to the art.

### 7. Plasmids

Disclosed herein is a plasmid used in a disclosed method. Disclosed herein is a plasmid comprising one or more disclosed isolated nucleic acid molecules.

For example, in an aspect, a disclosed plasmid can comprise an isolated nucleic acid molecule comprising the sequence set forth in any one of SEQ ID NO:01-SEQ ID NO:02, or a fragment thereof. In an aspect, a disclosed plasmid can comprise an isolated nucleic acid molecule encoding the sequence set forth in SEQ ID NO:03 or SEQ ID NO:04, or a fragment thereof. In an aspect, a disclosed plasmid can comprise an isolated nucleic acid molecule comprising the sequence set forth in SEQ ID NO:17 or SEQ ID NO:18, or a fragment thereof. In an aspect, a disclosed plasmid can comprise an isolated nucleic acid molecule comprising the sequence set forth in SEQ ID NO:35 or SEQ ID NO:36, or a fragment thereof. In an aspect, a disclosed plasmid can comprise an isolated nucleic acid molecule comprising the sequence set forth in SEQ ID NO:38 or SEQ ID NO:39, or a fragment thereof.

In an aspect, a disclosed plasmid can comprise a nucleic acid sequence for a disclosed fluorescent label and/or fluorescent tag. In an aspect, a disclosed fluorescent label and/or fluorescent tag can comprise green fluorescent protein (EGFP), mEmerald, enhanced yellow fluorescent protein (EYFP), mApple, TdTomato, mCherry, miRFP670, any known fluorescent label or tag, or any combination thereof. Fluorophores and fluorescent labels are known.

### 8. Kits

Disclosed herein is a kit comprising one or more disclosed isolated nucleic acid molecules, one or more disclosed vectors, one or more disclosed cells, one or more disclosed plasmids, or any combination thereof.

Disclosed herein is a kit comprising an isolated nucleic acid molecule comprising a nucleic acid sequence encoding glutaryl-CoA dehydrogenase. Disclosed herein is a kit comprising an isolated nucleic acid molecule comprising a nucleic acid sequence encoding one or more element of a gene editing system. Disclosed herein is a kit comprising an isolated nucleic acid molecule comprising a nucleic acid sequence encoding one or more element of a gene editing system, wherein the one or more elements comprises an endonuclease and a sgRNA directed at a target sequence in the target gene of interest. Disclosed herein is a kit comprising an isolated nucleic acid molecule comprising a nucleic acid sequence encoding one or more element of a gene editing system, wherein the one or more elements comprises an Cas9 endonuclease and a sgRNA directed at aminoadipate-semialdehyde synthase.

Disclosed herein is a kit comprising a viral vector comprising a disclosed isolated nucleic acid molecule. Disclosed herein is a kit comprising a viral vector comprising a disclosed isolated nucleic acid molecule comprising a nucleic acid sequence encoding glutaryl-CoA dehydrogenase, or a disclosed isolated nucleic acid molecule comprising a nucleic acid sequence encoding aminoadipate-semialdehyde synthase, or a nucleic acid sequence encoding green fluorescent protein (GFP).

Disclosed herein is a kit comprising a viral vector comprising a nucleic acid sequence encoding a glutaryl-CoA dehydrogenase, wherein the nucleic acid sequence comprise the sequence set forth in SEQ ID NO:01 or a fragment thereof, or comprising a viral vector comprising a nucleic acid sequence encoding a glutaryl-CoA dehydrogenase, wherein the nucleic acid sequence comprise the sequence set forth in SEQ ID NO:02 or a fragment thereof.

Disclosed herein is a kit comprising a viral vector comprising a nucleic acid sequence encoding a glutaryl-CoA dehydrogenase, wherein the nucleic acid sequence comprise the sequence set forth in SEQ ID NO:17 or a fragment thereof, or comprising a viral vector comprising a nucleic acid sequence encoding a glutaryl-CoA dehydrogenase, wherein the nucleic acid sequence comprise the sequence set forth in SEQ ID NO:18 or a fragment thereof.

Disclosed herein is a kit comprising a viral vector comprising an isolated nucleic acid molecule comprising a nucleic acid sequence encoding one or more element of a gene editing system. Disclosed herein is a kit comprising a viral vector comprising an isolated nucleic acid molecule comprising a nucleic acid sequence encoding one or more element of a gene editing system, wherein the one or more elements comprises an endonuclease and a sgRNA directed at a target sequence in the target gene of interest.

Disclosed herein is a kit comprising a viral vector comprising an isolated nucleic acid molecule comprising a nucleic acid sequence encoding one or more element of a gene editing system, wherein the one or more elements comprises an Cas9 endonuclease and a sgRNA directed at a target sequence in the glutaryl-CoA dehydrogenase gene.

Disclosed herein is a kit comprising a viral vector comprising an isolated nucleic acid molecule comprising a nucleic acid sequence encoding one or more element of a gene editing system, wherein the one or more elements comprises an Cas9 endonuclease and a sgRNA directed at a target sequence in the aminoadipate-semialdehyde synthase gene. Disclosed herein is a kit comprising a recombinant AAV vector comprising an isolated nucleic acid molecule comprising a nucleic acid sequence encoding one or more element of a gene editing system. Disclosed herein is a kit comprising a recombinant AAV vector comprising an isolated nucleic acid molecule comprising a nucleic acid sequence encoding one or more element of a gene editing system, wherein the one or more elements comprises an endonuclease and a sgRNA directed at a target sequence in a target gene of interest.

Disclosed herein is a kit comprising a viral vector comprising the sequence set forth in SEQ ID NO:19. Disclosed herein is a kit comprising a viral vector comprising the sequence set forth in SEQ ID NO:20. Disclosed herein is a kit comprising a viral vector comprising the sequence set forth in SEQ ID NO:21.

Disclosed herein is a kit comprising cells comprising a disclosed isolated nucleic acid molecule or a disclosed plasmid. Disclosed herein is a kit comprising cells transfected by one or more disclosed nucleic acid molecules. Disclosed herein is a kit comprising cells transduced by one or more disclosed vectors.

Disclosed herein is a kit comprising cells having a Gcdh^{-/-} genotype. Disclosed herein is a kit comprising cells having a Aass^{-/-} genotype. Disclosed herein is a kit comprising cells having a Gcdh^{-/-} and Aass^{-/-} genotype. Disclosed herein is a kit comprising cells demonstrating a Gcdh^{-/-} genotype following transduction with a disclosed viral vector. Disclosed herein is a kit comprising cells demonstrating a Aass^{-/-} genotype following transduction with a disclosed viral vector. Disclosed herein is a kit comprising cells demonstrating a Gcdh^{-/-} and Aass^{-/-} genotype following transduction with a disclosed viral vector.

Disclosed herein is a kit comprising cells having a GCDH^{-/-} genotype. Disclosed herein is a kit comprising cells having a AASS^{-/-} genotype. Disclosed herein is a kit comprising cells having a GCDH^{-/-} and AASS^{-/-} genotype. Disclosed herein is a kit comprising cells demonstrating a GCDH^{-/-} genotype following transduction with a disclosed viral vector. Disclosed herein is a kit comprising cells demonstrating a AASS^{-/-} genotype following transduction with a disclosed viral vector. Disclosed herein is a kit comprising cells demonstrating a GCDH^{-/-} and AASS^{-/-} genotype following transduction with a disclosed viral vector.

Disclosed herein is a kit comprising cells transfected by a disclosed plasmid. Disclosed herein is a kit comprising cells transduced by a vector comprising the sequence set forth in SEQ ID NO:19. Disclosed herein is a kit comprising cells transduced by a vector comprising the sequence set forth in SEQ ID NO:20. Disclosed herein is a kit comprising cells transduced by a vector comprising the sequence set forth in SEQ ID NO:21. Disclosed herein is a kit comprising one or more disclosed compositions and/or components and/or agents that can be used in any disclosed method.

Disclosed herein is a kit comprising one or more disclosed compositions and/or components and/or agents that can be used in validating and/or characterizing a disclosed composition (such as, for example, a disclosed isolated nucleic acid molecule, a disclosed plasmid, a disclosed viral vector, or any combination thereof). In an aspect, validating and/or characterizing can comprise using an animal model such as mice and/or C. elegans.

In an aspect of a disclosed kit, a disclosed fluorescent label or a fluorescent tag. In an aspect, a disclosed fluorescent label or disclosed fluorophore can comprise enhanced green fluorescent protein (EGFP), mEmerald, enhanced yellow fluorescent protein (EYFP), mApple, TdTomato, mCherry, miRFP670, any known fluorescent label or tag, or any combination thereof. Fluorophores and fluorescent labels are known in the art.

In an aspect, a disclosed kit can comprise at least two components constituting the kit. Together, the components constitute a functional unit for a given purpose (such as, for example, performing any aspect of a disclosed method including preparing the components used in a disclosed method). Individual member components can be physically packaged together or separately. For example, a kit comprising an instruction for using the kit can or cannot physically include the instruction with other individual member components. Instead, the instruction can be supplied as a separate member component, either in a paper form or an electronic form which can be supplied on computer readable memory device or downloaded from an internet website, or as recorded presentation. In an aspect, a kit for use in a disclosed method can comprise one or more containers holding a disclosed composition, a disclosed pharmaceutical formulation, a disclosed therapeutic agent, and a label or package insert with instructions for use. In an aspect, suitable containers include, for example, bottles, vials, syringes, blister pack, etc. The containers can be formed from a variety of materials such as glass or plastic. The container can hold a disclosed composition, a disclosed pharmaceutical formulation, a disclosed therapeutic agent, or a combination thereof, and can have a sterile access port (for example the container can be an intravenous solution bag or a vial having a stopper pierceable by a hypodermic injection needle). The label or package insert can indicate that a disclosed composition, a disclosed viral vector, a disclosed nucleic acid molecule, a disclosed cell, or a combination thereof, can be used in a disclosed method. A kit can comprise additional components necessary for administration such as, for example, other buffers, diluents, filters, needles, and syringes.

In an aspect, a disclosed kit can be used (i) to restore liver-specific modulation of lysine catabolism, (ii) to restore one or more aspects of lysine homeostasis in a subject's liver, (iii) to reduce or decrease the level of toxic catabolites in the liver and/or brain of a subject, (iv) to restore the metabolic flux from glutaryl-CoA to crotonyl-CoA in a subject's liver, (v) to improve motor performance (e.g., strength, gait, balance, coordination, and combinations thereof) of a subject, (vi) to improve memory function of a subject, (vii) to reduce anxiety in a subject, (viii) to reduce and/or prevent neurological sequelae (e.g., neonatal macrocephaly, subdural hematomas, acute retinal hemorrhage, encephalopathy, striatal necrosis, and combinations thereof), (ix) to improve and/or reduce and/or eliminate vascular dysfunction in a subject, (x) to improve a subject's quality of life, (xi) to increase and/or prolong a subject's life span, (xii) to increase a subject's survivability, or (xiii) to effect any combination thereof. In an aspect, a disclosed kit can be used treat and/or prevent Glutaric Aciduria Type-1 in a subject. In an aspect, a disclosed kit can be used to improve and/or diminish and/or ameliorate one or more symptoms associated Glutaric Aciduria Type-1 in a subject. In an aspect, a disclosed kit can be used to improve and/or diminish and/or ameliorate one or more pathologies associated with Glutaric Aciduria Type-1 in a subject.

### B. Methods Employing GCDH Nucleic Acid Molecules

### 1. Methods of Restoring the Expression of Glutaryl-CoA Dehydrogenase

Disclosed herein is a method of restoring the expression of glutaryl-CoA dehydrogenase, the method comprising administering to a subject in need thereof a therapeutically effective amount of a vector viral vector comprising a nucleic acid sequence encoding glutaryl-CoA dehydrogenase, wherein expression of the nucleic acid sequence generates a functional glutaryl-CoA dehydrogenase.

Disclosed herein is a method of restoring the expression of glutaryl-CoA dehydrogenase, the method comprising administering to a subject in need thereof a therapeutically effective amount of a vector viral vector comprising a nucleic acid sequence encoding glutaryl-CoA dehydrogenase, wherein expression of the nucleic acid sequence generates a functional glutaryl-CoA dehydrogenase in the subject's liver.

Disclosed herein is a method of restoring the expression of glutaryl-CoA dehydrogenase, the method comprising administering to a subject in need thereof a therapeutically effective amount of a vector viral vector comprising the nucleic acid sequence set forth in SEQ ID NO:01 or SEQ ID NO:02, wherein expression of the nucleic acid sequence generates a functional glutaryl-CoA dehydrogenase. Disclosed herein is a method of restoring the expression of glutaryl-CoA dehydrogenase, the method comprising administering to a subject in need thereof a therapeutically effective amount of a vector viral vector comprising the nucleic acid sequence set forth in SEQ ID NO:01 or SEQ ID NO:02, wherein expression of the nucleic acid sequence generates a functional glutaryl-CoA dehydrogenase in the subject's liver.

Disclosed herein is a method of restoring the expression of glutaryl-CoA dehydrogenase, the method comprising administering to a subject in need thereof a therapeutically effective amount of a vector viral vector comprising the nucleic acid sequence set forth in SEQ ID NO:17 or SEQ ID NO:18, wherein expression of the nucleic acid sequence generates a functional glutaryl-CoA dehydrogenase. Disclosed herein is a method of restoring the expression of glutaryl-CoA dehydrogenase, the method comprising administering to a subject in need thereof a therapeutically effective amount of a vector viral vector comprising the nucleic acid sequence set forth in SEQ ID NO:17 or SEQ ID NO:18, wherein expression of the nucleic acid sequence generates a functional glutaryl-CoA dehydrogenase in the subject's liver.

Disclosed herein is a method of restoring the expression of glutaryl-CoA dehydrogenase, the method comprising treating a subject in need thereof by administering to the subject a therapeutically effective amount of a vector viral vector comprising a nucleic acid sequence encoding glutaryl-CoA dehydrogenase, wherein expression of the nucleic acid sequence generates a functional glutaryl-CoA dehydrogenase. Disclosed herein is a method of restoring the expression of glutaryl-CoA dehydrogenase, the method comprising treating a subject in need thereof by administering to the subject a therapeutically effective amount of a vector viral vector comprising a nucleic acid sequence encoding glutaryl-CoA dehydrogenase, wherein expression of the nucleic acid sequence generates a functional glutaryl-CoA dehydrogenase in the subject's liver.

Disclosed herein is a method of restoring the expression of glutaryl-CoA dehydrogenase, the method comprising treating a subject in need thereof by administering to the subject a therapeutically effective amount of a vector viral vector comprising the nucleic acid sequence set forth in SEQ ID NO:01 or SEQ ID NO:02, wherein expression of the nucleic acid sequence generates a functional glutaryl-CoA dehydrogenase. Disclosed herein is a method of restoring the expression of glutaryl-CoA dehydrogenase, the method treating a subject in need thereof by administering to the subject a therapeutically effective amount of a vector viral vector comprising the nucleic acid sequence set forth in SEQ ID NO:01 or SEQ ID NO:02, wherein expression of the nucleic acid sequence generates a functional glutaryl-CoA dehydrogenase in the subject's liver.

Disclosed herein is a method of restoring the expression of glutaryl-CoA dehydrogenase, the method treating a subject in need thereof by administering to the subject a therapeutically effective amount of a vector viral vector comprising the nucleic acid sequence set forth in SEQ ID NO:17 or SEQ ID NO:18, wherein expression of the nucleic acid sequence generates a functional glutaryl-CoA dehydrogenase. Disclosed herein is a method of restoring the expression of glutaryl-CoA dehydrogenase, the method treating a subject in need thereof by administering to the subject a therapeutically effective amount of a vector viral vector comprising the nucleic acid sequence set forth in SEQ ID NO:17 or SEQ ID NO:18, wherein expression of the nucleic acid sequence generates a functional glutaryl-CoA dehydrogenase in the subject's liver.

Disclosed herein is a method of restoring the expression of glutaryl-CoA dehydrogenase, the method treating a subject in need thereof by administering to the subject a therapeutically effective amount of a vector viral vector comprising the sequence set forth in SEQ ID NO:19, wherein expression of the nucleic acid sequence generates a functional glutaryl-CoA dehydrogenase. Disclosed herein is a method of restoring the expression of glutaryl-CoA dehydrogenase, the method treating a subject in need thereof by administering to the subject a therapeutically effective amount of a vector viral vector comprising the sequence set forth in SEQ ID NO:19, wherein expression of the nucleic acid sequence generates a functional glutaryl-CoA dehydrogenase in the subject's liver.

In an aspect, a disclosed method can restore normal lysine catabolism in the subject's liver.

In an aspect, a disclosed method can further comprise deleting and/or disrupting one or more other catabolic genes. In an aspect, a disclosed method can further comprise deleting and/or disrupting the aminoadipate-semialdehyde synthase gene, α-aminoadipic semialdehyde gene, the kynurenine aminotransferase 2 gene, the dehydrogenase E1 and transketolase domain-containing protein 1 gene, the L-lysine alpha-oxidase gene, the ketimine reductase mu-crystallin protein gene, the peroxisomal sarcosine oxidase gene, or the pyrroline-5-carboxylate reductase gene, or any combination thereof.

In an aspect, the expression of glutaryl-CoA dehydrogenase can be restored in the subject's liver. In an aspect of a disclosed method, the disclosed subject in need thereof can have or can have been diagnosed with a glutaric aciduria type 1 (GA-1). In an aspect, a subject can be male or female. In an aspect, a subject can be an adult, a teenager, an adolescent, a child, a toddler, a baby, or an infant.

In an aspect of a disclosed method, the disclosed subject's GA-1 related and/or associated behavior can be modulated. In an aspect of a disclosed method, the disclosed subject's GA-1 related and/or associated physiology can be modulated.

In an aspect of a disclosed method, administering a disclosed viral vector can be administered systemically or directly. In an aspect, administering a disclosed viral vector molecule can comprise oral administration, intravenous administration, intratumoral administration, intraperitoneal administration, or any combination thereof. In an aspect, administering a disclosed viral vector can be administered by any method of administration disclosed herein. In an aspect, a disclosed viral vector can be administered via multiple routes either concurrently or sequentially. A skilled clinician can determine the best route of administration for a subject at a given time.

In an aspect of a disclosed method, administering a disclosed viral vector can be targeted to the subject's liver, wherein expression of the nucleic acid sequence generates a functional glutaryl-CoA dehydrogenase.

In an aspect, a disclosed nucleic acid sequence can comprise only the sequence for the functional domains.

In an aspect of a disclosed method, administering a disclosed viral vector can comprise a single dose, or in multiple doses (such as 2, 3, 4, 5, 6, 7, 8, 9 or 10 doses) as needed for the desired therapeutic results. In an aspect, multiple doses can be administered via the same route or via differing routes of administration. In an aspect, a disclosed viral vector can be administered via multiple routes of administration.

In an aspect, a disclosed viral vector can comprise a recombinant AAV vector. In an aspect, a disclosed AAV vector can comprise AAVcc.47. In an aspect, a disclosed AAV vector can comprise AAV8.

In an aspect, a therapeutically effective amount of disclosed vector can comprise a range of about 1×10¹⁰ vg/kg to about 2×10¹⁴·vg/kg. In an aspect, for example, a disclosed vector can be administered at a dose of about 1×10¹¹ to about 8×10¹³ vg/kg or about 1×10¹² to about 8×10¹³ vg/kg. In an aspect, a disclosed vector can be administered at a dose of about 1×10¹³ to about 6×10¹³ vg/kg. In an aspect, a disclosed vector can be administered at a dose of at least about 1×10¹⁰, at least about 5×10¹⁰, at least about 1×10¹¹, at least about 5×10¹¹, at least about 1×10¹², at least about 5×10¹², at least about 1×10¹³, at least about 5×10¹³, or at least about 1×10¹⁴ vg/kg. In an aspect, a disclosed vector can be administered at a dose of no more than about 1×10¹⁰, no more than about 5×10¹⁰, no more than about 1×10¹¹, no more than about 5×10¹¹, no more than about 1×10¹², no more than about 5×10¹², no more than about 1×10¹³, no more than about 5×10¹³, or no more than about 1×10¹⁴ vg/kg. In an aspect, a disclosed vector can be administered at a dose of about 1×10¹² vg/kg. In an aspect, a disclosed vector can be administered at a dose of about 1×10¹¹ vg/kg. In an aspect, a disclosed vector can be administered in a single dose, or in multiple doses (such as 2, 3, 4, 5, 6, 7, 8, 9 or 10 doses) as needed for the desired therapeutic results (such as for example, restoring the expression of GA-1).

In an aspect, a disclosed method can further comprise monitoring the subject for adverse effects following the administering step and/or the treating step. In an aspect, wherein in the absence of adverse effects, the method can further comprise continuing to treat the subject and/or continuing to monitor the subject. In an aspect, wherein in the presence of adverse effects, the method can further comprise modifying one or more steps of the method. In an aspect, modifying can comprise modifying the treating step, modifying the administering step, or both.

In an aspect, modifying the treating step can comprise changing the amount of the vector administered to the subject, changing the frequency of administration of the vector, changing the duration of administration of the vector, changing the route of administration of the vector, or any combination thereof. In an aspect, modifying the administering step can comprise changing the amount of the vector administered to the subject, changing the frequency of administration of the vector, changing the duration of administration of the vector, changing the route of administration of the vector, or any combination thereof.

In an aspect of a disclosed method, a disclosed CRISPR/Cas9 editing system can be applied to one or more amino acids of any enzyme in the lysine catabolism pathways (i.e., the pipecolate pathway and the saccharopine pathway). In an aspect, these enzymes can include, but are not limited to aminoadipate-semialdehyde synthase, α-aminoadipic semialdehyde, the kynurenine aminotransferase 2, the dehydrogenase E1 and transketolase domain-containing protein 1, the L-lysine alpha-oxidase, the ketimine reductase mu-crystallin protein, the peroxisomal sarcosine oxidase, or the pyrroline-5-carboxylate reductase, or any combination thereof. In an aspect, for example, a disclosed CRISPR/Cas9 editing system can be applied to one or more amino acids in a disclosed enzyme comprising the sequence set forth in SEQ ID NO:03 or SEQ ID NO:04 or SEQ ID NO:37 or SEQ ID NO:40.

In an aspect, a disclosed method can further comprise administering to the subject a therapeutically effective amount of a therapeutic agent. Therapeutic agents are known. In an aspect, a disclosed method can further comprise administering to the subject a therapeutically effective amount of one or more immune modulators. In an aspect, the one or more immune modulators comprise methotrexate, rituximab, intravenous gamma globulin, Tacrolimus, prednisolone or a prednisolone analog, SVP-Rapamycin, bortezomib, or a combination thereof.

In an aspect, a disclosed method can further comprise monitoring the subject for adverse effects following the administering of one or more therapeutic agents and/or the administering of one or more immune modulators. In an aspect, wherein in the absence of adverse effects following the administering of one or more therapeutic agents and/or immune modulators, the method can further comprise continuing to treat the subject and/or continuing to monitor the subject. In an aspect, wherein in the presence of adverse effects, the method can further comprise modifying one or more steps of the method. In an aspect, modifying can comprise modifying the administering step of one or more therapeutic agents and/or immune modulators. In an aspect, modifying the administering step can comprise changing the amount of one or more therapeutic agents and/or immune modulators administered to the subject, changing the frequency of administration of one or more therapeutic agents and/or immune modulators, changing the duration of administration of the vector, changing the route of administration of one or more therapeutic agents and/or immune modulators, or any combination thereof.

In an aspect, a disclosed method can further comprise monitoring the subject's metabolic and/or physiologic improvement following the administering and/or treating step and/or following the administering and/or treating steps. In an aspect, a clinician can measure and/or determine the subject's metabolic and/or physiologic status over time to identify one or more improvements and/or one or more diminishments. In an aspect of a disclosed method, a clinician can use the subject's metabolic and/or physiologic status and/or the trend of the subject's metabolic and/or physiological status and/or trend to make a treatment decision and/or to modify an aspect of a disclosed method and/or to continue treating the subject and/or continue to administer a disclosed vector, a disclosed composition, a disclosed therapeutic agent, and/or a disclosed immune modulator, or any combination thereof. In an aspect, metabolic and/or physiologic data can inform the clinician.

In an aspect, techniques to monitor, measure, and/or assess the restoring of the expression of glutaryl-CoA dehydrogenase can comprise qualitative (or subjective) means as well as quantitative (or objective) means. These means are known to the skilled. In an aspect, a disclosed method can comprise subjecting the subject to one or more invasive or non-invasive diagnostic assessments. Diagnostic assessments are known to the art. In an aspect, a disclosed non-invasive diagnostic assessment can comprise x-rays, computerized tomography (CT) scans, magnetic resonance imaging (MRI) scans, ultrasounds, positron emission tomography (PET) scans, or any combination thereof. In an aspect, a disclosed invasive diagnostic assessment can comprise a tissue biopsy or exploratory surgery.

In an aspect, a disclosed increase and/or a disclosed improvement can comprise a 10%, 20%, 30%, 40%, 50%, 60%, 70%, 80%, 90%, 100%, or any amount of an increase and/or improvement when compared to a control subject (such as, for example, a subject that has not received a disclosed treatment (e.g., administration of a disclosed viral vector)). In an aspect, a disclosed increase and/or a disclosed improvement can comprise a 10-20%, 20-30%, 30-40%, 40-50%, 50-60%, 60-70%, 70-80%, 80-90%, or 90-100% or any amount of an increase and/or improvement when compared to a control subject (such as a subject that has not received a disclosed treatment (e.g., administration of a disclosed viral vector)).

In an aspect, a disclosed decrease and/or a disclosed reduction can comprise a 10%, 20%, 30%, 40%, 50%, 60%, 70%, 80%, 90%, 100%, or any amount of a decrease and/or reduction when compared to a control subject (such as, for example, a subject that has not received a disclosed treatment (e.g., administration of a disclosed viral vector)). In an aspect, a disclosed decrease and/or a disclosed reduction can comprise a 10-20%, 20-30%, 30-40%, 40-50%, 50-60%, 60-70%, 70-80%, 80-90%, or 90-100% or any amount of a decrease and/or reduction when compared to a control subject (such as a subject that has not received a disclosed treatment (e.g., administration of a disclosed viral vector)).

In an aspect, a disclosed method of restoring the expression of glutaryl-CoA dehydrogenase can comprise repeating an administering step one or more times. In an aspect, a disclosed method of restoring the expression of glutaryl-CoA dehydrogenase can comprise repeating a treating step one or more times.

In an aspect, a disclosed method of restoring the expression of glutaryl-CoA dehydrogenase can comprise restoring liver-specific modulation of lysine catabolism, (ii) restoring one or more aspects of lysine homeostasis, (iii) reducing and/or decreasing the level of toxic catabolites in the liver and/or brain of a subject, (iv) restoring the metabolic flux from glutaryl-CoA to crotonyl-CoA, (v) improving motor performance (e.g., strength, gait, balance, coordination, and combinations thereof) of a subject, (vi) improving memory function of a subject, (vii) reduce anxiety in a subject, (viii) reducing and/or preventing neurological sequelae (e.g., neonatal macrocephaly, subdural hematomas, acute retinal hemorrhage, encephalopathy, striatal necrosis, and combinations thereof), (ix) improving and/or reducing and/or eliminating vascular dysfunction in a subject, (x) improving a subject's quality of life, (xi) increasing and/or prolong a subject's life span, (xii) increasing a subject's survivability, or (xiii) any combination thereof.

In an aspect, a disclosed method of restoring the expression of glutaryl-CoA dehydrogenase can comprise restoring liver-specific modulation of lysine catabolism in the subject's liver, (ii) restoring one or more aspects of lysine homeostasis in the subject's liver, (iii) reducing and/or decreasing the level of toxic catabolites in the liver and/or brain of a subject, (iv) restoring the metabolic flux from glutaryl-CoA to crotonyl-CoA in the subject's liver, or (v) any combination thereof.

In an aspect, a disclosed method of restoring the expression of glutaryl-CoA dehydrogenase can comprise treating and/or preventing Glutaric Aciduria Type-1 disease progression in a subject.

In an aspect, a disclosed method of restoring the expression of glutaryl-CoA dehydrogenase can comprise improving and/or diminishing and/or ameliorate one or more symptoms associated Glutaric Aciduria Type-1 disease progression in a subject.

In an aspect, a disclosed method of restoring the expression of glutaryl-CoA dehydrogenase can comprise improving and/or diminishing and/or ameliorate one or more pathologies associated with Glutaric Aciduria Type-1 disease progression in a subject.

In an aspect, a disclosed method of restoring the expression of glutaryl-CoA dehydrogenase can comprise restoring one or more aspects of cellular homeostasis and/or cellular functionality in a subject.

In an aspect, a disclosed method of restoring the expression of glutaryl-CoA dehydrogenase can comprise reprogramming a metabolic pathway. In an aspect, a disclosed metabolic pathway can comprise lysine catabolism. In an aspect, a disclosed metabolic pathway can comprise a disclosed pipecolate pathway and/or a disclosed saccharopine pathway.

2. Methods of Treating and/or Preventing GA-1 Disease Progression

Disclosed herein is a method of treating and/or preventing GA-1 disease progression, the method comprising administering to a subject in need thereof a therapeutically effective amount of a vector viral vector comprising a nucleic acid sequence encoding glutaryl-CoA dehydrogenase, wherein expression of glutaryl-CoA dehydrogenase is restored.

Disclosed herein is a method of treating and/or preventing GA-1 disease progression, the method comprising administering to a subject in need thereof a therapeutically effective amount of a vector viral vector comprising a nucleic acid sequence encoding glutaryl-CoA dehydrogenase, wherein expression of glutaryl-CoA dehydrogenase is restored in the subject's liver.

Disclosed herein is a method of treating and/or preventing GA-1 disease progression, the method comprising administering to a subject in need thereof a therapeutically effective amount of a vector viral vector comprising the nucleic acid sequence set forth in SEQ ID NO:01 or SEQ ID NO:02, wherein expression of glutaryl-CoA dehydrogenase is restored. Disclosed herein is a method of treating and/or preventing GA-1 disease progression, the method comprising administering to a subject in need thereof a therapeutically effective amount of a vector viral vector comprising the nucleic acid sequence set forth in SEQ ID NO:01 or SEQ ID NO:02, wherein expression of glutaryl-CoA dehydrogenase is restored in the subject's liver.

Disclosed herein is a method of treating and/or preventing GA-1 disease progression, the method comprising administering to a subject in need thereof a therapeutically effective amount of a vector viral vector comprising the nucleic acid sequence set forth in SEQ ID NO:17, wherein expression of glutaryl-CoA dehydrogenase is restored. Disclosed herein is a method of treating and/or preventing GA-1 disease progression, the method comprising administering to a subject in need thereof a therapeutically effective amount of a vector viral vector comprising the nucleic acid sequence set forth in SEQ ID NO:18, wherein expression of glutaryl-CoA dehydrogenase is restored.

Disclosed herein is a method of treating and/or preventing GA-1 disease progression, the method comprising treating a subject in need thereof by administering to the subject a therapeutically effective amount of a vector viral vector comprising a nucleic acid sequence encoding glutaryl-CoA dehydrogenase, wherein expression of glutaryl-CoA dehydrogenase is restored. Disclosed herein is a method of treating and/or preventing GA-1 disease progression, the method comprising treating a subject in need thereof by administering to the subject a therapeutically effective amount of a vector viral vector comprising a nucleic acid sequence encoding glutaryl-CoA dehydrogenase, wherein expression of glutaryl-CoA dehydrogenase is restored in the subject's liver.

Disclosed herein is a method of treating and/or preventing GA-1 disease progression, the method comprising treating a subject in need thereof by administering to the subject a therapeutically effective amount of a vector viral vector comprising the nucleic acid sequence set forth in SEQ ID NO:01 or SEQ ID NO:02, wherein expression of glutaryl-CoA dehydrogenase is restored. Disclosed herein is a method of treating and/or preventing GA-1 disease progression, the method treating a subject in need thereof by administering to the subject a therapeutically effective amount of a vector viral vector comprising the nucleic acid sequence set forth in SEQ ID NO:01 or SEQ ID NO:02, wherein expression of glutaryl-CoA dehydrogenase is restored in the subject's liver.

Disclosed herein is a method of treating and/or preventing GA-1 disease progression, the method comprising treating a subject in need thereof by administering to the subject a therapeutically effective amount of a vector viral vector comprising the nucleic acid sequence set forth in SEQ ID NO:17 or SEQ ID NO:18, wherein expression of glutaryl-CoA dehydrogenase is restored. Disclosed herein is a method of treating and/or preventing GA-1 disease progression, the method treating a subject in need thereof by administering to the subject a therapeutically effective amount of a vector viral vector comprising the nucleic acid sequence set forth in SEQ ID NO:17 or SEQ ID NO:18, wherein expression of glutaryl-CoA dehydrogenase is restored in the subject's liver.

Disclosed herein is a method of treating and/or preventing GA-1 disease progression, the method treating a subject in need thereof by administering to the subject a therapeutically effective amount of a vector viral vector comprising the sequence set forth in SEQ ID NO:19, wherein expression of glutaryl-CoA dehydrogenase is restored.

In an aspect, a disclosed method can restore normal lysine catabolism in the subject's liver.

In an aspect, a disclosed method can further comprise deleting and/or disrupting one or more other catabolic genes. In an aspect, a disclosed method can further comprise deleting and/or disrupting the aminoadipate-semialdehyde synthase gene, α-aminoadipic semialdehyde gene, the kynurenine aminotransferase 2 gene, the dehydrogenase E1 and transketolase domain-containing protein 1 gene, the L-lysine alpha-oxidase gene, the ketimine reductase mu-crystallin protein gene, the peroxisomal sarcosine oxidase gene, or the pyrroline-5-carboxylate reductase gene, or any combination thereof.

In an aspect of a disclosed method, the disclosed subject in need thereof can have or can have been diagnosed with a glutaric aciduria type 1 (GA-1). In an aspect, a subject can be male or female. In an aspect, a subject can be an adult, a teenager, an adolescent, a child, a toddler, a baby, or an infant.

In an aspect of a disclosed method, the disclosed subject's GA-1 related and/or associated behavior can be modulated. In an aspect of a disclosed method, the disclosed subject's GA-1 related and/or associated physiology can be modulated.

In an aspect of a disclosed method, administering a disclosed viral vector can be administered systemically or directly. In an aspect, administering a disclosed viral vector molecule can comprise oral administration, intravenous administration, intratumoral administration, intraperitoneal administration, or any combination thereof. In an aspect, administering a disclosed viral vector can be administered by any method of administration disclosed herein. In an aspect, a disclosed viral can be administered via multiple routes either concurrently or sequentially. A skilled clinician can determine the best route of administration for a subject at a given time.

In an aspect of a disclosed method, administering a disclosed viral vector can be targeted to the subject's liver, wherein expression of the nucleic acid sequence generates a functional glutaryl-CoA dehydrogenase.

In an aspect, a disclosed nucleic acid sequence can comprise only the sequence for the functional domains.

In an aspect of a disclosed method, administering a disclosed viral vector can comprise a single dose, or in multiple doses (such as 2, 3, 4, 5, 6, 7, 8, 9 or 10 doses) as needed for the desired therapeutic results. In an aspect, multiple doses can be administered via the same route or via differing routes of administration. In an aspect, a disclosed viral vector can be administered via multiple routes of administration.

In an aspect, a disclosed viral vector can comprise a recombinant AAV vector. In an aspect, a disclosed AAV vector can comprise AAVcc.47. In an aspect, a disclosed AAV vector can comprise AAV8.

In an aspect, a therapeutically effective amount of disclosed vector can comprise a range of about 1×10¹⁰ vg/kg to about 2×10¹⁴·vg/kg. In an aspect, for example, a disclosed vector can be administered at a dose of about 1×10¹¹ to about 8×10¹³ vg/kg or about 1×10¹² to about 8×10¹³ vg/kg. In an aspect, a disclosed vector can be administered at a dose of about 1×10¹³ to about 6×10¹³ vg/kg. In an aspect, a disclosed vector can be administered at a dose of at least about 1×10¹⁰, at least about 5×10¹⁰, at least about 1×10¹¹, at least about 5×10¹¹, at least about 1×10¹², at least about 5×10¹², at least about 1×10¹³, at least about 5×10¹³, or at least about 1×10¹⁴ vg/kg. In an aspect, a disclosed vector can be administered at a dose of no more than about 1×10¹⁰, no more than about 5×10¹⁰, no more than about 1×10¹¹, no more than about 5×10¹¹, no more than about 1×10¹², no more than about 5×10¹², no more than about 1×10¹³, no more than about 5×10¹³, or no more than about 1×10¹⁴ vg/kg. In an aspect, a disclosed vector can be administered at a dose of about 1×10¹² vg/kg. In an aspect, a disclosed vector can be administered at a dose of about 1×10¹¹ vg/kg. In an aspect, a disclosed vector can be administered in a single dose, or in multiple doses (such as 2, 3, 4, 5, 6, 7, 8, 9 or 10 doses) as needed for the desired therapeutic results (such as for example, restoring the expression of GA-1).

In an aspect, a disclosed method can further comprise monitoring the subject for adverse effects following the administering step and/or the treating step. In an aspect, wherein in the absence of adverse effects, the method can further comprise continuing to treat the subject and/or continuing to monitor the subject. In an aspect, wherein in the presence of adverse effects, the method can further comprise modifying one or more steps of the method. In an aspect, modifying can comprise modifying the treating step, modifying the administering step, or both.

In an aspect, modifying the treating step can comprise changing the amount of the vector administered to the subject, changing the frequency of administration of the vector, changing the duration of administration of the vector, changing the route of administration of the vector, or any combination thereof. In an aspect, modifying the administering step can comprise changing the amount of the vector administered to the subject, changing the frequency of administration of the vector, changing the duration of administration of the vector, changing the route of administration of the vector, or any combination thereof.

In an aspect of a disclosed method, a disclosed CRISPR/Cas9 editing system can be applied to one or more amino acids of any enzyme in the lysine catabolism pathways (i.e., the pipecolate pathway and the saccharopine pathway). In an aspect, these enzymes can include, but are not limited to aminoadipate-semialdehyde synthase, α-aminoadipic semialdehyde, the kynurenine aminotransferase 2, the dehydrogenase E1 and transketolase domain-containing protein 1, the L-lysine alpha-oxidase, the ketimine reductase mu-crystallin protein, the peroxisomal sarcosine oxidase, or the pyrroline-5-carboxylate reductase, or any combination thereof. In an aspect, for example, a disclosed CRISPR/Cas9 editing system can be applied to one or more amino acids in a disclosed enzyme comprising the sequence set forth in SEQ ID NO:03 or SEQ ID NO:04 or SEQ ID NO:37 or SEQ ID NO:40.

In an aspect, a disclosed method can further comprise administering to the subject a therapeutically effective amount of a therapeutic agent. Therapeutic agents are known. In an aspect, a disclosed method can further comprise administering to the subject a therapeutically effective amount of one or more immune modulators. In an aspect, the one or more immune modulators comprise methotrexate, rituximab, intravenous gamma globulin, Tacrolimus, prednisolone or a prednisolone analog, SVP-Rapamycin, bortezomib, or a combination thereof.

In an aspect, a disclosed method can further comprise monitoring the subject for adverse effects following the administering of one or more therapeutic agents and/or the administering of one or more immune modulators. In an aspect, wherein in the absence of adverse effects following the administering of one or more therapeutic agents and/or immune modulators, the method can further comprise continuing to treat the subject and/or continuing to monitor the subject. In an aspect, wherein in the presence of adverse effects, the method can further comprise modifying one or more steps of the method. In an aspect, modifying can comprise modifying the administering step of one or more therapeutic agents and/or immune modulators. In an aspect, modifying the administering step can comprise changing the amount of one or more therapeutic agents and/or immune modulators administered to the subject, changing the frequency of administration of one or more therapeutic agents and/or immune modulators, changing the duration of administration of the vector, changing the route of administration of one or more therapeutic agents and/or immune modulators, or any combination thereof.

In an aspect of a disclosed method, a disclosed CRISPR/Cas9 editing system can be applied to one or more amino acids of any enzyme in the lysine catabolism pathways (i.e., the pipecolate pathway and the saccharopine pathway). In an aspect, these enzymes can include, but are not limited to aminoadipate-semialdehyde synthase, α-aminoadipic semialdehyde, the kynurenine aminotransferase 2, the dehydrogenase E1 and transketolase domain-containing protein 1, the L-lysine alpha-oxidase, the ketimine reductase mu-crystallin protein, the peroxisomal sarcosine oxidase, or the pyrroline-5-carboxylate reductase, or any combination thereof. In an aspect, for example, a disclosed CRISPR/Cas9 editing system can be applied to one or more amino acids in a disclosed enzyme comprising the sequence set forth in SEQ ID NO:03 or SEQ ID NO:04 or SEQ ID NO:37 or SEQ ID NO:40.

In an aspect, a disclosed method can further comprise monitoring the subject's metabolic and/or physiologic improvement following the administering and/or treating step and/or following the administering and/or treating steps. In an aspect, a clinician can measure and/or determine the subject's metabolic and/or physiologic status over time to identify one or more improvements and/or one or more diminishments. In an aspect of a disclosed method, a clinician can use the subject's metabolic and/or physiologic status and/or the trend of the subject's metabolic and/or physiological status and/or trend to make a treatment decision and/or to modify an aspect of a disclosed method and/or to continue treating the subject and/or continue to administer a disclosed vector, a disclosed composition, a disclosed therapeutic agent, and/or a disclosed immune modulator, or any combination thereof. In an aspect, metabolic and/or physiologic data can inform the clinician.

In an aspect, techniques to monitor, measure, and/or assess the restoring of the expression of glutaryl-CoA dehydrogenase can comprise qualitative (or subjective) means as well as quantitative (or objective) means. These means are known to the skilled person. In an aspect, a disclosed method can comprise subjecting the subject to one or more invasive or non-invasive diagnostic assessments. Diagnostic assessments are known to the art. In an aspect, a disclosed non-invasive diagnostic assessment can comprise x-rays, computerized tomography (CT) scans, magnetic resonance imaging (MRI) scans, ultrasounds, positron emission tomography (PET) scans, or any combination thereof. In an aspect, a disclosed invasive diagnostic assessment can comprise a tissue biopsy or exploratory surgery.

In an aspect, a disclosed increase and/or a disclosed improvement can comprise a 10%, 20%, 30%, 40%, 50%, 60%, 70%, 80%, 90%, 100%, or any amount of an increase and/or improvement when compared to a control subject (such as, for example, a subject that has not received a disclosed treatment (e.g., administration of a disclosed viral vector)). In an aspect, a disclosed increase and/or a disclosed improvement can comprise a 10-20%, 20-30%, 30-40%, 40-50%, 50-60%, 60-70%, 70-80%, 80-90%, or 90-100% or any amount of an increase and/or improvement when compared to a control subject (such as a subject that has not received a disclosed treatment (e.g., administration of a disclosed viral vector)).

In an aspect, a disclosed decrease and/or a disclosed reduction can comprise a 10%, 20%, 30%, 40%, 50%, 60%, 70%, 80%, 90%, 100%, or any amount of a decrease and/or reduction when compared to a control subject (such as, for example, a subject that has not received a disclosed treatment (e.g., administration of a disclosed viral vector)). In an aspect, a disclosed decrease and/or a disclosed reduction can comprise a 10-20%, 20-30%, 30-40%, 40-50%, 50-60%, 60-70%, 70-80%, 80-90%, or 90-100% or any amount of a decrease and/or reduction when compared to a control subject (such as a subject that has not received a disclosed treatment (e.g., administration of a disclosed viral vector)).

In an aspect, a disclosed method of treating and/or preventing GA-1 disease progression can comprise repeating an administering step one or more times. In an aspect, a disclosed method of restoring the expression of glutaryl-CoA dehydrogenase can comprise repeating a treating step one or more times.

In an aspect, a disclosed method of treating and/or preventing GA-1 disease progression can comprise restoring liver-specific modulation of lysine catabolism, (ii) restoring one or more aspects of lysine homeostasis, (iii) reducing and/or decreasing the level of toxic catabolites in the liver and/or brain of a subject, (iv) restoring the metabolic flux from glutaryl-CoA to crotonyl-CoA, (v) improving motor performance (e.g., strength, gait, balance, coordination, and combinations thereof) of a subject, (vi) improving memory function of a subject, (vii) reduce anxiety in a subject, (viii) reducing and/or preventing neurological sequelae (e.g., neonatal macrocephaly, subdural hematomas, acute retinal hemorrhage, encephalopathy, striatal necrosis, and combinations thereof), (ix) improving and/or reducing and/or eliminating vascular dysfunction in a subject, (x) improving a subject's quality of life, (xi) increasing and/or prolong a subject's life span, (xii) increasing a subject's survivability, or (xiii) any combination thereof.

In an aspect, a disclosed method of treating and/or preventing GA-1 disease progression can comprise restoring expression of glutaryl-CoA dehydrogenase. In an aspect, a disclosed method of treating and/or preventing GA-1 disease progression can comprise improving and/or diminishing and/or ameliorate one or more symptoms associated Glutaric Aciduria Type-1 in a subject. In an aspect, a disclosed method of treating and/or preventing GA-1 can comprise improving and/or diminishing and/or ameliorate one or more pathologies associated with Glutaric Aciduria Type-1 in a subject. In an aspect, a disclosed method of treating and/or preventing GA-1 disease progression can comprise restoring one or more aspects of cellular homeostasis and/or cellular functionality in a subject. In an aspect, a disclosed method of treating and/or preventing GA-1 disease progression can comprise reprogramming a metabolic pathway. In an aspect, a disclosed metabolic pathway can comprise lysine catabolism. In an aspect, a disclosed metabolic pathway can comprise a disclosed pipecolate pathway and/or a disclosed saccharopine pathway.

### C. Methods Employing CRISPR Based Nucleic Acid Molecules

### 1. Methods of Reprogramming a Metabolic Pathway

Disclosed herein is a method of reprogramming a metabolic pathway, the method comprising administering to a subject in need thereof a therapeutically effective amount of a viral vector comprising an isolated nucleic acid molecule comprising a nucleic acid sequence encoding one or more element of a gene editing system, wherein the expression of the nucleic acid molecule disrupts the expression and/or function of the target gene.

Disclosed herein is a method of reprogramming a metabolic pathway, the method comprising administering to a subject in need thereof a therapeutically effective amount of a viral vector comprising a nucleic acid sequence encoding an endonuclease and a sgRNA directed at a target sequence in a target gene of interest, wherein the expression of the nucleic acid molecule disrupts the expression and/or function of the target gene.

Disclosed herein is a method of reprogramming a metabolic pathway, the method comprising administering to a subject in need thereof a therapeutically effective amount of a viral vector comprising the sequence set forth in SEQ ID NO:20 or SEQ ID NO:21.

Disclosed herein is a method of reprogramming a metabolic pathway, the method comprising administering to a subject in need thereof a therapeutically effective amount of a viral vector comprising a nucleic acid sequence encoding a Cas9 endonuclease and a sgRNA directed at a target sequence in the aminoadipate-semialdehyde synthase gene, wherein the expression of the nucleic acid molecule disrupts the expression and/or function of aminoadipate-semialdehyde synthase gene.

Disclosed herein is a method of reprogramming a metabolic pathway, the method comprising administering to a subject in need thereof a therapeutically effective amount of a viral vector comprising a nucleic acid sequence encoding a Cas9 endonuclease and a sgRNA directed at a target sequence in the aminoadipate-semialdehyde synthase gene, wherein the Cas9 endonuclease comprises the sequence of SEQ ID NO:28, and wherein the sgRNA of the first viral vector comprises the sequence set forth in SEQ ID NO:07-SEQ ID NO:09, wherein the expression of the nucleic acid molecule disrupts the expression and/or function of aminoadipate-semialdehyde synthase gene.

Disclosed herein is a method of reprogramming a metabolic pathway, the method comprising administering to a subject in need thereof a therapeutically effective amount of a viral vector comprising the sequence comprising a nucleic acid sequence encoding a Cas9 endonuclease and a sgRNA directed at a target sequence in the glutaryl-CoA dehydrogenase gene, wherein the expression of the nucleic acid molecule disrupts the expression and/or function of the target the glutaryl-CoA dehydrogenase gene.

Disclosed herein is a method of reprogramming a metabolic pathway, the method comprising administering to a subject in need thereof a therapeutically effective amount of a viral vector comprising an isolated nucleic acid molecule comprising a nucleic acid sequence encoding one or more element of a gene editing system, wherein the target gene comprises the aminoadipate-semialdehyde synthase gene, α-aminoadipic semialdehyde gene, the kynurenine aminotransferase 2 gene, the dehydrogenase E1 and transketolase domain-containing protein 1 gene, the L-lysine alpha-oxidase gene, the ketimine reductase mu-crystallin protein gene, the peroxisomal sarcosine oxidase gene, or the pyrroline-5-carboxylate reductase gene, and wherein the expression of the nucleic acid molecule disrupts the expression and/or function of the target gene.

Disclosed herein is a method of reprogramming a metabolic pathway, the method comprising administering to a subject in need thereof a therapeutically effective amount of a viral vector comprising a nucleic acid sequence encoding an endonuclease and a sgRNA directed at a target sequence in a target gene of interest, wherein the target gene comprises the aminoadipate-semialdehyde synthase gene, α-aminoadipic semialdehyde gene, the kynurenine aminotransferase 2 gene, the dehydrogenase E1 and transketolase domain-containing protein 1 gene, the L-lysine alpha-oxidase gene, the ketimine reductase mu-crystallin protein gene, the peroxisomal sarcosine oxidase gene, or the pyrroline-5-carboxylate reductase gene, and wherein the expression of the nucleic acid molecule disrupts the expression and/or function of the target gene.

Disclosed herein is a method of reprogramming a metabolic pathway, the method comprising administering to a subject in need thereof a therapeutically effective amount of a first viral vector comprising an isolated nucleic acid molecule comprising a nucleic acid sequence encoding one or more element of a gene editing system, and administering a therapeutically effective amount of a second viral vector comprising an isolated nucleic acid molecule comprising a nucleic acid sequence encoding one or more element of a gene editing system.

Disclosed herein is a method of reprogramming a metabolic pathway, the method comprising administering to a subject in need thereof a therapeutically effective amount of a first viral vector comprising a nucleic acid sequence encoding an endonuclease and a sgRNA directed at a target sequence in a target gene of interest, and administering a therapeutically effective amount of a second viral vector comprising a nucleic acid sequence encoding an endonuclease and a sgRNA directed at the target gene of interest.

Disclosed herein is a method of reprogramming a metabolic pathway, the method comprising administering to a subject in need thereof a therapeutically effective amount of a viral vector comprising the sequence set forth in SEQ ID NO:20, and administering to the subject a therapeutically effective amount of a viral vector comprising the sequence set forth in SEQ ID NO:21.

Disclosed herein is a method of reprogramming a metabolic pathway, the method comprising administering to a subject in need thereof a therapeutically effective amount of a viral vector comprising a nucleic acid sequence encoding a Cas9 endonuclease and a sgRNA directed at a target sequence in the aminoadipate-semialdehyde synthase gene; and administering to the subject a therapeutically effective amount of a second viral vector comprising a nucleic acid sequence encoding a Cas9 endonuclease and a sgRNA directed at a target sequence in the aminoadipate-semialdehyde synthase gene.

Disclosed herein is a method of reprogramming a metabolic pathway, the method comprising administering to a subject in need thereof a therapeutically effective amount of a viral vector comprising a nucleic acid sequence encoding a Cas9 endonuclease and a sgRNA directed at a target sequence in the aminoadipate-semialdehyde synthase gene; and administering to the subject a therapeutically effective amount of a second viral vector comprising a nucleic acid sequence encoding a Cas9 endonuclease and a sgRNA directed at a target sequence in the aminoadipate-semialdehyde synthase gene, wherein the Cas9 endonuclease of the first viral vector and/or the Cas9 endonuclease of the second viral vector comprises the sequence of SEQ ID NO:28, and wherein the sgRNA of the first viral vector comprises the sequence set forth in SEQ ID NO:09 or SEQ ID NO:10, and wherein the sgRNA of the second viral vector comprises the sequence set forth in SEQ ID NO:07 or SEQ ID NO:08.

In an aspect, a disclosed sgRNA can be directed at any functional domain of a target sequence.

Disclosed herein is a method of reprogramming a metabolic pathway, the method comprising administering to a subject in need thereof a therapeutically effective amount of a viral vector comprising the sequence comprising a nucleic acid sequence encoding a Cas9 endonuclease and a sgRNA directed at a target sequence in the glutaryl-CoA dehydrogenase gene; and administering to a the subject a therapeutically effective amount of a second viral vector comprising the sequence comprising a nucleic acid sequence encoding a Cas9 endonuclease and a sgRNA directed at a target sequence in the glutaryl-CoA dehydrogenase gene.

In an aspect, a disclosed method can restore normal lysine catabolism in the subject's liver.

In an aspect, a disclosed method can further comprise deleting and/or disrupting one or more other catabolic genes. For example, in an aspect, a disclosed method can further comprise deleting and/or disrupting the aminoadipate-semialdehyde synthase gene, α-aminoadipic semialdehyde gene, the kynurenine aminotransferase 2 gene, the dehydrogenase E1 and transketolase domain-containing protein 1 gene, the L-lysine alpha-oxidase gene, the ketimine reductase mu-crystallin protein gene, the peroxisomal sarcosine oxidase gene, or the pyrroline-5-carboxylate reductase gene, or any combination thereof.

In an aspect of a disclosed method, the disclosed subject in need thereof can have or can have been diagnosed with a glutaric aciduria type 1 (GA-1). In an aspect, a subject can be male or female. In an aspect, a subject can be an adult, a teenager, an adolescent, a child, a toddler, a baby, or an infant.

In an aspect of a disclosed method, the disclosed subject's GA-1 related and/or associated behavior can be modulated. In an aspect of a disclosed method, the disclosed subject's GA-1 related and/or associated physiology can be modulated.

In an aspect of a disclosed method, administering a disclosed viral vector can be administered systemically or directly. In an aspect, administering a disclosed viral vector molecule can comprise oral administration, intravenous administration, intratumoral administration, intraperitoneal administration, or any combination thereof. In an aspect, administering a disclosed viral vector can be administered by any method of administration disclosed herein. In an aspect, a disclosed viral can be administered via multiple routes either concurrently or sequentially. A skilled clinician can determine the best route of administration for a subject at a given time.

In an aspect of a disclosed method, administering a disclosed viral vector can be targeted to the subject's liver, wherein expression of the nucleic acid sequence eliminates aminoadipate-semialdehyde synthase.

In an aspect, a disclosed nucleic acid sequence can comprise only the sequence for the functional domains.

In an aspect of a disclosed method, administering a disclosed viral vector can comprise a single dose, or in multiple doses (such as 2, 3, 4, 5, 6, 7, 8, 9 or 10 doses) as needed for the desired therapeutic results. In an aspect, multiple doses can be administered via the same route or via differing routes of administration. In an aspect, a disclosed viral vector can be administered via multiple routes of administration.

In an aspect, the first viral vector and the second viral vectors can be concurrently and/or sequentially administered to the subject. In an aspect, the first viral vector and the second viral vectors can be administered to the subject via the same route of administration and/or via a different route of administration.

In an aspect, a disclosed viral vector can comprise a recombinant AAV vector. In an aspect, a disclosed AAV vector can comprise AAVcc.47. In an aspect, a disclosed AAV vector can comprise AAV8.

In an aspect, a therapeutically effective amount of disclosed viral vector can comprise a range of about 1×10¹⁰ vg/kg to about 2×10¹⁴·vg/kg. In an aspect, for example, a disclosed vector can be administered at a dose of about 1×10¹¹ to about 8×10¹³ vg/kg or about 1×10¹² to about 8×10¹³ vg/kg. In an aspect, a disclosed vector can be administered at a dose of about 1×10¹³ to about 6×10¹³ vg/kg. In an aspect, a disclosed vector can be administered at a dose of at least about 1×10¹⁰, at least about 5×10¹⁰, at least about 1×10¹¹, at least about 5×10¹¹, at least about 1×10¹², at least about 5×10¹², at least about 1×10¹³, at least about 5×10¹³, or at least about 1×10¹⁴ vg/kg. In an aspect, a disclosed vector can be administered at a dose of no more than about 1×10¹⁰, no more than about 5×10¹⁰, no more than about 1×10¹¹, no more than about 5×10¹¹, no more than about 1×10¹², no more than about 5×10¹², no more than about 1×10¹³, no more than about 5×10¹³, or no more than about 1×10¹⁴ vg/kg. In an aspect, a disclosed vector can be administered at a dose of about 1×10¹² vg/kg. In an aspect, a disclosed vector can be administered at a dose of about 1×10¹¹ vg/kg. In an aspect, a disclosed vector can be administered in a single dose, or in multiple doses (such as 2, 3, 4, 5, 6, 7, 8, 9 or 10 doses) as needed for the desired therapeutic results (such as for example, reprogramming a metabolic pathway).

In an aspect, a therapeutically effective amount of disclosed second vector can comprise a range of about 1×10¹⁰ vg/kg to about 2×10¹⁴·vg/kg. In an aspect, for example, a disclosed second vector can be administered at a dose of about 1×10¹¹ to about 8×10¹³ vg/kg or about 1×10¹² to about 8×10¹³ vg/kg. In an aspect, a disclosed second vector can be administered at a dose of about 1×10¹³ to about 6×10¹³ vg/kg. In an aspect, a disclosed second vector can be administered at a dose of at least about 1×10¹⁰, at least about 5×10¹⁰, at least about 1×10¹¹, at least about 5×10¹¹, at least about 1×10¹², at least about 5×10¹², at least about 1×10¹³, at least about 5×10¹³, or at least about 1×10¹⁴ vg/kg. In an aspect, a disclosed second vector can be administered at a dose of no more than about 1×10¹⁰, no more than about 5×10¹⁰, no more than about 1×10¹¹, no more than about 5×10¹¹, no more than about 1×10¹², no more than about 5×10¹², no more than about 1×10¹³, no more than about 5×10¹³, or no more than about 1×10¹⁴ vg/kg. In an aspect, a disclosed second vector can be administered at a dose of about 1×10¹² vg/kg. In an aspect, a disclosed second vector can be administered at a dose of about 1×10¹¹ vg/kg. In an aspect, a disclosed vector can be administered in a single dose, or in multiple doses (such as 2, 3, 4, 5, 6, 7, 8, 9 or 10 doses) as needed for the desired therapeutic results (such as for example, reprogramming a metabolic pathway).

In an aspect, a disclosed method can further comprise monitoring the subject's metabolic and/or physiologic improvement following the administering step and/or following the administering steps. In an aspect, a clinician can measure and/or determine the subject's metabolic and/or physiologic status over time to identify one or more improvements and/or one or more diminishments. In an aspect of a disclosed method, a clinician can use the subject's metabolic and/or physiologic status and/or the trend of the subject's metabolic and/or physiological status and/or trend to make a treatment decision and/or to modify an aspect of a disclosed method and/or to continue treating the subject and/or continue to administer a disclosed vector, a disclosed composition, a disclosed therapeutic agent, and/or a disclosed immune modulator, or any combination thereof. In an aspect, metabolic and/or physiologic data can inform the clinician.

In an aspect, techniques to monitor, measure, and/or assess the reprogramming a metabolic pathway can comprise qualitative (or subjective) means as well as quantitative (or objective) means. These means are known to the skilled person and are discussed supra.

In an aspect, a disclosed method can comprise subjecting the subject to one or more invasive or non-invasive diagnostic assessments. Diagnostic assessments are known to the art. In an aspect, a disclosed non-invasive diagnostic assessment can comprise x-rays, computerized tomography (CT) scans, magnetic resonance imaging (MRI) scans, ultrasounds, positron emission tomography (PET) scans, or any combination thereof. In an aspect, a disclosed invasive diagnostic assessment can comprise a tissue biopsy or exploratory surgery.

In an aspect, a disclosed increase and/or a disclosed improvement can comprise a 10%, 20%, 30%, 40%, 50%, 60%, 70%, 80%, 90%, 100%, or any amount of an increase and/or improvement when compared to a control subject (such as, for example, a subject that has not received a disclosed treatment (e.g., administration of a disclosed viral vector or both disclosed viral vectors)). In an aspect, a disclosed increase and/or a disclosed improvement can comprise a 10-20%, 20-30%, 30-40%, 40-50%, 50-60%, 60-70%, 70-80%, 80-90%, or 90-100% or any amount of an increase and/or improvement when compared to a control subject (such as a subject that has not received a disclosed treatment (e.g., administration of a disclosed viral vector or both disclosed viral vectors)).

In an aspect, a disclosed decrease and/or a disclosed reduction can comprise a 10%, 20%, 30%, 40%, 50%, 60%, 70%, 80%, 90%, 100%, or any amount of a decrease and/or reduction when compared to a control subject (such as, for example, a subject that has not received a disclosed treatment (e.g., administration of a disclosed viral vector or both disclosed viral vectors)). In an aspect, a disclosed decrease and/or a disclosed reduction can comprise a 10-20%, 20-30%, 30-40%, 40-50%, 50-60%, 60-70%, 70-80%, 80-90%, or 90-100% or any amount of a decrease and/or reduction when compared to a control subject (such as a subject that has not received a disclosed treatment (e.g., administration of a disclosed viral vector or both disclosed viral vectors)).

In an aspect, a disclosed method can further comprise monitoring the subject for adverse effects following the administering step and/or following the administering steps. In an aspect, wherein in the absence of adverse effects, the method can further comprise continuing to treat the subject and/or continuing to monitor the subject. In an aspect, wherein in the presence of adverse effects, the method can further comprise modifying one or more steps of the method. In an aspect, modifying can comprise modifying the treating step, modifying the administering step, or both.

In an aspect, modifying the treating step can comprise changing the amount of the first vector and/or the second vector administered to the subject, changing the frequency of administration of the first vector and/or the second vector, changing the duration of administration of the first vector and/or the second vector, changing the route of administration of the first vector and/or the second vector, or any combination thereof. In an aspect, modifying the administering step can comprise changing the amount of the first vector and/or the second vector administered to the subject, changing the frequency of administration of the first vector and/or the second vector, changing the duration of administration of the first vector and/or the second vector, changing the route of administration of the first vector and/or the second vector, or any combination thereof.

In an aspect of a disclosed method, a disclosed CRISPR/Cas9 editing system can be applied to one or more amino acids of any enzyme in the lysine catabolism pathways (i.e., the pipecolate pathway and the saccharopine pathway). In an aspect, these enzymes can include, but are not limited to aminoadipate-semialdehyde synthase, α-aminoadipic semialdehyde, the kynurenine aminotransferase 2, the dehydrogenase E1 and transketolase domain-containing protein 1, the L-lysine alpha-oxidase, the ketimine reductase mu-crystallin protein, the peroxisomal sarcosine oxidase, or the pyrroline-5-carboxylate reductase, or any combination thereof. In an aspect, for example, a disclosed CRISPR/Cas9 editing system can be applied to one or more amino acids in a disclosed enzyme comprising the sequence set forth in SEQ ID NO:03 or SEQ ID NO:04 or SEQ ID NO:37 or SEQ ID NO:40.

In an aspect, a disclosed method can further comprise administering to the subject a therapeutically effective amount of a therapeutic agent. Therapeutic agents are known.

In an aspect, a disclosed method can further comprise administering to the subject a therapeutically effective amount of one or more immune modulators. In an aspect, the one or more immune modulators comprise methotrexate, rituximab, intravenous gamma globulin, Tacrolimus, prednisolone or a prednisolone analog, SVP-Rapamycin, bortezomib, or a combination thereof. Immune modulators are known to the art.

In an aspect, a disclosed method can further comprise monitoring the subject for adverse effects following the administering of one or more therapeutic agents and/or the administering of one or more immune modulators. In an aspect, wherein in the absence of adverse effects following the administering of one or more therapeutic agents and/or immune modulators, the method can further comprise continuing to treat the subject and/or continuing to monitor the subject. In an aspect, wherein in the presence of adverse effects, the method can further comprise modifying one or more steps of the method. In an aspect, modifying can comprise modifying the administering step of one or more therapeutic agents and/or immune modulators. In an aspect, modifying the administering step can comprise changing the amount of one or more therapeutic agents and/or immune modulators administered to the subject, changing the frequency of administration of one or more therapeutic agents and/or immune modulators, changing the duration of administration of the vector, changing the route of administration of one or more therapeutic agents and/or immune modulators, or any combination thereof.

In an aspect of a disclosed method, a disclosed CRISPR/Cas9 editing system can be applied to one or more amino acids of any enzyme in the lysine catabolism pathways (i.e., the pipecolate pathway and the saccharopine pathway). In an aspect, these enzymes can include, but are not limited to aminoadipate-semialdehyde synthase, α-aminoadipic semialdehyde, the kynurenine aminotransferase 2, the dehydrogenase E1 and transketolase domain-containing protein 1, the L-lysine alpha-oxidase, the ketimine reductase mu-crystallin protein, the peroxisomal sarcosine oxidase, or the pyrroline-5-carboxylate reductase, or any combination thereof. In an aspect, for example, a disclosed CRISPR/Cas9 editing system can be applied to one or more amino acids in a disclosed enzyme comprising the sequence set forth in SEQ ID NO:03 or SEQ ID NO:04 or SEQ ID NO:37 or SEQ ID NO:40.

In an aspect, a disclosed method of reprogramming a metabolic pathway can comprise repeating an administering step one or more times. In an aspect, a disclosed method of reprogramming a metabolic pathway can comprise repeating a treating step one or more times. For example, in an aspect, a disclosed method can repeat the administering of a first disclosed vector one or more times, can repeat the administering of a second disclosed vector one or more times, can repeat the administering of one or more therapeutic agents one or more times, can repeat the administering of one or more immune modulators one or more times, or any combination thereof.

In an aspect, a disclosed method of reprogramming a metabolic pathway can comprise restoring liver-specific modulation of lysine catabolism, (ii) restoring one or more aspects of lysine homeostasis, (iii) reducing and/or decreasing the level of toxic catabolites in the liver and/or brain of the subject, (iv) restoring the metabolic flux from glutaryl-CoA to crotonyl-CoA, (v) improving motor performance (e.g., strength, gait, balance, coordination, and combinations thereof) of the subject, (vi) improving memory function of the subject, (vii) reduce anxiety in the subject, (viii) reducing and/or preventing neurological sequelae (e.g., neonatal macrocephaly, subdural hematomas, acute retinal hemorrhage, encephalopathy, striatal necrosis, and combinations thereof), (ix) improving and/or reducing and/or eliminating vascular dysfunction in the subject, (x) improving the subject's quality of life, (xi) increasing and/or prolong the subject's life span, (xii) increasing a subject's survivability, or (xiii) any combination thereof.

In an aspect, a disclosed method of reprogramming a metabolic pathway can comprise treating and/or preventing Glutaric Aciduria Type-1 in a subject. In an aspect, a disclosed method of reprogramming a metabolic pathway can comprise improving and/or diminishing and/or ameliorate one or more symptoms associated Glutaric Aciduria Type-1 in a subject. In an aspect, a disclosed method of reprogramming a metabolic pathway can comprise improving and/or diminishing and/or ameliorate one or more pathologies associated with Glutaric Aciduria Type-1 in a subject. In an aspect, a disclosed method of reprogramming a metabolic pathway can comprise restoring one or more aspects of cellular homeostasis and/or cellular functionality in a subject.

In an aspect, a disclosed metabolic pathway can comprise lysine catabolism. In an aspect, a disclosed metabolic pathway can comprise a disclosed pipecolate pathway and/or a disclosed saccharopine pathway.

2. Methods of Treating and/or Preventing GA-1 Disease Progression

Disclosed herein is a method of treating and/or preventing GA-1 disease progression, the method comprising administering to a subject in need thereof a therapeutically effective amount of a viral vector comprising an isolated nucleic acid molecule comprising a nucleic acid sequence encoding one or more element of a gene editing system, wherein the expression of the nucleic acid molecule disrupts the expression and/or function of the target gene,

Disclosed herein is a method of treating and/or preventing GA-1 disease progression, the method comprising administering to a subject in need thereof a therapeutically effective amount of a first viral vector comprising a nucleic acid sequence encoding an endonuclease and a sgRNA directed at a target sequence in a target gene of interest, wherein the expression of the nucleic acid molecule disrupts the expression and/or function of the target gene

Disclosed herein is a method of treating and/or preventing GA-1 disease progression, the method comprising administering to a subject in need thereof a therapeutically effective amount of a viral vector comprising the sequence set forth in SEQ ID NO:20, wherein the expression of the nucleic acid molecule disrupts the expression and/or function of the target gene. Disclosed herein is a method of treating and/or preventing GA-1 disease progression, the method comprising administering to a subject in need thereof a therapeutically effective amount of a viral vector comprising the sequence set forth in SEQ ID NO:21, wherein the expression of the nucleic acid molecule disrupts the expression and/or function of the target gene.

Disclosed herein is a method of treating and/or preventing GA-1 disease progression, the method comprising administering to a subject in need thereof a therapeutically effective amount of a viral vector comprising a nucleic acid sequence encoding a Cas9 endonuclease and a sgRNA directed at a target sequence in the aminoadipate-semialdehyde synthase gene, wherein the expression of the nucleic acid molecule disrupts the expression and/or function of the target gene.

Disclosed herein is a method of treating and/or preventing GA-1 disease progression, the method comprising administering to a subject in need thereof a therapeutically effective amount of a viral vector comprising a nucleic acid sequence encoding a Cas9 endonuclease and a sgRNA directed at a target sequence in the aminoadipate-semialdehyde synthase gene, wherein the Cas9 endonuclease comprises the sequence of SEQ ID NO:28, and wherein the sgRNA comprises the sequence set forth in any one of SEQ ID NO:06-SEQ ID NO:10, wherein the expression of the nucleic acid molecule disrupts the expression and/or function of aminoadipate-semialdehyde synthase gene.

Disclosed herein is a method of reprogramming a metabolic pathway, the method comprising administering to a subject in need thereof a therapeutically effective amount of a viral vector comprising the sequence comprising a nucleic acid sequence encoding a Cas9 endonuclease and a sgRNA directed at glutaryl-CoA dehydrogenase; wherein the expression of the nucleic acid molecule disrupts the expression and/or function of the glutaryl-CoA dehydrogenase gene.

Disclosed herein is a method of treating and/or preventing GA-1 disease progression, the method comprising administering to a subject in need thereof a therapeutically effective amount of a viral vector comprising an isolated nucleic acid molecule comprising a nucleic acid sequence encoding one or more element of a gene editing system, wherein the expression of the nucleic acid molecule disrupts the expression and/or function of the target gene, wherein the target gene comprises the aminoadipate-semialdehyde synthase gene, α-aminoadipic semialdehyde gene, the kynurenine aminotransferase 2 gene, the dehydrogenase E1 and transketolase domain-containing protein 1 gene, the L-lysine alpha-oxidase gene, the ketimine reductase mu-crystallin protein gene, the peroxisomal sarcosine oxidase gene, or the pyrroline-5-carboxylate reductase gene.

Disclosed herein is a method of treating and/or preventing GA-1 disease progression, the method comprising administering to a subject in need thereof a therapeutically effective amount of a first viral vector comprising a nucleic acid sequence encoding an endonuclease and a sgRNA directed at a target sequence in a target gene of interest, wherein the expression of the nucleic acid molecule disrupts the expression and/or function of the target gene, wherein the target gene comprises the aminoadipate-semialdehyde synthase gene, α-aminoadipic semialdehyde gene, the kynurenine aminotransferase 2 gene, the dehydrogenase E1 and transketolase domain-containing protein 1 gene, the L-lysine alpha-oxidase gene, the ketimine reductase mu-crystallin protein gene, the peroxisomal sarcosine oxidase gene, or the pyrroline-5-carboxylate reductase gene.

Disclosed herein is a method of treating and/or preventing GA-1 disease progression, the method comprising administering to a subject in need thereof a therapeutically effective amount of a first viral vector comprising an isolated nucleic acid molecule comprising a nucleic acid sequence encoding one or more element of a gene editing system, and administering a therapeutically effective amount of a second viral vector comprising an isolated nucleic acid molecule comprising a nucleic acid sequence encoding one or more element of a gene editing system.

Disclosed herein is a method of treating and/or preventing GA-1 disease progression, the method comprising administering to a subject in need thereof a therapeutically effective amount of a first viral vector comprising a nucleic acid sequence encoding an endonuclease and a sgRNA directed at a target sequence in a target gene of interest, and administering a therapeutically effective amount of a second viral vector comprising a nucleic acid sequence encoding an endonuclease and a sgRNA directed at a target sequence in the target gene of interest.

Disclosed herein is a method of treating and/or preventing GA-1 disease progression, the method comprising administering to a subject in need thereof a therapeutically effective amount of a viral vector comprising the sequence set forth in SEQ ID NO:20, and administering to the subject a therapeutically effective amount of a viral vector comprising the sequence set forth in SEQ ID NO:21.

Disclosed herein is a method of treating and/or preventing GA-1 disease progression, the method comprising administering to a subject in need thereof a therapeutically effective amount of a viral vector comprising a nucleic acid sequence encoding a Cas9 endonuclease and a sgRNA directed at a target sequence in the aminoadipate-semialdehyde synthase gene; and administering to the subject a therapeutically effective amount of a second viral vector comprising a nucleic acid sequence encoding a Cas9 endonuclease and a sgRNA directed at a target sequence in the aminoadipate-semialdehyde synthase gene.

Disclosed herein is a method of treating and/or preventing GA-1 disease progression, the method comprising administering to a subject in need thereof a therapeutically effective amount of a viral vector comprising a nucleic acid sequence encoding a Cas9 endonuclease and a sgRNA directed at a target sequence in the aminoadipate-semialdehyde synthase gene; and administering to the subject a therapeutically effective amount of a second viral vector comprising a nucleic acid sequence encoding a Cas9 endonuclease and a sgRNA directed at target sequence in the aminoadipate-semialdehyde synthase gene, wherein the Cas9 endonuclease of the first viral vector and/or the Cas9 endonuclease of the second viral vector comprises the sequence of SEQ ID NO:28, and wherein the sgRNA of the first viral vector comprises the sequence set forth in SEQ ID NO:09 or SEQ ID NO:10, and wherein the sgRNA of the second viral vector comprises the sequence set forth in SEQ ID NO:07 or SEQ ID NO:08. In an aspect, a disclosed sgRNA can be directed at any functional domain of a target sequence.

Disclosed herein is a method of reprogramming a metabolic pathway, the method comprising administering to a subject in need thereof a therapeutically effective amount of a viral vector comprising the sequence comprising a nucleic acid sequence encoding a Cas9 endonuclease and a sgRNA directed at glutaryl-CoA dehydrogenase; and administering to a the subject a therapeutically effective amount of a second viral vector comprising the sequence comprising a nucleic acid sequence encoding a Cas9 endonuclease and a sgRNA directed at a target sequence in the glutaryl-CoA dehydrogenase gene.

In an aspect, a disclosed method can restore normal lysine catabolism in the subject's liver.

In an aspect, a disclosed method can further comprise deleting and/or disrupting one or more other catabolic genes. For example, in an aspect, a disclosed method can further comprise deleting and/or disrupting the aminoadipate-semialdehyde synthase gene, α-aminoadipic semialdehyde gene, the kynurenine aminotransferase 2 gene, the dehydrogenase E1 and transketolase domain-containing protein 1 gene, the L-lysine alpha-oxidase gene, the ketimine reductase mu-crystallin protein gene, the peroxisomal sarcosine oxidase gene, or the pyrroline-5-carboxylate reductase gene, or any combination thereof.

In an aspect of a disclosed method, the disclosed subject in need thereof can have or can have been diagnosed with a glutaric aciduria type 1 (GA-1). In an aspect, a subject can be male or female. In an aspect, a subject can be an adult, a teenager, an adolescent, a child, a toddler, a baby, or an infant.

In an aspect of a disclosed method, the disclosed subject's GA-1 related and/or associated behavior can be modulated. In an aspect of a disclosed method, the disclosed subject's GA-1 related and/or associated physiology can be modulated.

In an aspect of a disclosed method, administering a disclosed viral vector can be administered systemically or directly. In an aspect, administering a disclosed viral vector molecule can comprise oral administration, intravenous administration, intratumoral administration, intraperitoneal administration, or any combination thereof. In an aspect, administering a disclosed viral vector can be administered by any method of administration disclosed herein. In an aspect, a disclosed viral can be administered via multiple routes either concurrently or sequentially. A skilled clinician can determine the best route of administration for a subject at a given time.

In an aspect of a disclosed method, administering a disclosed viral vector can be targeted to the subject's liver, wherein expression of the nucleic acid sequence eliminates aminoadipate-semialdehyde synthase.

In an aspect, a disclosed nucleic acid sequence can comprise only the sequence for the functional domains.

In an aspect of a disclosed method, administering a disclosed viral vector and/or a disclosed viral vector can comprise a single dose, or in multiple doses (such as 2, 3, 4, 5, 6, 7, 8, 9 or 10 doses) as needed for the desired therapeutic results. In an aspect, multiple doses can be administered via the same route or via differing routes of administration. In an aspect, a disclosed viral vector can be administered via multiple routes of administration.

In an aspect, the first viral vector and the second viral vectors can be concurrently and/or sequentially administered to the subject. In an aspect, the first viral vector and the second viral vectors can be administered to the subject via the same route of administration and/or via a different route of administration.

In an aspect, a disclosed viral vector can comprise a recombinant AAV vector. In an aspect, a disclosed AAV vector can comprise AAVcc.47. In an aspect, a disclosed AAV vector can comprise AAV8.

In an aspect, a therapeutically effective amount of a disclosed first vector can comprise a range of about 1×10¹⁰ vg/kg to about 2×10¹⁴·vg/kg. In an aspect, for example, a disclosed first vector can be administered at a dose of about 1×10¹¹ to about 8×10¹³ vg/kg or about 1×10¹² to about 8×10¹³ vg/kg. In an aspect, a disclosed first vector can be administered at a dose of about 1×10¹³ to about 6×10¹³ vg/kg. In an aspect, a disclosed first vector can be administered at a dose of at least about 1×10¹⁰, at least about 5×10¹⁰, at least about 1×10¹¹, at least about 5×10¹¹, at least about 1×10¹², at least about 5×10¹², at least about 1×10¹³, at least about 5×10¹³, or at least about 1×10¹⁴ vg/kg. In an aspect, a disclosed first vector can be administered at a dose of no more than about 1×10¹⁰, no more than about 5×10¹⁰, no more than about 1×10¹¹, no more than about 5×10¹¹, no more than about 1×10¹², no more than about 5×10¹², no more than about 1×10¹³, no more than about 5×10¹³, or no more than about 1×10¹⁴ vg/kg. In an aspect, a disclosed first vector can be administered at a dose of about 1×10¹² vg/kg. In an aspect, a disclosed first vector can be administered at a dose of about 1×10¹¹ vg/kg. In an aspect, a disclosed first vector can be administered in a single dose, or in multiple doses (such as 2, 3, 4, 5, 6, 7, 8, 9 or 10 doses) as needed for the desired therapeutic results (such as for example, reprogramming a metabolic pathway).

In an aspect, a therapeutically effective amount of a disclosed second vector can comprise a range of about 1×10¹⁰ vg/kg to about 2×10¹⁴·vg/kg. In an aspect, for example, a disclosed second vector can be administered at a dose of about 1×10¹¹ to about 8×10¹³ vg/kg or about 1×10¹² to about 8×10¹³ vg/kg. In an aspect, a disclosed second vector can be administered at a dose of about 1×10¹³ to about 6×10¹³ vg/kg. In an aspect, a disclosed second vector can be administered at a dose of at least about 1×10¹⁰, at least about 5×10¹⁰, at least about 1×10¹¹, at least about 5×10¹¹, at least about 1×10¹², at least about 5×10¹², at least about 1×10¹³, at least about 5×10¹³, or at least about 1×10¹⁴ vg/kg. In an aspect, a disclosed second vector can be administered at a dose of no more than about 1×10¹⁰, no more than about 5×10¹⁰, no more than about 1×10¹¹, no more than about 5×10¹¹, no more than about 1×10¹², no more than about 5×10¹², no more than about 1×10¹³, no more than about 5×10¹³, or no more than about 1×10¹⁴ vg/kg. In an aspect, a disclosed second vector can be administered at a dose of about 1×10¹² vg/kg. In an aspect, a disclosed second vector can be administered at a dose of about 1×10¹¹ vg/kg. In an aspect, a disclosed first vector can be administered in a single dose, or in multiple doses (such as 2, 3, 4, 5, 6, 7, 8, 9 or 10 doses) as needed for the desired therapeutic results (such as for example, reprogramming a metabolic pathway).

In an aspect, a disclosed method can further comprise monitoring the subject's metabolic and/or physiologic improvement following the administering step and/or following the administering steps. In an aspect, a clinician can measure and/or determine the subject's metabolic and/or physiologic status over time to identify one or more improvements and/or one or more diminishments. In an aspect of a disclosed method, a clinician can use the subject's metabolic and/or physiologic status and/or the trend of the subject's metabolic and/or physiological status and/or trend to make a treatment decision and/or to modify an aspect of a disclosed method and/or to continue treating the subject and/or continue to administer a disclosed vector, a disclosed composition, a disclosed therapeutic agent, and/or a disclosed immune modulator, or any combination thereof. In an aspect, metabolic and/or physiologic data can inform the clinician.

In an aspect, techniques to monitor, measure, and/or assess the reprogramming a metabolic pathway can comprise qualitative (or subjective) means as well as quantitative (or objective) means. These means are known to the skilled person.

In an aspect, a disclosed method can comprise subjecting the subject to one or more invasive or non-invasive diagnostic assessments. Diagnostic assessments are known to the art. In an aspect, a disclosed non-invasive diagnostic assessment can comprise x-rays, computerized tomography (CT) scans, magnetic resonance imaging (MRI) scans, ultrasounds, positron emission tomography (PET) scans, or any combination thereof. In an aspect, a disclosed invasive diagnostic assessment can comprise a tissue biopsy or exploratory surgery.

In an aspect, a disclosed increase and/or a disclosed improvement can comprise a 10%, 20%, 30%, 40%, 50%, 60%, 70%, 80%, 90%, 100%, or any amount of an increase and/or improvement when compared to a control subject (such as, for example, a subject that has not received a disclosed treatment (e.g., administration of a disclosed viral vector or both disclosed viral vectors)). In an aspect, a disclosed increase and/or a disclosed improvement can comprise a 10-20%, 20-30%, 30-40%, 40-50%, 50-60%, 60-70%, 70-80%, 80-90%, or 90-100% or any amount of an increase and/or improvement when compared to a control subject (such as a subject that has not received a disclosed treatment (e.g., administration of a disclosed viral vector or both disclosed viral vectors)).

In an aspect, a disclosed decrease and/or a disclosed reduction can comprise a 10%, 20%, 30%, 40%, 50%, 60%, 70%, 80%, 90%, 100%, or any amount of a decrease and/or reduction when compared to a control subject (such as, for example, a subject that has not received a disclosed treatment (e.g., administration of a disclosed viral vector or both disclosed viral vectors)). In an aspect, a disclosed decrease and/or a disclosed reduction can comprise a 10-20%, 20-30%, 30-40%, 40-50%, 50-60%, 60-70%, 70-80%, 80-90%, or 90-100% or any amount of a decrease and/or reduction when compared to a control subject (such as a subject that has not received a disclosed treatment (e.g., administration of a disclosed viral vector or both disclosed viral vectors)).

In an aspect, a disclosed method can further comprise monitoring the subject for adverse effects following the administering step and/or following the administering steps. In an aspect, wherein in the absence of adverse effects, the method can further comprise continuing to treat the subject and/or continuing to monitor the subject. In an aspect, wherein in the presence of adverse effects, the method can further comprise modifying one or more steps of the method. In an aspect, modifying can comprise modifying the treating step, modifying the administering step, or both.

In an aspect, modifying the treating step can comprise changing the amount of the first vector and/or the second vector administered to the subject, changing the frequency of administration of the first vector and/or the second vector, changing the duration of administration of the first vector and/or the second vector, changing the route of administration of the first vector and/or the second vector, or any combination thereof. In an aspect, modifying the administering step can comprise changing the amount of the first vector and/or the second vector administered to the subject, changing the frequency of administration of the first vector and/or the second vector, changing the duration of administration of the first vector and/or the second vector, changing the route of administration of the first vector and/or the second vector, or any combination thereof.

In an aspect of a disclosed method, a disclosed CRISPR/Cas9 editing system can be applied to one or more amino acids of any enzyme in the lysine catabolism pathways (i.e., the pipecolate pathway and the saccharopine pathway). In an aspect, these enzymes can include, but are not limited to aminoadipate-semialdehyde synthase, α-aminoadipic semialdehyde, the kynurenine aminotransferase 2, the dehydrogenase E1 and transketolase domain-containing protein 1, the L-lysine alpha-oxidase, the ketimine reductase mu-crystallin protein, the peroxisomal sarcosine oxidase, or the pyrroline-5-carboxylate reductase, or any combination thereof. In an aspect, for example, a disclosed CRISPR/Cas9 editing system can be applied to one or more amino acids in a disclosed enzyme comprising the sequence set forth in SEQ ID NO:03 or SEQ ID NO:04 or SEQ ID NO:37 or SEQ ID NO:40.

In an aspect, a disclosed method can further comprise administering to the subject a therapeutically effective amount of a therapeutic agent. Therapeutic agents are known.

In an aspect, a disclosed method can further comprise administering to the subject a therapeutically effective amount of one or more immune modulators. In an aspect, the one or more immune modulators comprise methotrexate, rituximab, intravenous gamma globulin, Tacrolimus, prednisolone or a prednisolone analog, SVP-Rapamycin, bortezomib, or a combination thereof. Immune modulators are known to the art.

In an aspect, a disclosed method can further comprise monitoring the subject for adverse effects following the administering of one or more therapeutic agents and/or the administering of one or more immune modulators. In an aspect, wherein in the absence of adverse effects following the administering of one or more therapeutic agents and/or immune modulators, the method can further comprise continuing to treat the subject and/or continuing to monitor the subject. In an aspect, wherein in the presence of adverse effects, the method can further comprise modifying one or more steps of the method. In an aspect, modifying can comprise modifying the administering step of one or more therapeutic agents and/or immune modulators. In an aspect, modifying the administering step can comprise changing the amount of one or more therapeutic agents and/or immune modulators administered to the subject, changing the frequency of administration of one or more therapeutic agents and/or immune modulators, changing the duration of administration of the vector, changing the route of administration of one or more therapeutic agents and/or immune modulators, or any combination thereof.

In an aspect of a disclosed method, a disclosed CRISPR/Cas9 editing system can be applied to one or more amino acids of any enzyme in the lysine catabolism pathways (i.e., the pipecolate pathway and the saccharopine pathway). In an aspect, these enzymes can include, but are not limited to aminoadipate-semialdehyde synthase, α-aminoadipic semialdehyde, the kynurenine aminotransferase 2, the dehydrogenase E1 and transketolase domain-containing protein 1, the L-lysine alpha-oxidase, the ketimine reductase mu-crystallin protein, the peroxisomal sarcosine oxidase, or the pyrroline-5-carboxylate reductase, or any combination thereof. In an aspect, for example, a disclosed CRISPR/Cas9 editing system can be applied to one or more amino acids in a disclosed enzyme comprising the sequence set forth in SEQ ID NO:03 or SEQ ID NO:04 or SEQ ID NO:37 or SEQ ID NO:40.

In an aspect, a disclosed method of treating and/or preventing GA-1 disease progression can comprise repeating an administering step one or more times. In an aspect, a disclosed method of reprogramming a metabolic pathway can comprise repeating a treating step one or more times. For example, in an aspect, a disclosed method can repeat the administering of a first disclosed vector one or more times, can repeat the administering of a second disclosed vector one or more times, can repeat the administering of one or more therapeutic agents one or more times, can repeat the administering of one or more immune modulators one or more times, or any combination thereof.

In an aspect, a disclosed method of treating and/or preventing GA-1 disease progression can comprise restoring liver-specific modulation of lysine catabolism, (ii) restoring one or more aspects of lysine homeostasis, (iii) reducing and/or decreasing the level of toxic catabolites in the liver and/or brain of the subject, (iv) restoring the metabolic flux from glutaryl-CoA to crotonyl-CoA, (v) improving motor performance (e.g., strength, gait, balance, coordination, and combinations thereof) of the subject, (vi) improving memory function of the subject, (vii) reduce anxiety in the subject, (viii) reducing and/or preventing neurological sequelae (e.g., neonatal macrocephaly, subdural hematomas, acute retinal hemorrhage, encephalopathy, striatal necrosis, and combinations thereof), (ix) improving and/or reducing and/or eliminating vascular dysfunction in the subject, (x) improving the subject's quality of life, (xi) increasing and/or prolong the subject's life span, (xii) increasing a subject's survivability, or (xiii) any combination thereof.

In an aspect, a disclosed method of treating and/or preventing GA-1 disease progression can comprise restoring liver-specific modulation of lysine catabolism in the subject's liver, (ii) restoring one or more aspects of lysine homeostasis in the subject's liver, (iii) reducing and/or decreasing the level of toxic catabolites in the liver and/or brain of the subject, (iv) restoring the metabolic flux from glutaryl-CoA to crotonyl-CoA in the subject's liver, or (v) any combination thereof.

In an aspect, a disclosed method of treating and/or preventing GA-1 disease progression can comprise reprogramming a metabolic pathway in a subject. In an aspect, a disclosed metabolic pathway can comprise lysine catabolism. In an aspect, a disclosed metabolic pathway can comprise a disclosed pipecolate pathway and/or a disclosed saccharopine pathway. In an aspect, a disclosed method of treating and/or preventing GA-1 disease progression can comprise improving and/or diminishing and/or ameliorate one or more symptoms associated Glutaric Aciduria Type-1 in a subject. In an aspect, a disclosed method of treating and/or preventing GA-1 disease progression can comprise improving and/or diminishing and/or ameliorate one or more pathologies associated with Glutaric Aciduria Type-1 in a subject. In an aspect, a disclosed method of treating and/or preventing GA-1 disease progression pathway can comprise restoring one or more aspects of cellular homeostasis and/or cellular functionality in a subject.

### D. Methods Employing Hepatocyte Transplantation

1. Methods of Treating and/or Preventing GA-1 Disease Progression

Disclosed herein is a method of treating and/or preventing GA-1 disease progression, the method comprising administering to a subject in need thereof a therapeutically effective amount of hepatocytes, wherein the hepatocytes are GCDH^{+/+}/AASS^{+/+}, and wherein one or more aspects of metabolic function is restored.

Disclosed herein is a method of treating and/or preventing GA-1 disease progression, the method comprising restoring one or more aspects of metabolic function by administering to a subject in need thereof a therapeutically effective amount of hepatocytes, wherein the hepatocytes are GCDH^{+/+}/AASS^{+/+}.

Disclosed herein is a method of treating and/or preventing GA-1 disease progression, the method comprising restoring one or more aspects of lysine metabolism by administering to a subject in need thereof a therapeutically effective amount of hepatocytes, wherein the hepatocytes are GCDH^{+/+}/AASS^{+/+}.

In an aspect, a disclosed method can restore normal lysine catabolism in the subject's liver.

In an aspect, a disclosed method can further comprise deleting and/or disrupting one or more other catabolic genes.

In an aspect, a disclosed method can further comprise deleting and/or disrupting the aminoadipate-semialdehyde synthase gene, α-aminoadipic semialdehyde gene, the kynurenine aminotransferase 2 gene, the dehydrogenase E1 and transketolase domain-containing protein 1 gene, the L-lysine alpha-oxidase gene, the ketimine reductase mu-crystallin protein gene, the peroxisomal sarcosine oxidase gene, or the pyrroline-5-carboxylate reductase gene, or any combination thereof.

In an aspect of a disclosed method, the disclosed subject in need thereof can have or can have been diagnosed with a glutaric aciduria type 1 (GA-1). In an aspect, a subject can be male or female. In an aspect, a subject can be an adult, a teenager, an adolescent, a child, a toddler, a baby, or an infant.

In an aspect of a disclosed method, the disclosed subject's GA-1 related and/or associated behavior can be modulated. In an aspect of a disclosed method, the disclosed subject's GA-1 related and/or associated physiology can be modulated.

In an aspect of a disclosed method, administering a disclosed therapeutically effective amount of hepatocytes can be administered systemically or directly. In an aspect, administering a disclosed therapeutically effective amount of hepatocytes can comprise oral administration, intravenous administration, intratumoral administration, intraperitoneal administration, or any combination thereof. In an aspect, administering a disclosed therapeutically effective amount of hepatocytes can be administered by any method of administration disclosed herein. In an aspect, a disclosed therapeutically effective amount of hepatocytes can be administered via multiple routes either concurrently or sequentially. A disclosed therapeutically effective amount of hepatocytes can be administered directly into the subject's spleen and/or directly into the subject's liver. A skilled clinician can determine the best route of administration for a subject at a given time. In an aspect of a disclosed method, administering a disclosed therapeutically effective amount of hepatocytes can comprise a single dose, or in multiple doses (such as 2, 3, 4, 5, 6, 7, 8, 9 or 10 doses) as needed for the desired therapeutic results. In an aspect, multiple doses can be administered via the same route or via differing routes of administration. In an aspect, a disclosed therapeutically effective amount of hepatocytes can be administered via multiple routes of administration.

In an aspect, a disclosed nucleic acid sequence can comprise only the sequence for the functional domains.

In an aspect, a disclosed therapeutically effective amount of hepatocytes can comprise about 10×10⁷ to about 10×10¹⁰ hepatocytes.

In an aspect, a disclosed therapeutically effective amount can be infused through a portal-vein catheter. In an aspect, a disclosed infusion can occur over time. In an aspect, for example, a disclosed infusion time can comprise about 5 hours to about 25 hours, or about 5 hours to about 20 hours, or about 5 hours to about 15 hours, or about 5 hours to about 10 hours. In an aspect, a disclosed infusion time can comprise 1, 2, 3, 4, 5, 6, 7, 8, 9, 10, 11, 12, 13, 14, 15, 16, 17, 18, 19, 20, 21, 22, 23, 24, 25, or more hours.

In an aspect, a disclosed method can further comprise monitoring the subject's metabolic and/or physiologic improvement following the administering step and/or following the administering steps. In an aspect, a clinician can measure and/or determine the subject's metabolic and/or physiologic status over time to identify one or more improvements and/or one or more diminishments. In an aspect of a disclosed method, a clinician can use the subject's metabolic and/or physiologic status and/or the trend of the subject's metabolic and/or physiological status and/or trend to make a treatment decision and/or to modify an aspect of a disclosed method and/or to continue treating the subject and/or continue to administer disclosed hepatocytes, a disclosed vector, a disclosed composition, a disclosed therapeutic agent, and/or a disclosed immune modulator, or any combination thereof. In an aspect, metabolic and/or physiologic data can inform the clinician.

In an aspect, techniques to monitor, measure, and/or assess the reprogramming a metabolic pathway can comprise qualitative (or subjective) means as well as quantitative (or objective) means. These means are known to the skilled person and are discussed supra.

In an aspect, a disclosed method can comprise subjecting the subject to one or more invasive or non-invasive diagnostic assessments. Diagnostic assessments are known to the art. In an aspect, a disclosed non-invasive diagnostic assessment can comprise x-rays, computerized tomography (CT) scans, magnetic resonance imaging (MRI) scans, ultrasounds, positron emission tomography (PET) scans, or any combination thereof. In an aspect, a disclosed invasive diagnostic assessment can comprise a tissue biopsy or exploratory surgery.

In an aspect, a disclosed increase and/or a disclosed improvement can comprise a 10%, 20%, 30%, 40%, 50%, 60%, 70%, 80%, 90%, 100%, or any amount of an increase and/or improvement when compared to a control subject (such as, for example, a subject that has not received a disclosed treatment (e.g., administration of a disclosed viral vector or both disclosed viral vectors)). In an aspect, a disclosed increase and/or a disclosed improvement can comprise a 10-20%, 20-30%, 30-40%, 40-50%, 50-60%, 60-70%, 70-80%, 80-90%, or 90-100% or any amount of an increase and/or improvement when compared to a control subject (such as a subject that has not received a disclosed treatment (e.g., administration of a disclosed viral vector or both disclosed viral vectors)).

In an aspect, a disclosed decrease and/or a disclosed reduction can comprise a 10%, 20%, 30%, 40%, 50%, 60%, 70%, 80%, 90%, 100%, or any amount of a decrease and/or reduction when compared to a control subject (such as, for example, a subject that has not received a disclosed treatment (e.g., administration of a disclosed therapeutically effective amount of hepatocytes)). In an aspect, a disclosed decrease and/or a disclosed reduction can comprise a 10-20%, 20-30%, 30-40%, 40-50%, 50-60%, 60-70%, 70-80%, 80-90%, or 90-100% or any amount of a decrease and/or reduction when compared to a control subject (such as a subject that has not received a disclosed treatment (e.g., administration of a disclosed therapeutically effective amount of hepatocytes)).

In an aspect, a disclosed method can further comprise monitoring the subject for adverse effects following the administering step and/or following the administering steps. In an aspect, wherein in the absence of adverse effects, the method can further comprise continuing to treat the subject and/or continuing to monitor the subject. In an aspect, wherein in the presence of adverse effects, the method can further comprise modifying one or more steps of the method. In an aspect, modifying can comprise modifying the treating step, modifying the administering step, or both.

In an aspect, modifying the treating step can comprise changing the amount of hepatocytes administered to the subject, changing the frequency of administration of hepatocytes, changing the duration of administration of hepatocytes, changing the route of administration of hepatocytes, or any combination thereof.

In an aspect of a disclosed method, a disclosed CRISPR/Cas9 editing system can be applied to one or more amino acids of any enzyme in the lysine catabolism pathways (i.e., the pipecolate pathway and the saccharopine pathway). In an aspect, these enzymes can include, but are not limited to aminoadipate-semialdehyde synthase, α-aminoadipic semialdehyde, the kynurenine aminotransferase 2, the dehydrogenase E1 and transketolase domain-containing protein 1, the L-lysine alpha-oxidase, the ketimine reductase mu-crystallin protein, the peroxisomal sarcosine oxidase, or the pyrroline-5-carboxylate reductase, or any combination thereof. In an aspect, for example, a disclosed CRISPR/Cas9 editing system can be applied to one or more amino acids in a disclosed enzyme comprising the sequence set forth in SEQ ID NO:03 or SEQ ID NO:04 or SEQ ID NO:37 or SEQ ID NO:40.

In an aspect, a disclosed method can further comprise administering to the subject a therapeutically effective amount of a therapeutic agent. Therapeutic agents are known.

In an aspect, a disclosed method can further comprise administering to the subject a therapeutically effective amount of one or more immune modulators. In an aspect, the one or more immune modulators comprise methotrexate, rituximab, intravenous gamma globulin, Tacrolimus, prednisolone or a prednisolone analog, SVP-Rapamycin, bortezomib, or a combination thereof. Immune modulators are known to the art.

In an aspect, a disclosed method can further comprise monitoring the subject for adverse effects following the administering of one or more therapeutic agents and/or the administering of one or more immune modulators. In an aspect, wherein in the absence of adverse effects following the administering of one or more therapeutic agents and/or immune modulators, the method can further comprise continuing to treat the subject and/or continuing to monitor the subject. In an aspect, wherein in the presence of adverse effects, the method can further comprise modifying one or more steps of the method. In an aspect, modifying can comprise modifying the administering step of one or more therapeutic agents and/or immune modulators. In an aspect, modifying the administering step can comprise changing the amount of one or more therapeutic agents and/or immune modulators administered to the subject, changing the frequency of administration of one or more therapeutic agents and/or immune modulators, changing the duration of administration of the vector, changing the route of administration of one or more therapeutic agents and/or immune modulators, or any combination thereof.

In an aspect of a disclosed method, a disclosed CRISPR/Cas9 editing system can be applied to one or more amino acids of any enzyme in the lysine catabolism pathways (i.e., the pipecolate pathway and the saccharopine pathway). In an aspect, these enzymes can include, but are not limited to aminoadipate-semialdehyde synthase, α-aminoadipic semialdehyde, the kynurenine aminotransferase 2, the dehydrogenase E1 and transketolase domain-containing protein 1, the L-lysine alpha-oxidase, the ketimine reductase mu-crystallin protein, the peroxisomal sarcosine oxidase, or the pyrroline-5-carboxylate reductase, or any combination thereof. In an aspect, for example, a disclosed CRISPR/Cas9 editing system can be applied to one or more amino acids in a disclosed enzyme comprising the sequence set forth in SEQ ID NO:03 or SEQ ID NO:04 or SEQ ID NO:37 or SEQ ID NO:40.

In an aspect, a disclosed method of treating and/or preventing GA-1 disease progression can comprise repeating an administering step one or more times. In an aspect, a disclosed method of reprogramming a metabolic pathway can comprise repeating a treating step one or more times. For example, in an aspect, a disclosed method can repeat the administering of a disclosed therapeutically effective amount of hepatocytes one or more times, can repeat the administering of one or more therapeutic agents one or more times, can repeat the administering of one or more immune modulators one or more times, or any combination thereof.

In an aspect, a disclosed method of treating and/or preventing GA-1 disease progression can comprise restoring liver-specific modulation of lysine catabolism, (ii) restoring one or more aspects of lysine homeostasis, (iii) reducing and/or decreasing the level of toxic catabolites in the liver and/or brain of the subject, (iv) restoring the metabolic flux from glutaryl-CoA to crotonyl-CoA, (v) improving motor performance (e.g., strength, gait, balance, coordination, and combinations thereof) of the subject, (vi) improving memory function of the subject, (vii) reduce anxiety in the subject, (viii) reducing and/or preventing neurological sequelae (e.g., neonatal macrocephaly, subdural hematomas, acute retinal hemorrhage, encephalopathy, striatal necrosis, and combinations thereof), (ix) improving and/or reducing and/or eliminating vascular dysfunction in the subject, (x) improving the subject's quality of life, (xi) increasing and/or prolong the subject's life span, (xii) increasing a subject's survivability, or (xiii) any combination thereof.

In an aspect, a disclosed method of treating and/or preventing GA-1 disease progression can comprise restoring liver-specific modulation of lysine catabolism in the subject's liver, (ii) restoring one or more aspects of lysine homeostasis in the subject's liver, (iii) reducing and/or decreasing the level of toxic catabolites in the liver and/or brain of the subject, (iv) restoring the metabolic flux from glutaryl-CoA to crotonyl-CoA in the subject's liver, or (v) any combination thereof.

In an aspect, a disclosed method of treating and/or preventing GA-1 disease progression can comprise reprogramming a metabolic pathway in a subject. In an aspect, a disclosed metabolic pathway can comprise lysine catabolism. In an aspect, a disclosed metabolic pathway can comprise a disclosed pipecolate pathway and/or a disclosed saccharopine pathway.

In an aspect, a disclosed method of treating and/or preventing GA-1 disease progression can comprise improving and/or diminishing and/or ameliorate one or more symptoms associated Glutaric Aciduria Type-1 in a subject.

In an aspect, a disclosed method of treating and/or preventing GA-1 disease progression pathway can comprise restoring one or more aspects of cellular homeostasis and/or cellular functionality in a subject. In an aspect, a disclosed method of treating and/or preventing GA-1 disease progression, the method can further comprise administering a disclosed vector.

For example, in an aspect, a disclosed method of treating and/or preventing GA-1 disease progression can further comprise administering to the subject (i) a disclosed vector viral vector comprising a nucleic acid sequence encoding glutaryl-CoA dehydrogenase; (ii) a disclosed vector viral vector comprising the nucleic acid sequence set forth in SEQ ID NO:01 or SEQ ID NO:02; (iii) a disclosed vector viral vector comprising the nucleic acid sequence set forth in SEQ ID NO:17 or SEQ ID NO:18; (iv) a disclosed vector viral vector comprising the sequence set forth in SEQ ID NO:19; (v) a disclosed first viral vector comprising an isolated nucleic acid molecule comprising a nucleic acid sequence encoding one or more element of a gene editing system with a second disclosed viral vector comprising an isolated nucleic acid molecule comprising a nucleic acid sequence encoding one or more element of a gene editing system; (vi) first disclosed viral vector comprising a nucleic acid sequence encoding an endonuclease and a sgRNA directed at a target sequence in a target gene of interest and a second disclosed viral vector comprising a nucleic acid sequence encoding an endonuclease and a sgRNA directed at a target sequence in the target gene of interest; (vii) a disclosed viral vector comprising the sequence set forth in SEQ ID NO:20, and a disclosed viral vector comprising the sequence set forth in SEQ ID NO:21; (viii) a disclosed first viral vector comprising a nucleic acid sequence encoding a Cas9 endonuclease and a sgRNA directed at a target sequence in the aminoadipate-semialdehyde synthase gene and a second disclosed viral vector comprising a nucleic acid sequence encoding a Cas9 endonuclease and a sgRNA directed at a target sequence in the aminoadipate-semialdehyde synthase gene; (ix) a first disclosed viral vector comprising a nucleic acid sequence encoding a Cas9 endonuclease and a sgRNA directed at a target sequence in the aminoadipate-semialdehyde synthase gene and a second disclosed viral vector comprising a nucleic acid sequence encoding a Cas9 endonuclease and a sgRNA directed a target sequence in the aminoadipate-semialdehyde synthase gene, wherein the Cas9 endonuclease of the first viral vector and/or the Cas9 endonuclease of the second viral vector comprises the sequence of SEQ ID NO:28, and wherein the sgRNA of the first viral vector comprises the sequence set forth in SEQ ID NO:09 or SEQ ID NO:10, and wherein the sgRNA of the second viral vector comprises the sequence set forth in SEQ ID NO:07 or SEQ ID NO:08; (x) a disclosed first viral vector comprising the sequence comprising a nucleic acid sequence encoding a Cas9 endonuclease and a sgRNA directed at a target sequence in the glutaryl-CoA dehydrogenase gene and a disclosed second viral vector comprising the sequence comprising a nucleic acid sequence encoding a Cas9 endonuclease and a sgRNA directed at a target sequence in the glutaryl-CoA dehydrogenase gene; (xi) a disclosed viral vector comprising an isolated nucleic acid molecule comprising a nucleic acid sequence encoding one or more element of a gene editing system; or (xii) any combination thereof.

E. Methods Employing siRNA or mRNA Therapy

### 1. Methods of Reprogramming a Metabolic Pathway

Disclosed herein is a method of reprogramming a metabolic pathway, the method comprising administering to a subject in need thereof a therapeutically effective amount of a disclosed siRNA or a disclosed formulation comprising a disclosed siRNA.

Disclosed herein is a method of reprogramming a metabolic pathway, the method comprising administering to a subject in need thereof a therapeutically effective amount of a disclosed mRNA therapy or a disclosed formulation comprising a disclosed mRNA therapy.

Disclosed herein is a method of reprogramming a metabolic pathway, the method comprising administering to a subject in need thereof a therapeutically effective amount of siRNA targeting aminoadipate-semialdehyde synthase.

Disclosed herein is a method of reprogramming a metabolic pathway, the method comprising administering to a subject in need thereof a therapeutically effective amount of mRNA therapy targeting an aspect of the lysine catabolism pathway.

Disclosed herein is a method of reprogramming a metabolic pathway, the method comprising administering to a subject in need thereof a therapeutically effective amount of mRNA therapy targeting an aspect of the lysine catabolism pathway, wherein the aspect of the lysine catabolism pathway comprises the aminoadipate-semialdehyde synthase gene, α-aminoadipic semialdehyde gene, the kynurenine aminotransferase 2 gene, the dehydrogenase E1 and transketolase domain-containing protein 1 gene, the L-lysine alpha-oxidase gene, the ketimine reductase mu-crystallin protein gene, the peroxisomal sarcosine oxidase gene, or the pyrroline-5-carboxylate reductase gene, and wherein the expression of the nucleic acid molecule disrupts the expression and/or function of the target gene.

In an aspect, a disclosed siRNA can target or can be directed at one or more enzymes in the pipecolate pathway, the saccharopine pathway, or both. In an aspect, a disclosed siRNA can target or can be directed at the aminoadipate-semialdehyde synthase gene, α-aminoadipic semialdehyde gene, the kynurenine aminotransferase 2 gene, the dehydrogenase E1 and transketolase domain-containing protein 1 gene, the L-lysine alpha-oxidase gene, the ketimine reductase mu-crystallin protein gene, the peroxisomal sarcosine oxidase gene, or the pyrroline-5-carboxylate reductase gene, or any combination thereof. In an aspect, a disclosed siRNA can target a sequence in SEQ ID NO:35 or SEQ ID NO:36. In an aspect, a disclosed siRNA can target a sequence in SEQ ID NO:38 or SEQ ID NO:39. In an aspect, a disclosed siRNA can comprise the sequence set forth in SEQ ID NO:33 or SEQ ID NO:34.

In an aspect, a disclosed mRNA therapy can target or can be directed one or more enzymes in the pipecolate pathway, the saccharopine pathway, or both. In an aspect, a disclosed mRNA therapy can target or can be directed at the aminoadipate-semialdehyde synthase gene, α-aminoadipic semialdehyde gene, the kynurenine aminotransferase 2 gene, the dehydrogenase E1 and transketolase domain-containing protein 1 gene, the L-lysine alpha-oxidase gene, the ketimine reductase mu-crystallin protein gene, the peroxisomal sarcosine oxidase gene, or the pyrroline-5-carboxylate reductase gene, or any combination thereof.

In an aspect, the disclosed siRNA and/or the disclosed mRNA therapy can be encapsulated in lipid nanoparticles. Lipid nanoparticles are known to the skilled person. In an aspect, a disclosed lipid nanoparticle can comprise a commercially available formulation such as, for example, In vivo lipofectamine. In an aspect, the disclosed siRNA can be conjugated to GalNAc.

In an aspect of a disclosed method, the disclosed subject in need thereof can have or can have been diagnosed with a glutaric aciduria type 1 (GA-1). In an aspect, a subject can be male or female. In an aspect, a subject can be an adult, a teenager, an adolescent, a child, a toddler, a baby, or an infant.

In an aspect, a disclosed method using siRNA and/or mRNA therapy can restore normal lysine catabolism in the subject's liver.

In an aspect, a disclosed method can further comprise deleting and/or disrupting one or more other catabolic genes.

In an aspect, a disclosed method can further comprise deleting and/or disrupting the aminoadipate-semialdehyde synthase gene, α-aminoadipic semialdehyde gene, the kynurenine aminotransferase 2 gene, the dehydrogenase E1 and transketolase domain-containing protein 1 gene, the L-lysine alpha-oxidase gene, the ketimine reductase mu-crystallin protein gene, the peroxisomal sarcosine oxidase gene, or the pyrroline-5-carboxylate reductase gene, or any combination thereof.

In an aspect of a disclosed method, the disclosed subject's GA-1 related and/or associated behavior can be modulated. In an aspect of a disclosed method, the disclosed subject's GA-1 related and/or associated physiology can be modulated.

In an aspect of a disclosed method, administering a disclosed siRNA, a disclosed formulation comprising a disclosed siRNA, a disclosed mRNA therapy, or a disclosed formulation comprising a disclosed mRNA therapy, or any combination thereof can be administered systemically or directly. In an aspect, administering a disclosed siRNA, a disclosed formulation comprising a disclosed siRNA, a disclosed mRNA therapy, or a disclosed formulation comprising a disclosed mRNA therapy, or any combination thereof can comprise oral administration, intravenous administration, intratumoral administration, intraperitoneal administration, or any combination thereof. In an aspect, administering a disclosed siRNA, a disclosed formulation comprising a disclosed siRNA, a disclosed mRNA therapy, or a disclosed formulation comprising a disclosed mRNA therapy, or any combination thereof can be administered by any method of administration disclosed herein. In an aspect, a disclosed siRNA, a disclosed formulation comprising a disclosed siRNA, a disclosed mRNA therapy, or a disclosed formulation comprising a disclosed mRNA therapy, or any combination thereof can be administered via multiple routes either concurrently or sequentially. A skilled clinician can determine the best route of administration for a subject at a given time.

In an aspect of a disclosed method, administering a disclosed siRNA, a disclosed formulation comprising a disclosed siRNA, a disclosed mRNA therapy, or a disclosed formulation comprising a disclosed mRNA therapy, or any combination thereof can comprise a single dose, or in multiple doses (such as 2, 3, 4, 5, 6, 7, 8, 9 or 10 doses) as needed for the desired therapeutic results. In an aspect, multiple doses can be administered via the same route or via differing routes of administration. In an aspect, a disclosed siRNA, a disclosed formulation comprising a disclosed siRNA, a disclosed mRNA therapy, or a disclosed formulation comprising a disclosed mRNA therapy, or any combination thereof can be administered via multiple routes of administration.

In an aspect, a therapeutically effective amount of siRNA can comprise about 0.01 mg/kg to about 100 mg/kg, or about 0.5 mg/kg to about 75 mg/kg, or about 0.1 mg/kg to about 50 mg/kg, or any amount in that range. In an aspect, a therapeutically effective amount of siRNA can comprise about 0.2 mg/kg to about 50 mg/kg.

In an aspect, a therapeutically effective amount of mRNA therapy can comprise about 0.001 mg/kg to about 100 mg/kg, or about 0.050 mg/kg to about 75 mg/kg, or about 0.01 mg/kg to about 50 mg/kg, or any amount in that range. In an aspect, a therapeutically effective amount of siRNA can comprise about 0.01 mg/kg to about 50 mg/kg.

In an aspect, a disclosed method can further comprise monitoring the subject's metabolic and/or physiologic improvement following the administering step and/or following the administering steps. In an aspect, a clinician can measure and/or determine the subject's metabolic and/or physiologic status over time to identify one or more improvements and/or one or more diminishments. In an aspect of a disclosed method, a clinician can use the subject's metabolic and/or physiologic status and/or the trend of the subject's metabolic and/or physiological status and/or trend to make a treatment decision and/or to modify an aspect of a disclosed method and/or to continue treating the subject and/or continue to administer a disclosed vector, a disclosed composition, a disclosed therapeutic agent, and/or a disclosed immune modulator, or any combination thereof. In an aspect, metabolic and/or physiologic data can inform the clinician.

In an aspect, techniques to monitor, measure, and/or assess the reprogramming a metabolic pathway can comprise qualitative (or subjective) means as well as quantitative (or objective) means. These means are known to the skilled person and are discussed supra.

In an aspect, a disclosed method can comprise subjecting the subject to one or more invasive or non-invasive diagnostic assessments. Diagnostic assessments are known to the art. In an aspect, a disclosed non-invasive diagnostic assessment can comprise x-rays, computerized tomography (CT) scans, magnetic resonance imaging (MRI) scans, ultrasounds, positron emission tomography (PET) scans, or any combination thereof. In an aspect, a disclosed invasive diagnostic assessment can comprise a tissue biopsy or exploratory surgery.

In an aspect, a disclosed increase and/or a disclosed improvement can comprise a 10%, 20%, 30%, 40%, 50%, 60%, 70%, 80%, 90%, 100%, or any amount of an increase and/or improvement when compared to a control subject (such as, for example, a subject that has not received a disclosed treatment (e.g., administration of a disclosed siRNA, a disclosed formulation comprising a disclosed siRNA, a disclosed mRNA therapy, or a disclosed formulation comprising a disclosed mRNA therapy, or any combination thereof)). In an aspect, a disclosed increase and/or a disclosed improvement can comprise a 10-20%, 20-30%, 30-40%, 40-50%, 50-60%, 60-70%, 70-80%, 80-90%, or 90-100% or any amount of an increase and/or improvement when compared to a control subject (such as a subject that has not received a disclosed treatment (e.g., administration of a disclosed siRNA, a disclosed formulation comprising a disclosed siRNA, a disclosed mRNA therapy, or a disclosed formulation comprising a disclosed mRNA therapy, or any combination thereof)).

In an aspect, a disclosed decrease and/or a disclosed reduction can comprise a 10%, 20%, 30%, 40%, 50%, 60%, 70%, 80%, 90%, 100%, or any amount of a decrease and/or reduction when compared to a control subject (such as, for example, a subject that has not received a disclosed treatment (e.g., administration a disclosed siRNA, a disclosed formulation comprising a disclosed siRNA, a disclosed mRNA therapy, or a disclosed formulation comprising a disclosed mRNA therapy, or any combination thereof)). In an aspect, a disclosed decrease and/or a disclosed reduction can comprise a 10-20%, 20-30%, 30-40%, 40-50%, 50-60%, 60-70%, 70-80%, 80-90%, or 90-100% or any amount of a decrease and/or reduction when compared to a control subject (such as a subject that has not received a disclosed treatment (e.g., administration of a disclosed siRNA, a disclosed formulation comprising a disclosed siRNA, a disclosed mRNA therapy, or a disclosed formulation comprising a disclosed mRNA therapy, or any combination thereof)).

In an aspect, a disclosed method can further comprise monitoring the subject for adverse effects following the administering step and/or following the administering steps. In an aspect, wherein in the absence of adverse effects, the method can further comprise continuing to treat the subject and/or continuing to monitor the subject. In an aspect, wherein in the presence of adverse effects, the method can further comprise modifying one or more steps of the method. In an aspect, modifying can comprise modifying the treating step, modifying the administering step, or both.

In an aspect, modifying the treating step can comprise changing the amount of a disclosed siRNA, a disclosed formulation comprising a disclosed siRNA, a disclosed mRNA therapy, or a disclosed formulation comprising a disclosed mRNA therapy, or any combination thereof to the subject, changing the frequency of administration of a disclosed siRNA, a disclosed formulation comprising a disclosed siRNA, a disclosed mRNA therapy, or a disclosed formulation comprising a disclosed mRNA therapy, or any combination thereof, changing the duration of administration of a disclosed siRNA, a disclosed formulation comprising a disclosed siRNA, a disclosed mRNA therapy, or a disclosed formulation comprising a disclosed mRNA therapy, or any combination thereof, changing the route of administration of a disclosed siRNA, a disclosed formulation comprising a disclosed siRNA, a disclosed mRNA therapy, or a disclosed formulation comprising a disclosed mRNA therapy, or any combination thereof, or any combination thereof.

In an aspect, modifying the administering step can comprise changing the amount of a disclosed siRNA, a disclosed formulation comprising a disclosed siRNA, a disclosed mRNA therapy, or a disclosed formulation comprising a disclosed mRNA therapy, or any combination thereof administered to the subject, changing the frequency of administration of a disclosed siRNA, a disclosed formulation comprising a disclosed siRNA, a disclosed mRNA therapy, or a disclosed formulation comprising a disclosed mRNA therapy, or any combination thereof, changing the duration of administration of a disclosed siRNA, a disclosed formulation comprising a disclosed siRNA, a disclosed mRNA therapy, or a disclosed formulation comprising a disclosed mRNA therapy, or any combination thereof, changing the route of administration of a disclosed siRNA, a disclosed formulation comprising a disclosed siRNA, a disclosed mRNA therapy, or a disclosed formulation comprising a disclosed mRNA therapy, or any combination thereof, or any combination thereof.

In an aspect of a disclosed method, a disclosed CRISPR/Cas9 editing system can be applied to one or more amino acids of any enzyme in the lysine catabolism pathways (i.e., the pipecolate pathway and the saccharopine pathway). In an aspect, these enzymes can include, but are not limited to aminoadipate-semialdehyde synthase, α-aminoadipic semialdehyde, the kynurenine aminotransferase 2, the dehydrogenase E1 and transketolase domain-containing protein 1, the L-lysine alpha-oxidase, the ketimine reductase mu-crystallin protein, the peroxisomal sarcosine oxidase, or the pyrroline-5-carboxylate reductase, or any combination thereof. In an aspect, for example, a disclosed CRISPR/Cas9 editing system can be applied to one or more amino acids in a disclosed enzyme comprising the sequence set forth in SEQ ID NO:03 or SEQ ID NO:04 or SEQ ID NO:37 or SEQ ID NO:40.

In an aspect, a disclosed method can further comprise administering to the subject a therapeutically effective amount of a therapeutic agent. Therapeutic agents are known.

In an aspect, a disclosed method can further comprise administering to the subject a therapeutically effective amount of one or more immune modulators. In an aspect, the one or more immune modulators comprise methotrexate, rituximab, intravenous gamma globulin, Tacrolimus, prednisolone or a prednisolone analog, SVP-Rapamycin, bortezomib, or a combination thereof. Immune modulators are known to the art.

In an aspect, a disclosed method can further comprise monitoring the subject for adverse effects following the administering of one or more therapeutic agents and/or the administering of one or more immune modulators. In an aspect, wherein in the absence of adverse effects following the administering of one or more therapeutic agents and/or immune modulators, the method can further comprise continuing to treat the subject and/or continuing to monitor the subject. In an aspect, wherein in the presence of adverse effects, the method can further comprise modifying one or more steps of the method. In an aspect, modifying can comprise modifying the administering step of one or more therapeutic agents and/or immune modulators. In an aspect, modifying the administering step can comprise changing the amount of one or more therapeutic agents and/or immune modulators administered to the subject, changing the frequency of administration of one or more therapeutic agents and/or immune modulators, changing the duration of administration of the vector, changing the route of administration of one or more therapeutic agents and/or immune modulators, or any combination thereof.

In an aspect of a disclosed method, a disclosed CRISPR/Cas9 editing system can be applied to one or more amino acids of any enzyme in the lysine catabolism pathways (i.e., the pipecolate pathway and the saccharopine pathway). In an aspect, these enzymes can include, but are not limited to aminoadipate-semialdehyde synthase, α-aminoadipic semialdehyde, the kynurenine aminotransferase 2, the dehydrogenase E1 and transketolase domain-containing protein 1, the L-lysine alpha-oxidase, the ketimine reductase mu-crystallin protein, the peroxisomal sarcosine oxidase, or the pyrroline-5-carboxylate reductase, or any combination thereof. In an aspect, for example, a disclosed CRISPR/Cas9 editing system can be applied to one or more amino acids in a disclosed enzyme comprising the sequence set forth in SEQ ID NO:03 or SEQ ID NO:04 or SEQ ID NO:37 or SEQ ID NO:40.

In an aspect, a disclosed method of reprogramming a metabolic pathway can comprise repeating an administering step one or more times. In an aspect, a disclosed method of reprogramming a metabolic pathway can comprise repeating a treating step one or more times. For example, in an aspect, a disclosed method can repeat the administering a disclosed siRNA, a disclosed formulation comprising a disclosed siRNA, a disclosed mRNA therapy, or a disclosed formulation comprising a disclosed mRNA therapy, or any combination thereof, can repeat the administering of one or more therapeutic agents one or more times, can repeat the administering of one or more immune modulators one or more times, or any combination thereof.

In an aspect, a disclosed method of reprogramming a metabolic pathway can comprise restoring liver-specific modulation of lysine catabolism, (ii) restoring one or more aspects of lysine homeostasis, (iii) reducing and/or decreasing the level of toxic catabolites in the liver and/or brain of the subject, (iv) restoring the metabolic flux from glutaryl-CoA to crotonyl-CoA, (v) improving motor performance (e.g., strength, gait, balance, coordination, and combinations thereof) of the subject, (vi) improving memory function of the subject, (vii) reduce anxiety in the subject, (viii) reducing and/or preventing neurological sequelae (e.g., neonatal macrocephaly, subdural hematomas, acute retinal hemorrhage, encephalopathy, striatal necrosis, and combinations thereof), (ix) improving and/or reducing and/or eliminating vascular dysfunction in the subject, (x) improving the subject's quality of life, (xi) increasing and/or prolong the subject's life span, (xii) increasing a subject's survivability, or (xiii) any combination thereof.

In an aspect, a disclosed method of treating and/or preventing GA-1 disease progression can comprise restoring liver-specific modulation of lysine catabolism in the subject's liver, (ii) restoring one or more aspects of lysine homeostasis in the subject's liver, (iii) reducing and/or decreasing the level of toxic catabolites in the liver and/or brain of the subject, (iv) restoring the metabolic flux from glutaryl-CoA to crotonyl-CoA in the subject's liver, or (v) any combination thereof.

In an aspect, a disclosed method of reprogramming a metabolic pathway can comprise treating and/or preventing Glutaric Aciduria Type-1 in a subject. In an aspect, a disclosed method of reprogramming a metabolic pathway can comprise improving and/or diminishing and/or ameliorate one or more symptoms associated Glutaric Aciduria Type-1 in a subject. In an aspect, a disclosed method of reprogramming a metabolic pathway can comprise improving and/or diminishing and/or ameliorate one or more pathologies associated with Glutaric Aciduria Type-1 in a subject. In an aspect, a disclosed method of reprogramming a metabolic pathway can comprise restoring one or more aspects of cellular homeostasis and/or cellular functionality in a subject.

In an aspect, a disclosed metabolic pathway can comprise lysine catabolism. In an aspect, a disclosed metabolic pathway can comprise a disclosed pipecolate pathway and/or a disclosed saccharopine pathway.

For example, in an aspect, a disclosed method of reprogramming a metabolic pathway can further comprise administering to the subject (i) a disclosed vector viral vector comprising a nucleic acid sequence encoding glutaryl-CoA dehydrogenase; (ii) a disclosed vector viral vector comprising the nucleic acid sequence set forth in SEQ ID NO:01 or SEQ ID NO:02; (iii) a disclosed vector viral vector comprising the nucleic acid sequence set forth in SEQ ID NO:17 or SEQ ID NO:18; (iv) a disclosed vector viral vector comprising the sequence set forth in SEQ ID NO:19; (v) a disclosed first viral vector comprising an isolated nucleic acid molecule comprising a nucleic acid sequence encoding one or more element of a gene editing system with a second disclosed viral vector comprising an isolated nucleic acid molecule comprising a nucleic acid sequence encoding one or more element of a gene editing system; (vi) first disclosed viral vector comprising a nucleic acid sequence encoding an endonuclease and a sgRNA directed at a target sequence in a target gene of interest and a second disclosed viral vector comprising a nucleic acid sequence encoding an endonuclease and a sgRNA directed at a target sequence in the target gene of interest; (vii) a disclosed viral vector comprising the sequence set forth in SEQ ID NO:20, and a disclosed viral vector comprising the sequence set forth in SEQ ID NO:21; (viii) a disclosed first viral vector comprising a nucleic acid sequence encoding a Cas9 endonuclease and a sgRNA directed at a target sequence in the aminoadipate-semialdehyde synthase gene and a second disclosed viral vector comprising a nucleic acid sequence encoding a Cas9 endonuclease and a sgRNA directed at a target sequence in the aminoadipate-semialdehyde synthase gene; (ix) a first disclosed viral vector comprising a nucleic acid sequence encoding a Cas9 endonuclease and a sgRNA directed at a target sequence in the aminoadipate-semialdehyde synthase gene and a second disclosed viral vector comprising a nucleic acid sequence encoding a Cas9 endonuclease and a sgRNA directed at a target sequence in the aminoadipate-semialdehyde synthase gene, wherein the Cas9 endonuclease of the first viral vector and/or the Cas9 endonuclease of the second viral vector comprises the sequence of SEQ ID NO:28, and wherein the sgRNA of the first viral vector comprises the sequence set forth in SEQ ID NO:09 or SEQ ID NO:10, and wherein the sgRNA of the second viral vector comprises the sequence set forth in SEQ ID NO:07 or SEQ ID NO:08; (x) a disclosed first viral vector comprising the sequence comprising a nucleic acid sequence encoding a Cas9 endonuclease and a sgRNA directed at a target sequence in the glutaryl-CoA dehydrogenase gene and a disclosed second viral vector comprising the sequence comprising a nucleic acid sequence encoding a Cas9 endonuclease and a sgRNA directed at a target sequence in the glutaryl-CoA dehydrogenase gene; (xi) a disclosed viral vector comprising an isolated nucleic acid molecule comprising a nucleic acid sequence encoding one or more element of a gene editing system; or (xii) any combination thereof.

2. Methods of Treating and/or Preventing GA-1 Disease Progression

Disclosed herein is a method of treating and/or preventing GA-1 disease progression, the method comprising administering to a subject in need thereof a therapeutically effective amount of a disclosed siRNA and/or a disclosed formulation comprising a disclosed siRNA.

Disclosed herein is a method of treating and/or preventing GA-1 disease progression, the method comprising administering to a subject in need thereof a therapeutically effective amount of a disclosed mRNA therapy and/or a disclosed formulation comprising a disclosed mRNA therapy.

Disclosed herein is a method of treating and/or preventing GA-1 disease progression, the method comprising administering to a subject in need thereof a therapeutically effective amount of siRNA targeting aminoadipate-semialdehyde synthase

Disclosed herein is a method of treating and/or preventing GA-1 disease progression, the method comprising administering to a subject in need thereof a therapeutically effective amount of mRNA therapy targeting an aspect of the lysine catabolism pathway.

Disclosed herein is an siRNA that can target any part of the aminoadipate-semialdehyde synthase gene comprising the sequence set forth in SEQ ID NO:35 or SEQ ID NO:36. Disclosed herein is an siRNA that can target any part of an aminoadipate-semialdehyde synthase gene comprising the sequence set forth in SEQ ID NO:38 or SEQ ID NO:39. In an aspect, a targeted part of an AASS sequence can comprise about 15 to about 35 base pairs. In an aspect, a targeted part of an AASS sequence can comprise about 20 to about 30 base pairs. In an aspect, a targeted part of an AASS sequence can comprise about 20 to about 24 base pairs. In an aspect, a targeted part of an AASS sequence can comprise about 21 to about 22 base pairs. In an aspect, a disclosed siRNA effects the complete silencing of the aminoadipate-semialdehyde synthase gene. Disclosed herein is an siRNA that can target any part of an the aminoadipate-semialdehyde synthase sequence comprising the sequence set forth in SEQ ID NO:35 or SEQ ID NO:36. Disclosed herein is an siRNA that can target any part of the aminoadipate-semialdehyde synthase sequence comprising the sequence set forth in SEQ ID NO:38 or SEQ ID NO:39.

In an aspect, a disclosed mRNA therapy can target or can be directed one or more enzymes in the pipecolate pathway, the saccharopine pathway, or both.

In an aspect, the disclosed siRNA can be encapsulated in lipid nanoparticles. Lipid nanoparticles are known to the skilled person. In an aspect, a disclosed lipid nanoparticle can comprise a commercially available formulation such as, for example, In vivo lipofectamine. In an aspect, the disclosed siRNA can be conjugated to GalNAc.

In an aspect of a disclosed method, the disclosed subject in need thereof can have or can have been diagnosed with a glutaric aciduria type 1 (GA-1). In an aspect, a subject can be male or female. In an aspect, a subject can be an adult, a teenager, an adolescent, a child, a toddler, a baby, or an infant.

In an aspect of a disclosed method, the disclosed subject's GA-1 related and/or associated behavior can be modulated. In an aspect of a disclosed method, the disclosed subject's GA-1 related and/or associated physiology can be modulated.

In an aspect of a disclosed method, administering a disclosed siRNA, a disclosed formulation comprising a disclosed siRNA, a disclosed mRNA therapy, or a disclosed formulation comprising a disclosed mRNA therapy, or any combination thereof can be administered systemically or directly. In an aspect, administering a disclosed siRNA, a disclosed formulation comprising a disclosed siRNA, a disclosed mRNA therapy, or a disclosed formulation comprising a disclosed mRNA therapy, or any combination thereof can comprise oral administration, intravenous administration, intratumoral administration, intraperitoneal administration, or any combination thereof. In an aspect, administering a disclosed siRNA, a disclosed formulation comprising a disclosed siRNA, a disclosed mRNA therapy, or a disclosed formulation comprising a disclosed mRNA therapy, or any combination thereof can be administered by any method of administration disclosed herein. In an aspect, a disclosed siRNA, a disclosed formulation comprising a disclosed siRNA, a disclosed mRNA therapy, or a disclosed formulation comprising a disclosed mRNA therapy, or any combination thereof can be administered via multiple routes either concurrently or sequentially. A skilled clinician can determine the best route of administration for a subject at a given time.

In an aspect of a disclosed method, administering a disclosed siRNA can be targeted to the subject's liver, wherein expression of the nucleic acid sequence eliminates aminoadipate-semialdehyde synthase.

In an aspect, a disclosed nucleic acid sequence can comprise only the sequence for the functional domains.

In an aspect of a disclosed method, administering a disclosed siRNA, a disclosed formulation comprising a disclosed siRNA, a disclosed mRNA therapy, or a disclosed formulation comprising a disclosed mRNA therapy, or any combination thereof can comprise a single dose, or in multiple doses (such as 2, 3, 4, 5, 6, 7, 8, 9 or 10 doses) as needed for the desired therapeutic results. In an aspect, multiple doses can be administered via the same route or via differing routes of administration. In an aspect, a disclosed siRNA, a disclosed formulation comprising a disclosed siRNA, a disclosed mRNA therapy, or a disclosed formulation comprising a disclosed mRNA therapy, or any combination thereof can be administered via multiple routes of administration.

In an aspect, a disclosed method can further comprise monitoring the subject's metabolic and/or physiologic improvement following the administering step and/or following the administering steps. In an aspect, a clinician can measure and/or determine the subject's metabolic and/or physiologic status over time to identify one or more improvements and/or one or more diminishments. In an aspect of a disclosed method, a clinician can use the subject's metabolic and/or physiologic status and/or the trend of the subject's metabolic and/or physiological status and/or trend to make a treatment decision and/or to modify an aspect of a disclosed method and/or to continue treating the subject and/or continue to administer a disclosed vector, a disclosed composition, a disclosed therapeutic agent, and/or a disclosed immune modulator, or any combination thereof. In an aspect, metabolic and/or physiologic data can inform the clinician.

In an aspect, techniques to monitor, measure, and/or assess the reprogramming a metabolic pathway can comprise qualitative (or subjective) means as well as quantitative (or objective) means. These means are known to the skilled person. In an aspect, a disclosed method can comprise subjecting the subject to one or more invasive or non-invasive diagnostic assessments. Diagnostic assessments are known to the art. In an aspect, a disclosed non-invasive diagnostic assessment can comprise x-rays, computerized tomography (CT) scans, magnetic resonance imaging (MRI) scans, ultrasounds, positron emission tomography (PET) scans, or any combination thereof. In an aspect, a disclosed invasive diagnostic assessment can comprise a tissue biopsy or exploratory surgery.

In an aspect, a disclosed increase and/or a disclosed improvement can comprise a 10%, 20%, 30%, 40%, 50%, 60%, 70%, 80%, 90%, 100%, or any amount of an increase and/or improvement when compared to a control subject (such as, for example, a subject that has not received a disclosed treatment (e.g., administration of a disclosed siRNA, a disclosed formulation comprising a disclosed siRNA, a disclosed mRNA therapy, or a disclosed formulation comprising a disclosed mRNA therapy, or any combination thereof)). In an aspect, a disclosed increase and/or a disclosed improvement can comprise a 10-20%, 20-30%, 30-40%, 40-50%, 50-60%, 60-70%, 70-80%, 80-90%, or 90-100% or any amount of an increase and/or improvement when compared to a control subject (such as a subject that has not received a disclosed treatment (e.g., administration of a disclosed siRNA, a disclosed formulation comprising a disclosed siRNA, a disclosed mRNA therapy, or a disclosed formulation comprising a disclosed mRNA therapy, or any combination thereof)).

In an aspect, a disclosed decrease and/or a disclosed reduction can comprise a 10%, 20%, 30%, 40%, 50%, 60%, 70%, 80%, 90%, 100%, or any amount of a decrease and/or reduction when compared to a control subject (such as, for example, a subject that has not received a disclosed treatment (e.g., administration a disclosed siRNA, a disclosed formulation comprising a disclosed siRNA, a disclosed mRNA therapy, or a disclosed formulation comprising a disclosed mRNA therapy, or any combination thereof)). In an aspect, a disclosed decrease and/or a disclosed reduction can comprise a 10-20%, 20-30%, 30-40%, 40-50%, 50-60%, 60-70%, 70-80%, 80-90%, or 90-100% or any amount of a decrease and/or reduction when compared to a control subject (such as a subject that has not received a disclosed treatment (e.g., administration of a disclosed siRNA, a disclosed formulation comprising a disclosed siRNA, a disclosed mRNA therapy, or a disclosed formulation comprising a disclosed mRNA therapy, or any combination thereof)).

In an aspect, a disclosed method can further comprise monitoring the subject for adverse effects following the administering step and/or following the administering steps. In an aspect, wherein in the absence of adverse effects, the method can further comprise continuing to treat the subject and/or continuing to monitor the subject. In an aspect, wherein in the presence of adverse effects, the method can further comprise modifying one or more steps of the method. In an aspect, modifying can comprise modifying the treating step, modifying the administering step, or both.

In an aspect, modifying the treating step can comprise changing the amount of a disclosed siRNA, a disclosed formulation comprising a disclosed siRNA, a disclosed mRNA therapy, or a disclosed formulation comprising a disclosed mRNA therapy, or any combination thereof administered to the subject, changing the frequency of administration of a disclosed siRNA, a disclosed formulation comprising a disclosed siRNA, a disclosed mRNA therapy, or a disclosed formulation comprising a disclosed mRNA therapy, or any combination thereof, changing the duration of administration of a disclosed siRNA, a disclosed formulation comprising a disclosed siRNA, a disclosed mRNA therapy, or a disclosed formulation comprising a disclosed mRNA therapy, or any combination thereof, changing the route of administration of a disclosed siRNA, a disclosed formulation comprising a disclosed siRNA, a disclosed mRNA therapy, or a disclosed formulation comprising a disclosed mRNA therapy, or any combination thereof, or any combination thereof.

In an aspect of a disclosed method, a disclosed CRISPR/Cas9 editing system can be applied to one or more amino acids of any enzyme in the lysine catabolism pathways (i.e., the pipecolate pathway and the saccharopine pathway). In an aspect, these enzymes can include, but are not limited to aminoadipate-semialdehyde synthase, α-aminoadipic semialdehyde, the kynurenine aminotransferase 2, the dehydrogenase E1 and transketolase domain-containing protein 1, the L-lysine alpha-oxidase, the ketimine reductase mu-crystallin protein, the peroxisomal sarcosine oxidase, or the pyrroline-5-carboxylate reductase, or any combination thereof. In an aspect, for example, a disclosed CRISPR/Cas9 editing system can be applied to one or more amino acids in a disclosed enzyme comprising the sequence set forth in SEQ ID NO:03 or SEQ ID NO:04 or SEQ ID NO:37 or SEQ ID NO:40.

In an aspect, modifying the administering step can comprise changing the amount of a disclosed siRNA and/or a disclosed formulation comprising a disclosed siRNA, a disclosed formulation comprising a disclosed siRNA, a disclosed mRNA therapy, or a disclosed formulation comprising a disclosed mRNA therapy, or any combination thereof administered to the subject, changing the frequency of administration of a disclosed siRNA, a disclosed formulation comprising a disclosed siRNA, a disclosed mRNA therapy, or a disclosed formulation comprising a disclosed mRNA therapy, or any combination thereof, changing the duration of administration of a disclosed siRNA, a disclosed formulation comprising a disclosed siRNA, a disclosed mRNA therapy, or a disclosed formulation comprising a disclosed mRNA therapy, or any combination thereof, changing the route of administration of a disclosed siRNA, a disclosed formulation comprising a disclosed siRNA, a disclosed mRNA therapy, or a disclosed formulation comprising a disclosed mRNA therapy, or any combination thereof, or any combination thereof.

In an aspect, a disclosed method can further comprise administering to the subject a therapeutically effective amount of a therapeutic agent. Therapeutic agents are known.

In an aspect, a disclosed method can further comprise administering to the subject a therapeutically effective amount of one or more immune modulators. In an aspect, the one or more immune modulators comprise methotrexate, rituximab, intravenous gamma globulin, Tacrolimus, prednisolone or a prednisolone analog, SVP-Rapamycin, bortezomib, or a combination thereof. Immune modulators are known to the art.

In an aspect, a disclosed method can further comprise monitoring the subject for adverse effects following the administering of one or more therapeutic agents and/or the administering of one or more immune modulators. In an aspect, wherein in the absence of adverse effects following the administering of one or more therapeutic agents and/or immune modulators, the method can further comprise continuing to treat the subject and/or continuing to monitor the subject. In an aspect, wherein in the presence of adverse effects, the method can further comprise modifying one or more steps of the method. In an aspect, modifying can comprise modifying the administering step of one or more therapeutic agents and/or immune modulators. In an aspect, modifying the administering step can comprise changing the amount of one or more therapeutic agents and/or immune modulators administered to the subject, changing the frequency of administration of one or more therapeutic agents and/or immune modulators, changing the duration of administration of the vector, changing the route of administration of one or more therapeutic agents and/or immune modulators, or any combination thereof.

In an aspect, a disclosed method of treating and/or preventing GA-1 disease progression can comprise repeating an administering step one or more times. In an aspect, a disclosed method of treating and/or preventing GA-1 disease progression can comprise repeating a treating step one or more times. For example, in an aspect, a disclosed method can repeat the administering of a disclosed siRNA, a disclosed formulation comprising a disclosed siRNA, a disclosed mRNA therapy, or a disclosed formulation comprising a disclosed mRNA therapy, or any combination thereof, can repeat the administering of one or more therapeutic agents one or more times, can repeat the administering of one or more immune modulators one or more times, or any combination thereof.

In an aspect, a disclosed method of treating and/or preventing GA-1 disease progression can comprise restoring liver-specific modulation of lysine catabolism, (ii) restoring one or more aspects of lysine homeostasis, (iii) reducing and/or decreasing the level of toxic catabolites in the liver and/or brain of the subject, (iv) restoring the metabolic flux from glutaryl-CoA to crotonyl-CoA, (v) improving motor performance (e.g., strength, gait, balance, coordination, and combinations thereof) of the subject, (vi) improving memory function of the subject, (vii) reduce anxiety in the subject, (viii) reducing and/or preventing neurological sequelae (e.g., neonatal macrocephaly, subdural hematomas, acute retinal hemorrhage, encephalopathy, striatal necrosis, and combinations thereof), (ix) improving and/or reducing and/or eliminating vascular dysfunction in the subject, (x) improving the subject's quality of life, (xi) increasing and/or prolong the subject's life span, (xii) increasing a subject's survivability, or (xiii) any combination thereof.

In an aspect, a disclosed method of treating and/or preventing GA-1 disease progression can comprise restoring liver-specific modulation of lysine catabolism in the subject's liver, (ii) restoring one or more aspects of lysine homeostasis in the subject's liver, (iii) reducing and/or decreasing the level of toxic catabolites in the liver and/or brain of the subject, (iv) restoring the metabolic flux from glutaryl-CoA to crotonyl-CoA in the subject's liver, or (v) any combination thereof.

In an aspect, a disclosed method of treating and/or preventing GA-1 disease progression can comprise treating and/or preventing Glutaric Aciduria Type-1 in a subject. In an aspect, a disclosed method of treating and/or preventing GA-1 disease progression can comprise improving and/or diminishing and/or ameliorate one or more symptoms associated Glutaric Aciduria Type-1 in a subject. In an aspect, a disclosed method of treating and/or preventing GA-1 disease progression can comprise improving and/or diminishing and/or ameliorate one or more pathologies associated with Glutaric Aciduria Type-1 in a subject. In an aspect, a disclosed method of treating and/or preventing GA-1 disease progression can comprise restoring one or more aspects of cellular homeostasis and/or cellular functionality in a subject. In an aspect, a disclosed metabolic pathway can comprise lysine catabolism. In an aspect, a disclosed metabolic pathway can comprise a disclosed pipecolate pathway and/or a disclosed saccharopine pathway.

For example, in an aspect, a disclosed method of treating and/or preventing GA-1 disease progression can further comprise administering to the subject (i) a disclosed vector viral vector comprising a nucleic acid sequence encoding glutaryl-CoA dehydrogenase; (ii) a disclosed vector viral vector comprising the nucleic acid sequence set forth in SEQ ID NO:01 or SEQ ID NO:02; (iii) a disclosed vector viral vector comprising the nucleic acid sequence set forth in SEQ ID NO:17 or SEQ ID NO:18; (iv) a disclosed vector viral vector comprising the sequence set forth in SEQ ID NO:19; (v) a disclosed first viral vector comprising an isolated nucleic acid molecule comprising a nucleic acid sequence encoding one or more element of a gene editing system with a second disclosed viral vector comprising an isolated nucleic acid molecule comprising a nucleic acid sequence encoding one or more element of a gene editing system; (vi) first disclosed viral vector comprising a nucleic acid sequence encoding an endonuclease and a sgRNA directed at a target sequence in a target gene of interest and a second disclosed viral vector comprising a nucleic acid sequence encoding an endonuclease and a sgRNA directed at a target sequence in the target gene of interest; (vii) a disclosed viral vector comprising the sequence set forth in SEQ ID NO:20, and a disclosed viral vector comprising the sequence set forth in SEQ ID NO:21; (viii) a disclosed first viral vector comprising a nucleic acid sequence encoding a Cas9 endonuclease and a sgRNA directed at a target sequence in the aminoadipate-semialdehyde synthase gene and a second disclosed viral vector comprising a nucleic acid sequence encoding a Cas9 endonuclease and a sgRNA directed at a target sequence in the aminoadipate-semialdehyde synthase gene; (ix) a first disclosed viral vector comprising a nucleic acid sequence encoding a Cas9 endonuclease and a sgRNA directed at a target sequence in the aminoadipate-semialdehyde synthase gene and a second disclosed viral vector comprising a nucleic acid sequence encoding a Cas9 endonuclease and a sgRNA directed at a target sequence in the aminoadipate-semialdehyde synthase gene, wherein the Cas9 endonuclease of the first viral vector and/or the Cas9 endonuclease of the second viral vector comprises the sequence of SEQ ID NO:28, and wherein the sgRNA of the first viral vector comprises the sequence set forth in SEQ ID NO:09 or SEQ ID NO:10, and wherein the sgRNA of the second viral vector comprises the sequence set forth in SEQ ID NO:07 or SEQ ID NO:08; (x) a disclosed first viral vector comprising the sequence comprising a nucleic acid sequence encoding a Cas9 endonuclease and a sgRNA directed at a target sequence in the glutaryl-CoA dehydrogenase gene and a disclosed second viral vector comprising the sequence comprising a nucleic acid sequence encoding a Cas9 endonuclease and a sgRNA directed at a target sequence in the glutaryl-CoA dehydrogenase gene; (xi) a disclosed viral vector comprising an isolated nucleic acid molecule comprising a nucleic acid sequence encoding one or more element of a gene editing system; or (xii) any combination thereof.

### VII. EXAMPLES

The Examples that follow are illustrative of specific aspects of the invention, and various uses thereof. They set forth for explanatory purposes only and are not to be taken as limiting the invention.

The work presented herein demonstrates that the liver directly contributes to toxic accumulation of catabolites in the brain as part of the GA-1 disease pathology. This is surprisingly and unexpected and therefore challenges that traditionally held view in the art. To this end, the three methods of redressing the dysfunctional lysine catabolism pathway in the liver also demonstrated the ability to reverse GA-1 disease pathology in the brain.

### A. Materials and Methods Employed in Specific Examples

### 1. Generation of Single (Gcdh−/−) and Double (Gcdh−/−/Aass−/−) Knockout Mouse Models.

Gcdh^{-/-} single knockout and Gcdh^{-/-}/Aass^{-/-} double knockout mouse strains were generated by injecting either C57B6 or TIRF (transgene free Ir2g^{-/-}, Rag2^{-/-}, Fah^{-/-}) zygotes (Bissig-Choisat B, et al. (2021) JHEP Rep. 3:100281) with CRISPR/Cas9 gene editing mRNA as we described previously (Barzi M, et al. (2017) Nat Commun. 8:39), which is incorporated by reference in its entirety for the methods and materials related to the generation and characterizations of these knockout mice. The following sgRNAs were designed using Benchling online software (https://www.benchling.com) and injected at the same time with the Cas9 mRNA. A listing of sgRNA sequences is set forth in Table 1.

TABLE 1
S. pyogenes sgRNA Sequences.

| S. pyogenes | | NGG PAM | |
|---|---|---|---|
| sgRNA Name | 5′ to 3′ Sequence | Sequence | Strand |
| mGcdh | GCCGCTCCTGGCAGTAGTTA | CGG | − |
| Exon 3 | (SEQ ID NO: 05) | | |
| mGcdh | GTGTGTCGTCGGTGGCCTAT | GGG | + |
| Exon 5 | (SEQ ID NO: 06) | | |
| mAass | GTGCAGGCTGTCCGTGATGC | TGG | + |
| Exon 6 | (SEQ ID NO: 07) | | |
| mAass | GAAACTTCTCTTAATTCGTG | GGG | − |
| Exon 7 | (SEQ ID NO: 09) | | |

F0 mice were analyzed by PCR followed by Sanger sequencing using the PCR primers set forth in Table 2:

TABLE 2
PCR Primers for Gedh and Aass.

| Primer Name | 5′ to 3′ Sequence |
|---|---|
| mGcdh For | TTATCCCCAGGGTCAGAAG (SEQ ID NO: 13) |
| mGcdh Rev | CCAGACCGACATCTGAC (SEQ ID NO: 14) |
| mAass For | TAGAGAGAACGGGCAGGATGT (SEQ ID NO: 15) |
| mAass Rev | TCTACGGGATCGTACACACCA (SEQ ID NO: 16) |

Further offspring genotyping was performed by Transnetyx (Cordoba, TN).

2. Experiments with Knockout Mice.

Single (Gcdh^{-/-}) and double (Gcdh^{-/-}/Aass^{-/-}) knockout mice were maintained under a standard 12-hour dark/light cycle with water and regular chow provided ad libitum. For the Adeno-Associated virus (AAV) experiments, 3-week-old and 6-day-old Gcdh^{-/-} pups were injected with a single i.p, dose of AAV expressing either the murine Gcdh cDNA or with two CRISPR/Cas9 gene editing tools targeting Aass, both regulated by a liver specific promoter (HLP). Two weeks after injection or when pups reached the weaning age, mice were exposed to high protein diet (70% Casein diet, Envigo Teklad Custom diet Catalog #TD.06723). Urine, blood and liver and brain tissue (where described) were collected 4 or 5 days after challenging the mice with high protein diet. Mouse body weights were measured throughout the experiment and all mouse tissues were harvested for further analysis at the experimental endpoints. All animal experiments were approved by the Institutional Animal Care and Use Committee.

### 3. Hepatocyte Isolation.

To obtain primary hepatocytes for transplantation experiments, mouse livers were perfused using a modified two-step collagenase perfusion method as described previously (Maeso-Diaz R, et al. (2022) Aging Cell. 21:e13530). Quality of isolated hepatocytes was assessed by trypan blue staining of perfusate and used if viability was >90%. Freshly isolated hepatocytes were transplanted into mice the same day (<8 hours after isolation).

### 4. Hepatocyte Transplantation.

Transplantation of healthy hepatocytes (Gcdh^{-/-}) expressing td-Tomato red fluorescent reporter (mT/mG mouse strain, B6.129(Cg)-Gt(ROSA)26Sor^{tm4(ACTB-tdTomato,-EGFP)Luo/J}, The Jackson Laboratory, Catalog #007676) and diseased (Gcdh^{-/-}) hepatocytes was performed as previously described (Bissig-Choisat B, et al. (2016) Nature. 6:7339) into TIRF background single (Gcdh^{-/-}) or double (Gcdh^{-/-}/Aass^{-/-}) knockout mouse strains (host strains), respectively. In brief, 1×10⁶ hepatocytes were injected into the spleen of 2-month-old mice. Immediately after transplantation, selection pressure towards transplanted hepatocytes was applied by withdrawing the drug nitisinone (NTBC) from the drinking water. After 2 weeks, mice were put back on nitisinone for 3 days before a second withdrawal (cycling). Mice were kept without nitisinone for 6 months to assure a good repopulation with transplanted hepatocytes. Before starting the 70% casein diet challenge, mice were put back on nitisinone to assure no interference with tyrosinemia of the TIRF strain.

### 5. AAV Vector Cloning and AAV Virus Production.

Murine Gcdh cDNA was cloned by replacing EmGFP of 1162-pAAV-HLP-EmGFP-SpA plasmid (provided from William Lagor, Addgene Catalog #109313) using XbaI-MluI restriction enzymes. Murine Aass Sa sgRNAs oligonucleotides were annealed and ligated into 1313.1-pAAV-U6-SA-BbsI-MluI-gRNA-HLP-OLLAS-spA vector (provided by William Lagor, Addgene Catalog #109314) digested with BbsI restriction enzyme. AAVs were produced as previously described (Nelson C E, et al. (2019) Nat Med. 25:427-432). A listing of sgRNA sequences is set forth in Table 3.

TABLE 3
S. aureus sgRNA Sequences.

| S. aureus | | NNGR PAM | |
|---|---|---|---|
| sgRNA Name | 5′ to 3′ Sequence | Sequence | Strand |
| mAass | GTCCCTGTGAAGACAAACGTT | AAGG | − |
| Exon 6 | (SEQ ID NO: 08) | | |
| mAass | CTTGTGAGTATGTGGAGCCCC | ACGA | + |
| Exon 7 | (SEQ ID NO: 10) | | |

6. siRNA Experiments.
siRNA Preparation.

siRNA injection solution was prepared following In vivo fectamine 3.0 Reagent Complexation protocol (Thermosfisher Scientific, Catalog #IVF3001). In brief, siRNA duplex (Ambion, Catalog #4457308, ID #s 78304) was first diluted in RNAse free water to a concentration of 250 μM, aliquoted and stored at −80° C. siRNA duplex solution was diluted in 1:1 in complexation buffer and then mixed 1:1 with In vivo fectamine 3.0 Reagent, vortexed, and incubated at 50° C. for 30 minutes. The complex was diluted 1:6 with RNAse PBS 1×pH 7.4. A listing of sgRNA sequences is set forth in Table 4.

TABLE 4
Aass siRNA.

| Sense 5′ → 3′ | Antisense 5′ → 3′ |
|---|---|
| sequence | sequence |
| GGAGUCUUGAUGAACAUAAtt | UUAUGUUCAUCAAGACUCCca |
| (SEQ ID NO: XX) | (SEQ ID NO: XX) |

7. siRNA Injection.

Aass siRNA (8 mg/kg) solution was injected into the tail vein of 3-week-old Gcdh/mice and put on high protein diet 48 hours later. Mice were harvest postmortem for expression of AASS in the liver using AASS immunostaining as described elsewhere.

### 8. Neurobehavioral Studies

Mouse motor activity studies were performed by the Mouse Behavioral and Neuroendocrine Core Facility at Duke University.

### 9. Spontaneous Motor Activity.

Spontaneous motor activity was monitored in the open field (21 cm×21 cm×30 cm) over 30 min in an automated Omnitech Digiscan apparatus (AccuScan Instruments, Columbus, OH) (Fukui M, et al. (2007) J Neurosci. 27:10520-10529). The AccuScan software scored motor activities as horizontal or vertical beam-breaks to determine the total distance traveled, vertical activity, velocity of movement, and time spent in the center zone of the arena.

### 10. Accelerating Rotarod.

Balance and coordination were evaluated on an accelerating (4-40 rpm over 5-min) rotorod (Med-Associates, St. Albans, VT) as described (Taylor G A, et al. (2008) Genes Brain Behav. 7:786-795). Motor performance was examined over 4 successive 5-min trials that were separated by 20-30 min each. A given trial was terminated when the mouse fell from the rod or when 300 sec had elapsed, and these times were recorded as the latency to fall.

### 11. Grip Strength.

The strength of the front and rear paws to grip a bar was analyzed with a mouse grip-strength meter (San Diego Instruments, San Diego, CA) and was expressed as units of g-force (Wang X, et al. (2011) Hum Mol Genet. 20:3093-3108).

### 12. Statistical Analysis.

The data for motor performance are presented as means±SEM. The data were analyzed by one-way ANOVA, repeated-measures ANOVA (RMANOVA), and multivariate ANOVA (MANOVA), followed by Bonferroni corrected pair-wise comparisons. A p<0.05 was considered statistically significant, a trend was considered p<0.10.

### 13. Metabolite Analysis.

a. Blood Acylcarnitine (C5-DC).

Whatman 903 protein saver cards (Sigma-Aldrich), d₃-Acetylcarnitine (d₃-C2, Sigma-Aldrich), d₃-Propionylcarnitine (d₃-C3, Sigma-Aldrich), d₃-Butyrylcarnitine (d₃-C4, Sigma-Aldrich), d₃-Octanoylcamitine (d₃-C8, Sigma-Aldrich), and d₃-Palmitoylcarnitine (d₃-C16, Sigma-Aldrich) were used. General solvents and reagents were purchased from Sigma-Aldrich (St. Louis, MO) or VWR (Radnor, PA). In-house deionized water (diH₂O) was used in the preparation of mobile phases or for dilutions.

i. Sample Preparation.

Whole blood (16 μL) was pipetted onto two 3/16″ diameter circles of cotton fiber filter paper and allowed to dry overnight in a microcentrifuge tube. Following that, 6 μL of internal standard (IS) mixture (5 μmol/L d₃-C2, 1 μmol/L d₃-C3, 1 μmol/L d₃-C4, 1 μmol/L d₃-C8, 2 μmol/L d₃-C16 in methanol:diH₂O 50:50 (v/v)) was added to the tube along with 400 μL of methanol (MeOH). The microcentrifuge tubes were then placed on an orbital shaker for 30 minutes at ambient temperature. The entire volume of liquid was then transferred to a 0.2 μm filter tube and centrifuged at 16,380 g for 2 minutes. An aliquot (200 μL) of the filtered supernatant was transferred to a 96 well round bottom plate and evaporated to dryness under nitrogen at 40° C. After drying, 70 μL of 3M MeOH-Hydrochloric Acid was added to each specimen, an adhesive cover was placed over each plate, and the samples incubated in an oven for 15 minutes at 50° C. Samples were dried under a stream of nitrogen at 40° C. and reconstituted in a matrix of MeOH:diH₂O 85:15 (v:v) and analyzed by electrospray ionization-tandem mass spectrometry (ESI-MS/MS) (Millington D S, et al. (2011) Methods Mol. Biol. 708:55-72; Lepage N, et al. (2010). Methods Mol. Biol. 603:9-25).

### (a) LC-MS/MS Instrument and Analysis.

Acylcarnitines in whole blood were analyzed as methyl esters using stable isotope dilution ESI-MS/MS. Derivatized samples were analyzed by flow injection analysis (FIA) and detected using a precursor ion scan of m/z 99. Samples were analyzed using a TQ Detector tandem quadrupole mass spectrometer equipped with an Acquity Classic system (Waters Corporation, Milford, MA). A FIA was performed over 2.5 minutes using MeOH:diH₂O 80:20 (v:v), which allowed for elution of the sample between 0.2 minutes and 1.0 minutes, with a wash out period between 1.0 minutes and 2.2 minutes, followed by a re-equilibration period from 2.2 minutes to 2.5 minutes.

### (b) Data Processing.

The raw data was processed using Neolynx® (Waters Corp.). The ratio of ion intensities of acylcarnitine species and its specified deuterated IS are multiplied by the nominal concentration of the IS (5 μmol/L, 1 μmol/L, 1 μmol/L, 1 μmol/L, 2 μmol/L). Concentrations of standards are given in units of μmol/L. Glutarylcarnitine (C5-DC), with m/z 304, was measured against octanoyl-L-carnitine-d₃ (d₃-C8) with m/z 305. Propionylcarnitine (C3), with m/z 232, and acetylcarnitine (C2), with m/z 218, are each compared to their own deuterated IS (d₃-C2 and d₃-C3).

b. Blood Amino Acids.
ii. Analysis of Amino Acids by LC-MS/MS.

Whatman 903 protein saver cards (Sigma-Aldrich), Kairos amino acid internal standard set (100+), amino acid calibrator set (100+), amino acid quality control set (100+), and well as AccQ-Tag Ultra derivatization kit were purchased from Waters Corporation (Milford, MA). LC-MS grade acetonitrile, methanol, formic acid, acids and bases were purchased from Sigma-Aldrich (St. Louis, MO) or VWR (Radnor, PA). In-house deionized water (diH₂O) was used in the preparation of mobile phases or for dilutions. Whole blood (12 μL) was pipetted onto a ¼″ diameter circle of cotton fiber filter paper and allowed to dry overnight in a microcentrifuge tube. Plasma amino acids were analyzed using a modification of the Kairos Amino Acid method. Equal volumes (50 μL) of plasma and an internal standard solution containing a mixture of [¹³C, ¹⁵N]-labeled amino acids were combined. Protein was precipitated using 50 μL 10% sulfosalicylic acid and removed by centrifugation. The supernatant was added to a borate buffer, mixed with the 6-aminoquinolyl-N-hydroxysuccinimidyl carbamate (AQC) derivatization reagent, incubated at 55° C. for 10 minutes, and diluted with dH₂O. Plasma amino acid-AQC derivatives were analyzed using a Waters Acquity I-Class UPLC coupled to a Waters Xevo TQ-S micro mass spectrometer. Amino acids were separated on a 1.6 μm 2×150 mm Cortecs UPLC column by gradient elution over 9.5 minutes, with 0.1% formic acid in aqueous acetonitrile as the mobile phase. Analytes were detected by selected reaction monitoring in positive ion mode. Peak area ratios of amino acids and their corresponding internal standard were converted to a concentration by means of a 6- or 7-point 1/x weighted calibration curve. Details of the acquisition parameters are provided in Table 5 below.

TABLE 5
SRM Transitions, Detailed View.

| | | | | | | | | |
|---|---|---|---|---|---|---|---|---|
| | Prnt | Dau | Dwell | Cone | Col | Delay | | |
| Ch | (Da) | (Da) | (s) | (V) | (eV) | (s) | Compound | Comments |

Function 1

| | | | | | | | | |
|---|---|---|---|---|---|---|---|---|
| 1 | 232.1 | 171.05 | 0.004 | 30 | 20 | Auto | Ethanolamine | |
| 2 | 246.1 | 171.05 | 0.004 | 30 | 20 | Auto | Glycine | |
| 3 | 249.1 | 171.05 | 0.004 | 30 | 20 | Auto | Glycine-IS | 13C2, 15N |

Function 2

| | | | | | | | | |
|---|---|---|---|---|---|---|---|---|
| 1 | 244.1 | 171.05 | 0.004 | 30 | 15 | Auto | Lysine | |
| 2 | 248.1 | 171.05 | 0.004 | 30 | 15 | Auto | Lysine-IS | 13C6, 15N2 |

Function 3

| | | | | | | | | |
|---|---|---|---|---|---|---|---|---|
| 1 | 252.1 | 171.05 | 0.004 | 30 | 13 | Auto | OH-Lysine | |
| 2 | 260.1 | 171.05 | 0.004 | 30 | 20 | Auto | Sarcosine, β-Alanine, | Isomers |
| | | | | | | | Alanine | |
| 3 | 264.1 | 171.05 | 0.004 | 30 | 20 | Auto | Alanine-IS | 13C3, 15N |
| 4 | 332.1 | 171.05 | 0.004 | 30 | 20 | Auto | α-Aminoadipic Acid | |

Function 4

| | | | | | | | | |
|---|---|---|---|---|---|---|---|---|
| 1 | 274.2 | 171.05 | 0.004 | 30 | 20 | Auto | α,β,δ-Aminobutyric | Isomers |
| | | | | | | | acid | |

Function 5

| | | | | | | | | |
|---|---|---|---|---|---|---|---|---|
| 1 | 276.1 | 171.05 | 0.004 | 30 | 20 | Auto | Serine | |
| 2 | 280.1 | 171.05 | 0.004 | 30 | 20 | Auto | Serine-IS | 13C3, 15N |
| 3 | 296.1 | 171.05 | 0.004 | 30 | 20 | Auto | Taurine | |

Function 6

| | | | | | | | | |
|---|---|---|---|---|---|---|---|---|
| 1 | 282.1 | 171.05 | 0.004 | 30 | 15 | Auto | Cystathionine | |
| 2 | 286.1 | 171.05 | 0.004 | 30 | 20 | Auto | Proline | |
| 3 | 292.1 | 171.05 | 0.004 | 30 | 20 | Auto | Proline-IS | 13C5, 15N |
| 4 | 473.2 | 171.05 | 0.004 | 30 | 25 | Auto | Ornithine | |
| 5 | 480.2 | 171.05 | 0.004 | 30 | 25 | Auto | Ornithine-IS | 13C5, 15N2 |

Function 7

| | | | | | | | | |
|---|---|---|---|---|---|---|---|---|
| 1 | 288.1 | 171.05 | 0.051 | 30 | 20 | Auto | Valine | |
| 2 | 294.1 | 171.05 | 0.051 | 30 | 20 | Auto | Valine-IS | 13C5, 15N |

Function 8

| | | | | | | | | |
|---|---|---|---|---|---|---|---|---|
| 1 | 290.1 | 171.05 | 0.004 | 30 | 20 | Auto | Threonine | |
| 2 | 295.1 | 171.05 | 0.004 | 30 | 20 | Auto | Threonine-IS | 13C4, 15N |

Function 9

| | | | | | | | | |
|---|---|---|---|---|---|---|---|---|
| 1 | 291.1 | 171.05 | 0.004 | 30 | 15 | Auto | Cystine | |
| 2 | 295.1 | 171.05 | 0.004 | 30 | 15 | Auto | Cystine-IS | 13C6, 15N2 |

Function 10

| | | | | | | | | |
|---|---|---|---|---|---|---|---|---|
| 1 | 302.1 | 171.05 | 0.004 | 30 | 20 | Auto | OH-Proline | |
| 2 | 303.1 | 171.05 | 0.004 | 30 | 20 | Auto | Asparagine | |
| 3 | 309.1 | 171.05 | 0.004 | 30 | 20 | Auto | Asparagine-IS | 13C4, 15N2 |
| 4 | 312.1 | 171.05 | 0.004 | 30 | 15 | Auto | Phosphoethanolamine | Coll. E |

Function 11

| | | | | | | | | |
|---|---|---|---|---|---|---|---|---|
| 1 | 302.1 | 171.05 | 0.031 | 30 | 20 | Auto | Iso, Allo-Iso, Leucine | Isomers |
| 2 | 305.1 | 171.05 | 0.031 | 30 | 20 | Auto | Homocystine | |
| 3 | 309.1 | 171.05 | 0.031 | 30 | 20 | Auto | Leucine, IsoL-IS | 13C6, 15N |
| 4 | 379.15 | 171.05 | 0.031 | 30 | 25 | Auto | Kynurenine | |

Function 12

| | | | | | | | | |
|---|---|---|---|---|---|---|---|---|
| 1 | 304.1 | 171.05 | 0.004 | 30 | 20 | Auto | Aspartic Acid | |
| 2 | 309.1 | 171.05 | 0.004 | 30 | 20 | Auto | Aspartic Acid-IS | 13C4, 15N |
| 3 | 346.1 | 171.05 | 0.004 | 30 | 20 | Auto | Citrulline | |
| 4 | 360.15 | 171.05 | 0.004 | 30 | 20 | Auto | Homocitrulline | |
| 5 | 372.15 | 171.05 | 0.004 | 30 | 20 | Auto | S-Sulfocysteine | |

Function 13

| | | | | | | | | |
|---|---|---|---|---|---|---|---|---|
| 1 | 317.1 | 171.05 | 0.004 | 30 | 20 | Auto | Glutamine | |
| 2 | 324.1 | 171.05 | 0.004 | 30 | 20 | Auto | Glutamine-IS | 13C5, 15N2 |

Function 14

| | | | | | | | | |
|---|---|---|---|---|---|---|---|---|
| 1 | 318.1 | 171.05 | 0.004 | 30 | 20 | Auto | Glutamic acid | |
| 2 | 324.1 | 171.05 | 0.004 | 30 | 20 | Auto | Glutamic acid-IS | 13C5, 15N |

Function 15

| | | | | | | | | |
|---|---|---|---|---|---|---|---|---|
| 1 | 320.1 | 171.05 | 0.051 | 30 | 20 | Auto | Methionine | |
| 2 | 326.1 | 171.05 | 0.051 | 30 | 20 | Auto | Methionine-IS | 13C5, 15N |

Function 16

| | | | | | | | | |
|---|---|---|---|---|---|---|---|---|
| 1 | 326.1 | 171.05 | 0.004 | 30 | 20 | Auto | Histidine | |
| 2 | 335.1 | 171.05 | 0.004 | 30 | 20 | Auto | Histidine-IS | 13C6, 15N3 |

Function 17

| | | | | | | | | |
|---|---|---|---|---|---|---|---|---|
| 1 | 336.1 | 171.05 | 0.031 | 30 | 20 | Auto | Phenylalanine | |
| 2 | 346.1 | 171.05 | 0.031 | 30 | 20 | Auto | Phenylalanine-IS | 13C9, 15N |

Function 18

| | | | | | | | | |
|---|---|---|---|---|---|---|---|---|
| 1 | 345.1 | 171.05 | 0.004 | 30 | 20 | Auto | Arginine | |
| 2 | 355.1 | 171.05 | 0.004 | 30 | 20 | Auto | Arginine-IS | 13C6, 15N4 |

Function 19

| | | | | | | | | |
|---|---|---|---|---|---|---|---|---|
| 1 | 352.1 | 171.05 | 0.004 | 30 | 20 | Auto | Tyrosine | |
| 2 | 362.1 | 171.05 | 0.004 | 30 | 20 | Auto | Tyrosine-IS | 13C9, 15N |
| 3 | 382.15 | 171.05 | 0.004 | 30 | 20 | Auto | 3-O-MDP | |

Function 20

| | | | | | | | | |
|---|---|---|---|---|---|---|---|---|
| 1 | 375.15 | 171.05 | 0.031 | 30 | 20 | Auto | Tryptophan | |
| 2 | 388.15 | 171.05 | 0.031 | 30 | 20 | Auto | Tryptophan-IS | 13C11, 15N2 |

Details of the calibrator concentrations are provided in Table 6 provided below.

TABLE 6
Calibrator Concentrations

| Calibrator | Nominal | Cystine |
|---|---|---|
| Level | Concentration (μM) | Concentration (μM) |
| 1 | 2.5 | 1.25 |
| 2 | 10 | 5 |
| 3 | 50 | 25 |
| 4 | 125 | 62.5 |
| 5 | 250 | 125 |
| 6 | 500 | 250 |
| 7* | 2000 | N/A |

Notes:
*Calibrator 7 only contains the following amino acids: serine, threonine, glutamine, glycine, alanine, isoleucine, leucine, valine, phenylalanine, tyrosine.

c. Tissue Organic Acids
iii. Sample Preparation.

100 mg of liver and brain tissue were homogenized in 0.5 mL of distilled deionized water using Tyssue Lyser (Qiagen) following 30 cycles of sonication at power (Vibra Cell—Sonics). 70 mg of sulfosalicylic acid was added to each sample and let stand at room temperature for 5 minutes. Samples were centrifuged at—for 20 minutes and supernatant was transferred to a glass stoppered tube for urine organic acid analysis.

iv. Glutaric Acid and 3-OH Glutaric Acid Measurements.

Glutaric acid (GA) and 3-OH-glutaric acid (3-OH-GA) were measured as previously described (sauer S W, et al. (2006) J. Neurochem. 97:889-910) using GC/MS with a stable-isotope dilution assay. In brief, internal standards of d4-GA and d5-3-OH-GA (each 1 nmol) were added to 2 mg of tissue homogenate. Samples were acidified to pH<1 with 100 lmol H₂SO₄. The reaction mixture was diluted with 1 mL NaHCO₃ (20 mmol/L) and, subsequently, the ionic strength of the solvent was increased by adding an excess of NaCl. Organic acids were extracted twice using ethylacetate and then supernatants were dried under nitrogen at 65° C. Finally, samples were derivated with N-methyl-Ntrimethylsilylheptafluorbutyramid. GC/MS analysis was carried out on a DB5-MS capillary column (25 m×0.25 mm inner diameter, film thickness 0.25 lm) obtained from J & W, (Agilent Technologies, Boblingen, Germany) which was installed in a Hewlett-Packard 6890 series GC and Hewlett-Packard Engine 5972 A mass spectrometer (Agilent Technologies). The mass spectrometer operated under electron impact in a single ion monitoring mode for enhanced sensitivity as previously described (Schor D S, et al. (2002) J Chromatogr B Analyt Technol Biomed Life Sci. 780:199-204). Four-point calibration curves were acquired for GA and 3-OH-GA in a range of 0-20 nmol using 1 nmol of d4-GA and 1 nmol of d5-3-OH-GA as internal standards. The following fragments were used for the quantification: m/z 217 and 259 (3-OH-GA), m/z 218 and 262 (deuterium labelled 3-OH-GA standard), m/z 261 and 158 (GA), and m/z 265 and 161 (deuterium-labelled GA standard). GA and 3-OHGA concentrations in all samples were subsequently normalized to the protein content.

### 14. Immunohistochemistry.

Paraffin-embedded slides were deparaffinated, rehydrated and treated with antigen retrieval citrate buffer (pH 6.0) for 30 minutes at 98° C. degrees. Endogenous peroxidase was quenched using 3% hydrogen peroxidase solution (Signa-Aldrich, Catalog #88697) and biotin was blocked with Avidin/Biotin kit following manufacturer's instructions (Vector Laboratories, Catalog #SP-2001). After blocking with serum (PK-4001), samples were incubated overnight at 4° C. with either rabbit anti-RFP (Rockland, Catalog #600-401-379) or rabbit anti-AASS (Sigma-Aldrich, Catalog #HPA020728) primary antibodies diluted 1:100 in antibody diluent buffer (Abcam, Catalog #Ab64211) and incubated overnight at 4° C. Slides were washed twice with PBS 1× for 15 minutes and incubated with the anti-rabbit biotinylated secondary antibody at room temperature for 30 minutes and staining was developed with DAB kit (Vector Laboratories, Catalog #SK-4100). Counterstaining was performed using hematoxylin solution (Richard-Allan Scientific, Catalog #7211) and bluing solution (Richard-Allan Scientific, Catalog #7301). Cytoseal (Epredia, Catalog #8312-4) was used for mounting the slides.

### 15. Western Blot.

20 g of fresh frozen liver and brain tissues were homogenized with 1.2 mL of RIPA buffer (Sigma Aldrich, Catalog #R0278) containing protease inhibitors (Roche, Catalog #04693159001). 20 μL (corresponding to 10 μg of protein) of homogenized samples was pre-mixed with loading buffer, heated and loaded in a polyacrylamide pre-made gel (NuPAGE 4-12% Bis Tris Gel Invitrogen, Catalog #NPO336BOX) and transferred to a PDVF membrane (Millipore, Catalog #IPVH00010). After blocking (EveryBlot Blocking Buffer, Biorad, Catalog #12010020) for 30 min. membranes were incubated at 4° C. overnight with primary antibodies diluted in PBS-T. Rabbit Anti-AASS (Sigma-Aldrich, Catalog #HPA020728), anti-GCDH (Sigma-Aldrich, Catalog #HPA020728) and beta-Actin (Sigma Aldrich, Catalog #A1978) diluted 1:1,000. After washed, membranes were incubated with donkey anti-rabbit HRP secondary antibody (Jackson Immunoresearch, Catalog #711-035-152) diluted 1:5,000 for 1 hour at room temperature. The images were obtained by incubating the membranes with Super Signal West Fempto solution (Thermofisher, Catalog #34096).

### 16. Histopathology.

Selected tissues (liver, lung, heart, kidney, spleen, brain) were evaluated by a board-certified veterinary pathologist (JE) in a masked fashion without knowledge of allocation group. Mouse brains were sectioned in the parasagittal plane. Following identification of three lesions in initial screening, (meningeal hemorrhage; hippocampal vacuolation; and nephropathy), the pathologist graded changes in brains and kidneys as normal, minimal, mild, moderate or severe (0-4) using a semi-quantitative scale.

### 17. Flux Studies.

### a. [2-¹⁵N]Lysine Metabolic Flux Study.

The position-specifically labeled lysine ([2-15N]Lysine) is employed to investigate the AASS-catabolized metabolic flux. The catabolism of lysine is via the saccharopine (liver and kidney) and pipecolate (brain) pathways. Only the saccharopine pathway leads to the labeled 2-aminoadipic-4-semialdehyde and 2-aminoadipate (AASA) metabolites from [2-15N]Lysine. ¹⁵N at carbon-2 of lysine is lost in the first step of pipecolate pathway. Thus, by measuring the labeling of 2-aminoadipate, the AASS-mediated metabolic flux from lysine to AASA and 2-aminoadipate (AAA) can be assessed. This metabolic flux approach is used to assess the relative metabolic change induced by AASS deletion. The deletion of AASS is expected to block the labeling of AAA from [2-15N]Lysine.

b. [¹³C₆] Lysine Metabolic Flux Study.

To measure the relative metabolic flux from lysine to acetyl-CoA, [¹³C₆] Lysine is employed. The downstream metabolites, such as acetyl-CoA or tricarboxylic acid cycle (TCA cycle) metabolites, are labeled by [¹³C₆] Lysine. By measuring the labeling of acetyl-CoA or TCA cycle metabolites (citrate, 2-ketoglutarate, succinate, fumarate, and malate etc.), the change of relative catabolic flux of Dlysine can be estimated.

c. Analytical Experiments for Lysine Catabolites.

The labeling and concentration of organic acids (3-OH-GA, 2-aminoadipate, 2-oxoadipate, and GA), amino acids, and TCA cycle intermediates in tissue, urine, and plasma are analyzed by GC-MS. The labeling and concentration of acylcaritine (plasma and tissue (liver and brain)) and acyl-CoA (tissue) is analyzed by LC-MS/MS.

TABLE 7
Listing of Sequences and Sequence Identifiers.

| Seq. Identifier | Description (source) |
|---|---|
| SEQ ID NO: 01 | Glutaryl-CoA Dehydrogenase (GCDH) (human) (genomic) |
| SEQ ID NO: 02 | Glutaryl-CoA Dehydrogenase (GCDH) (mouse) (genomic) |
| SEQ ID NO: 03 | Glutaryl-CoA Dehydrogenase (GCDH) (human) (protein) |
| SEQ ID NO: 04 | Glutaryl-CoA Dehydrogenase (GCDH) (mouse) (protein) |
| SEQ ID NO: 05 | sgRNA for mGcdh Exon 3 (S. pyogenes) |
| SEQ ID NO: 06 | sgRNA for mGcdh Exon 5 (S. pyogenes) |
| SEQ ID NO: 07 | sgRNA for mAass Exon 6 (S. pyogenes) |
| SEQ ID NO: 08 | sgRNA for mAass Exon 6 (S. pyogenes) |
| SEQ ID NO: 09 | sgRNA for mAass Exon 7 (S. pyogenes) |
| SEQ ID NO: 10 | sgRNA for mAass Exon 7 (S. pyogenes) |
| SEQ ID NO: 11 | TracrRNA for Aass Exon 7 |
| SEQ ID NO: 12 | TracrRNA for Aass Exon 6 |
| SEQ ID NO: 13 | FOR Primer for mGcdh (artificial) |
| SEQ ID NO: 14 | REV Primer for mGcdh (artificial) |
| SEQ ID NO: 15 | FOR Primer for mAass (artificial) |
| SEQ ID NO: 16 | REV Primer for mAass (artificial) |
| SEQ ID NO: 17 | GCDH cDNA (human) |
| SEQ ID NO: 18 | Gcdh cDNA (mouse) |
| SEQ ID NO: 19 | AAV-mGcdh Full Vector (artificial) |
| SEQ ID NO: 20 | pAAV-CRISPR-Aass-Exon6-sgRNA Full Vector (artificial) |
| SEQ ID NO: 21 | pAAV-CRISPR-Aass-Exon7-sgRNA Full Vector (artificial) |
| SEQ ID NO: 22 | 5′ ITR (artificial) |
| SEQ ID NO: 23 | 3′ ITR (artificial) |
| SEQ ID NO: 24 | PolyA (artificial) |
| SEQ ID NO: 25 | PolyA (artificial) |
| SEQ ID NO: 26 | HLP Promoter |
| SEQ ID NO: 27 | U6 Promoter |
| SEQ ID NO: 28 | hSaCas9 (S. aureus) |
| SEQ ID NO: 29 | HA Tag (artificial) |
| SEQ ID NO: 30 | Nucleoplasmin NLS (artificial) |
| SEQ ID NO: 31 | OLLAS Tag (artificial) |
| SEQ ID NO: 32 | NLS (SV40) |
| SEQ ID NO: 33 | siRNA for Aminoadipate-Semialdehyde Synthase |
| SEQ ID NO: 34 | siRNA for Aminoadipate-Semialdehyde Synthase |
| SEQ ID NO: 35 | Aminoadipate-Semialdehyde Synthase (human) (genomic) |
| SEQ ID NO: 36 | AASS cDNA (human) |
| SEQ ID NO: 37 | Aminoadipate-Semialdehyde Synthase (human) (protein) |
| SEQ ID NO: 38 | Aminoadipate-Semialdehyde Synthase (mouse) (genomic) |
| SEQ ID NO: 39 | Aass cDNA (mouse) |
| SEQ ID NO: 40 | Aminoadipate-Semialdehyde Synthase (mouse) (protein) |

### B. Specific Examples

Glutaric Aciduria type I (GA-1) is an inborn error of metabolism with a severe neurological phenotype caused by the deficiency of Glutaryl-CoA dehydrogenase (GCDH), the last enzyme of lysine catabolism. The state of the art indicates that the toxic catabolites in the brain are produced locally and do not cross the blood brain barrier. The experiments disclosed herein, which use knockout mice and liver cell transplantation, demonstrated that toxic GA-1 catabolites in the brain originated in the liver. Moreover, the characteristic brain and lethality phenotype of the GA-1 mouse model can be rescued by two different liver directed gene therapy approaches. These experiments question current pathophysiological understanding of GA-1 and demonstrate for the first time a targeted therapy for this devastating disorder.

The essential amino acid lysine is a building block of proteins but is also catabolized to Glutaryl-CoA, which eventually enters the tricarboxylic acid cycle and generates energy. If the conversion to Glutaryl-CoA is inhibited by the deficiency of the Glutaryl-CoA dehydrogenase (GCDH), then toxic catabolites such as glutaric acid (GA) and 3-hydroxy-glutaric acid (3-OH-GA) accumulate (FIG. 1A).

These intermediates accumulate in the brain and kidney where they cause clinical symptoms, a disorder known as glutaric aciduria type I (GA-1) (Goodman S I, et al. (1975) Biochem Med. 12:12-21). In the brain, striatal injury leads to complex movement disorders and subdural or other hemorrhages. Infection, fasting, or other physiological stress can trigger an encephalopathic crisis with poor prognosis. In the kidney, chronic renal failure can be observed typically in older patients. The most critical phase of this disorder are the first six years of life, and since early treatment clearly reduces the high mortality and morbidity, GA-1 is included in most countries' newborn screens.

Currently, the standard of care for GA-1 patients is strict dietary restriction of lysine and carnitine supplementation, in addition to emergency support during decompensation. Despite early diagnosis and prospective care, 33-25% of all patients suffer-long term neurological disabilities (Strauss K A, et al. (2003) Am J Med Genet C Semin Med Genet. 121C:53-70; Boy N, et al. (2021) Genet Med. 23:13-21). Currently there is no specific therapy available for GA-1, possibly related to our poor understanding of this devastating disorder.

### Example 1

### Rescue of Glutaric Aciduria Type 1 in Mice by Liver Directed Therapy

Among the scientific community, it is widely accepted that toxic catabolites accumulate locally and do not cross the blood brain barrier. GA and 3-OH-GA are believed to be retained in the brain with limited efflux possibly as reactive Acyl-CoA species (FIG. 1A), thereby leading to an intoxication of neurons. Hence the cornerstone of current therapy is substrate reduction, e.g., restriction of lysine, which enters the brain via the solute carrier family 7-member 1 (SLC7A1) transporter. Interestingly, lysine is predominantly catabolized in the liver, but besides a transient increase of transaminases during encephalopathic crises, there are no reports of liver dysfunction or failure of GA-1 patients.

To elucidate the role of the liver in the pathophysiology of GA-1 and challenge the current paradigm of local accrual of toxic catabolites, a few transplantation experiments with the mouse model of GA-1, the Gcdh^{-/-} mouse (Zinnanti W J, et al. (2006) Brain. 129:899-910) were performed as described herein. These mice die on high protein diet only after a few days. A Gcdh^{-/-} mouse was created by deletion of the gene in TIRF zygotes (see methods). This strain can be repopulated with exogenous hepatocytes and thereby replace the host liver.

First, healthy hepatocytes (Gcdh^{-/-}) were transplanted into Gcdh^{-/-} mice and put them on high protein diet after liver repopulation. As expected, the non-transplanted Gcdh^{-/-} mice died only after a few days on casein diet. However, about half of the transplanted mice survived the dietary challenge (FIG. 1B). Remaining transplanted mice were harvested after 170 days and analyzed for GA-1 catabolites in the liver and brain (FIG. 1C).

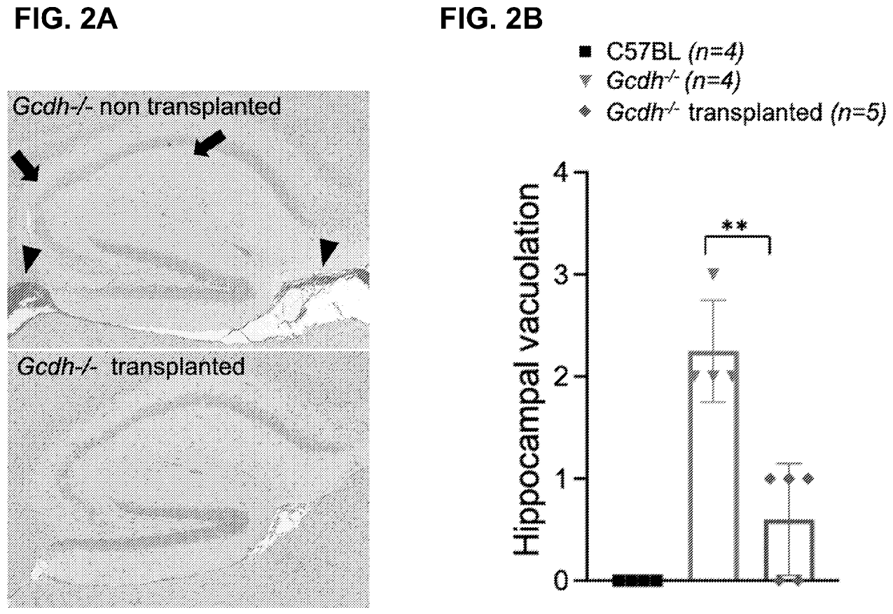

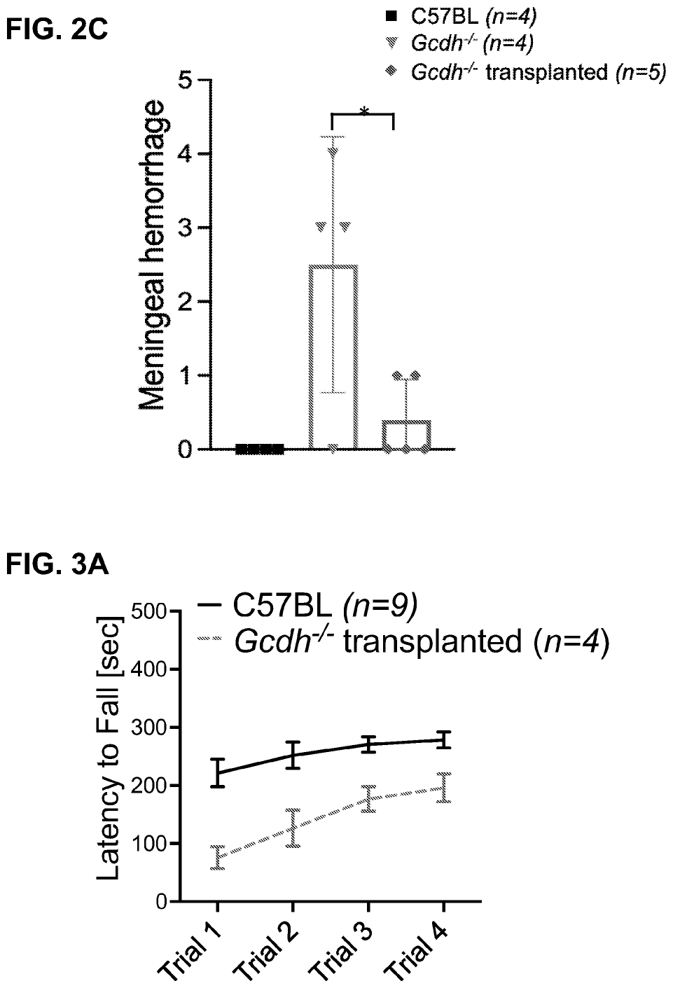

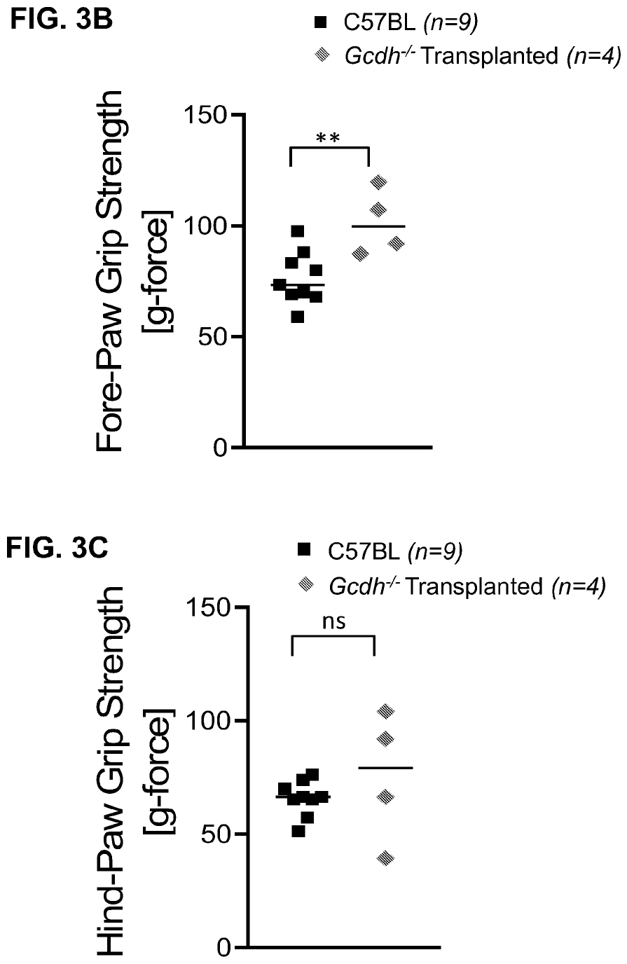

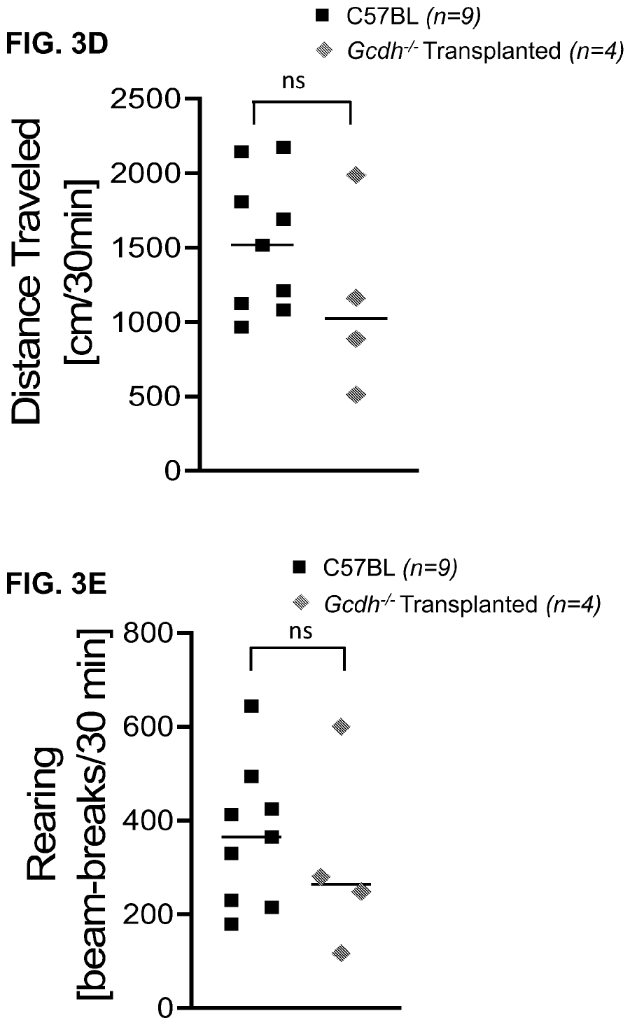

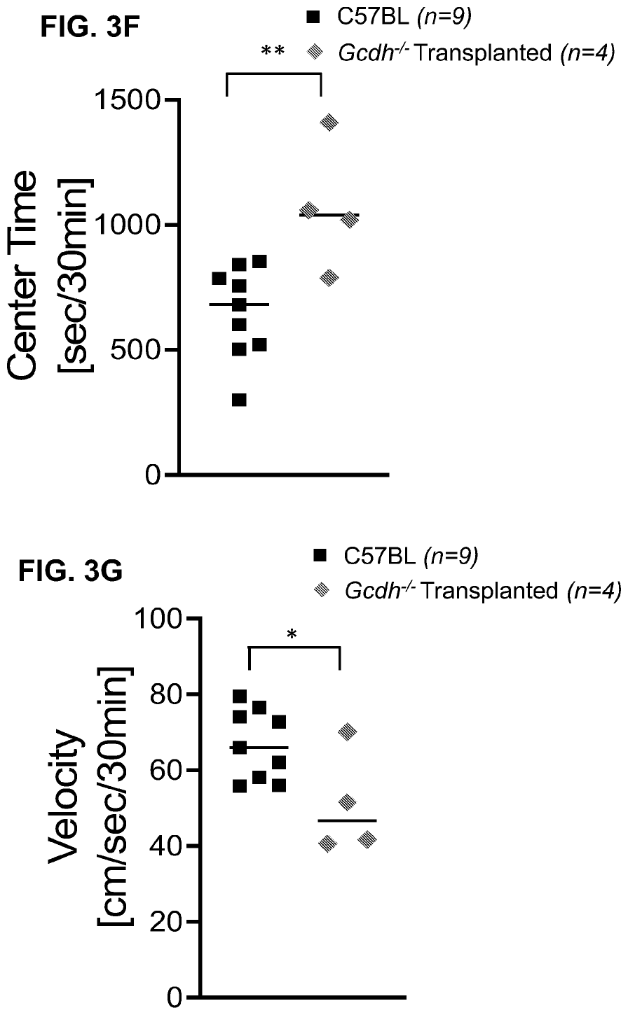

Surprisingly, not only the liver but also in the brain toxic GA-1 catabolites were similar to healthy mice, despite the Gcdh deficiency in the brain. Moreover, the typical neuronal vacuolation in the Gcdh^{-/-} mouse model was not observed in transplanted mice (FIG. 2A, FIG. 2B, FIG. 2C) and motor performance was comparable to C57B6 wild-type mice (FIG. 3A-FIG. 3G).

Immunostaining of transplanted but expired mice revealed unsuccessful transplantation in all 6 mice, while all surviving mice had an almost complete repopulation with healthy hepatocytes (FIG. 1D and FIG. 1E). This experiment indicates that the biochemical and histological phenotype in the brain of Gcdh^{-/-} mice can be reverted by restoration of a normal lysine catabolism in the liver.

### Example 2

### Double Knockout Mice Showed No Obvious Disease Phenotype

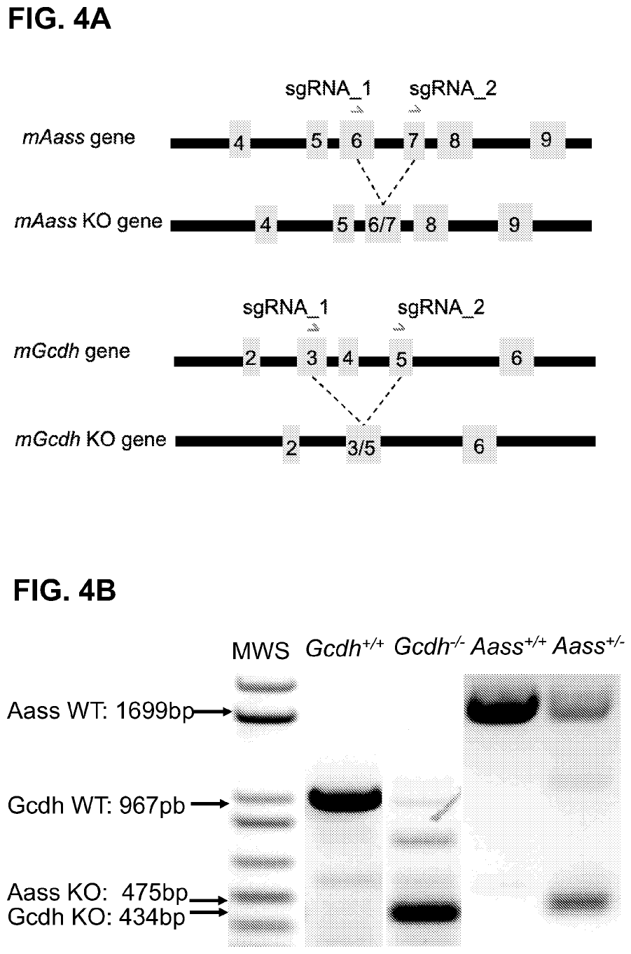

To get further inside into the disease mechanism, whether a Gcdh-deficient liver can lead to accumulation of toxic catabolites in a Gcdh^{-/-} brain, which has no flux through the lysine catabolism pathway and cannot produce locally the catabolites, was examined. To answer this question, a double knockout mouse was generated. This double knockout contained a Gcdh deletion as well as a deletion of aminoadipic semialdehyde synthase (Aass), which is the first enzyme in the lysine catabolic pathway, (FIG. 4A, FIG. 4B). When these double knockout mice (Gcdh^{-/-}/Aass^{-/-}) were exposed to high protein diet, they survived, had no obvious disease phenotype, and demonstrated only a small elevation of toxic catabolites (FIG. 1F, FIG. 1G). The latter finding agreed with a previous report showing that the pipecolate pathway only generates small amounts of toxic catabolites (Leandro J, et al. (2020) J Inherit Metab Dis. 43:1154-1164).

### Example 3

Double Knockout Mice Died Quickly After Transplanted with Diseased Hepatocytes

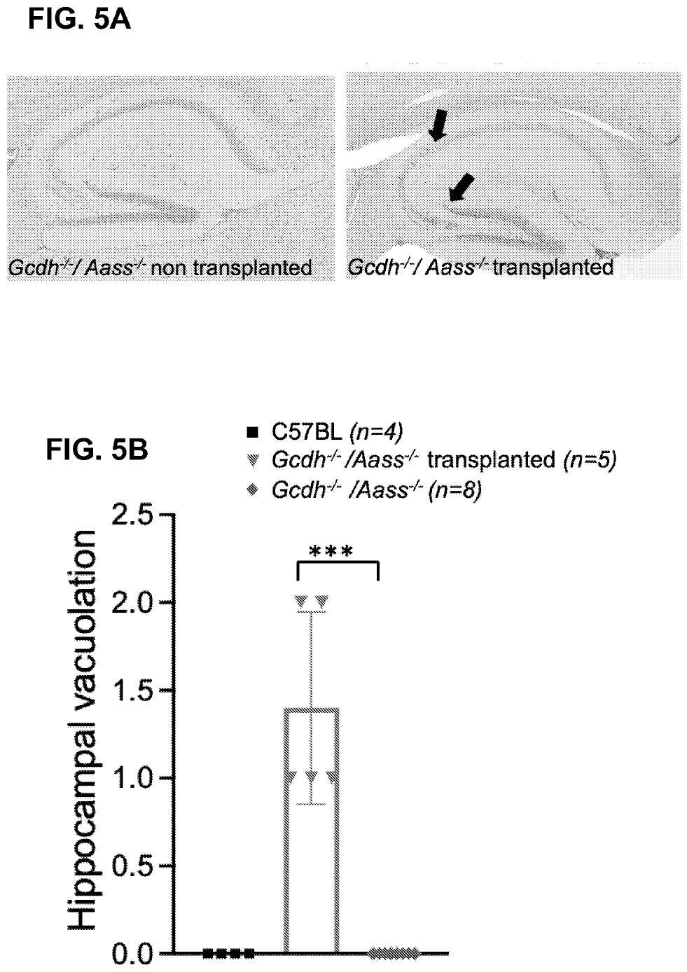

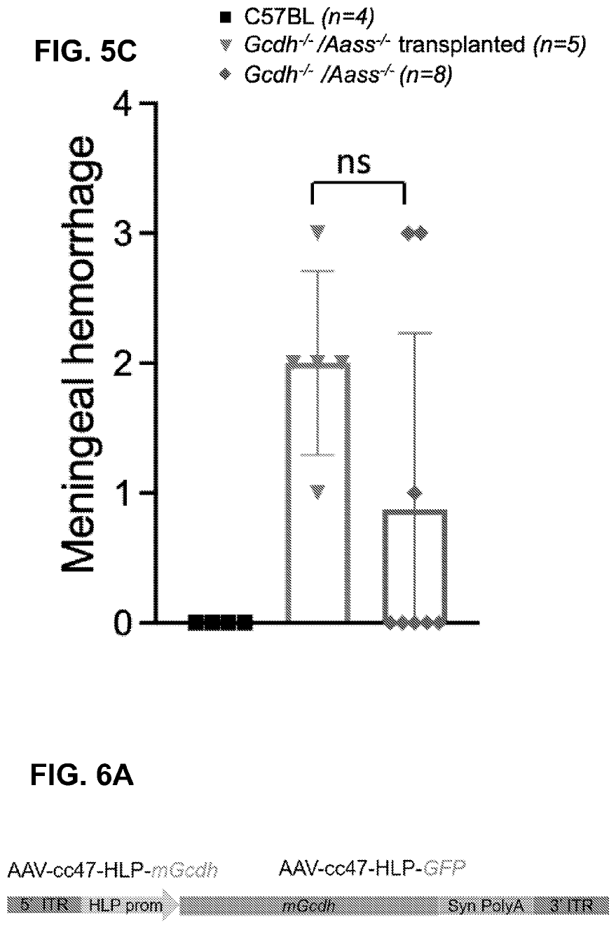

Next, diseased GA-1 hepatocytes (Gcdh^{-/-}/Aass^{+/+}) were transplanted into double knockout mice (Gcdh^{-/-}/Aass^{-/-}). Interestingly, the transplanted mice only survived a few days on high protein diet (FIG. 111) and accumulated already—after a few days—high amounts of catabolites in the liver and brain (FIG. 1I). Only mice with high repopulation of diseased hepatocytes could trigger death on high protein diet (FIG. 1J and FIG. 1K). Similar to non-transplanted Gcdh^{-/-} mice, transplanted double knockout mice developed increase vacuolation in the brain (FIG. 5A, FIG. 5B, and FIG. 5C).

In summary, these transplantation experiments indicate that hepatic lysine catabolism directly impacts the accumulation of toxic catabolites in the brain, e.g., reduction or increase of catabolites in the absence of functional lysine catabolism in the brain (summarized in FIG. 1L

These results were unexpected and surprising in view of the fact that there have been no disease symptoms and no accumulation of catabolites in the brain of either the fruit bat (McMillan T A, et al. (1988) J Biol Chem. 263:17258-17261) or the liver-specific GA-1 mouse model (Sauer S W, et al. (2006) J Neurochem. 97:899-910), which both have a Gcdh-deficiency in the liver only.

Moreover, isotope tracing experiments in C57B6 mice demonstrated only very limited accumulation in the brain after intraperitoneal injection of GA-1 catabolites (Sauer S W, et al. (2006) J Neurochem. 97:899-910).

However, these studies used animals that had a functional lysine catabolism in the brain (Gcdh^{+/+}/Aass^{+/+}). In these animals, the lack of neurotoxicity can be explained by a reduced flux of GA-1 catabolites across the blood brain barrier or an efficient detoxification of GA-1 catabolites in the brain even when originating from the liver. The data presented herein support the latter interpretation and is compatible with all previous observations in bats or mice. Also, data from the human Genotype-Tissue Expression (GTEx) project shows abundant expression of all lysine catabolic genes in relevant brain areas of humans (Consortium GT. (2015) Science. 348:648-660).

### Example 4

### Gcdh^{-/-} Mice Demonstrated a Rescue of Lethality Upon Expression of Gcdh in the Liver

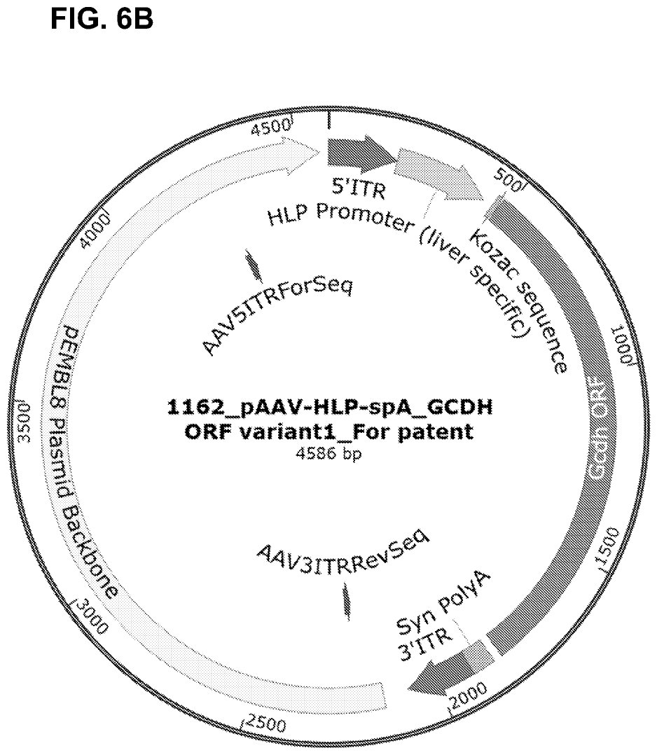

In addition to identifying mechanistic inside into the unclear GA-1 pathomechanism (Pankowicz F P, et al (2017) Gut. 66:1329-1340), the transplantation studies disclosed herein generated a scientific rationale for a liver directed therapeutic approach. Accordingly, an Adeno-Associated Virus (AAV) based gene therapy vector was generated. This AAV vector expressed a wild-type copy of the Gcdh gene or green fluorescent protein (GFP) as a control under a liver specific promoter. (FIG. 6A, FIG. 6B).

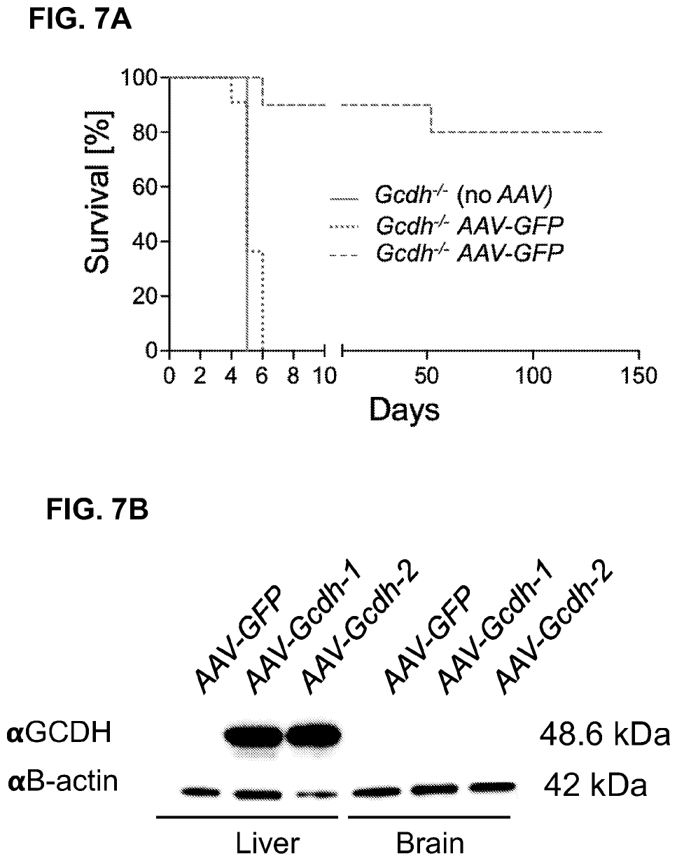

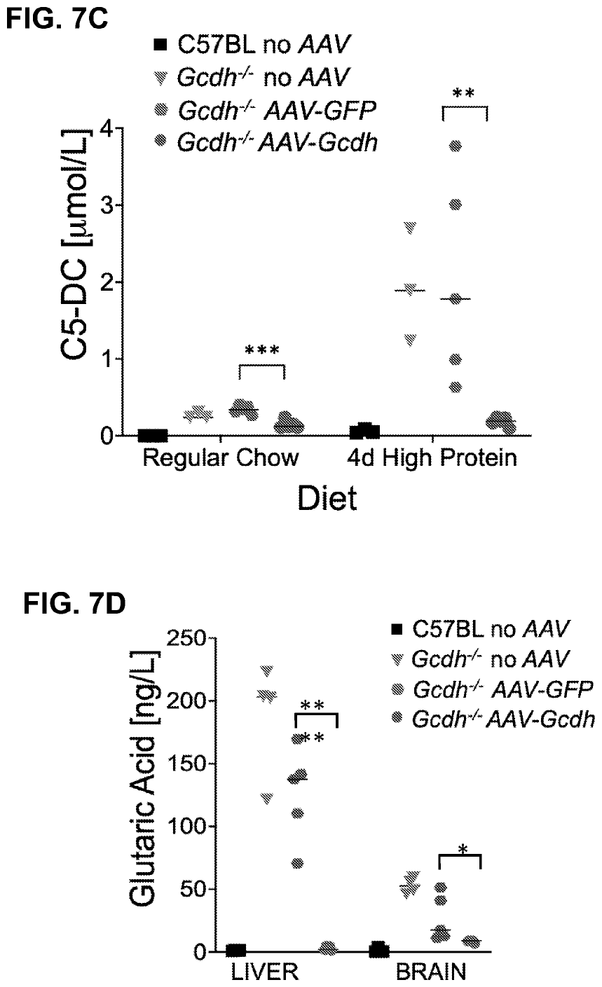

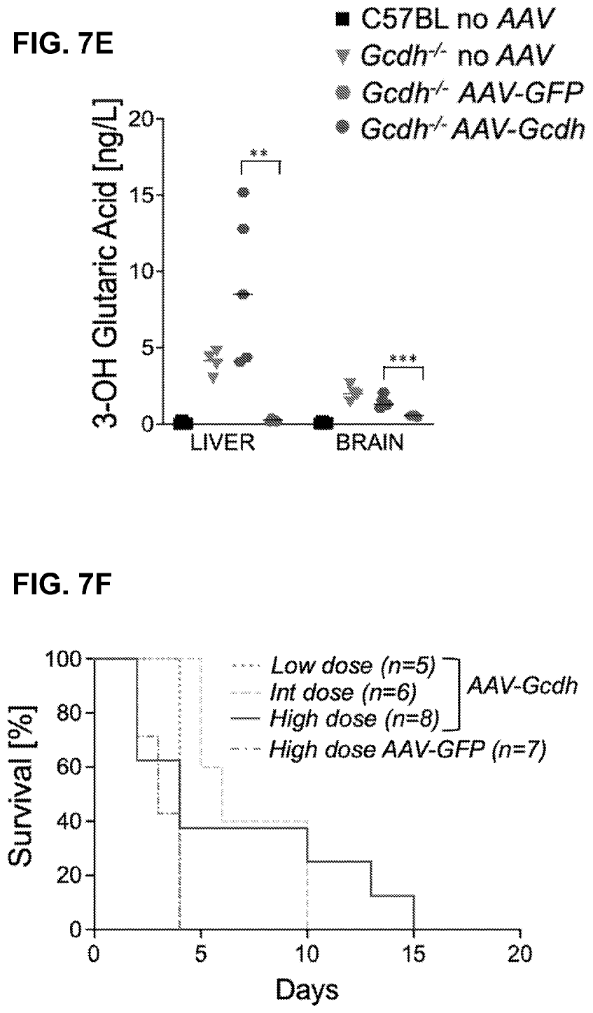

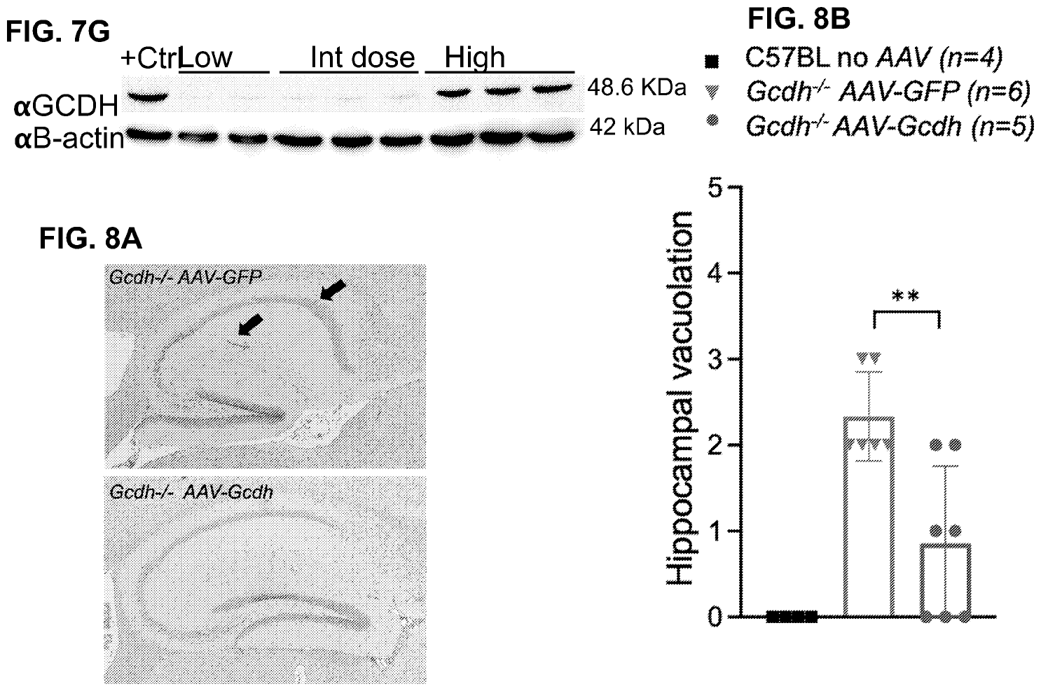

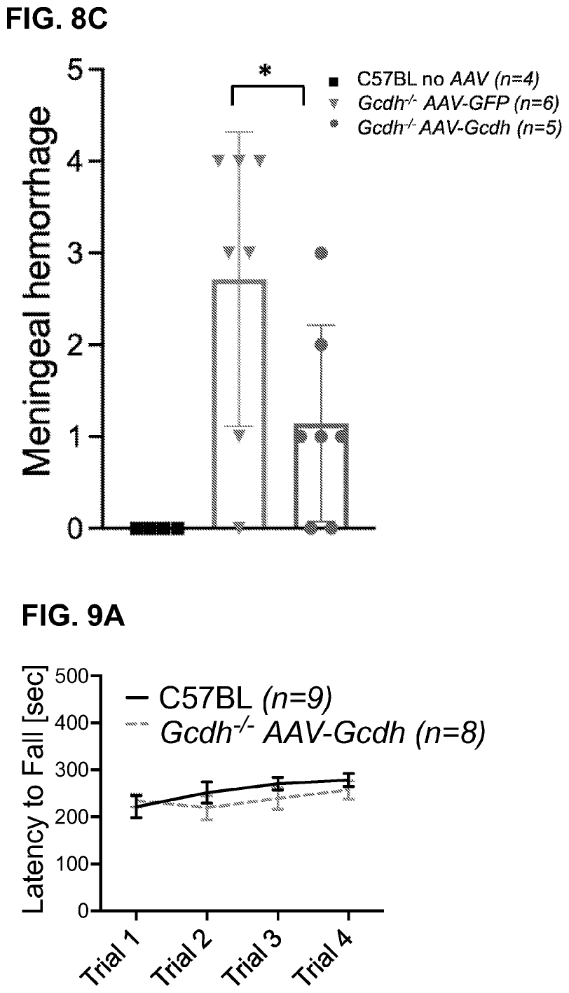

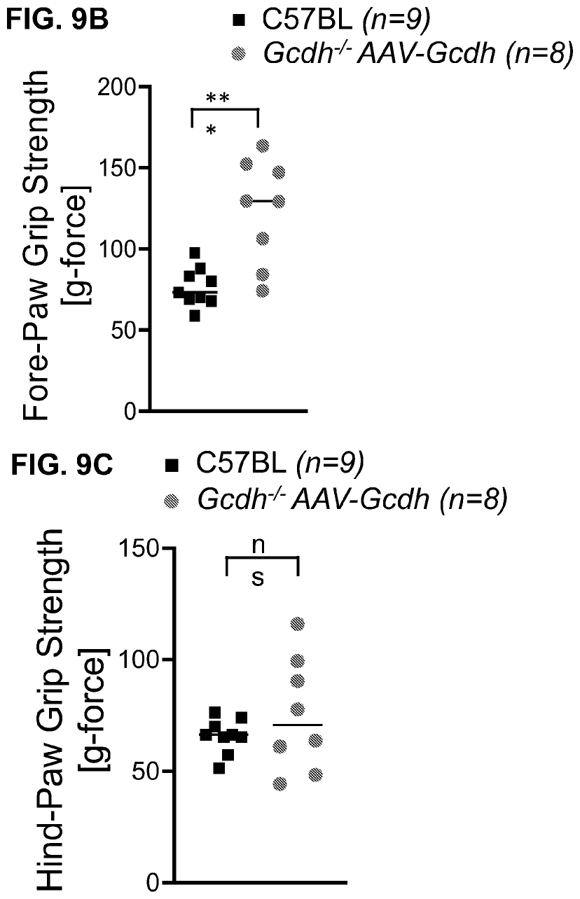

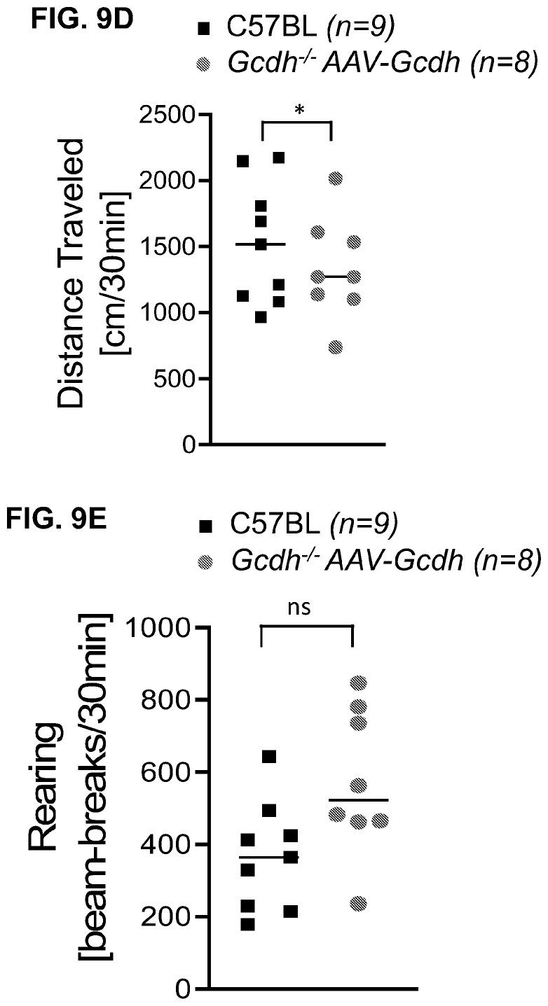

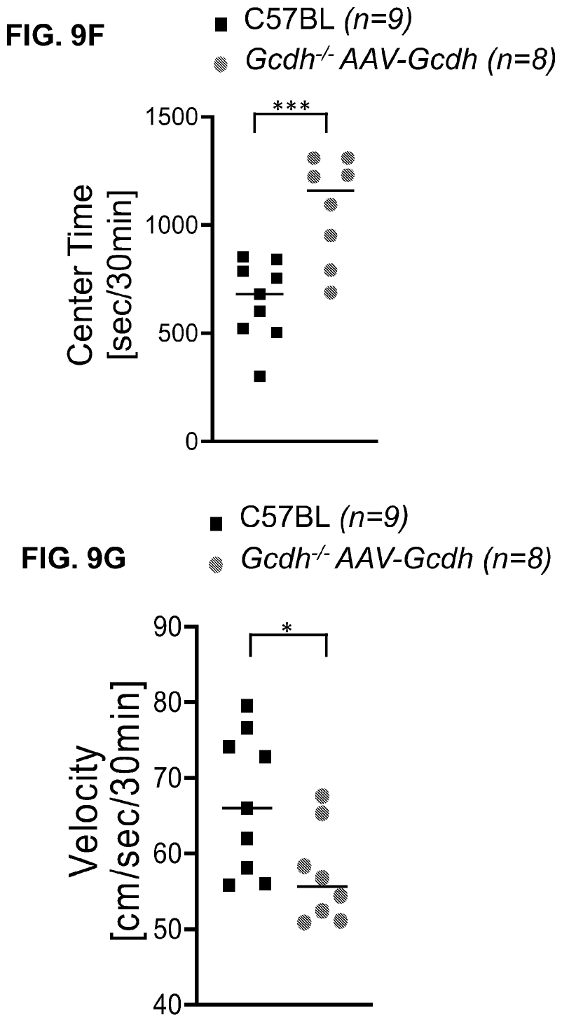

3-week-old Gcdh^{-/-} mice were intravenously injected with AAV at a dose of 1.5×10¹² vg/mouse. Two weeks after injection, mice were put on high protein diet. As shown in FIG. 7A, most mice demonstrated a rescue of lethality upon expression of Gcdh in the liver but not in the brain (FIG. 7B). Glutarylcarnitine (C5-DC) levels in the blood (FIG. 7C) as well as GA (FIG. 7D) and 3-OH-GA (FIG. 7E) levels in brain and liver indicated a biochemical reduction of toxic metabolites upon treatment with AAV-Gcdh. Neuronal vacuolation and meningeal hemorrhage were also decreased in AAV-Gcdh treated compared to untreated Gcdh^{-/-} mice (FIG. 8A-FIG. 8C). Motor performance of treated mice was comparable to C57B6 wild-type mice (FIG. 9A-FIG. 9G).

Since GA-1 patients ideally need to be treated when neonates, neonatal Gcdh^{-/-} mice were injected with a low dose (3×10¹¹ vg/mouse), and intermediate (7.5×10¹¹ vg/mouse), or high dose (1.5×10¹² vg/mouse) of AAV-Gcdh. After weaning, the treated mice were exposed to high protein diet. Although a dose dependent therapeutic effect was observed, the neonatal data was not as pronounced as was the data generated with the older Gcdh^{-/-} mice (FIG. 7F). This difference can be attributed to a dose dependent effect, but also to the overall lower expression of Gcdh in the liver (FIG. 7G) of the neonatally injected Gcdh^{-/-} mice compared to a treatment after weaning. The difference can also be attributed to dilution of the AAV or to silencing upon growth of the liver despite injection of much higher dose per weight. Nevertheless, the data indicate that repetition of AAV administration in can improve the therapeutic effect in neonatal Gcdh^{-/-} mice.

### Example 5

### AAV-CRISPR Therapy Resulted in a Dose Dependent Deletion in Aass in the Liver

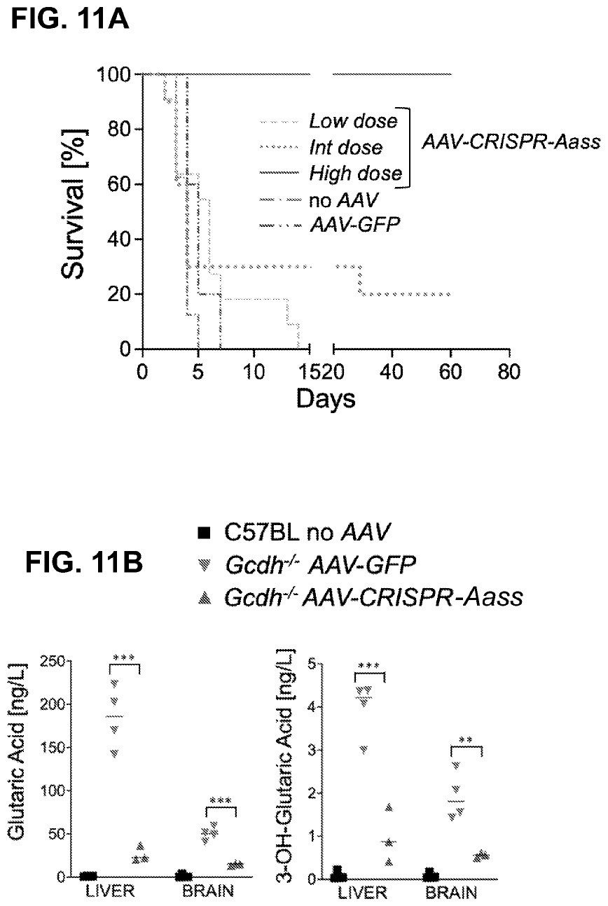

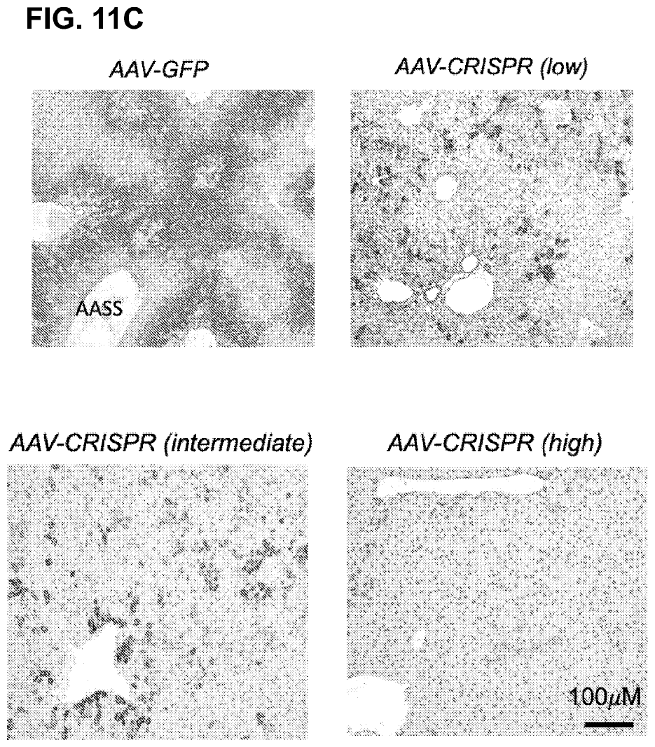

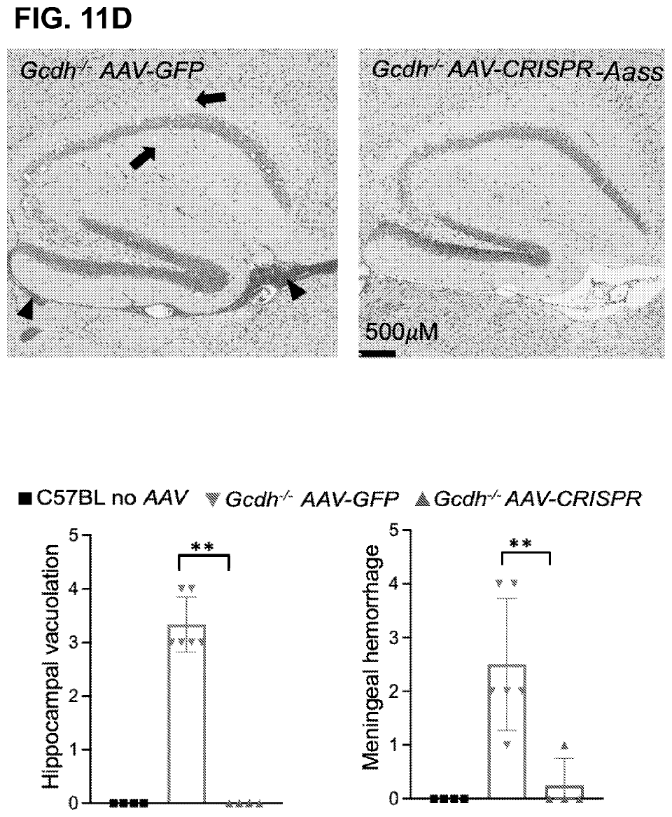

Instead, to explore an alternative therapy in Gcdh^{-/-} pups and leverage the determination that deletion of Aass could be therapeutic, a recombinant AAV gene therapy vector expressing CRISPR/Cas9 effector molecules targeting and thereby deleting Aass were developed. (FIG. 10A-FIG. 10D). Neonatal Gcdh^{-/-} mice were intravenously injected with a low dose (2.4×10¹¹ vg/mouse), an intermediate dose (6×10¹¹ vg/mouse), or a high dose (1×10¹² vg/mouse) of AAV. The high dose of AAV-CRISPR rescued all six injected pups from intoxication on high protein (FIG. 11A).

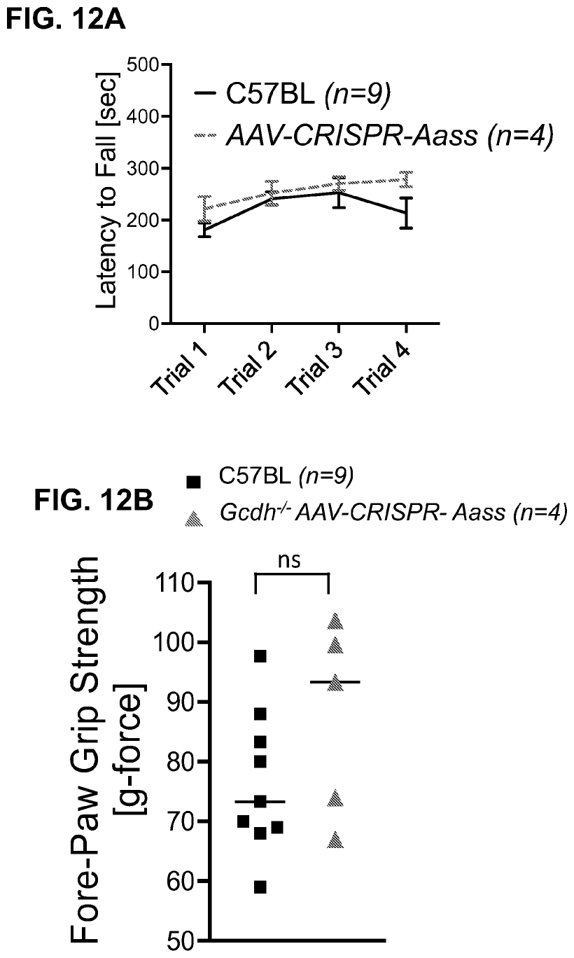

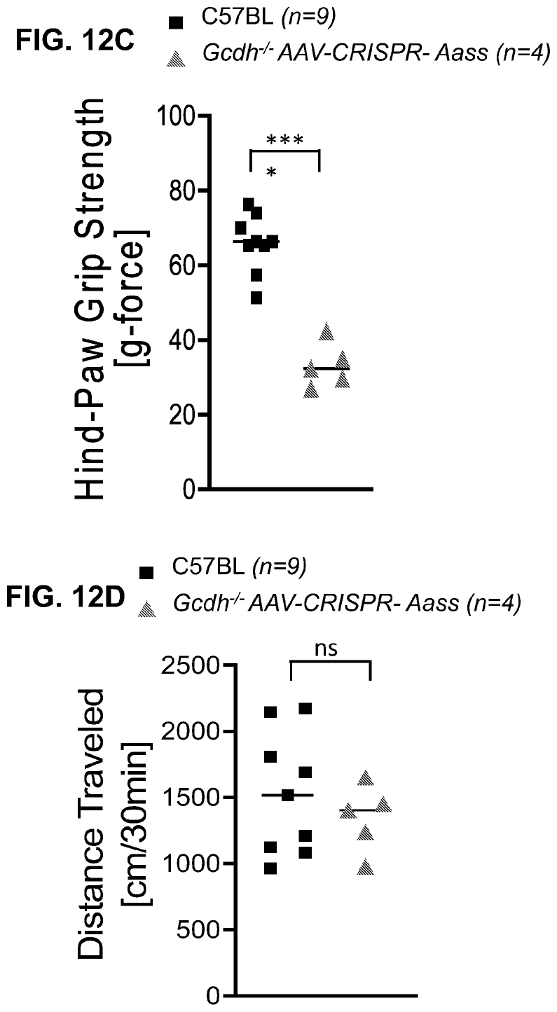

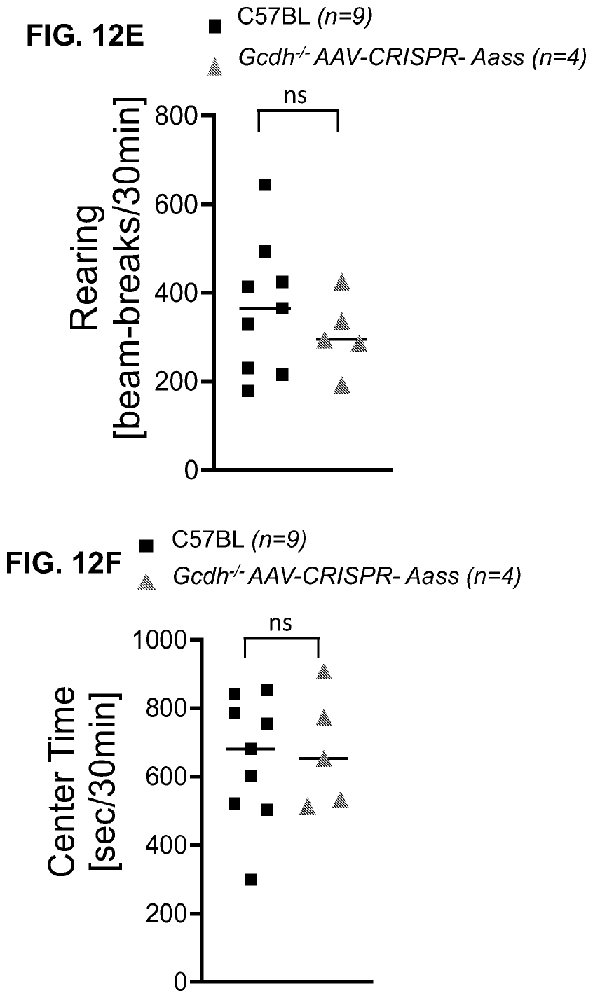

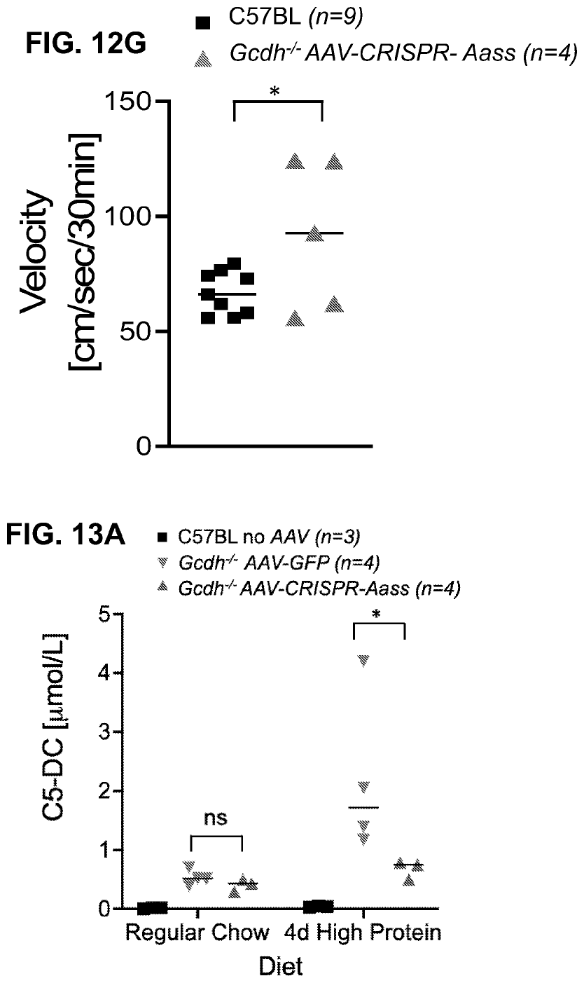

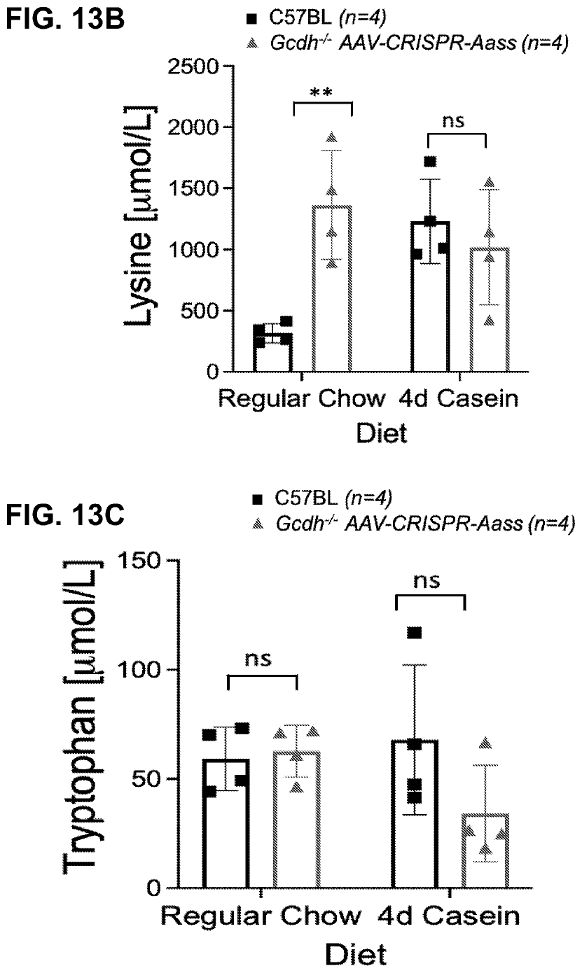

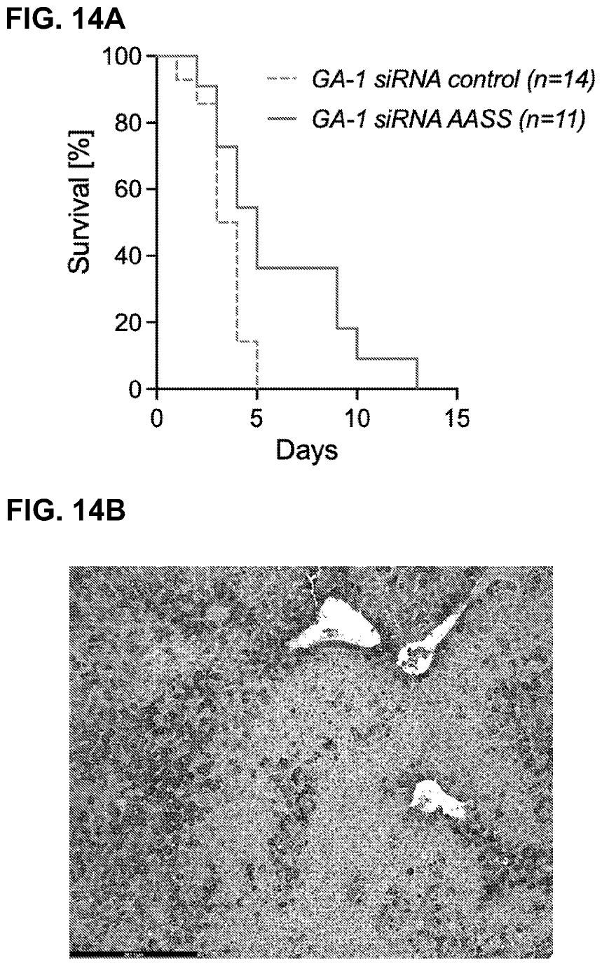

Biochemical analysis revealed a significant reduction of toxic catabolites in liver and brain (FIG. 11B) after 60 days on high protein diet compared to AAV-GFP treated mice after only 4 days on high protein. AAV-CRISPR therapy resulted in a dose dependent deletion in Aass in the liver (FIG. 11C). Most importantly, hippocampal vacuolation and subdural hemorrhage could not be observed in AAV-CRISPR treated Gcdh^{-/-} mice in contrast to clear pathological alterations in non-treated mice only after 4 days of high protein diet exposure (FIG. 11D). Before harvesting the AAV-CRISPR treated Gcdh^{-/-} mice. neurobehavioral testing was performed. A mild difference was detected in the hind paw grip strength compared to wild-type C57B/6J mice. The other tests showed no difference. (FIG. 12A-FIG. 12G). Interestingly, lysine levels in the serum were not significantly increased (FIG. 13A-FIG. 13C) despite the lack of hepatic lysine catabolism. In summary, these results demonstrate that Aass deletion in the liver is therapeutic for Gcdh^{-/-} mice.

The data demonstrate that that both liver directed therapies presented here could complement current dietary approach particularly for patients with compliance issues or for the prevention of long-term sequalae. The transplantation experiments described herein conclusively demonstrate that the liver directly contributes to toxic accumulation and therapeutic reduction of toxic catabolites in the brain.

### Summary of Experiments

The work presented herein demonstrates that the liver directly contributes to toxic accumulation of catabolites in the brain as part of the GA-1 disease pathology. This is surprisingly and unexpected and therefore challenges that traditionally held view in the art. To this end, the three methods of redressing the dysfunctional lysine catabolism pathway in the liver also demonstrated the ability to reverse GA-1 disease pathology in the brain.

Also, all therapeutic approaches described here are less sensitive to non-compliance of patients. Hence if patients (or parents) are not adhering to a strict daily control of the current standard of care (diet & supplements), then metabolic decompensation might occur. Proposed therapeutic approaches are either curing the patient (hepatocyte or whole liver transplantation or gene therapy) or non-daily dosing (twice a week up to every second month) is likely for an siRNA approach. Hence such therapeutic approaches should allow patients to live or go in areas with limited access to emergency care and specialist metabolic clinics, which is the only way to mitigate currently encephalopathic crisis and metabolic decompensation.

Most importantly, with current standard of care 25-33% of these patients continue to develop acute and long-term neurological complications. (Strauss K A, et al. (2003) Am J Med Genet C Semin Med Genet. 121C: 53-70; Sauer S W, et al. (2006) J Neurochem. 97:899-910). Gcdh^{-/-} mice successfully treated with described therapies do not have any severe or acute neurological complications.

What is claimed is:

1. An isolated nucleic acid molecule, comprising: a nucleic acid sequence encoding glutaryl-CoA dehydrogenase.

2. The isolated nucleic acid molecule of claim 1, wherein the nucleic acid sequence encoding a glutaryl-CoA dehydrogenase comprises a sequence having about 50%, about 55%, about 60%, about 65%, about 70%, about 75%, about 80%, about 85%, about 90%, about 95%, or more identity to the sequence set forth in SEQ ID NO:17 or SEQ ID NO:18.

3. The isolated nucleic acid molecule of claim 1, wherein the nucleic acid sequence encoding a glutaryl-CoA dehydrogenase comprises the functional domains.

4. An isolated nucleic acid molecule, comprising: a nucleic acid sequence encoding one or more elements of a gene editing system, wherein the one or more elements comprise an Cas9 endonuclease and a sgRNA directed at a target sequence within the aminoadipate-semialdehyde synthase gene.

5. The isolated nucleic acid molecule of claim 4, wherein the target sequence within the aminoadipate-semialdehyde synthase gene renders a complete knockout.

6. The isolated nucleic acid molecule of claim 4, wherein the Cas9 comprises any Cas9.

7. The isolated nucleic acid molecule of claim 4, wherein the Cas9 endonuclease comprises a sequence having at least 80%, at least 85%, at least 90%, or at least 95% identity to the set forth in SEQ ID NO:28.

8. The isolated nucleic acid molecule of claim 4, wherein the sgRNA comprises the sequence of any one of SEQ ID NO:07-SEQ ID NO:10.

9. The isolated nucleic acid molecule of claim 4, wherein the Cas9 endonuclease comprises the sequence set forth in SEQ ID NO:28, and wherein the sgRNA comprise the sequence of any one of SEQ ID NO:07-SEQ ID NO:10.

10. A viral vector, comprising: the isolated nucleic acid of claims 1-2.

11. The viral vector of claim 10, further comprising a promoter operably linked to the nucleic acid sequence encoding glutaryl-CoA dehydrogenase.

12. The viral vector of claim 11, wherein the promoter operably linked to the Cas9 endonuclease comprises a liver specific promoter.

13. The viral vector of claim 12, wherein the liver specific promoter comprises the sequence set forth in SEQ ID NO:26.

14. A viral vector, comprising: the isolated nucleic acid molecule of any one of claims 4-9.

15. The viral vector of claim 14, further comprising a promoter operably linked to the Cas9 endonuclease.

16. The viral vector of claim 15, wherein the promoter operably linked to the Cas9 endonuclease comprises a liver specific promoter.

17. The viral vector of claim 16, wherein the liver specific promoter comprises the sequence set forth in SEQ ID NO:26.

18. The viral vector of claim 15, further comprising a promoter operably linked to the sgRNA.

19. The viral vector of claim 18, wherein the promoter operably linked to the sgRNA comprises a U6 promoter.

20. The viral vector of claim 19, wherein the U6 promoter comprises the sequence set forth in SEQ ID NO:27.

21. The viral vector of claims 10-20, wherein the vector comprises an adeno-associated viral (AAV) vector.

22. The viral vector of claim 21, wherein the AAV vector comprises AAV8 or AAVcc47.

23. A viral vector, comprising: the sequence set forth in SEQ ID NO: 19.

24. A viral vector, comprising: the sequence set forth in SEQ ID NO:20.

25. A viral vector, comprising: the sequence set forth in SEQ ID NO:21.

26. A method of modulating lysine catabolism in the liver, the method comprising:
administering to a subject in need thereof a therapeutically effective amount of siRNA, wherein the siRNA silences a gene in one or more lysine catabolism pathways in the liver of the subject, and/or
administering to a subject in need thereof a therapeutically effective amount of the viral vector of any one of claims 10-13, wherein expression of the nucleic acid sequence generates a functional glutaryl-CoA dehydrogenase in the liver of the subject.

27. A method of modulating lysine catabolism in the liver, the method comprising:
administering to a subject in need thereof a therapeutically effective amount of siRNA, wherein the siRNA silences a gene in one or more lysine catabolism pathways in the liver of the subject, and/or
administering to a subject in need thereof a therapeutically effective amount of mRNA therapy, wherein expression of the encoded nucleic acid sequence generates a functional glutaryl-CoA dehydrogenase in the liver of the subject.

28. The method of claim 26 or claim 27, wherein the gene in one or more lysine catabolism pathways comprises the aminoadipate-semialdehyde synthase gene, α-aminoadipic semialdehyde gene, the kynurenine aminotransferase 2 gene, the dehydrogenase E1 and transketolase domain-containing protein 1 gene, the L-lysine alpha-oxidase gene, the ketimine reductase mu-crystallin protein gene, the peroxisomal sarcosine oxidase gene, or the pyrroline-5-carboxylate reductase gene.

29. A method of restoring the expression of glutaryl-CoA dehydrogenase, the method comprising:
administering to a subject in need thereof a therapeutically effective amount of the viral vector of any one of claims 10-13, wherein expression of the nucleic acid sequence generates a functional glutaryl-CoA dehydrogenase.

30. The method of claim 29, wherein the expression of glutaryl-CoA dehydrogenase is restored in the subject's liver.

31. The method of claim 29, wherein normal lysine catabolism is restored.

32. The method of claim 29, wherein the subject has been diagnosed with glutaric aciduria type 1 (GA-1).

33. The method of claim 29, wherein administering the viral vector comprises intravenous administration or intrahepatic administration.

34. The method of claim 29, wherein a therapeutically effective amount of the viral vector comprises a range of about 1×10¹⁰ vg/kg to about 2×10¹⁴·vg/kg.

35. The method of claim 29, further comprising monitoring the subject for adverse effects following the administering step.

36. The method of claim 35, wherein in the presence of adverse effects, the method further comprises modifying one or more steps of the method.

37. The method of any of claims 29-36, further comprising administering to the subject a therapeutically effective amount of a therapeutic agent.

38. The method of any of claims 29-37, further comprising administering to the subject a therapeutically effective amount of a one or more immune modulators.

39. The method of claim 38, wherein the one or more immune modulators comprise methotrexate, rituximab, intravenous gamma globulin, Tacrolimus, prednisolone or a prednisolone analog, SVP-Rapamycin, bortezomib, or a combination thereof.

40. The method of any of claims 29-40, further comprising monitoring the subject's metabolic and/or physiologic improvement.

41. The method of any of claims 29-40, wherein liver-specific modulation of lysine catabolism is restored, wherein one or more aspects of lysine homeostasis is restored in the subject's liver, wherein the level of toxic catabolites in the liver is reduced, wherein the level of toxic catabolites in the brain is reduced, wherein the metabolic flux from glutaryl-CoA to crotonyl-CoA is restored in the subject's liver, wherein the subject's motor performance is improved, wherein the subject's memory function is improved, wherein the subject's anxiety is reduced and/or prevented, wherein the subject's neurological sequelae are reduced and/or prevented, wherein the subject's vascular dysfunction is improved, wherein the subject's quality of life is improved, wherein the subject's life span is increased, wherein the subject's survivability is increased, or any combination thereof.

42. A method of reprogramming a metabolic pathway, the method comprising:
administering to a subject in need thereof a therapeutically effective amount of a viral vector comprising a nucleic acid sequence encoding a Cas9 endonuclease and a sgRNA directed at a target sequence in the aminoadipate-semialdehyde synthase gene.

43. The method of claim 42, wherein normal lysine catabolism is restored.

44. The method of claim 42, wherein the viral vector comprises the viral vector of any one of claims 14-20.

45. The method of claim 42, wherein administering the viral vector comprises intravenous administration or intrahepatic administration.

46. The method of claim 42, wherein a therapeutically effective amount of the viral vector comprises a range of about 1×10¹⁰ vg/kg to about 2×10¹⁴·vg/kg.

47. The method of claim 42, further comprising monitoring the subject for adverse effects following the administering step.

48. The method of claim 47, wherein in the presence of adverse effects, the method further comprises modifying one or more steps of the method.

49. The method of any of claims 42-48, further comprising administering to the subject a therapeutically effective amount of a therapeutic agent.

50. The method of any of claims 42-49, further comprising administering to the subject a therapeutically effective amount of a one or more immune modulators.

51. The method of claim 50, wherein the one or more immune modulators comprise methotrexate, rituximab, intravenous gamma globulin, Tacrolimus, prednisolone or a prednisolone analog, SVP-Rapamycin, bortezomib, or a combination thereof.

52. The method of any of claims 42-51, further comprising monitoring the subject's metabolic and/or physiologic improvement.

53. The method of claim 42, wherein the expression of the nucleic acid sequences eliminates the functionality of aminoadipate-semialdehyde synthase in the subject's liver.

54. The method of any of claims 42-53, wherein liver-specific modulation of lysine catabolism is restored, wherein one or more aspects of lysine homeostasis is restored in the subject's liver, wherein the level of toxic catabolites in the liver is reduced, wherein the level of toxic catabolites in the brain is reduced, wherein the metabolic flux from glutaryl-CoA to crotonyl-CoA is restored in the subject's liver, wherein the subject's motor performance is improved, wherein the subject's memory function is improved, wherein the subject's anxiety is reduced and/or prevented, wherein the subject's neurological sequelae are reduced and/or prevented, wherein the subject's vascular dysfunction is improved, wherein the subject's quality of life is improved, wherein the subject's life span is increased, wherein the subject's survivability is increased, or any combination thereof.

55. The method of any of claims 42-53, wherein the metabolic pathway that is reprogrammed comprises a lysine metabolic pathway.

56. The method of claim 55, wherein the lysine metabolic pathway comprises the pipecolate pathway and/or the saccharopine pathway.

57. A method of treating and/or preventing GA-1 disease progression, the method comprising:
administering to the liver of a subject in need thereof a therapeutically effective amount of hepatocytes, wherein the hepatocytes are GCDH^{+/+}/AASS^{+/+}.

58. The method of claim 57, wherein liver-specific modulation of lysine catabolism is restored, wherein one or more aspects of lysine homeostasis is restored in the subject's liver, wherein the level of toxic catabolites in the liver is reduced, wherein the level of toxic catabolites in the brain is reduced, wherein the metabolic flux from glutaryl-CoA to crotonyl-CoA is restored in the subject's liver, wherein the subject's motor performance is improved, wherein the subject's memory function is improved, wherein the subject's anxiety is reduced and/or prevented, wherein the subject's neurological sequelae are reduced and/or prevented, wherein the subject's vascular dysfunction is improved, wherein the subject's quality of life is improved, wherein the subject's life span is increased, wherein the subject's survivability is increased, or any combination thereof.


SEQUENCE LISTING (40 sequences)

SEQ ID NO: 1 - DNA, 8840 bp, Homo sapiens
  source 1..8840: mol_type = other DNA
agttgcactgtagcctcggcagtgaaccgggaggtactaccaggtaaggaaggtgcggtagccccagccgtgggtgagaggagctccgctctgacacccccgctcctgtaggtcgccgtcgttgctccgctcgctctgagagagcatggccctgagaggcgtctccgtgcggctgctgagccgcggacccggcctgcacgtccttcgcacgtgggtctcgtcggcggcgcagaccggtcagtgtggggtcgggagtgtggagggaaggagggaggaactgggggtttagggactttccggggtgactttcccgttctgtgcttgcagagaaaggcgggagaacacagagccaactggctaagtgtaaggacctctggtcgcaccgtgtgtctgctgcccctgttcagctgtctgtctgccgcaggtggactctgtcccagaatccgagagctgcccgagcggggtggcagggtcgtggccagggtcagaggcactaaggcagtgagtgcgctgtgcctgcggggccggagaaaagtcacctgatcagtctcgcttgcagctcgcactagccggggggcgacatgggtgttggggggtagggctgatgagggtccgagaagggagggcacagtgatcttgcggactggaccgaggcgaattccccttcccagcctcgcgtcccgagtttgactggcaggacccgctggtgctggaggagcagctgaccacagatgagatcctcatcagggacaccttccgcacctactgccaggagagactcatgcctcgcatcctgttggccaatcgcaacgaaggtgggcgggctggtgggtgccctgagactgctcctccgcctggagccatagccaccccacctcaaggcccctctgtccttggggctggggcttcctgtggcctaggcctgggcctgaatttgggcactggtccctttgcagtttttcatcgggagatcatttcggagatgggggagttgggtgtgctgggccccaccatcaaaggtaggaacaagtatctctccacacactgcagaaccctctgtattctgaaagcctcttcctccttccctccctccctttcttccttccttctctttcttttcttttccttttctttcctttcttcttcccccccaacagagtctggctctgttgcccaggctggagtgcagtggcacgatcttggctcactgcaaattctgcctcccaggctcaagcgattctcctgcctccacccctctagtagctgggattacaggtatgtgccaccatgcctggctaatttttgtatttttagtagagacagggtttcactgtgttggccaggctggtctcaaactcctgacctcaggtgatccgcccacctcagcctcctaaagtgctgggattacaggcatgagccaccacgttcagccttctttttgagatggagtttcgctcttgttgtccaggctggagtgcagtgatgcaatcttggctcactgcagcctccacctcccgggtttaagtgattatcctgcctcaggctcccgagtagctgggattacaggcgtccgccaccacgcctggctaatttttgtatttttagtagaggtggggtttcaccgtgttggccaggctagtctcgaactcctgacctcaggtgatccacccgcctcagcctcctgattacaggtgtgagccaccgttgcccggcccttttcttttttttttttttttgagatggagtttcgctctgtcacccagcctggattactggattacagtggtgcgatcctggctcactgcagtttcctcctcctaggttcaagcaattctgccacctcagccttctgagtagctgggattacaggggtgcaccaccacgcccagctaatttttgtattttttagtagaaatgggatttcaccatgttggccaggctggtcttgaactcctgacctcaggtgatccacccacctcggcctcccaaagtgctgggattataaacgtgagccaccgtgcctggcccctttgtttctttttttagagacagggtctcactgtgttgcccaggctgttctcaagtgatcctcttgccttactgaaagcccccttctttccctaagccacaatttcccagtctgtaaagtggggctgttgtcccaccctctgaaagtggctgtggagatgaaatgaataaacctcagctagggccagcttggtgcctgcctccttgtgtgtccttattcagccctgtctcttgggtcttagctgggcagggccctgttctctattgtcctgctttcccctcctactaccaccaggatatggctgtgctggggtttcgtctgtggcctatgggctcctggcccgagagctggagcgggtggacagtggctacaggtcggcgatgagtgtccagtcctccctcgtcatgcaccctatctatgcctatggcagcgaggaacagcggcagaagtacctgccccagctgggtgagtggctgcccatggggcctggtggaaggaagacagtctctgaggtctggaactcaagggtggggctgtcccctgagcctattctgtccctatctcaaagatagcataagtggccacctggacccccgccagaccctgggcttcacctggagatctgatccctggccagcctgactgtccccctctgtgaccaccgtcatctccctatgctttctgtgttccccagtccagcccaaagtttaaagtccaccaggttcctgctggccatttgcagtggctcacacctataatcccagcactttgggagggtgaagtgagaagatcccttgagcccaagagttcgaaaccagcctgggcaacgtaaggagaccccatgtctattagaaaaacaaaaaaaggaaagagcctatgtgacctgcgctaagtggacgttggccctcttccgtggtgtctcggaggtgttcagctgcttcaagatgaagctgaacatctccttcccagccactggctgccagaaactcattgaagtggacgatgaacgcaaactttgtactttttatgagaagcgtatggccacagaagttgctgttgacgctctgggtgaagaatggaagggttacgtggtcggaatcagtggtgggaacaataaacaaggtttccccttgaaacagggtgtcttgacccatggccgtgtccacttgctactgagtaaggggcattcctattacagaccaaggagaactggagaaagaaagagaagatcagttcatggttgcatcgtggatgccaatctgagtgttctcaacttggttattgtaaaaaaaaggagagaagggtattcctggactgactgagactatgatgcctcgtcacctggggcccagtacagctagcagaatccgtaaacttttcagtctctctaaagaaaatgatgtctgccagtatgttgtaaaaaagcccttaaacaaagaaggtaagaaacctaggaccaaagcacccaagattcagcgccttgtcactccacatgtcctgcagcacaaacagcggcgtattgctctgaagcagccgcatattaagaaaaataaagaagaggctgcagaatatgctaaacttttggccaagagaatgaaggaggctaagaagcaccagggacaaatcgtgaagagacgcagactgtcctctctgcgagcttccacttctaagtctgaatccagtcagaaataagattttttgagtaacaaataagatcagattcgcaaaaaaaaaaaaaaacccacaaggccctgtgtgaccaggcctctgctgacctttccacctcatctctggccactcatgtgatcctcccagccagacttagctacttgaaattctccagaagtaccaccagttctttacacatctgttccctctcccagcactgctcttccccacagcctcctcctgcttaactcctcacacccttccagtttggccaactccttcctgattccctgggtccccctccccatcttggcattgtgatcatgactttggggtaacctgtctcttgaaagcagggccagatctaacttagttatgaggtctgactcaggggcgagggtaattttttttttctttttttgagacaaagtcccgctccgtcccccaggctgaagtgcagtggtacaatcttggctcactgcaacctgcggttccccagttcaagcaattcctgtgcctcggcctcccaattagctgggattgcaggtgcttgctacccgtgcctggctaatttttgtatttttagtagagatggtgtttcactatgttggccaggctggtctcgaactccagacctcaagtgatccatctgccttggcctcccaaagtgctgagattacatgtgtgagccacctcgactggcctaattttttgtatttttagtagagatggggtttcaccatattggtcaggctggattttttttcttttttgagatggagtcttgctctgttgcccaggctgtagtgcagtggcatgatcttggctcactgcaacctctcctgcccgggttcaagcgattctcctgcctcagtctcctgagtagctgggattacaggcacccaccaccacatctggctatttttttttttttttttttttaagtagagatggggtttcaccatgttggccaggctggtctggaactcctgacctcaattgatccacctgccttggcctcctaaagtgctgggatgacaggcgtgagccactgcaccccgccacgaggataatttttgagtaaggggatgtatcagggaccaggcagccttgtgactttgtcttgtgcctgcagccaagggggagctcctgggctgcttcgggctcacagagcccaacagcggaagtgaccccagcagcatggagaccagagcccactacaactcatccaacaagagctacaccctcaatgggaccaagacctggtaagggttctgggtggtgggcaggtggtgaacaggggcaaaggggcactggtcagacccctcaccgactgttccatccccaggatcacgaactcgcctatggccgatctgtttgtagtgtgggctcggtgtgaagatggctgcattcggggcttcctgctggagaaggggatgcggggtctctcggcccccaggatccagggcaagttctcgctgcgggcctcagccacaggcatgatcatcatggacggtgtggaggtgccagaggagaatgtgctccctggtgcatccagcctgggggtaagtggcagccactttgggaatgggtgttgggtcacctgcggatgcggctttgtcaggcaggctccgtgctggggacgcggctccctgtgcctgtggagcccacacagtggtgattcttactcagccggactcgctgacgtgctgaaaactgcccccatttggtgaccgtctcgctcatcccggctctgcccgggacacatgggcctgaaccagctcagtcatttgactcacagtgcatcttctggcatccgtcagcctcctggctctgagcatcgaacccagatgccaggctgggtgggactgtgtgcaaaccgagtgagcaggcaccgagcttcagtgccagggccatctgtgatgtgaaccacaacctgagtccccctgcgtggggtggctggggaggaggctttccctgcttcagagttggttctgcataggccctcttggtgtctcttgggtgggcctgaggcgccatctcaaccctacagggtcccttcggctgcctgaacaacgcccggtacggcatcgcgtggggcgtgcttggagcttcggagttctgcttgcacacagcccggcagtacgccctcgacaggtgtgtgagggctgcagtgagattctctgggggtgtggggcagcttgggtttcactctctataccatgggtgactccccagcccccacccaccaggcctgagttccttgctctggaatgaccagtgacgtccttctgagcagctgtgggctgagtcaacggcagggccagggcaagcttgggggcactgaggcagcctgggaaggcgtcctggagcagggggccccaggacagggacggggtgggagagtgggcctcccctcgctcttaccctgccattgcccatgtaggatgcagtttggtgtcccactggccaggaaccagctgattcagaagaagctggcagacatgctcactgagattaccctgggccttcacgcctgcctgcagctcggccgcttgaaggaccaggacaagtaggggctgtgtggtgggggcggggggatggcagcggtggctggaggaccttgtgtccttcctggagagaaaggtccttcctgcctggtggccctggggacctgaaccttctgctgtccctcttgtccttgatgggctgggctgaggacagccccactggtccctcattgggagcttggctgcatcagggaatccccaccccgggctaggtttgcttggagcatcgggatgccaggatccccagtccttgttaccctcatgtgccactcccagggctgcccccgagatggtttctctgctgaagaggaataactgtgggaaagccctggacatcgcccgccaggcccgagacatgctgggggggaatgggatttctgacgagtatcacgtgatccggcacgccatgaacctggaggccgtgaacacctacgaaggtaggagctggacctcagagggctcactgaggcctcagtgtctggggagggggtacagggaggtgggacggggacaggtctgagtccaactccacctatcactaagtgacagtgtgacctggggcgagccttacccctctgtccctcatctggagttactcacatggggactgtggaagtgaagtgcttcatgcagcacacagaggccccatcaggccttgcgaggggctcccagctctttctcccacatgctcgggaggagggatgttcctgagacaggtaagctccgaaggcagcccaggggtgaggcccgactcctagcaggctggtggacgcaagggagtgagcgaagctacacgcaggaatcaacgctccattttgttaagagacaaaagtcatcttcagatgacggtgtttgcaccctgtaactgatggtgatttacatgggggtccagaaccccttgcttggtgggaagacagatgtggaaaaagatacttaaaaaaaaaaaaaaagtgcaaacccagcatgcgctatgtagaaatagagggaggtatgaggaccccccgggcagagatgcttccagacagagagaacagcaaatgcaggaggggtgccttccctgcgcaccttgcaaccccagatcttgtctctatctggcatctggggggtggggggatcctacaaatgtgagaatgaccctgaggacaagcagcgggcgccatgagatggttttatgcagagaagggatgagtcagatttggtgctttaggggctcggcgcagtggctcacacctgtaatcccagcgctttgggaggctaaggcaggaggattgcttgagcccaggagttagaagttatgatgagctatgatcaccccactgcactccagcctgggtgacagagtgagaccctatctatatttaaaaaaaataggtttgtactttaggaagtaaacagaattggctctaaaaaggaggctacaatctgccttctgcttcaaggagctggggaggagcaaaggatgagacaccagggcatggggtgtgggattctctgggagaggcggcttcagatgggcagaggtcagctgaggcaagtgactgctcttccgatgtgctgtccctcctatccctcccgagggtaagaaagggcttgctgtgtttccttggagctcaggggtgctttcagcctctgtggcaaggaagcgtggccaggttgaaaatcaaacatttaataaactgtggggctgggggtggggagaacttgccaagggtggggagcacgaagcctgggcctatggcaggggctggacttgctacattttgggagttcagcacaaggagctttgggtttttgtttttttctgccaagatgcattatagaaccatgaaaaacaatttctcagtgtggcccaaatggtggcctcacagtcttctcctggagactgtcactaaggcgtgagtctcccggcagctgtcagcattcaccatctctgttggtctgtacttctgaagcagtggcctggggatacatgaaaattgattttaaagggaagttgtgagctatgaaaactccaaaccgactctgtattaatcttgtccaggtacacatgacattcacgccctgatccttgggagagctatcacgggaatccaggcgttcacggccagcaagtgagccgctccatcaggggcccgaaactctcaagcccctttctggagagatgcctggctggaccgtaggagcgctgtgctctgagcttagaaagggaggtggcggatggagtgggaagtgagagacactgatttttaaatatcaaaatttcccttctgaagtcgttcagatgtgttccttaaaaagaagatggaattctctgtagagcgtctcaatccacttttaaccatggatgagagcagactccatttaccctgaaatagcagcttctcttgagaggagagtgacatggaagcaactccgtctgctgcagctgaccccctcacactgagttcacagtgcgccctccctccctcccatctgggggtagtgccttatgctgggtgttggagcagagtgagggagaggaaaataaagacctgcacatctgaccccaaggtgtcaggccggtttactggtaa

SEQ ID NO: 2 - DNA, 16363 bp, Mus musculus
  source 1..16363: mol_type = other DNA
acggccccgtgggattcgggggccccggccccaaatcctgcgtctcctcagaccgccaccactgcgggagggagcgtactgcttgaatggtaggcactgggtacctggccctcaaacaaccctggctggaaacaccgttggtgcctgtcatttacccacacggttcccctaacccctttctcagctccagacaactccattaggtcttgctagagacagctaagacacttaccaagaatagtattcaggatgggagaaggggttgcagagttctgagtcaaaggggacagcttcgactccgttttccctagcctttccctgtcttcacatagcccagaaaattacagctcaaagcctggaaatcccaggacacctttatctggacctttgtctctatgagcctagtctcttaagatatagagcttagaattctgcttcctgttggagaaataggcctcaccttgatctcagacccctagaacatctcccccaaacaaggctcagaggctctcccactcctccacccccaaaaaacagagcagaggatattaaagtaagatagacgccccacccctaagcactatgccctatgcaagaccacctgccccctcccacacactcacactggccccaccttgaggaagtcctcctgggtgtccaggaactgtcccagggtggtgagtgtactgatggagggtaagacagtctcagctgaggccatggctggctggtgtccaccctggacctcaggcctcctctctctcttctgcctcatgggctatgaggctaggaaagtttcttggagggctcctgtctgcccacatctgattggctgtcttccctgataagacccaggcaaagcaaaacggcaaaggaggggtgtgtgcgatatgtgtgtggtgtgcttctttttttttttttttaaggttttcgaaacagggtttctctgtgtaactgagccctggctgtcctggactggcttatgtagaccaggctggcctagaactcacagacatctgcctacctctgtctcctgagtgctgggattaaagttgtgatgtgtcactttcaaggaatagacctcaccctggcctgttttcaaactggtccctgaaaaccaaccctgtatgtaaagtcaatcagatgtcaaggaataggggcttcaaagttttaggattcaggttgggtaaagaaaagtcctggtcgctgctccccacccccaccccacctcccacaggactgagaagacccctctggtttggtttcaagtctctctccaaggagtggaccaggaaggatagatttcttgggaaaggtctttgggaccctttgacccccatggagaccactgcagtccttactctcccagacaacagcagcaacggtgctagtggcgggccttggcaccggctccctttcaggcattatcagacacaccagatgtttcaggccgctatcaggtagggggcccagaacaaccatagctctgttcctccttgagcagaagagctgggggttggtgccacaaattattttagctttgaattgctctgttgtgttgaggtctgagctgtttagcaaaaggagtcccctcaaatcagcccactctggggctctgaggaccctcactggctactgcatggagctgaaggggatggctaggaagtttacagtgtggtgggctaggacccatagatggagctgcagctagtatgccgttcagaagagctcagccctgactctgcctaacggctcattgtgtgcgggaccccatgatagatggcctgctattcccatacttctattctggtgccctatgagtcagggtagggaaatggggtgtcacgggtacaataatatcacacatacaccatcattgctcggtgtccataggggacatcagatccattatagaaaatggtacctagtcaggcatggtagtgatgcctttaatcccagcacttggaaagcagaggctggtggatctctatgaatttgaaaggtctgtttggtctacataataagttccaggacaaccagggctacatagtctttgtctcgaaagacgacaacaagggtaaaacagtgctggggacttagcttcatggagtgcttgcaccgtattaactttaaaagaaaagcataaagacctaggttcagatccctggcactgacacaaaagccaagtgtcactgtgcatgtctaagactagtgctagacagatggaaacgggaagatacctggtgctcactggatagtcagcctagccgatcaggtccagtgtcaggcccagcataggaaataaggtaaagaaaacgaaggaagagaccatcaaccctgaccgctgggctgcacgcatgctcaagcacacactagacacacgccccaacaatcagtgtgctaagacgagtaaccggaggacaacaggctaatggaggagggcaggcaaccatggaaggttacatgcctcattattttgtctatataattgccagaacaggtcaggacgagcagtgcttcccagaggctggggcaggagactgctaggagacaaagtctcatttgggaggacagaaacattctgaaactcagtagaagtgtttgtcacaggacactgtggtgctacattgcccactgaatctgcatttatttattgattgattgattgattgattgtatgagtacactgttgctgtccccagacacaccagaagagtccatcatatcccattacagatgattatgagccacaaccaaagaagacattttactggggcttgctcacattttcataggtgtagtccattatcatagtggggagcatggtggcatgaaggcaggagctggtccaataactgagagaaacatcctgagagagagagagagactttgaacctcaaagccaacctccagcgacacacttcccaattcttacaaccctttcaaaaagtgccactctctggtaattaggcattcaatatatgagcctttgggggccattctgggatttattgtttttgttttttcaagacaaagtctctgtgtagcctggaacttgtttagcagaccaggctgacctcgaactccctctgcttctagaatgctcggattaaaggtgtatgccaccaccatccagctacggggactattctttttatttaattattttattattttatttatgtatatgagtacactgtcactgtcttcagacacagcagaagagggcgttgggtcccattacagatggttgtgagccaccatgtggttgcttgtaattgaactcaggacctctggaagagcaaacagggtgctctttcccacttctctccagtcccctattaggaccatccttaaacctccacagaacctttgataggaaagttttcttatatagccccacctgcacagggcatagtcagattcacaggaattaggggttgaagaggccaagggctgcaggataagggaggtgaccaagtggagaactatacatagtgtcagaaagcccagggtgatgatgcagtccctgttgtataaggggagagtcagaaggttttgttttactgttaagggtgagagagtgtgtgctctaaacaggaccagtaagagaaaggggagaacccaaagagacgataaactacctaagccagtctatttagccacccagacctagcgtctctcctacccaagtgataaagttacgtttgctttctctgcagctcaattttctttctttcacggactttcaatctttcatctgccaggaaaagacctcaagatcccctagacaaccaccagcaccccattagagcaggatctcgtgagatcgtcgtaaagggtgtagctcagagcatgtgcttagcatgcactctggggcagagcatgtgcttagcatgcaccagctctggtttgatgttggggtttgaattaaggtgaattatgtgaagataaagtgaatttgagaaggggcctaggaacatctggtgttgatgtttctcaagaaggatttgtgttacaacataatgtatgtttcaactcctagtggaagggacccaggagtgcctgagaccttcctccatctctagagcatagtcagggtaaatgatgttcccacacctcagatgtttttctgaagacaaataacattgttgtgctttatacaaccttgagcatgctggctgtgagcagccttctctgttaccaaaggagctctaataataaaaatgagccttgtcctgtaaattgggcttaggtgctgggctgtgtaaccaccgtgatttcctgcttcagttcttttggcattaataagaaaggctttagctagcctctcacaaataaataaataaataaataaataaataaataaataaataaataaataaagataataataaaaccaaacaactacaattacagaaaaattaaaacaaaagtttccaaagagaatagccacacccaaatagaaaaccaccttgctgagtactgcctagatacacagtaactaacagagaatacaagcttacactgaactatgtagacaagaaaccatcaagagagccaggtgtggtggcacaggcctggaatcccagctctctggaagcagaggctggcagttctgcaagtggccaacctggtctacaaagtgagttcctggacagccagggcggaacagagaaacctgtctcaaaacaaaacaaacaaataaaagccatcaaaagggttggagacaagcctggcaggctggaaaaagccttaaatcccagcactcaggaggcagatacaggcagatctctgagttaaagatcagcctggtctaaagagcgagatccaagacagccagggctacagggagaaaccctggagggggcagggctggaggctttgcagaggattccaggcatccacagagggtttctttgattaacaatccaggctgtcctggaactctttgtaaaccaggctggccttgaactcacaaaaatctgcctgcctctgccaccatacctggctataaataaatctttaaaacaatattttaaaccctgtaagactgagctgggtttgtagttcagaggtagagtgcctgcttatcctgtgctaggtgctaagtttgattccccaggatagaagggagggaagagggaggcagggactcatgtttcattcacatgaacagtcagagtttctgtcttctctcagtcacccacagtcaacagagacataggaacagtcaggactcaagcactggggtttgttgtgaggtgttcaccagagaagactgctcggatataagttcaagccaatagaaggtctttattagctggctgatgtctacactgagtgttcagggtctcagtgtagccatgagcctttttcagggtgagcttttttttttttttttagatttatttattattatatgtaagtacactgtagctgtcttcggacgcaccagaagagggcatcagatctcattacggatggttgtaagccaccatgtggttgctgggatttgaactcaggatctctggaagagcaatcagtgctcttaaccgctgaaccatcatctctctagccccctagggtgagcttttaatcataaaaaacatgtcctgggttggcacacttaggttaacaggaacaggcctttggcaggtatttctgtctgcctgctgagttctacagactgaatggcacttccatcatggagtcagttgtgctaaggtctggagtcctgtttcaggttgaggttgtaaatcaatgctggagggcttgtttagtgtttctgaggccctgggtttaatccatagtagtagcaggagaaggagggccacagaaggtaaaggtatttcccaccaagcctgaaaacctcagttcctccttgggactcatagtggtgggagtgagctgacacacacaggagtgacttggtatgtacatacacagtaaacaaatacaactttctgaggaggaggaaaccatgtaggagctggagaattggccctagtggtgaagagcactcgacagagcccaggggtggtggcatacacctctagtcccagcacttgtaaggaggaggcagagccaggtgcatctttgagttcaaggccagcatggtctatagagtgaattccaggacagccaggtaaagtggaaacttctcgtttctctggagggttggttgtgtcaaatgatgttttgctggggcacacaggtgaaaggatgtcttgttaaagcaggcaggtgaagaagtgttttcctggggtggacacagatgaaaggatgctttgctatggcagacatgtgaaaggccacatgatgaaggagtataaatatgactccacagatagtgggagagagagctggagcattggtttgctttgctccacctcagtcttcttcactaatgacatgcttatattggttcgccttatatagtgttgttgagctccacttgtggcacagccattgagagaaactcacccaagaacttatgaggttcctgtggcagcttgccactgaagcagccgtggccagttggcaagcctttcggcttcctcaggattggactacagctgctggttcatgcgtggtgtctgcctgcagagaggatgaaactactgctgctgcttcctttttggtatttgccacagggctgaactgctgccaaagaagattgagctagcccccgaagaactcttgctgtagggatccacttcctccatatcctaataactttcccactacctctggtgagtggtgggcaaggggagagcttgaatgtttattaaaataggctgtaaaaaaatttatacctacagtcagggctacacagagagaccctatctcgtaaaacaaacttcgatggactgatcttgactgttctagcaagatagaaggtcataagtctaatttatattcctgtttttgtaatcaaacttccttattccactcaaaaagatgactagggcaactataagacatatgttaaaactacgcccttcaggatatctataatattgtggagtttgactttataagccttttgtagatctggctaggggagagtgctctcaggtgcagccctcatcccacactggaagccatacctaaataagcctcttcttattaaaatttatttatggttatggtgaatctctaaggaacctgactgtaacagaggccctgtgttaaatgggcctgcctgagagactgtccagcaggtcaaggcacttgtcaccaagcctaaacacctaaggtggatccctaggacacccatagtggaaggcgaaatgactcttgaaagttgtcctttgtcctcctaaacatgggacatggagagctgtgcatacattgcgtgctctctctctctctctctcaccccccacactaaatagataaatgtaattaaaaaatgaaaacaatgccgggcgtggtggcacacgcctttaatcccagcactcgggaggcagaggcaggcagatttctgagttggaggccagcctggtctacaaagtgagttccaggacagccagagctacacagagaaaccctgtctcgaaaaaccaaaaaaaaaaaaaaaaaaaaaaaaaaaaaaaaaaaaagaagaagaagaagaagaagaaaagaaaagaaaagaaaacaactggaggaatatagtttagttcagtgatgcagtatttgttgagcttgagtagaatgctaggtttgcttcctagcacttcacacacacacacacacacaccacaagatacacttgtaaagatgactatcatagtagctttcacccacacacagctacaagaggagacatagccagttattttattttatttatttattttttttgagacagggtttctctgtatagctctggctgtcctggaactcagtctgtacaccaggctggcctcaagctcagaaatccacctgcccctacctcccaagtactgggattaaaggcgtgtgccaccactacccagctacatagccagttacaagatagaaatacaaccccataaaatgcgttacaggaaagtccaaatgacaagttgctgccctcagactttgacaactgagaagaggatcagaagaatttattactcacaagcccagaggagaaacctactgttggtctgagcaattgcacagatgggtttctataacatttatagttataacattattgctaagccaataatcttcattggtgccttaaactaaaaaccataaaagggtcgatgtaagtctataaggggaagtctctctgtgtggttggtcagtcaagcacttttgagaatcagatattggagctatggttagagctttctctttttgaaggacaattgttagatggctgctttgtcaggataaccagttactatttctcaggagagtttatacagttatgcccccccttcttttctattcttgtcttaattttctccaaatcagtgcatactccaatagaaactgtaacatccaagacacccagttataacatattctagattattctgcacaacacccagaaggtaggagaaccacacaggtacacagaaatgcagttactttgccacagaagatcacatccttccacgaagtacacacaaagcaacaagttgggatggggctcaccacctttctctctgcaggcaccccatttgcggatttcgcaaggtctggcctccaggcgttcttattgaacttgggatcccgcttctcaggctctgggcaactagctaaccaggaatgggcggggcagaaatacagttggccaatcgggtttgcacacttgtttccgccccttcctggttctgctgtttaaaggagagacctgcctgggcatggaagaacccgcttgcaaaaccaggcagctgccttggctcttttaaaaggcagagccacagaggaggcggtgttctgggaggaaccaaggaggaaccaatgagcaacccctattaggcctagtgtgctggtcccggagcacagcccgcaagcaccaggtatgaagggtatggggtgggagaaggctggtccccgctgacgctcagcccttccaggttatggttcctgcaatgctcaccggctacatgtccctgagaggagtctccgcgcggttgctgagccgcaggtccggcctgcgcttcccgcgttttccacgtacctggagctcggcggccgcccacaccggtcagtgcaaggctctggatgggtgggcagggagacacaggacactttctcagtgtggcgttctcgtcctttgtttgcagagaagacacagatccgaccggccaaatgtaagatcacctacccacctattcccagtccagctgtttctctgacccatgctagtccatgaatgcaaggacttctgggaagggaggctgggttatccccagggtcagaagtctaaagtagagggtatttgtttctacttggctggagaggggttgcgcttgtcagactgttttgaagccagaattcacctggagcgagatggggggcggggaatgggtacagcttaaaaatgtgaaagaggataaggggttctggctgactcgacccccatactctacccagcctctcgccccgtgtttgactggaaggacccactcatactggaggagcagctgactgccgatgagaaactgatcagggacaccttccgtaactactgccaggagcggcttatgtctcgaattctgctggctaatcgaaatgaaggtgccttggtggaagggtgggcatgctgggtttcacaaccatacatgatttgcatgtgctgcagtccctggagtcagggactccatgagactgagacagggtctgattctgacacccatctctgcagtttttcacagggacattgtgtatgagatgggggagctgggcgtgttgggacccaccattaaaggtaaggacatttctgtgtatactatgcagcctatacccacctacaacatcttgaaggaccatcccttttcctcagtcacgcatttcatctgtaaaatgggcaatggttatctctcctgaaaacagctgtgggtttgagaggaacaaagtcctaaccagggcagccccctgcccacttccagaagggccatattacagccctactcagtcccagggctgatctttggcctttggtctacccgttcttcttttgcaccagggtatggctgtgctggtgtgtcgtcggtggcctatgggctcctgacccgagagcttgagagggtggacagtggctacaggtcgatgatgagtgttcagtcctcccttgtcatgcaccccatctatacctatgggagcgaggaacagcgacagaaatatctgcccggactgggtaagcagctgttgccaagaggaggaaagtcagatgtcggtctggaatgcaggtcccgcctggtgtccctccacagcctgccttctttcctacagaccacctgtgtccatgtcaggtcctaggactcagttgtcttccttgatctcctgccagcactgtcggttcctctctccccccaaccctttgattttacctctggtcaccatcatctccccttggatttgttttctaggcctctcacccctgtccaaagtttaaagtcttaactgctaaccagatccacgaggctcatgagcggacctttcctctacttctttggctactcagattttatttgtagatatggtctcactgtgtagccctgtttggctgggagctttgtagaccagtagcatcaaactcaaaggtctacatgcctcggtctcccgaggagggacagcttggattaaaggcatatactatcatgcctatccttctggctactattctgattctgattgctcagatactggggattctccagtcaccttggtgacaactgttcctcataccctccttcaaactggatgtggtggcatacccctctaatcccagcagtctagaggccagcttggtctctgtagtgagttgttataggacagccaggggtacacagtgagaccacctcaaaacaaacaaacaagccaggtgccaccacacctttaatcccagcacttgggaggcaaaggcaggcagatctctgaatacaaggtcagcctggtttacagagtgagttccaggacagctagagctaagaaaccctgcctcaaaaaacaaacaagggctggagagatggctcagtcgttgagaacactgactgctcttctagaggtcctgagttcaatttccagcaattccatggtagctcacaaacatctgtaatgggatcccatgtcctcttctggtgtgtctgaagacagctacagtgtactcatataaataacataaataaatcttaaaaaataaacaaaagcaagtaacaagaaaaaaaaactctcaaacttcataccttccttcagctaaacctcttagtgcccttcaagatactcttgccaacccctttctttctggcatccttcacattataatttgattatggaccattttctcagcagctcactgagtgccatgatgcagggtgtatctgcctttattcaggtctgactcagtgagaagataatttatcaggagataaatgagaccctctcttgtgcttacagccaagggtgaacttctgggctgctttggacttacagagcccaaccatgggagtgacccaggtggcatggagaccagagctcgccacaatccatcaaaccagagctacactctcagtgggaccaagacctggtgagagacccaggtagactggtgggtgagaaggtgtgtacatggaagcagcagtcagatctctctataactctctggcctccaggatcaccaactcccctgtggctgacctatttatagtgtgggctcggtgtgaggataactgtattcggggcttcatactggagaagggtatgcggggcctctcagcccctaggattgaaggaaagttctccttgcgggcctcggctaccggtatgatcatcatggacagtgtggaagtgcctgaggagaatgtgctgcctaatgtatccagcctggcggtatgtggtaggcaccttgggctctagtaatggggcacctgctggctcacctttctcaccttttctattgattgattgtatatgagtatactgtatctgtacagataatcgctagccttcatctagttgttgggaattgaatttaggagctctgctcgatccagtcaaccctgctcgctccagcaggccctactcactcagtccctgctcactctggcccaaagatttatttattattataaataagtacactgtagctgtcttcagacgcaccagaagagggcgtcggatctcattaagggtggttgtggggccaccatgtggttgctgggatttgaactcaggaccttcagaagagcagtcagtgttctttttttttttaaagatttatttatttattatatgtaagtacactgtagctgtcttctgacacaccagaagagggagtcagatcttgttatggatggttgtgagccaccatgtggttgctgggatttgaactaaggacctttggaagagcagtcgggtgctcttacctgctgagccatctcaccagccccagtcagtgttcttaatggctgagccatctctccagccctttctcaccttttcaagtgtagagcaaacatagtccttgtggttatggagcattgtgaagttagtgatcctccctgttcacagatgtgctaagttcacctttccagttaccacttagctcattatctctgcccaaaatgcatcttggccttggaccacttactactctgtatgcatgagtagcttcctctcctgccagcctcttagatgtgagcactgaggtcaggttccatgttgtataggatggcttgcacatacagagtggaaaaccactgaacaaggcttgatgcatccaagttttggggctcacaccctggggggggtgttaaatggctgcagatccctttgggacacttgagagatggcttttccttcagttggttcttcccaggccttcagttgactgagacctcttgtcaatcttatagggcccttttggctgccttaacactgcccgctatggcatcacatggggtgtgttgggagctgctgagttctgtttgcacacagcccggcagtatgccctagacaggtatgtgggctgctataacacctgagggtgtggaggattttggggttccccaacacccaccaagtttaagttctctgctctctgagatggcccaggacatcttgagcagttaaaatgttgatgtgacagcagggccagcatgaactaggaagcctcttgaactatgggtcttaatgtgactttgcctgtcctaggatccagtttggagtccccttggccaggaaccagctggttcagaagaagttggcagacatgctcactgagatcacactgggactccatgcttgtttgcaacttggccgtttaaaggatcaggacaagtatggattatatactattgctgaatctctaggccctttctgagatccttcccaaatggtggccccagagatataagtttctccccacttccccttttcttttttcttttttcttttttttttttaagatttatttatttattatatgtaagtacactgtagctgtcctcagacactccagaagagggagtcagatctcgttacggatggttgtgagccaccatgtggttgctgggatttgaactcctgaccttcggaagagcagtcgggtgctcttacccactgagccatctcgccagccccccttttcttttttcaagagaggtttcctctatgtacccttggctgtcctggaactcactctgtagaccaggttggccttgtacccccagagatctgcctgcctcttcctcctgagttctggggttaaaggcatgtgccaccactgctcagtgttccccttggttttgatgggctggatcagacacagacttgccagttggacatgtgctctatcttctaggtgtggtcaggacatgagacccaaagtttataacatatgtgttgttcccagggctaccccagagatggtctccatgttgaagagaaacaactgtggaaaagccctggatattgcccgccaggcacgagacatcctgggaggaaatgggatttctgatgagtaccatgtcatccggcatgctatgaatctggaggcagtgaacacttatgaaggtcagagctgggctcagggaggagggtgtaacagtgacctaggggagggagacagcacagctgagtccaccagctgtcaccaagtggcttcaactctaccactctgtcctttacttggagttagttgttcagagcttgtgggaatgaatgtgtaagggagtgactacatgacttagagatcccaataagaagaccctgagatgctcctgaccttgttctcatcatgggatgggacatgaagggggacacagtcccaagagcaaggtgccaagagaggtatgtgaggaagagaacagcacacattatttgtatcactactttatgttcagagaaaaaaactgtttgtaaactaccatgcacaagggcatgtacccatggcttaggacaaacttggaaaaagacatgaaaaattggagggctaaaatataaaggagggctgtggggatggctcaatgagcccatcagttgccataaaagcaagagaacctgcgattaagtccccacagtctttgtaaaagccagcacagtaacatgtgtctgtaacccagtgcttctaggtaaaatgggaaactcaggcacgaatccttagaagctctcagaccagctgacctgggtacagagtagtcaataacaacgtctcctgtcttaaatgaggaccataccctggcctccccatgtgcacctaaacacacacacctttgaccaatattaaaaaggggggggggaatagaatcccctgtgatgcatgctaagcaggagcaccagcttcacgggcagggaagcttctagacagaaaaggcagaatatatgggatatcttccctgcctgccccaatgacaggatttgaacctagggaaagccccacagccttcagtggttatcttaggctactggatgaaacactccagaaacagtgagagggaggggcgaggtgagagcctacaatatgagcataatttgacacggatcagatttaggcttttggaaataagtgatagtatttgactgtgtaaaaaggaaactatggttttggctgctgtccctgaaggtgggaaagagcaaaggaccagccatgtttttgcatagatgatcatcagtctttccaattctggaataaagtatgaccagggtgaaatcaaaacatttaatcaactacagatgtgggttgggagaatttgctaaggcaaggcttatggcagggtcaaaccttgtcacactacggtaattagcacaaagagcattggtggctttgtgttgggttacagaactataaaaaacaatatcagcatgcctcaaacagcagcctctgtgccttctcacattgagatggaagcctcttagcagaagtcagcattcaccatctttgtaattctgttcttcgcaagtgggaacctagggcagcatgaggaatgactttaaaggtaagttatgagctgaagattccaaaccaactctaacattcatctcacacaggtacacatgacattcatgctctgatccttgggagagcaattactgggattcaggcattcacggttggcaagtgaaactacttcaacagagaaatactttgccctgagtctgctcaagaagaaatgtatggacacaacttttatggtggaaagtgagacactgatttgagattcaggacatttctttctggaagacattcagatatgctctatagaaagaagatgaaattaattctctgtatggccctcagccctttttaactggatgaaatcagcctccacctaccctgttttaagttttttggaactgggtgagacaaaggggagtaacacaggagccactgagatggagcagaccttgtgagaaggtgcaatggactttacagtttaccttgcctctatcccaggggcagtgccttctatgtgcactgaacagataagagatgaaaataaagagcctgtgccttactcctaggactggactaggtctactggtaaccatggagaggtggtttggggaatttctcataggagggataaacatgcctgggggttgtgccatgctggaatagcacacagagtttgagctagacattctgactggcaggggacactttgtcctttccctcatgacacagctaactacacaataaacaacatggacaata

SEQ ID NO: 3 - protein, 438 aa, Homo sapiens
MALRGVSVRLLSRGPGLHVLRTWVSSAAQTEKGGRTQSQLAKSSRPEFDWQDPLVLEEQLTTDEILIRDTFRTYCQERLMPRILLANRNEVFHREIISEMGELGVLGPTIKGYGCAGVSSVAYGLLARELERVDSGYRSAMSVQSSLVMHPIYAYGSEEQRQKYLPQLAKGELLGCFGLTEPNSGSDPSSMETRAHYNSSNKSYTLNGTKTWITNSPMADLFVVWARCEDGCIRGFLLEKGMRGLSAPRIQGKFSLRASATGMIIMDGVEVPEENVLPGASSLGGPFGCLNNARYGIAWGVLGASEFCLHTARQYALDRMQFGVPLARNQLIQKKLADMLTEITLGLHACLQLGRLKDQDKAAPEMVSLLKRNNCGKALDIARQARDMLGGNGISDEYHVIRHAMNLEAVNTYEGTHDIHALILGRAITGIQAFTASK

SEQ ID NO: 4 - protein, 447 aa, Mus musculus
MVPAMLTGYMSLRGVSARLLSRRSGLRFPRFPRTWSSAAAHTEKTQIRPAKSSRPVFDWKDPLILEEQLTADEKLIRDTFRNYCQERLMSRILLANRNEVFHRDIVYEMGELGVLGPTIKGYGCAGVSSVAYGLLTRELERVDSGYRSMMSVQSSLVMHPIYTYGSEEQRQKYLPGLAKGELLGCFGLTEPNHGSDPGGMETRARHNPSNQSYTLSGTKTWITNSPVADLFIVWARCEDNCIRGFILEKGMRGLSAPRIEGKFSLRASATGMIIMDSVEVPEENVLPNVSSLAGPFGCLNTARYGITWGVLGAAEFCLHTARQYALDRIQFGVPLARNQLVQKKLADMLTEITLGLHACLQLGRLKDQDKATPEMVSMLKRNNCGKALDIARQARDILGGNGISDEYHVIRHAMNLEAVNTYEGTHDIHALILGRAITGIQAFTVGK

SEQ ID NO: 5 - RNA, 20 nt, Streptococcus pyogenes
  source 1..20: mol_type = other RNA
gccgctcctggcagtagtta

SEQ ID NO: 6 - RNA, 20 nt, Streptococcus pyogenes
  source 1..20: mol_type = other RNA
gtgtgtcgtcggtggcctat

SEQ ID NO: 7 - RNA, 20 nt, Streptococcus pyogenes
  source 1..20: mol_type = other RNA
gtgcaggctgtccgtgatgc

SEQ ID NO: 8 - RNA, 21 nt, Streptococcus pyogenes
  source 1..21: mol_type = other RNA
gtccctgtgaagacaaacgtt

SEQ ID NO: 9 - RNA, 20 nt, Streptococcus pyogenes
  source 1..20: mol_type = other RNA
gaaacttctcttaattcgtg

SEQ ID NO: 10 - RNA, 21 nt, Streptococcus pyogenes
  source 1..21: mol_type = other RNA
cttgtgagtatgtggagcccc

SEQ ID NO: 11 - RNA, 63 nt, synthetic construct
  source 1..63: mol_type = other RNA
cagaatctactaaaacaaggcaaaatgccgtgtttatctcgtcaacttgttggcgagattttt

SEQ ID NO: 12 - RNA, 63 nt, synthetic construct; identical to SEQ ID NO: 11
  source 1..63: mol_type = other RNA

SEQ ID NO: 13 - DNA, 19 bp, synthetic construct
  source 1..19: mol_type = other DNA
ttatccccagggtcagaag

SEQ ID NO: 14 - DNA, 17 bp, synthetic construct
  source 1..17: mol_type = other DNA
ccagaccgacatctgac

SEQ ID NO: 15 - DNA, 21 bp, synthetic construct
  source 1..21: mol_type = other DNA
tagagagaacgggcaggatgt

SEQ ID NO: 16 - DNA, 21 bp, synthetic construct
  source 1..21: mol_type = other DNA
tctacgggatcgtacacacca

SEQ ID NO: 17 - DNA, 1852 bp, Homo sapiens
  source 1..1852: mol_type = other DNA
agttgcactgtagcctcggcagtgaaccgggaggtactaccaggtcgccgtcgttgctccgctcgctctgagagagcatggccctgagaggcgtctccgtgcggctgctgagccgcggacccggcctgcacgtccttcgcacgtgggtctcgtcggcggcgcagaccgagaaaggcgggagaacacagagccaactggctaagtcctcgcgtcccgagtttgactggcaggacccgctggtgctggaggagcagctgaccacagatgagatcctcatcagggacaccttccgcacctactgccaggagagactcatgcctcgcatcctgttggccaatcgcaacgaagtttttcatcgggagatcatttcggagatgggggagttgggtgtgctgggccccaccatcaaaggatatggctgtgctggggtttcgtctgtggcctatgggctcctggcccgagagctggagcgggtggacagtggctacaggtcggcgatgagtgtccagtcctccctcgtcatgcaccctatctatgcctatggcagcgaggaacagcggcagaagtacctgccccagctggccaagggggagctcctgggctgcttcgggctcacagagcccaacagcggaagtgaccccagcagcatggagaccagagcccactacaactcatccaacaagagctacaccctcaatgggaccaagacctggatcacgaactcgcctatggccgatctgtttgtagtgtgggctcggtgtgaagatggctgcattcggggcttcctgctggagaaggggatgcggggtctctcggcccccaggatccagggcaagttctcgctgcgggcctcagccacaggcatgatcatcatggacggtgtggaggtgccagaggagaatgtgctccctggtgcatccagcctggggggtcccttcggctgcctgaacaacgcccggtacggcatcgcgtggggcgtgcttggagcttcggagttctgcttgcacacagcccggcagtacgccctcgacaggatgcagtttggtgtcccactggccaggaaccagctgattcagaagaagctggcagacatgctcactgagattaccctgggccttcacgcctgcctgcagctcggccgcttgaaggaccaggacaaggctgcccccgagatggtttctctgctgaagaggaataactgtgggaaagccctggacatcgcccgccaggcccgagacatgctgggggggaatgggatttctgacgagtatcacgtgatccggcacgccatgaacctggaggccgtgaacacctacgaaggtacacatgacattcacgccctgatccttgggagagctatcacgggaatccaggcgttcacggccagcaagtgagccgctccatcaggggcccgaaactctcaagcccctttctggagagatgcctggctggaccgtaggagcgctgtgctctgagcttagaaagggaggtggcggatggagtgggaagtgagagacactgatttttaaatatcaaaatttcccttctgaagtcgttcagatgtgttccttaaaaagaagatggaattctctgtagagcgtctcaatccacttttaaccatggatgagagcagactccatttaccctgaaatagcagcttctcttgagaggagagtgacatggaagcaactccgtctgctgcagctgaccccctcacactgagttcacagtgcgccctccctccctcccatctgggggtagtgccttatgctgggtgttggagcagagtgagggagaggaaaataaagacctgcacatctgaccccaaggtgtcaggccggtttactggtaa

SEQ ID NO: 18 - DNA, 1341 bp, Mus musculus
  source 1..1341: mol_type = other DNA
atggttcctgcaatgctcaccggctacatgtccctgagaggagtctccgcgcggttgctgagccgcaggtccggcctgcgcttcccgcgttttccacgtacctggagctcggcggccgcccacaccgagaagacacagatccgaccggccaaatcctctcgccccgtgtttgactggaaggacccactcatactggaggagcagctgactgccgatgagaaactgatcagggacaccttccgtaactactgccaggagcggcttatgtctcgaattctgctggctaatcgaaatgaagtttttcacagggacattgtgtatgagatgggggagctgggcgtgttgggacccaccattaaagggtatggctgtgctggtgtgtcgtcggtggcctatgggctcctgacccgagagcttgagagggtggacagtggctacaggtcgatgatgagtgttcagtcctcccttgtcatgcaccccatctatacctatgggagcgaggaacagcgacagaaatatctgcccggactggccaagggtgaacttctgggctgctttggacttacagagcccaaccatgggagtgacccaggtggcatggagaccagagctcgccacaatccatcaaaccagagctacactctcagtgggaccaagacctggatcaccaactcccctgtggctgacctatttatagtgtgggctcggtgtgaggataactgtattcggggcttcatactggagaagggtatgcggggcctctcagcccctaggattgaaggaaagttctccttgcgggcctcggctaccggtatgatcatcatggacagtgtggaagtgcctgaggagaatgtgctgcctaatgtatccagcctggcgggcccttttggctgccttaacactgcccgctatggcatcacatggggtgtgttgggagctgctgagttctgtttgcacacagcccggcagtatgccctagacaggatccagtttggagtccccttggccaggaaccagctggttcagaagaagttggcagacatgctcactgagatcacactgggactccatgcttgtttgcaacttggccgtttaaaggatcaggacaaggctaccccagagatggtctccatgttgaagagaaacaactgtggaaaagccctggatattgcccgccaggcacgagacatcctgggaggaaatgggatttctgatgagtaccatgtcatccggcatgctatgaatctggaggcagtgaacacttatgaaggtacacatgacattcatgctctgatccttgggagagcaattactgggattcaggcattcacggttggcaag

SEQ ID NO: 19 - DNA, 4586 bp, synthetic construct
  source 1..4586: mol_type = other DNA
cagctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctacgtagccatgctctggtgtttgctgcttgcaatgtttgcccattttagggtggacacaggacgctgtggtttctgagccagggggcgactcagatcccagccagtggacttagcccctgtttgctcctccgataactggggtgaccttggttaatattcaccagcagcctcccccgttgcccctctggatccactgcttaaatacggacgaggacagggccctgtctcctcagcttcaggcaccaccactgacctgggacagtgaatctctagaggatccggtaccgaggagatctgccgccgcgatcgccatggttcctgcaatgctcaccggctacatgtccctgagaggagtctccgcgcggttgctgagccgcaggtccggcctgcgcttcccgcgttttccacgtacctggagctcggcggccgcccacaccgagaagacacagatccgaccggccaaatcctctcgccccgtgtttgactggaaggacccactcatactggaggagcagctgactgccgatgagaaactgatcagggacaccttccgtaactactgccaggagcggcttatgtctcgaattctgctggctaatcgaaatgaagtttttcacagggacattgtgtatgagatgggggagctgggcgtgttgggacccaccattaaagggtatggctgtgctggtgtgtcgtcggtggcctatgggctcctgacccgagagcttgagagggtggacagtggctacaggtcgatgatgagtgttcagtcctcccttgtcatgcaccccatctatacctatgggagcgaggaacagcgacagaaatatctgcccggactggccaagggtgaacttctgggctgctttggacttacagagcccaaccatgggagtgacccaggtggcatggagaccagagctcgccacaatccatcaaaccagagctacactctcagtgggaccaagacctggatcaccaactcccctgtggctgacctatttatagtgtgggctcggtgtgaggataactgtattcggggcttcatactggagaagggtatgcggggcctctcagcccctaggattgaaggaaagttctccttgcgggcctcggctaccggtatgatcatcatggacagtgtggaagtgcctgaggagaatgtgctgcctaatgtatccagcctggcgggcccttttggctgccttaacactgcccgctatggcatcacatggggtgtgttgggagctgctgagttctgtttgcacacagcccggcagtatgccctagacaggatccagtttggagtccccttggccaggaaccagctggttcagaagaagttggcagacatgctcactgagatcacactgggactccatgcttgtttgcaacttggccgtttaaaggatcaggacaaggctaccccagagatggtctccatgttgaagagaaacaactgtggaaaagccctggatattgcccgccaggcacgagacatcctgggaggaaatgggatttctgatgagtaccatgtcatccggcatgctatgaatctggaggcagtgaacacttatgaaggtacacatgacattcatgctctgatccttgggagagcaattactgggattcaggcattcacggttggcaagtaaacgcgtgcggccgcaccggttcgcgaataaaagatctttattttcattagatctgtgtgttggttttttgtgtgatgcagctacgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcagctggcgtaatagcgaagaggcccgcaccgatcgcccttcccaacagttgcgcagcctgaatggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccccgatagacggtttttcgccctttgacgctggagttcacgttcctcaatagtggactcttgttccaaactggaacaacactcaaccctatctcggtctattcttttgatttataagggatttttccgatttcggcctattggttaaaaaatgagctgatttaacaaaaatttaacgcgaattttaacaaaatattaacgtttataatttcaggtggcatctttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaatagtggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagtaatggtaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctgcggttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatg

SEQ ID NO: 20 - DNA, 6966 bp, synthetic construct
  source 1..6966: mol_type = other DNA
cagctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctacgtagccatgctctggtgtacaaaaaagcaggctttaaaggaaccaattcagtcgactggatccggtaccaaggtcgggcaggaagagggcctatttcccatgattccttcatatttgcatatacgatacaaggctgttagagagataattagaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccgtccctgtgaagacaaacgttgttttagtactctggaaacagaatctactaaaacaaggcaaaatgccgtgtttatctcgtcaacttgttggcgagatttttttctagaaagctttgtttgctgcttgcaatgtttgcccattttagggtggacacaggacgctgtggtttctgagccagggggcgactcagatcccagccagtggacttagcccctgtttgctcctccgataactggggtgaccttggttaatattcaccagcagcctcccccgttgcccctctggatccactgcttaaatacggacgaggacagggccctgtctcctcagcttcaggcaccaccactgacctgggacagtgaatcaccggtgccaccatggccccaaagaagaagcggaaggtcggtatccacggagtcccagcagccaagcggaactacatcctgggcctggacatcggcatcaccagcgtgggctacggcatcatcgactacgagacacgggacgtgatcgatgccggcgtgcggctgttcaaagaggccaacgtggaaaacaacgagggcaggcggagcaagagaggcgccagaaggctgaagcggcggaggcggcatagaatccagagagtgaagaagctgctgttcgactacaacctgctgaccgaccacagcgagctgagcggcatcaacccctacgaggccagagtgaagggcctgagccagaagctgagcgaggaagagttctctgccgccctgctgcacctggccaagagaagaggcgtgcacaacgtgaacgaggtggaagaggacaccggcaacgagctgtccaccaaagagcagatcagccggaacagcaaggccctggaagagaaatacgtggccgaactgcagctggaacggctgaagaaagacggcgaagtgcggggcagcatcaacagattcaagaccagcgactacgtgaaagaagccaaacagctgctgaaggtgcagaaggcctaccaccagctggaccagagcttcatcgacacctacatcgacctgctggaaacccggcggacctactatgagggacctggcgagggcagccccttcggctggaaggacatcaaagaatggtacgagatgctgatgggccactgcacctacttccccgaggaactgcggagcgtgaagtacgcctacaacgccgacctgtacaacgccctgaacgacctgaacaatctcgtgatcaccagggacgagaacgagaagctggaatattacgagaagttccagatcatcgagaacgtgttcaagcagaagaagaagcccaccctgaagcagatcgccaaagaaatcctcgtgaacgaagaggatattaagggctacagagtgaccagcaccggcaagcccgagttcaccaacctgaaggtgtaccacgacatcaaggacattaccgcccggaaagagattattgagaacgccgagctgctggatcagattgccaagatcctgaccatctaccagagcagcgaggacatccaggaagaactgaccaatctgaactccgagctgacccaggaagagatcgagcagatctctaatctgaagggctataccggcacccacaacctgagcctgaaggccatcaacctgatcctggacgagctgtggcacaccaacgacaaccagatcgctatcttcaaccggctgaagctggtgcccaagaaggtggacctgtcccagcagaaagagatccccaccaccctggtggacgacttcatcctgagccccgtcgtgaagagaagcttcatccagagcatcaaagtgatcaacgccatcatcaagaagtacggcctgcccaacgacatcattatcgagctggcccgcgagaagaactccaaggacgcccagaaaatgatcaacgagatgcagaagcggaaccggcagaccaacgagcggatcgaggaaatcatccggaccaccggcaaagagaacgccaagtacctgatcgagaagatcaagctgcacgacatgcaggaaggcaagtgcctgtacagcctggaagccatccctctggaagatctgctgaacaaccccttcaactatgaggtggaccacatcatccccagaagcgtgtccttcgacaacagcttcaacaacaaggtgctcgtgaagcaggaagaaaacagcaagaagggcaaccggaccccattccagtacctgagcagcagcgacagcaagatcagctacgaaaccttcaagaagcacatcctgaatctggccaagggcaagggcagaatcagcaagaccaagaaagagtatctgctggaagaacgggacatcaacaggttctccgtgcagaaagacttcatcaaccggaacctggtggataccagatacgccaccagaggcctgatgaacctgctgcggagctacttcagagtgaacaacctggacgtgaaagtgaagtccatcaatggcggcttcaccagctttctgcggcggaagtggaagtttaagaaagagcggaacaaggggtacaagcaccacgccgaggacgccctgatcattgccaacgccgatttcatcttcaaagagtggaagaaactggacaaggccaaaaaagtgatggaaaaccagatgttcgaggaaaagcaggccgagagcatgcccgagatcgaaaccgagcaggagtacaaagagatcttcatcaccccccaccagatcaagcacattaaggacttcaaggactacaagtacagccaccgggtggacaagaagcctaatagagagctgattaacgacaccctgtactccacccggaaggacgacaagggcaacaccctgatcgtgaacaatctgaacggcctgtacgacaaggacaatgacaagctgaaaaagctgatcaacaagagccccgaaaagctgctgatgtaccaccacgacccccagacctaccagaaactgaagctgattatggaacagtacggcgacgagaagaatcccctgtacaagtactacgaggaaaccgggaactacctgaccaagtactccaaaaaggacaacggccccgtgatcaagaagattaagtattacggcaacaaactgaacgcccatctggacatcaccgacgactaccccaacagcagaaacaaggtcgtgaagctgtccctgaagccctacagattcgacgtgtacctggacaatggcgtgtacaagttcgtgaccgtgaagaatctggatgtgatcaaaaaagaaaactactacgaagtgaatagcaagtgctatgaggaagctaagaagctgaagaagatcagcaaccaggccgagtttatcgcctccttctacaacaacgatctgatcaagatcaacggcgagctgtatagagtgatcggcgtgaacaacgacctgctgaaccggatcgaagtgaacatgatcgacatcacctaccgcgagtacctggaaaacatgaacgacaagaggccccccaggatcattaagacaatcgcctccaagacccagagcattaagaagtacagcacagacattctgggcaacctgtatgaagtgaaatctaagaagcaccctcagatcatcaaaaagggcaaaaggccggcggccacgaaaaaggccggccaggcaaaaaagaaaaagggatcctacccatacgatgttccagattacgctagcggcttcgccaacgagcttggacccaggttgatgggaaagtaagaattcctagagcgaataaaagatctttattttcattagatctgtgtgttggttttttgtgtgatgcagctacgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaccgcagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttaccattactacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccactattgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaagatgccacctgaaattataaacgttaatattttgttaaaattcgcgttaaatttttgttaaatcagctcattttttaaccaataggccgaaatcggaaaaatcccttataaatcaaaagaatagaccgagatagggttgagtgttgttccagtttggaacaagagtccactattgaggaacgtgaactccagcgtcaaagggcgaaaaaccgtctatcggggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcccattcgccattcaggctgcgcaactgttgggaagggcgatcggtgcgggcctcttcgctattacgc

SEQ ID NO: 21 - DNA, 6966 bp, synthetic construct
  source 1..6966: mol_type = other DNA
cagctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctacgtagccatgctctggtgtacaaaaaagcaggctttaaaggaaccaattcagtcgactggatccggtaccaaggtcgggcaggaagagggcctatttcccatgattccttcatatttgcatatacgatacaaggctgttagagagataattagaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacacccttgtgagtatgtggagccccgttttagtactctggaaacagaatctactaaaacaaggcaaaatgccgtgtttatctcgtcaacttgttggcgagatttttttctagaaagctttgtttgctgcttgcaatgtttgcccattttagggtggacacaggacgctgtggtttctgagccagggggcgactcagatcccagccagtggacttagcccctgtttgctcctccgataactggggtgaccttggttaatattcaccagcagcctcccccgttgcccctctggatccactgcttaaatacggacgaggacagggccctgtctcctcagcttcaggcaccaccactgacctgggacagtgaatcaccggtgccaccatggccccaaagaagaagcggaaggtcggtatccacggagtcccagcagccaagcggaactacatcctgggcctggacatcggcatcaccagcgtgggctacggcatcatcgactacgagacacgggacgtgatcgatgccggcgtgcggctgttcaaagaggccaacgtggaaaacaacgagggcaggcggagcaagagaggcgccagaaggctgaagcggcggaggcggcatagaatccagagagtgaagaagctgctgttcgactacaacctgctgaccgaccacagcgagctgagcggcatcaacccctacgaggccagagtgaagggcctgagccagaagctgagcgaggaagagttctctgccgccctgctgcacctggccaagagaagaggcgtgcacaacgtgaacgaggtggaagaggacaccggcaacgagctgtccaccaaagagcagatcagccggaacagcaaggccctggaagagaaatacgtggccgaactgcagctggaacggctgaagaaagacggcgaagtgcggggcagcatcaacagattcaagaccagcgactacgtgaaagaagccaaacagctgctgaaggtgcagaaggcctaccaccagctggaccagagcttcatcgacacctacatcgacctgctggaaacccggcggacctactatgagggacctggcgagggcagccccttcggctggaaggacatcaaagaatggtacgagatgctgatgggccactgcacctacttccccgaggaactgcggagcgtgaagtacgcctacaacgccgacctgtacaacgccctgaacgacctgaacaatctcgtgatcaccagggacgagaacgagaagctggaatattacgagaagttccagatcatcgagaacgtgttcaagcagaagaagaagcccaccctgaagcagatcgccaaagaaatcctcgtgaacgaagaggatattaagggctacagagtgaccagcaccggcaagcccgagttcaccaacctgaaggtgtaccacgacatcaaggacattaccgcccggaaagagattattgagaacgccgagctgctggatcagattgccaagatcctgaccatctaccagagcagcgaggacatccaggaagaactgaccaatctgaactccgagctgacccaggaagagatcgagcagatctctaatctgaagggctataccggcacccacaacctgagcctgaaggccatcaacctgatcctggacgagctgtggcacaccaacgacaaccagatcgctatcttcaaccggctgaagctggtgcccaagaaggtggacctgtcccagcagaaagagatccccaccaccctggtggacgacttcatcctgagccccgtcgtgaagagaagcttcatccagagcatcaaagtgatcaacgccatcatcaagaagtacggcctgcccaacgacatcattatcgagctggcccgcgagaagaactccaaggacgcccagaaaatgatcaacgagatgcagaagcggaaccggcagaccaacgagcggatcgaggaaatcatccggaccaccggcaaagagaacgccaagtacctgatcgagaagatcaagctgcacgacatgcaggaaggcaagtgcctgtacagcctggaagccatccctctggaagatctgctgaacaaccccttcaactatgaggtggaccacatcatccccagaagcgtgtccttcgacaacagcttcaacaacaaggtgctcgtgaagcaggaagaaaacagcaagaagggcaaccggaccccattccagtacctgagcagcagcgacagcaagatcagctacgaaaccttcaagaagcacatcctgaatctggccaagggcaagggcagaatcagcaagaccaagaaagagtatctgctggaagaacgggacatcaacaggttctccgtgcagaaagacttcatcaaccggaacctggtggataccagatacgccaccagaggcctgatgaacctgctgcggagctacttcagagtgaacaacctggacgtgaaagtgaagtccatcaatggcggcttcaccagctttctgcggcggaagtggaagtttaagaaagagcggaacaaggggtacaagcaccacgccgaggacgccctgatcattgccaacgccgatttcatcttcaaagagtggaagaaactggacaaggccaaaaaagtgatggaaaaccagatgttcgaggaaaagcaggccgagagcatgcccgagatcgaaaccgagcaggagtacaaagagatcttcatcaccccccaccagatcaagcacattaaggacttcaaggactacaagtacagccaccgggtggacaagaagcctaatagagagctgattaacgacaccctgtactccacccggaaggacgacaagggcaacaccctgatcgtgaacaatctgaacggcctgtacgacaaggacaatgacaagctgaaaaagctgatcaacaagagccccgaaaagctgctgatgtaccaccacgacccccagacctaccagaaactgaagctgattatggaacagtacggcgacgagaagaatcccctgtacaagtactacgaggaaaccgggaactacctgaccaagtactccaaaaaggacaacggccccgtgatcaagaagattaagtattacggcaacaaactgaacgcccatctggacatcaccgacgactaccccaacagcagaaacaaggtcgtgaagctgtccctgaagccctacagattcgacgtgtacctggacaatggcgtgtacaagttcgtgaccgtgaagaatctggatgtgatcaaaaaagaaaactactacgaagtgaatagcaagtgctatgaggaagctaagaagctgaagaagatcagcaaccaggccgagtttatcgcctccttctacaacaacgatctgatcaagatcaacggcgagctgtatagagtgatcggcgtgaacaacgacctgctgaaccggatcgaagtgaacatgatcgacatcacctaccgcgagtacctggaaaacatgaacgacaagaggccccccaggatcattaagacaatcgcctccaagacccagagcattaagaagtacagcacagacattctgggcaacctgtatgaagtgaaatctaagaagcaccctcagatcatcaaaaagggcaaaaggccggcggccacgaaaaaggccggccaggcaaaaaagaaaaagggatcctacccatacgatgttccagattacgctagcggcttcgccaacgagcttggacccaggttgatgggaaagtaagaattcctagagcgaataaaagatctttattttcattagatctgtgtgttggttttttgtgtgatgcagctacgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaccgcagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttaccattactacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccactattgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaagatgccacctgaaattataaacgttaatattttgttaaaattcgcgttaaatttttgttaaatcagctcattttttaaccaataggccgaaatcggaaaaatcccttataaatcaaaagaatagaccgagatagggttgagtgttgttccagtttggaacaagagtccactattgaggaacgtgaactccagcgtcaaagggcgaaaaaccgtctatcggggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcccattcgccattcaggctgcgcaactgttgggaagggcgatcggtgcgggcctcttcgctattacgc

SEQ ID NO: 22 - DNA, 185 bp, synthetic construct
  source 1..185: mol_type = other DNA
cagctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctacgtagccatgctctgg

SEQ ID NO: 23 - DNA, 173 bp, synthetic construct
  source 1..173: mol_type = other DNA
tacgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcagctg

SEQ ID NO: 24 - DNA, 61 bp, synthetic construct
  source 1..61: mol_type = other DNA
tcgcgaataaaagatctttattttcattagatctgtgtgttggttttttgtgtgatgcagc

SEQ ID NO: 25 - DNA, 58 bp, synthetic construct
  source 1..58: mol_type = other DNA
cgaataaaagatctttattttcattagatctgtgtgttggttttttgtgtgatgcagc

SEQ ID NO: 26 - DNA, 252 bp, synthetic construct
  source 1..252: mol_type = other DNA
tgtttgctgcttgcaatgtttgcccattttagggtggacacaggacgctgtggtttctgagccagggggcgactcagatcccagccagtggacttagcccctgtttgctcctccgataactggggtgaccttggttaatattcaccagcagcctcccccgttgcccctctggatccactgcttaaatacggacgaggacagggccctgtctcctcagcttcaggcaccaccactgacctgggacagtgaatc

SEQ ID NO: 27 - DNA, 241 bp, synthetic construct
  source 1..241: mol_type = other DNA
gagggcctatttcccatgattccttcatatttgcatatacgatacaaggctgttagagagataattagaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggac

SEQ ID NO: 28 - DNA, 3207 bp, synthetic construct
  source 1..3207: mol_type = other DNA
atggccccaaagaagaagcggaaggtcggtatccacggagtcccagcagccaagcggaactacatcctgggcctggacatcggcatcaccagcgtgggctacggcatcatcgactacgagacacgggacgtgatcgatgccggcgtgcggctgttcaaagaggccaacgtggaaaacaacgagggcaggcggagcaagagaggcgccagaaggctgaagcggcggaggcggcatagaatccagagagtgaagaagctgctgttcgactacaacctgctgaccgaccacagcgagctgagcggcatcaacccctacgaggccagagtgaagggcctgagccagaagctgagcgaggaagagttctctgccgccctgctgcacctggccaagagaagaggcgtgcacaacgtgaacgaggtggaagaggacaccggcaacgagctgtccaccaaagagcagatcagccggaacagcaaggccctggaagagaaatacgtggccgaactgcagctggaacggctgaagaaagacggcgaagtgcggggcagcatcaacagattcaagaccagcgactacgtgaaagaagccaaacagctgctgaaggtgcagaaggcctaccaccagctggaccagagcttcatcgacacctacatcgacctgctggaaacccggcggacctactatgagggacctggcgagggcagccccttcggctggaaggacatcaaagaatggtacgagatgctgatgggccactgcacctacttccccgaggaactgcggagcgtgaagtacgcctacaacgccgacctgtacaacgccctgaacgacctgaacaatctcgtgatcaccagggacgagaacgagaagctggaatattacgagaagttccagatcatcgagaacgtgttcaagcagaagaagaagcccaccctgaagcagatcgccaaagaaatcctcgtgaacgaagaggatattaagggctacagagtgaccagcaccggcaagcccgagttcaccaacctgaaggtgtaccacgacatcaaggacattaccgcccggaaagagattattgagaacgccgagctgctggatcagattgccaagatcctgaccatctaccagagcagcgaggacatccaggaagaactgaccaatctgaactccgagctgacccaggaagagatcgagcagatctctaatctgaagggctataccggcacccacaacctgagcctgaaggccatcaacctgatcctggacgagctgtggcacaccaacgacaaccagatcgctatcttcaaccggctgaagctggtgcccaagaaggtggacctgtcccagcagaaagagatccccaccaccctggtggacgacttcatcctgagccccgtcgtgaagagaagcttcatccagagcatcaaagtgatcaacgccatcatcaagaagtacggcctgcccaacgacatcattatcgagctggcccgcgagaagaactccaaggacgcccagaaaatgatcaacgagatgcagaagcggaaccggcagaccaacgagcggatcgaggaaatcatccggaccaccggcaaagagaacgccaagtacctgatcgagaagatcaagctgcacgacatgcaggaaggcaagtgcctgtacagcctggaagccatccctctggaagatctgctgaacaaccccttcaactatgaggtggaccacatcatccccagaagcgtgtccttcgacaacagcttcaacaacaaggtgctcgtgaagcaggaagaaaacagcaagaagggcaaccggaccccattccagtacctgagcagcagcgacagcaagatcagctacgaaaccttcaagaagcacatcctgaatctggccaagggcaagggcagaatcagcaagaccaagaaagagtatctgctggaagaacgggacatcaacaggttctccgtgcagaaagacttcatcaaccggaacctggtggataccagatacgccaccagaggcctgatgaacctgctgcggagctacttcagagtgaacaacctggacgtgaaagtgaagtccatcaatggcggcttcaccagctttctgcggcggaagtggaagtttaagaaagagcggaacaaggggtacaagcaccacgccgaggacgccctgatcattgccaacgccgatttcatcttcaaagagtggaagaaactggacaaggccaaaaaagtgatggaaaaccagatgttcgaggaaaagcaggccgagagcatgcccgagatcgaaaccgagcaggagtacaaagagatcttcatcaccccccaccagatcaagcacattaaggacttcaaggactacaagtacagccaccgggtggacaagaagcctaatagagagctgattaacgacaccctgtactccacccggaaggacgacaagggcaacaccctgatcgtgaacaatctgaacggcctgtacgacaaggacaatgacaagctgaaaaagctgatcaacaagagccccgaaaagctgctgatgtaccaccacgacccccagacctaccagaaactgaagctgattatggaacagtacggcgacgagaagaatcccctgtacaagtactacgaggaaaccgggaactacctgaccaagtactccaaaaaggacaacggccccgtgatcaagaagattaagtattacggcaacaaactgaacgcccatctggacatcaccgacgactaccccaacagcagaaacaaggtcgtgaagctgtccctgaagccctacagattcgacgtgtacctggacaatggcgtgtacaagttcgtgaccgtgaagaatctggatgtgatcaaaaaagaaaactactacgaagtgaatagcaagtgctatgaggaagctaagaagctgaagaagatcagcaaccaggccgagtttatcgcctccttctacaacaacgatctgatcaagatcaacggcgagctgtatagagtgatcggcgtgaacaacgacctgctgaaccggatcgaagtgaacatgatcgacatcacctaccgcgagtacctggaaaacatgaacgacaagaggccccccaggatcattaagacaatcgcctccaagacccagagcattaagaagtacagcacagacattctgggcaacctgtatgaagtgaaatctaagaagcaccctcagatcatcaaaaagggc

SEQ ID NO: 29 - DNA, 27 bp, synthetic construct
  source 1..27: mol_type = other DNA
tacccatacgatgttccagattacgct

SEQ ID NO: 30 - DNA, 48 bp, synthetic construct
  source 1..48: mol_type = other DNA
aaaaggccggcggccacgaaaaaggccggccaggcaaaaaagaaaaag

SEQ ID NO: 31 - DNA, 42 bp, synthetic construct
  source 1..42: mol_type = other DNA
agcggcttcgccaacgagcttggacccaggttgatgggaaag

SEQ ID NO: 32 - DNA, 21 bp, synthetic construct
  source 1..21: mol_type = other DNA
ccaaagaagaagcggaaggtc

SEQ ID NO: 33 - RNA, 21 nt, synthetic construct
  source 1..21: mol_type = other RNA
ggagtcttgatgaacataatt

SEQ ID NO: 34 - RNA, 21 nt, synthetic construct
  source 1..21: mol_type = other RNA
ttatgttcatcaagactccca

SEQ ID NO: 35 - DNA, 70701 bp, Homo sapiens
  source 1..70701: mol_type = other DNA
agtttcttctaggaattcggagcctcccctgcagcgactcggaagattcgaggcggcgggggacaagtcggcgccccagagcggacgaggtgaggagaggagaggtccccgcggagagctgagcagacgcgcgggtgggctcagcctgtgagatgcggtggagatagcgggccgggctcgggctgtcgctgggaggggcccgcgagcctggccttcgggtcccggcgccgcgaggcgaccccggccgggccgagagagggagaaggcggcgagcattcgggcctccctgcgaggaatggagggagagcggaaacgcagagagtttcactgcgaggaagggagggagagcggtcagcgcagctccagcatttctaagtagaaaggcgagggattgcaattcggatttcttttctttcttttttggcggacagtggtggtgctgtggctggggaataaagaactgatgaagctgaggaaacaacggcttgttaatttgtggacggcgtgcttagaggggacggctggctgagggggcggtggatggagatttcggggtggttctttcccccttccctccccacgcccagcttagccaaactgatcacaaacttctctccttgtccatcttgggctgcccactctccgagacacagacccattctccagcaaggatcggagatgggtggaagttgggcgccttgcaagtctgggtttcctgaactcttcagcccctgcgggactatgcagcccattgtgagcgctcgcgcgggacggggcgcggggagaagcagtttacccaccctcctcggttgaatggggctgaatgtctgtctatcctgacagcttcagaagaagaaatgtgcttttcagacattgatttcaggggaatgggggtggggggagcggaagagagagagagaaagagcgctcgagcgcgagagagaatgaattagttcagtaacttgaacaaggattaacattttttgatgttgtgggttatcagtggggaaggtgcagctgtgagatgtagttatacacaggacggaatggtccttgactgaagctgcggctttccaatactgtattaaaaaaaatcccaaaacactgatggcactttggtgtttctgggtttgtctgttttgtcccctgcactcccctcttctttttttcccatgaagatgagtgaatctagggctacatgacttgactgtgtaacctggtggccattctgcaaaatgagttaggtggacaaaaattgaagccaatttacctgaatcattttcccactcttcagataatcatatttgttatcagtttcacttgttattgatgatggcgtgatcagaactttggtttatttgtcattgaattttttttctcactagtcgtttaacagaccttggttcggaattttggaaatgatctttggtgtttaagaagccaccatttaacaaaacattttcatttttttcatatattcttttattaattttctcatagaaataacattacaaacagtggtaagttttcttgggaaggctacaaaagattctatgcaattcatgaaatcatggaaacaaacatttggaatggaaaataaccccaaaacattattaccacattcatctgatctaaggtgtcatctattgtaagatgcatccctatttcactgatgctaaaatgtgtaaaaaaattgtcttaaatttgatgaaatacaacattaaaatggtaatttatttatcttgaacatgcatgcctgaagtttgaagttgaagagagggcttattgggctctaattattttttttccaaaataatgtaagggtgggggattaatatggagccatcgtgaaggtcttactacagtgtgacagaggaaagaaatgtgagtctactgttagcggcgtggttgtgctcgcttttgaggagtaggcaagcctggaggctttttcttttcttaaaatttattgtttatgacacacagcaatgctcagcactttatgcatattagctcaattaaacgtcttaagagccctaagaggtagaggttaatatgattctcattttgcgcatgatgcacagacaggttaagttgccagccaaggtcacagagtaagtttggccgctgaggaacccaggagagaaaccagtattcctttgaggtatccatcttctgttgcaagctcttggttcctggtccatgctattaatgcaggtagcttaggtgaatgtccactctttcttgattgtgtttttggtattttgtttccctgtttggtgtactttagctagaaattcataagtcagagtgcctatttatgttctacatttctaatgtttttgtaattctcagtgtccagttactactatgcttaatagaatcagaatatcagagcctgaaagggactttagtattatatttttgtctgggtaaggaaactgaagtcaagaggaggaaccctacatctagttatgtgtcccagctcccatgtctaagttttcccactatactgtactatatttcacgacatttgttgatctatgagatcttggaagagaatcaagtttttttttttttttttttttttttttgcagggttagggggatgtgagtctcttattaatgtgtctattttatctgtttgtttgtttgtttgttttcttgctgcataggtctttataatgtttattgaatttgctttttcatttattcattctttcttaaaacacttgctgttttcccatgtgccagtcattgggaatacagagatgagtaaaacacaactcatacacatttgcagtttagcaaagagacagacaagtaagcagataagcattttggactagactccgatttagaactaactttgtattccatttcaatgaagctaaaattataagctagagaccaggagaaatatgtgtcatgtttatggatgactttgagtaggtgatgggctggagaaaatatgaaaattctcagaccagattagttaacactaatagacctcctcccccaaagtctaccactttcttcttaatccagtaaagtattttatagagggtttaacatggaagcattaaaagaatgtacccacgcacaggaagtcaaagtatgaccagtgttaatgcaagcccaagcctagttctagcaagccttgaaattgggagggatttggattagttctaggccatcatctggtgctgtgttaattctggaactcttttgaacctctccaaaaaatattatggtttggatacttagtgtaggccaagactcatagccatagagctgccatacttatttacagacaccagtgcttcttgggaaaccatggattttaatatatacttgtattttttttttttttttagttacttggaagggtgaatataaataattagcgctgttcatccttgaacttattttcttcatctaaagactacattacctcattcttagtctctcaatatttaatcacaatatatgcctgattattgcaaataatttgtcattcagaaatcccccgtgtgatttgatagttttggtttgagtcattgaattatgctgtagtgaaattattttaaatatctaaaatagatggacaaagagcacgttcctaactgtggggattaaagttggaacatttggccttaatttcttcaaaaatgaatgtgtgatccttcatacatatcccaaaagaagtaacagaaattaagaggtgcaatgcttagttcattctttttttttttttttttttttttgagactgagtctcgctctgtcgcccaggctggagtgcagtggtacgaactcggctcactgcaagctccgcctcccgggttccacgccattctccagcctcagtctcccgagtagctgggactacaggcgcccgccaccgaccctggctaattttttgtatttttagtagagacggggtttcaccgtgtcagccaggacggtctcgacctcctgacctcgtgatccgcctgcctcagcctcccaaagtgctgggattacaggtgtgagccaccgcacccagccgcttagttcattcttaagctcttgtaatgcttgtgcattgttttgcttaggtaggtgatctataagcttaaaaagtattctgattgggttggtgctatggaaagggttgaagagacttaaagaattttagctatttgatggaagagtaaaaagctctatgaaaatctcagctctcttcatctaccccgttcccttataaaaactgcaaaaattaaatacatcatttgtttgtttgtttgtttgtttgtttgtttgagacagtctcactttgttgtccaggtgggagtgtaatggcatgatcttcacttactgcaaacttcgcctcctgggctcaagtgattttcatgcttcagcctcccgagtagctgggactaccagcatgcgccaccacgcccagttaatttttgtatttttagtagagatggggttttgcatgttgcctaggctggtctcgaactcctcaggtgatctgcccaccctggcctcccaaagtgctggcattataggcataagccactgtgcttggccaaacacatcattttaaggcataaatattgcctgaaaagtagcagagagaaaaatcttgatcgagttacatttaaaaagttataccaacgagaacagacttactgtgacatccattatgataagtcataaaaatacataatagtggcttggaaaatgctttctataaaacagaaaaaaatcccctatttctatgggcctcactattctcatttgtaatactagattttagattacattctatcagtcccactttgactctaatatttggatataatcaataattaataattcactatcagatattgatcagcaccatttaatttgataaaactaatcctgaacatgggcagggattggattactgcttctttatatctttaccaagtgcatcatgccgtgcatatcttaagtagcccccaaatatttttttaattaaagagaatcaaaagcatttgtcccatattccgtgtacaaaagattttattttgataactctatcaagagactttctggatttcattataatttaaatattagaaaatatttgtgctataagcaaatgactagctgtttttatattaatatgtgtaattgtacaaaagaccactttgctcacactctgaaaggacctttctaaccacacatgagattctctgtcctgagagtggcttttcttaaatataatattttataaatacattttacagaatctgctttgctttggttactccatgtgatttagaatgatggtgtactcagtgaccataggacagctcatggtcattgaaagaaaatgggatgtgtatggagtgccgagctctgtctctgtgtccttttttttctctctctctctatctgtctctgtcattttcctttcccttttctggaaaataataaaaaaatacaaagaacattcaagcatgttctttcttctgattctcatgtttttgttgcaaagttagcaaattaaaagattaagtcttgccaccttgagagatggaaagataagacagtatcatagagtatgttctcctattgctactgggcaaccttcactattatcttcctgtctcagtacatagtagttccttcttatccaagttttcgctttccatggtttcagttacccactgtcaatctggtctgaaaataggtgaataaagtacactaagatattgtgagagagaaagagagagtgagagagaccacatttacataacttttattacagtatattgtcataattattctattttattattgttgctagtctcttacagtgcccaatttataaattaaactatatcataggtatgtatgtataggaaaaaacatagtatatatagggtttgggactatcagagatttcaagcatccactgggcttcttggaatgtattacccacagataaacagggtctactgtatctcgctgcaattcactataatactcccatgacttttagagacatttcaaattcctcatcaattttaggtaaataaaaatgtataccttagtggtgttctgttttacaaaaacagagaagaaatcttttaagtttcatttttatatttgaaatgttaaccttcatgtaacaaagggaagactgttacaattcatcagaggaagggtcttgtaccccacccttgcctgtgtgacacttctctttctctattttcctcctctttgttctttctagctctttttttccccctctctgcctcctgcttttctacatcctccttgcttgtgttacaagctgttatcagcacccgacattgtcgggtaacctcactagagctggtggtgagtaaaccctgatcttccctccttccttcattgaaggcattccagaaatatacattgaatttacttcctgatattcttgaaaaataccacaaaatccaggacagaaaaagctaaagcagcctccgttggcatttccatgaaatactagacacctgcagtgcaattcagaaattctcactatttcctatttcttgaagaccttagaagcaaatgaactgtggatcttgaaagttctatgctgagaccaagttttaaggttcaagcagtaactagcagttatactataatcaaaccattttttttctaagcacccagaaatctagaatttatcttggcaggtactcaggaatgattttcagtataaagcaggaaaaggggtcaagagaggcacggcatgctacggggagaactgttaattcactgtgactgttgaaggcaaagccatatggatctgatttctttctgaaacaagagttggctggagttaacagcatgtgttggatctatatggtgtgaatctgtaggctttggcaaagaagagaaaattccaggtgttaaacattaaactaggaatcctgcttctgcttaaaagcaacagatcctatatgattccagttcagtttcttcatctataaaatggggatatgtatctacttaataagatttttgcaaggaggaaataagatgatatccataaagtgctcagcatagagcttggcatgtgatgcttaatcaatggtaactactgttacatttgtttaaataatgaaagttacagaaattattatttaagccttccatgcaaaaagtgaaagtgaaagtagcttcccaaataaactgaagctcagtcccaggcacttctgtctttttccctagtcttccaagatagttggcctttgcaagtatatgtgtgtatgttacaagtgactgtctgtatttgtgtctgtgtgtgtgtctactgctttcccgccctatcccagacaaagaaggtggtgggagaaggagcagggattattactgagtgagggatgagtgaggttaacattcaagcttgggctagaggggagggattaaattatctccacatgttcctcctgatggagtcaagaatttcaactcataataaaagttacaggggtatgtagtcacctgtgaattcatgaaacacagcccctgctgggaagatgggatggtttctggacatttgtgtatgttcaaaggaatcactatctttggaattggttttattagaaactgagggattcaggttctagtttttcttttggcttcgagaagactgaatgttatgggtttgggattgagtttaatgcaatctattttaaaaaacagaagatatgaataaacaaaaaatagatgcagtatataacagtcgtttactgaaatgttgataatctattttgcatgcatattttgccctgaataaaagcctaaactcctaatcatcagcagtttctcttgctgtgtgcagctttttttcttcttggccatcttcctaaagccttcttagctggactcttcgctgtttggtctgttgtctgattctgttgtaaagtgtcctataaatttactgtctaggtagcaagtagttgtttgcccccaggaaatgtacttgtatgcttaattgtttggtcaagccttccccatccatcaagctggtgctcttccaagattgcagtctgatgtaaaataaatgtgtttcatgcaagcttttctatatgttgttagcggtgatgggtagaaaatttctggaaggtataaaaatcattgaatccttatattttgctgccaagtttacatggaaaactaagttttatcattcaaatgatccagcaaatcatcatctgaaatgacatttggctacaaaattgaagttggatagaatccaattgaagttgggtagaatactgttaagcctggatatttatgctttaaaaatttacataagatttattctgcataggaataaatgaacctgtatataaaaattaggagcatctgggtgaaaataaaatgtttactagtgtcactttaatttctttaatttggtttaaagtattatttccaccgtattctacgcttcctccagctgagggaaaaaataactacttttagtagatgatatctttcaatcattctctccttcatatgcagccctggttctgggactagggggacaagacacttcttgtaccaagagaatgctgctttgagtagagtttattcatgagaatataccttgcctgccacattgagagaagcccgttacattcatcatagtaagcctttagtaactatttcttgattgactgaatgagtggaattacacaatcttctcacttgaatagcagttggaaaactggttgaggcaggtcggggtttaatacaacagcagacggccatgtatggagagaaaacccaggaagaggaactggcatgtttagagcaacaaaggtgaacagtgaaggtgagctggactctaggaagcagagtgatttaactctgcttcttaatcttctaacacctctggttaaagaataggtttgtttggtcgattggttttctttttttttttttaatactttaagttctagggtacatgtgcacgacgtgcaggtttgttacagatgtatacatgtgccatgttggtgtgctgcacccgttaactcgtcatttacattaggtgtatctcctaatgctatccctcacccctccccgcaccccatgacaggccccagtgtgtgatgttccccaccctgtgtccaagtgttcccattgttcaattcccacctgtgagtgagaacataaggtgtttggtgttctgtccttgtgatagtttgctcagaatgatggtttccagcttcatccatgtccctacaaaggacatgaactcatccttttttatggctgcatagtattccatggtgtatatgtgccacattttcttaatccagtctatcactgatggacatttgggttggttccaagtctttgctattgtgaatagtgccacaataaatatacatgtgcatgtgtctttatagcagcatgatttctaatcctttgggtatataccagtaatgagatggctgggtcaaatggtatttctagttctagatccttgaggaatcgccacattgtcttccacaatggttgaactagtttacacaggttgattggttttcttaaatttccaatctgttgaggaccaatacttttgtaaaatccggtaacaataaattactagaaaaaatgatcttgtggcttgacatggtggatcacccctgtaatcccagtaatttgttataaattaaaaaaaagaacttagccaggtgggtggttgtgcacctgtagtcctagctactagggaggctgaggctggaggatcacttgagcccagactgcagtaagcaatgatctcaccactgcattttagcattgacagagtgagacttcatgtctaaaaataaataaataaatatgatcatgtgatattaaggcttctaaatgcttactttctatttatgtacacatctaattgtgaaccagtttttttgttttttgttttttgttttaaaaaaacagttcagtgcagttcactgactgacactagggcacactgagaaacacttgttttgggaatggtcagagatgaactggaaagaggttggcactaaattgtgaagaatcttgtataccattataaggatttcaaactttacgccatcaaaatttagaccaatctgaaagtcttgctctttattattttcctggatttgcattgtagattaatttgtttaaaacttttgtgccgaatagatgtttgataaaatgtgtatttaaaaagctattttagcttagcagttattggatggtgtcacacacgttgctctatactttttaaaattttgcagttaaataatcttgtttttgaaaacttggctttaaaggaacacttgacatcccagttttcccttccctcttctctaccttgtgtctgttttcataagagcgggttgtattttacctcagatttctcaggagaccagaactgatctgccagccttgctgccttttgttgctctgctctttacattgtctttacagtcaccaggtgtcaagatgctgcaagtacataggactggactgggcaggctgggggtcagcctctccaagggtcttcaccacaaagctgtgttggccgtccggagggaggatgtgaacgcctgggagagaagggccccgctagctcccaagcacatcaaaggcatcaccaatctgggatacaaggtcttgatacagccttcgaatcggcgggccattcatgataaggtgagtatttccaatatttttatgtgagaataaaatgaacctgcatatgaaaattaaggagcatctgagtgaaaataaaatgtttgctagtgtcactttgatttctttactttgattaaaagtattattttcaccctatgcttactccagctgaggaataaaagaacttacttttaggagatgttatttttcaatcattctctccttcatgtacagccctggttctgggactagggggataagactccattcttgcacccagagaatgctcctttgagaatgtaccttgcctgccactctgagagaagcctgtcatgttcatcattccacttgctcttcaaaataatattatggtataggtttgactcattcctattttagcaatatacatatatatgtatacttttcctttcctgagccattttaaagtagatcacagacatcataactcattactattaaatgctccaatgtgcatctcctcagaataagccatgttttctacataaccacaatatgactttcatacctaagaaattaataattcataatatcatcttatatagtctatgttcagattccccattgcctcaaatgccttttattttaattgaggcttaatgaagctttattgcattttattattgtgtgtctttagttctttcaatctagaacagcacatccctttctgattttgtttcaatgatattgacattgacatggactctagaccccaagacttacagagtgtcctgctactgggcacgaataatctggttgttttctcgtgaatagattatattctaattatattctatatgttatattcatgccaaaaatgtttggcatgaatacaacataagtgatatatgttcttcccatggcatcacatcaaaggcacaaaacatctggtcgtcccatggttggcaatgtgtggtcacttggttgaggtggttacagtgagagctcttcattgttaaagtctatatcaccctttgtaattactgagtcatctgtggtatggtattttaagatctgtgatgttcctattcctcaaataactttacaactaatcatgtcagcctccattgataatctttgcctggatcagtttttacatggagacttgtgaagtgatgcttttctaattttatcactcccctaagtttgtttgctgacattcttctttagaaaagaggttttcttttctttctcttccactcctccacttttttgagttgtgaactgttctattacagtgcattgaactgtttaccaaactgtgaattctgtaattcagtgtttaaacaatttctttaaaaaactttcttatcattgcatttaaagccttgttatgaggagtttatatatacatcttcatgttgacaagatgaatcttaggatgttgcttaaaagatgagttgtgaactgttttattacagtgaactgaattgtttatcaaagtgtggactcaaatttgctattgcattatgcttctttaatttttgcttttttgagtatattttggaagcatggacctttttcattcaatccatttaccattattctttttaatgtctgaattatcccaaaattggttagtgtgagtccttccaagggggcttctctgtcctttggatgtgaatccatttacctttgagctttcttgtttcttaggataacaaattgtcctagtctcaccttgtacctttcctgccccacatcaggaaattaataatttcaccaagaagacttgttttcttttattatatagagaccaagatctggtatctgatgttaagcatgtttattgttactgagatactgttgctcttaggctctattagtgtacagagtttagaaatatacacaaacagttgcccacacatatatatatatattatttaatcatgagttaatactgatactaaaagacctccagaggaaaaattaatgtccaggaaaattaaaatttaacattgcacaactttttacctttaaaattttatatttgcatctgttttctccaacagtaaaaatcttaattctttaatattaatgcatattgtttgctttatcctatgatatgtataaatagtgtcaggatcacaatactattattacaactaatagtaagcccactaaatgaagtttactatttctttgattttttttttttgcctttagaatatatctgacaatgaatattcagagtgttgtattaaaaaacttatttgaattttttttggtgtggtttcgtcatcaatctaatatccatctggatttatatattattgtttctattcagttttaatgtttgctttcccatttttgatttaattttgtttttcaaagtagaacatttacaaattccagaaatgaagtcatgttttcattttaatcccctacatccaattctcacccaccccaaagaggttacttttgtttctgatctatcattgttttttcttttttttttttttgcaaaaataaacacctgtgtgtgtatttacatatttatttcccttttgttcttatacagaaggctgtctactatatacactcttttaaccttgctttctttctttagtaacaacatatcttgaacattgctctatgtaagttcttagagagatttcatttcatttcatggctgtgtgataagtactccattgtacagatttaccatggtttatttaccaatctactacaaatgagaaaatggactgtttttgaagaaaccagggagaaattgtatattcatcctttaactatctctgttattcatctctactttcctttgaggtagattcctaactaaacttatcttttgttcaattgctcttttaattctgcttgggttatacctctttatttaaaaatcagatatgtctgtctgttgcttccatgtactagttatgcaatctcaattaagtttgtaagcctcaggtttcccattgattgaatgaggattataattgtacttacctcatgagagttgtatgaaatttaaatgagataattcatgcaaagagcttagcaaagtacatgtaatttattaatacatatcaatgattgttacttattacccaataagtcttgtattagccatgatgacattcaacctacatttccaaatcggtttactcgtatgttgtaaatgtctctttacttctctctttgtatttttatatatgccttcaatgcctcttactgatattaccatcttgtgtttgccatcttcatttctctaaatttcaggtagcctttcagattaatttaaattctcatatttctttggtatcctacctttcagattaatttaattttcatatttttttgatcatcctacttaggaattatctcaatatcagttaaagtagcatatcacttagatactgctgatcatgttttgtatttttcatttactccaatccagagtactttttactatattttgtgagctcctttatacaagggatcttgacaatctttccttgtatcttagcatctcatctctagcacggactaatgtacagtaaggatagtaataaatactaactgggtaaacactgaaaaataaatgtataattatgtcttatcttttaaaaatacaactaataaatcacaatttggcatttaatatttcagtgtactgttggtacaaaatatacatttcacaagtatgcttaagtcacatgtgctagtgcatatgaccaaattataaactcttcttaaacatacatatatatatatatgctgtgttttctaattgtttaatgctgtaagagttgacatgatagatttatctttagccaacaagtacaaacattacaagtagttgttctattatttgttctaattcatacagattttattccttcaaaagatttgttgtgcctttgctacacattttgctcaagaaaacatggatattacaaatcaggataataacctttaatcataattacaatttcaggactatgtcaaagctggtggcattcttcaggaggatatttctgaagcttgtctaattttaggagttaaaagacctccagaggaaaaattaatgtccaggaagacttatgcatttttctcccacacaataaaagctcaggaggccaatatgggcttgttggatgagattctaaaacaggtaattagtgattaatatttataaatattaatgtagaaaaataatattttttacttttaacccccaaatggagttttagtcttcttttcttctttagtttaagagaaacattgcattccttttatattctgaaatggaaagaatgataaaagctcttaccctaaacaatttaccaatttataaggtcaattggccatatactatcaaatagaattttagaactgaaaataacctcagggatcatataatctactaccttgtttttcagatgggatttttttttgagatggagtcttgctctgtcatccaggctggagtgcagtggcacaatcttggctcactgcaacctccgcctcccaggttcaggtgattctcctgcctcagcctcctgaggagctgggactacaggcgccagccaacacgcccagctaattttttgtatttttagtagagatggggtttcactgtgttagccaggatcgtctcaatctcctgacctcgtgatccgcccacctcagccttccaaagtgctgggattacaggcatgagccaccgtgcctggctgagacaatttttacatttattcacattcataagggaaaatgcatttttgagatagaaatactatgaaaaaaactagcagctgtttaaaacacaagatattcatttaacaaatatctattcagtgctgatgctgtgtctgacactgtttgttccaaggaaagagccaaagagtaaagtatctttattttcacaaagcttacattctagtgggaggagacagataatcaatgaataagacatctaatatccacttgggaatatgtgtgatgaagataaatataatattgaaggagaaaaggagtcctgggcactgggaggtgatgatgctattttaagtaggatggtcagggaggaccccattgtgaaagtggcattgagcaacaatctaaaggaggtgaaggaccgagccaatacatctgaaatgagtcttttaagcagacagattcgagattgagtgtggggttagagtacaccatgttcagaaatgcttattttgtctcttgtgtcaacaatggttggaggtgggaggaggagagcaacattactaagaaggctgctaacctaggctggtctagactgcaagtcacaagtgatggtggcctgcacctggaagaaatggtggaagagaaagttgtgaaatgtggacaaatcttggatatgttttagaggtggatcctacaggattcactgatgacataaaggtggaatatgaaaaaaggagagcagtcaggaatgactccaggttttggcctgagctacttgaagcaattgtcatggaatgacatgatgccattacaagagaaaaaggcttcggtgggcaaatcaagagttgagtttagggtctgccaagtttaatattctattagacattcaaagggataatctaagagggctgttggatatgagtctgaagctcaggagggcggcttctcctgacatacattttgtagttgtgagagaccaggtggtgtctagagccacgagactggatgagcctaggaagggagtatagaaaaagaagtctaaggtccacatcttccagatgttagagctaggatccgagattagggcagaggattggagggttcataaacagtaaaagcaatggaaaatacctattatataggagtgtaaaatacttccaattgattccaaatacagtagaccagggtgacagttaatttgtagtatttctaaagaataagttcaaaagtaattaatggaaataatactataggaagaatgttccactttaagcaaataaagtaattataaattactttaggaactttttttgttattttaacttttgttcattataagaaaattaaaaaaaactgacctaacagggctgcttttgggcagcattttgtaaccaattacttggagttggaagaatgataaacattcttaaattcatgaacataataaaaattatatgatctttgaggaaataaaattactgtttaaaatagtgtataattacaatacactaatatgttttaagcatgaattactaatgctttcctgagagaggaaaggttttcaaggaaagaggaagaatatgtttttagtgaaggcaaggaagataaatgacataaaggtggaatatgagaaaaggagaggagtcaggaatcactccaggttttatcttgagctactcgaaggaatcctgtactacttttaataggaaggttacctgtaaaaggaaggagagggaaaaagaaaaagagaggagaaggaggaggagagagaaaaatatctgtgcttggacttgagagagagtgttatttcccaggttgaaagagagtgttattttccaggtagaagagacctgagtgtacttagaatggttgagaaagaatgtaaagatggatgatgcaagaaagaggtaatagttcaagaagagtggtcttggaggacatgagagaagctgggatctaggtcacagagattcgtctcaaaatggatctgttttatttgaccagaaggaaggatgtaaggatgagaggggctttagacatgtttatagatggtgctgggtgggtcggggcagaaaggtaaaggaaattctacctttattatttcattgcttaccatcaaggggggtatagcacctgctacaaacattttttcatttctaattacaagcctaccattggtttgctacctggcgttttctaaatatttcatggtatacctgatggtagcttctgttagcaagttaaggtgtatttcagaggaaaaaatgtgaaatatttacttgctcatatatgtctttacttgttaaaattaagcttaatgggtccagttgaaatttttttatcctcttttcaggaaattcgccttattgattatgagaaaatggtggatcatagaggagtacgggtagtggcatttggacagtgggctggtgtggcaggtaagtatgccagggcttacatcacccacttcctaaattggcttggggaataactgtgagttttctcttttctgcggcaaggggataggagtgtttatttccatggaaaaaattaaaatgaaggtatgctgaaggatgatgaatacaagtggaataaagtatgaagataaatggttaaaaatattttattgcaaaaattcaaaggaaaaagcagggagaaggataacaactacaaacaagctctgatttttaggtagttcctttcgaaaggctggaaggagataccagttcggtaatgagacattgagaattagaagatagggagaaatagaaagggaaaaaaggtccaggggcaggaattttgtccagttaattctttgaaccttgtttggttaagaaacaccacaatatgttgtagctctgtttcagatcagactacttgcaggtctaataaggaaggtcatgacttcggtactatttgtttgaccttgttatataaaaatagaagattccttatgtaaaaaataaaagtagaggttcctcttcaaagactttcgtccccatttaattaggaataaatagtaacttctcttaggagcaaaatttattcagacctgtgcttctaaatatttgccccggcaggcttatactggtccaagcaagcattaagtcatagcctgttcctcttccttatttaaaagtgtttttacctttctcagcattccacaagttacttcctccttcctttgttctcctctacctttgcctcttttaaaaagttctaagttgctagccaataggaacgaatacagaacgtgaggtctcgttccagccaatggaaaccggacacagcagtagggtggacgtgtcaggttataaatgaccctgtctcttttgttcggtgtattcttgtggcaaaactgctggtgagtgtaccctttctgcaggaagtaaaaatggccttactaaataaattaaatttatgttcatgtgctgtttctttacggcaccagggaacaagcatttcaaacacttattatggcttatgtcattctgcttattattaatattatacttgcatccacctgctttttaaataataaatctaagcaaaaatgtgaaattcaaaaaattcttttacttaaaaccaattcaggcagggtatggtggctcatgcctgtaatcccagcactttgggaggctgaggcgggcggatcatttgaggtcaggagtttgagaccagcctgaccaatatggtgaaatcctgtctctactaaaaatacaaaaattagccgttggtggtgcatgtctataatcccagctactcagaaggttgaggcatgagaattgcttgaacccaggaggcagagtttgcagtgagccgagatcacgccactgcactccagtttaggcaacagagcaaaactctgtctttaaaaagaaacaaacaaattcattgtttttattcactcacctcatatctttcctcacccaacacccaaatcagctctattaaactaacagaaaattaaatttcttagtataaaagcagtaatagagagacctctgatgactatgattcctagggcctttcaggactcatccagatgagggttttatttacctagacagaattaatacattggaattaagttgatttatttagaagtcaccatattcagaagtaggaccacatccatgaataggatgtggttcatgttcttagagaagcattcaagcaagtaaacacaaatacaatgctagtgctaattgctataagaaaggtatttgagactgggtgcggtggctcacacctgtaatcccagcacttcgggaggccgaagagggcagatcacctgaggtccagagttcaagaccagcctggccaacatggtgaaaccccttctctactaaaaatacaaaaattagccgggcgtgatggtgggtgcctgtaatcccagctacttgggaggctaaagcaggagaattgcttgaatctgagaggtggaggttgcaggaagccgagatcatgccattgcactccagcctgggagacagaataagactccatctcaaaaaacaaacaaacaaacacacaaaaaacaaacaaaacaaaaggtatttgaacttggctgttgcaaaagctttcttttaaaaagtgcctttgaaggtgaggtctgaagaatgattaaaaatgattcagggaagtaggagaggacagtacaagtgtgcaaagctcaaagtgaagagggggaaaataatagttttgaggaatagaaataagttcagtataattagagtgtatatggaaataactatatgtggttcttataagtatttagaaggtggaattcacaggatgtggtgactgattggatgtaggagtgagggggtgagggagacactttaaagatgacatgcaggttaccaacttggacagtgattcagttctttcagtacccacaggaatggaggaagaatttggaggaagggagtaaatgagttctgtataggacaatgtattagtagattacatactaagttgctataatggagactccacaaaacagagcttacataagctagaagtccaggagtgagaagtctagggatggtggaatgagaggaagcctttgctattgtcaacacttggctttcatttctaaggtgacccttcaggttgctattttccagtcagtggaaaggggagaaaggaagtaaggtaaatagcttcctttctaaggatgtgaccaagaatctgaacatattatttctgctcatacccattggtcaaaattgactgagaaatgtaatctataactgggcaactatgtctgctgcaaaaaccatgtgccctccatctgtgaaagattaactgaggtcagacttactatcctgtcattaaccaactagaaaaattgaatgagatatagaaaacaatgttatcagacattggaccacaggcagtacagacttgtgatccttgatagatggaaaatagatgaggtgagctctatgttcaccctggctttctgtctggagaaattatcagatggtatgaatatggaaatgtgacacaaagcttggtgaactcaataagctgaggagataggagttggggagcctgagttggctggaattggcaacacatagtaccaaacagtgaggagctatgcagaaatggtgttccagaaatatgcttaggggtcttgctaagtggaggatgaaaattcatgagactgccttaggaacaatttgtggaaaagaacaattgccagggaactctatggaaaactgttctcagagtttgtcccatcagccagagtggagagacttaactgaatgtataaggctttcagggatgccctgaagtgtgtggctaaaccagctcttaataaagcctactctacattcattcctataaaagcttaaaaatgagcctaatgtctactttgagagagatgcaattagcacacaagcagattaaaacagctataataaatgttcagtatgttcacgttttaaatggaaacataaacaaaatcaggagagaaatagaggaaatcagtccatctacctatcaatcaatagataccacggacgaaaatctatacataagcagaggattagagaacatatatagattttcttctgtagtgtctattgattgatagatacatggtggatagattgatttcctctactcctcattttgtttgtgttttcatttaatccttgagcacattgaccagcatctctgtcatttcttgtttctgggattgtttccgttgttgattatctccaggttaatgttccattgcaagactcataattttttactgaatgccagacagtgttcattttacattgttgatagattttgttgttttccttttaggagcattgggctttgttctaataggcaattgtttgtggattacattgattctcttaaggcttgttttcctttcacctgcctctcttccttctgggactccaaatacacacgtattagagcatttgttcaccacctgtcacttaaattctgtttttttttctcagtctttttctctctttatgctccattttgtttaatttctactgcagtgaggaaggaagaaagtaaagaaatcatgaccggaacattcccatatcttgataaaaattataaatacacagataagtgaagttcaataaaccccaagccaaaaatagaaataaaaaatcacaaagaaaatgacagtagaaccatcataattgtcttgctaaaaaataatgataaaaacattgaaaataaatgggaaaagggaaccatatacatagaataactacgataaacatcaaacttctaatcagaaacagtttaagctagcagaaagtggagcaacatctttaaagtgctgaaagaaaacaaacaacttataattttatgcacagtacaatatatttccaaagacaatatcttttcaatatctttctggacatctttctagaaaatgaagattaaacacagattttctggtcaaatgaaagctgataaaattcactgacatagaagtatttaaaaaatttctcaagcagaaggaaatgaagtcagttgaaaacatggattgaattaaaggaataaagaacactacaaatggtagaatgtaaacaaacataaaaaacatcatatcttattttaaaacttttctttagggctgggcccagtcgctcactttgggagtccagggcaggctgattgcttgagctcaggagtttgagaccagcctgggcacacgatgaagtcccatctctacaaaaaaatgcaagagtagctgggcttggcggtgcgtgcctgtcatcccagctgcttgggaggctgaggtgggaggattgcttgagtctgggaggttgaggctgcagtgaactgtgttcatgccactacactccagcctgggtgacaaagtgagttaactgtctcaaaaaacaaaaaaattctttaaaagataattgatagtttcaaatgaaacaatgtattgtgatattcataacacaggtagaaataaaacttatgtcaccaataatgatggacaggtgaaaaggaatagaaccatacttttgtaacaagtttattttattataccggaagtagtataatattacttgatagttgactataataagataaagatgcatatggtaacctagaacaatgattaaaaataaaacaaggctctaaagctaataagccaataatggatgtaaaattcatactaagaaatacacagttcaagcaaaaatacaagaaaaagggcattaaaaaaacaaagtacaaatgaaataagaacacaaacagcaacatggtagctttaaatttagctatatcaatagttacattaatggcctaaaaactcccagataggctatagaatgagtcacaaaacaatttttgataaatttgaaagtctgaaatcatacagtatgtattctgactgtaatagaattgacctagaagtttttatagaaagatatttggaaattttccaaatatttgggagatattcaatgtaattttgaataactgatggattaagaagaaatcacaggaaaattagaagatatttttaagagaataaaaatttaaagtatagcttatcaaaatgtgtggggtataaagtagcaattagaaagaaatttacagcatttaattcttttattagagaagacagtttttaaattaataatttgtgcttccactataagaagctagaaaatcaaagagaaattaaatcaaacgcaagtagaaagaaataaataataaagatacagagaaaattaatcaaactaaaacaggaaaagaatagagaaaatcaatgaaaccaaaacctggttctctgaaaaatatcaataaaattaattaacatctaggtagactgatagaaaaagagagaagacaatatttactaagaaggaatactagggggccatcactatatcttttaaatattaaaaggataaaggaatattatgaataggtcaatgaatttggcaaccttgatgaactagataaattttcaaaatgtacaaattaccaaaatcagctcaagaagaaattgaaaacttaagtatcaaggtacaaactaaattccactaataggcatttattaagagaaatacaatattttatacaaagaatttttttatagcaactccatttataatagcctggaaacaacccaaatgatgagcagagaaatcaactgaaatagaaatgggaaactcttcaacaataaaagactatactgatacacacaaaatatgtatgaatctcaaaaacacgatgctgagtaaaagccgacacattgtaaaagagtacaattgacccttgaacaacacgagtttgaactgcatgagtccgcttatattcatgggatgcaaaatccacctttatcgggggccaccttttcatacaggcaggtgtcacagaactgactgtgggacttgagtatgcccagatattaacatgtgtaggggtcttagaacgaaccccctgcatatactgagagacgactgcttaaaattttgtttatatgtaactttagaagaacttaatctatagagagagaaagatcagtagttgtggagaccaggagttggtgaaatactctgtatcttgatcatggcggtaataacacagatgtgtatatttgttaacagttaccaaactatacattttaaatgggtgcattatgttgtatgtaaactatttctttataagttgactttagaaaaacgacaaaagaaaatttggtagggatgtttgttgggggtgaggatggggaatggaggagcatggaaagttctttcctaaaaagaataaggaaagaatggatgctcaggtctttgctgcagggatgctgaattttagctttctgaagaacatccaaattgagacagacataaaaaggtggctggctaagttttgaaatcaaaagaaagatctgttttcaagagagagacttgaagtcgtctgcatatagatagtacttgaaatttaggactagatgagattacctaagggacaaatgtagatatggaagaaaatagagcccaggacataatcctgagggtgaataatataaaaggaattgccacaggaagagggtcctacagaaacgtaggattaaaaatttcaagttgggatgctgaggggcactcttcgtttcaacaaagaagaggtctgcattgccaagtacttctgatctgatctacaatgtgttcactgagtttagctatttaggtcactaaatagatgatgggagccctagggttaatggtgactttgtttttgtttgttcatttgtttttgttatgggaggcatgggagcatgtttaaatgctgataagaatgaagcaatagggaaggatagtgaggggagtaactcagtaataaattccttgggtgacaggaagggatgggatccggagccaaggtggagagattagccttagataagaagagaaacattcttcctattttatcaggattgaaggtggaaggaataggtggggatgcatttgggttagtgcatttgatggcagaaagttgagaggatggatacaatctactgaggcctcttctaaatctgagatgcatgatctgtgcttctgcagggcaccatgcagattggagaacgagcagagagaacagcttaaattcttcccaactaatagtctttctattggtctgattctgtaacaggaatgatcaacattttacatggaatgggtttaaggctccttgctttgggacatcacacaccttttatggtaagagatgtttattttgctaatttttttgaaaacacatctcacaccttaataataaaaaaaatgctgagctggtggactaacttgagttgtgtatgtttgttttcagcacattggcatggctcataactacaggaatagcagtcaggctgtgcaagctgtccgtgatgctggctatgaaatatctttgggtttgatgcctaagtcaataggacccttaacatttgtgttcacaggaactggtaatgtttctaaggtaacttttacctgacttccttttgtaaaacaatccaaaactttgaaactaacccaaatgatgtgggcagccattttgtgggtatttcaaatggactggagattctattttaggcacttttcttaccaatgtatttttctcatagtgaaaagtagtaatgatctaaatatgtgtgtatgtgtgtatgtgtatgtgtgtgtatatatatgtgtgtgtgtatgtgtaagtgtatatgtgtgtgtatatctatacacatacatacacacatatagcttttagtttattgcaagctatcggagtcttggaatactttttcttcgtttctttttctttttttaaaagagataattcagccagacatggtggctcacacctataatcccagtgctttgggaggccgaggcaggcggatcacctgaggtcaggagttcgaggccaacctggccaatatggtaaaacaccacctataccaaaaatacaaaaattagccaggcatggtgacgcacacccataatcccggctactaaggaggctgaagcaggagaatcgcttgaacccgggaggcaaatgttgcagtgagccgagattgcaccactgcactccagcctgggtgacagagcgagactctgtctcaaaataaaataaataaataaataaataaataaatagataaataaataaataagataattcattgttaacagtgtaaaggtctgttctgagggtgatctgttttacctccatcaccaaaatgtccacgagtttgaaagtgacctcacgataaccattttttaagactacttggagttttgcaaacaagatgttaggaagattcatttaagactggaattcatgctttcttccttctctggtggttttaagaatgtgtttgtaatctcctatagaaacttgtcattgtaaaataattcctgcttcaaagaaaatattttacaaataggatatcattcttattatattgtgaggtaagcatctgattatttcttttgtaggaaatataatttccccatttgaaataatttctgcaatggatctaaaataaatggaaatatttttccctcatttctaaacactcttatcgatactctgagtagcttaagtaaaaaagaaaaaaattaaagccctttctaagtgtgctgttctaacaggaataaaagctagctagcaggctggttgtaagcaactcatctgtatttctgtgttgcagtcctggttgtagggaaggcaagtggagctatgtaattgttgggaaaatgatcagatctttgcagaaaaccatgttggttctttttctattgatttttggatttttactcatgtgttatttgtagggagcccaagcaatctttaatgagctaccttgtgaatatgtggagccccatgaattaaaagaagtttcccaaactggaggtaagggacaggagatgactaacttttaatgtttttaacagtctatgttataatattttatcaccataaatttttattatttgtcacccactttactaatttctttctccttttcaagacctcagaaaagtgtatgggacggtgttaagtcgtcatcatcatcttgtcaggaaaacagatgctgtgtatgatcctgcagagtatgacaaacatccggagcgctacataagtcgttttaatactgatgtgagtatcatattcaccttttaagttggttgcttatgatacagtgtaggtaggctattttcaaggagagaaattatgaattgtacaatggctttcagttttcctgttgtgagtcctttgggtatcatgatttggcctttggagctatcatcttttatggtatttggagtagtctcgcttagtgcttttggtggtttctgtggatacactctttgtttcactgtctccatttctgcttcaggtctacatgtgcccaatagcgctaagccatttcctgtgcgttactgttttctgtatttatcctggactagttagcactgtgggcagagcataatgcctatgacgtttggtctcaacatggccgcaaattgcaaccataatctcagccagcaatcttgaaatgcatgccattgattatcagggtgagggttaaatagcgttcctatataagtgcagaccatccccactgaatgaaaaagctattacacttacaaatcaaatccttcgatgcgacacacatcttataagaaataaaatatatttgtgtgctctatagtcattggccatgcaacattcttcccccagataaactaacaaaccaaaaaatacataaaacaaaaaatgcatgcatttattgtgaaaaagaatgctttaaataattttaaatatgtttgaccaacataacattttcagattctggtatgaagtttagcccttatatcgtgcagggcatgttagggaaaagaattcttaccttcagcttgagatgtttgaagaagaaaaattatgaacatagtttcaaactgaaaaatttggaatctaagaaatctttattccttctcatttccccaatcacagaagtattggtgattggatgcatcctaaaactgatactgacctagttgccatgtatgcatgtatgtagcacattgctccatagagcttacagatgttatccgatttaatcttcacagcattcctttgaggaatggggtgcgtattatccccatctggcaagtaagaaaaatagagcccagggaagttgaggtagcatgatttaggtcataccctactgatattagagctggaaatataattttaggtccaccgttttttgtttgttttgtttttgtcctttcatttttcttcggcatgcaatacattatagaatgaacacactaatgtatttatagtcatatatttctctatcatagggtaattctatactgaaatctgggtggctatttaatatctttgcctcaaaagatggacactgacatggacaataagaaaatcaattagaagttacatttaattatttctcatagaatttactgatagctttctttcagtatactgtattaaaataggctattttctattttcttgaacctggaacttgcattacagattgcaccctatacaacttgcttaattaatggaatctactgggaacaaaacactcctcgcctcctaacccgccaagatgctcagagtctcctggctccgggcaagttctcacctgctggtgtggaaggctgccctgcattaccacacaagtaaggactctacttcagcaacgatagtagttttgaccagctgcttccattattagaggatcaaatatcacatatcagaggacttgatttctaatgactacttctctgccttatctttcttgacctcttggcagtactcaacactgttaaccatctctccttttttttttaaaattttattgtatatatttatctttagagatggggtcttgttatgtttcccaggctggtctaaatctctggaccccaagcagacctcctgccttggcttcccaaggtactgggattataggcatgagccaccatgcccagcccacctccacttcttgaaacattcttcttttgtcttctgtgatgccccatgcagctctgaagctatttgctcagagaagtcttctcttgtttctccattctcatctctatctctagtctgttactctgttttttgttttgaaattcctcatttattttctcgtgtttatgatttttctccctcttcctagaacttaatctccttgaggatagggggtctctttctcttggccacaagcacctagtacgttgcctgatatataataggcattgaaataattattaaatattgaattaattaattaatatttgtggaagagtaaaagatatgtctgcttttctaggcatgtaactggcatcaagtaaatgccaagggaatgctaggagcaacaacatcaccagggtttagaagaaagcagaagcattgggcgcagcaaaccttaaagggaggaatgaggagaacaagaattccaagtgaaaggattggtgcttaataaatatggagctaccaaagcatatttagggtgctgaatgtgtcagtctagctctacattggttcatgtatgcaagtggtaattggagattgggcagaaaaattgtagtcaactttttgaatgctggactgaaacttttgttcgtaattctagagatttatgtgtaaccacatgtaaaactatacagtactaagcagggtggaggtagcataattgaacctttctgagttatttagagataactatgttaactttagtctgttaatgatttctctaggaaagcaattttctgcgtttgtttccttttttcttttctttctttctttctttttttttttttttttggagactaaaattcatactgggaagattctctgtatcacagaacagtgcagagtcctgaagaatgagctataggaaaaagaccccaaaagcctggaaaatatttgggatccacattgaaaattgttccaaactcctttttttttagtcttgttggagacttcagaatttcatcctctcatctaagtattgatgttattttcagactcgtggcaatatgtgacatttcagctgacacaggagggtctatagagtttatgactgagtgtacaacaatagagcatcccttttgcatgtatgatgcagaccagcatattattcatgacaggtgagtttgctaaagttgttagatgatattcctcacttagttacattgatatactactctaaaattctcagtattttacatctttatgaagttatgaaaatttggagagatatactcaaacatactaaaatttggggaaaaaaacatgcacatttgaacacacacacatacacaaatatatttaaatgtgagatgggttgctgaagaaggaaaacgttttgtttgaaaagttttgtttattttcgcgtatacttttatacaaaattacaaatgataatgaagcaaatatagtggagtagacatctggaaaagtcagaacttacaaataatgttttgtgcttctgaataaaccacattgctgcttttagtgttgaaggctcggggatcctgatgtgttccattgacaatttgccggcacagctcccaattgaagctacagaatgctttggagacatgctttacccttatgttgaagaaatggtaagcagactggtaatatcagtaacaactctgtggctttaaattgaataattgagtaattctgtgaatgttctggtcttctgtatctaagaaatctgctgtaaaatgaaatgccagcccttccctgaaatacttactgatactattttatttgacatattctgcttacttgtctcttggggtataggatgatatcactgccttcatgcttaattacaattacatctctttcccattataggtcttttattaagaatgttcaccacgtgtgagataaaacatgctttatttttaattttttgccatctatgtcagttcatattggggcactcttattgtgccgtaataaccaggtctgaaaaattagaaatatagaacatttttattgggttctggtcactaatagttaagtgacagcttatgttgaggtcatgtatttttcttctgtgatcattgattagctaatgaaataccatgttatctaaattccattttctttatcaaagaaaatttttcttatttcagtttttatcctaagcattcatagccttcctgctttatttttatttcagactgtctgcctctgccaatgattccaatataaagaatggctcttactcttttcatctatatcttccctcataagctcttacactaaaatagaacaatagttagtggggcaagaataaatgaaatttccagtctgtgatcctcttttctttcaaatagtattccattttgaccctttctttttcctattggaatatgtcaacagggagatgtagcaggcttgagaaaatgcaggcagcaggcactgtcatggttagacatgatattgcacctactaggtgcaggaagtgttcaaagtactaaatatattttaattttaagtactcatgaaaaccgtattaggtaaataccatcattaaatccattttaccatcattaaatccattataaaacaaatacagagaagttaaggacgtgttgctgatacagtcagtgctggatctgcaatgtgtctggagttggtgctgaggcattaaagaagcaggagaaggagagtacaaatgccaggctttgctggagatatcaacttgctttataaccttgagcagggcactcacttcctttctgagcctcagatatttcatctgtaacatgaggtggttgtaatgaatacataatcttattttattttattttatttttttgagacagattctcgccctgttgcccaggctggagtgcagtgatgcaattgcggctcactgcaacctccacctcccaggttcaagcgattcttcctgcctcagcctcccgagtagccaggaccacaggtgtgtgccaccacgcctggctaatttttgtagtttttagtagagatggggtttcaccatgttggccaggctggtcttgaactcctgacctcgtgatccacccgcctcggcctcccaaagtgctggtattacaggcgtgagccaccgcgcccagccacgaatacataatcttaacagtttctctacttctaaaattttataatggtaaatgtttaagtggccaaactgtatgtatataaaaaatacaataatatatttaccaaggtgtttaccatttttgatgttcttcattcatttccctttggcatcatttccctgcagactgaataacttaatttatcacttcttacagtacaggtttgctgatgacagattctcttagttgtcctttatctgaaaatgtcttcatattctctattctgctaatcagacatttctgtgcatttttaatttccgatgttctattttcagttctagaatttccattgggttcttttttaaaaattttgttttggtattttctctttttctgcagagagtttctatcttttcattaattatgagtgtatttttcgttattgcatggaacatagttataaaaaccactttacaatgttcatctcatacttccaactttctgggtcaatttgtggttgaactttattgattgtcttttcctttgagaattggtcatagtcttctggttttcatagactgaataattctggattatggcctggatgatgtggatattatgtacatgtatcaaatgcattttctgtattcattgagaagatcattttttccttttctctgttaatatgtgaattgtgtcaattggttttcaaatatcaaactaacttcacagtcttaggataaatccaacttggttgtatgttaaccttttatttatgactaagttctggttgctaagaaatataccaacatttattaatctattttaccattggtagacatttggggttgtggagatatatatatatatatatctccacattattttgttgagtttgcacatagatttatcttgtttaggattattaacatctatgttcattagagagcttaatctataactttattttattataaggtctttgtcaggtttggatattaagtttatgttggcttgaaaacaagttagagagtatttccttatttttggtctcaggaacattttacatgagattagtgttagttctacttgatacaattcattagcaaaactatttgggttggtagcatttttgtttgtttctttttttagttttaagggtaagttttaaaattatagattacattttatttaaaaacttaagaatgttcagatttttatttttctttgtcaattttggtatgcttattttctaggaatttatatttcatctaagttgtaaactttattgtcattttttttttagtaataaccttattaactctttaatagattctgtagtgatttctctttttcctgatgttggttgggtatgccttctgttttttcttaatctgttttacctggacttgatcaattttattagtcatcgcaaaaaatcagttcaactttggtgatcatctctattttatatcattttctgttttcttaatttataatcttacctatactatattctttattttactttattgagagcttagttaacctttcttttcataacttcttgcaataagtgcttaggtaattgttttcctgtgtttctctttagcaatatgtgcattttaaagccattagatttactttgagtacagcccatttttgattgtattatctcttgttttgttctttgactttcttgtatattctgagtattacttccttgtgagacgtatagtttgcaaatactttttcccattctgttgtttcttcactcaattgatcttttgtttgtttgttttgctgtgcagaagctttttagtttgatgtaattccatttgtctgtttttgggttttgtgcttgtgcttttgagttcttatccaaaaaattcttaaccaggtcaatgtaatgaagcatttcccctgtgttttttttttcttctagtagtttcatagttttaggtcttccatttaagtgtttaatccattttgagttgatttgtttatatgatgagaaatagggggtctagttttattcttctgaatgtagatatccaattttcccagcaccatttattgaagagactcttcttcccttaatgtttgttcttgacacttttctccaaaactggtggatatagatgcatggatttctttctgggcttgcttttctgttccattggtttatgtgtctgctgttatgccagtaccatgctgttttggttactatagctttgtagtatattttgaagtcaggtagtgtgatgcctccagcttcgttctttttgctaaatattgtgtgacgatttggggtcttttgtgatttcatactagttttaggattgtcttttctatttctgtgaagattatcattttttttttttttttggtaaggattgccctgaattggtagattgctttgggtagtatgggcattttaacaatataaattcttccattccatgaacatgggaatatctttacatttgtttttgttttcttcaatgtctttcatcaatgttttacagtttttagtatagagatctttcatttccttggttaaatttattcttatgcatcttatttttgctgtggctattggaaatgggattttttttcagattgttcacagttggcatatagaaatgctactaatttttgtatgttgattttgcattctgcaattttactaaatttatcagttccaacagtgttttgatggagtctttagggttttctatatataagatttcgtcatcttcaaacaaggacaatttgacttcctgctttccaatttgggtacctttgttttctttttttttgcctagttacactgactagaacatccagtactatgttgaatagtagcatccttttcttgttccatatcttagagaaaaagctttcaattttccatgatcagtatgatgttagctgtgggtctgtcaaatatggcttttattatgttgaggtatgtaccctgtatgccaaatttgatgaaagtttttatcataaggtgatgttgaatttgtcaaatatttttttctgtttctgttggaataatcatatggtttttgtccttcattctgtcaatgtgataaattatgtttattaatttgcatatgttgaaccattcttgcacccctggaatgaatcccatttattatggtgaatgctcttgtaaatatgctgttaaatttggttttctagtattttgttgaaaatttttgtatctatgttcttcagggatattggcctgaagtttactttttttgttacgtccttgtctgattttggtatcacaataacactggccttatagaattagtttggaagaattccctcctccttgatattctggaatagtttgagaagaattggtattaaatcttctttaaatgtttggtagagttcagtagtgaaaccatcaggccctgggctagtctttgatgggagactttttattactgattcaatcttgctgcttgttatcgattcgcctattcagattgtttatttttttctggttcaatcttggtaggttgtatgtgtccaggaatttgttcatttcttctaggttttccaagtttttggcatatcattgttcataatgctctcttatgatcctttgtatttctgtggtataagttgtaatgtctcctttttcatctgtgtttttttttttttttttgagttgtgggtgatttgttatgcaataatgttgcagaagctaatacatacttaaaaaaaacttttaggttcaggggtacatgtgcaggtttgttctataggtaaacttgtgtcacgaggggtggatgtgcagattatttcatcaccagggtattaagcctactacccaatagtcattttttgtgctcctctcccttctccctcaggaaggccccagtgtgtgttgttcccctctatgtgtctatgtgtcctcatcatttagctcctgcttataagggagagcatgcagtatttggttttctgttcctgcattaatttgctaaggataatggcttctagctctatccatgttcctgcaaaagacatgatctcattcttttttatggcttcatagtattccatggtgtaccacatgttctttatacagtctatcatttataggcatttaggttgagtccatgtctctgctatttacaatagcgctccaatgagcactctttacaacagcatgtgcatgtgtatgtgtccttatgatagaacattttatattcctttgggtatattacccagtaatggattgctgggttgaagagtagttctgtttttagctctttgaggaattgccacactgctttccacaatggttgaactgatttttactcccaccaacagtgtataagcattcgtttttctccacaacctcaccagcatctgttatgttttgaccttttagtaatatccattctgactagtgtgagacggtatctcattgtggttttgatttgcatttctttaattatcagtgatgttgaatttgttttcatatgcttgttgactgcatgtatgtcttcaattgaaaagtgtctgttcatgtcctttgcccagcttttaatggggttgtttgttttttcttgtaaatttaagtttttatagaagctagatattagacttttgtcagatgcatagtttgcaaatattttctcccattctgtagattgtttactctgttcatagtttcttttgctgtgcaaaacccccttagtttaattagatcccatttgttgcatttgtttttggtgtcttcatcatgaaatctttgctagttcccatgtccagaatggttttgcctagatcatcttccagggtttttataattttgggttttacatttcattctttaatccactttgagttgatttacaccattttatatgctgtaaggaagtgatccagttttaatcttctgcatatagctagccagttatcccagcaccatttattaaacagggagtcctttcctcattgtttgtttttgtcagctttgttgaagatcagatggttgaagatgtgtggccttatttttgagctctctattctgttttattggtctttgtgtctggttttgtacgagtcttatgctgttttggttactgtagccctgtaatatagtttgaagtcaggtagcatgatacctccagctctgtttttttttgtttaggattgccttggctattctgcttctttttggttccatatgaattttaaaatagtttctttctagttctatgaagaatgtcatgggtaatttgataagaatagcattgaatctataaattgctttgggcagtatggccattttaataacattgattccccttgaaaactggcaacaaggatgccctcttttaccacttgcatccaacatagtattggaagtcctggccagaacaatcaggcaagagaaagaaataaaaagcattcaaataggaagagaggaagtcaaattatccctttttgcagatgacatgattctatatctagacaaccccctagtcttagcccaaaaattaagctgataaacaatttcagcaaagtttcaggatacaaaatcaacatacaaaaatcactagcatttcaacacaccaacaacagtcatctaaaattttattcatttgagtcttttctccttaggcagacacagggtttgttgattttgtttaccttttcaaacaaactctttgtttcattgatcttttatattttttaagtctctattttgtttatgtcttctctaaccatcattatttctttgtgtctactaattttgggttcagtttgttcctgtttttcttggtccttgaggtgcaacattgggttatctgagatctttgttcttttttgaaataggcatttcaaaattgctataaacttccttctgagtactgcttttgctgtatcccataggttttggtaggttgtgttttcattttcatttgtctctagaaatttttaaatttcccttttagtttctctattgactccttgattgatgaggattatgttgtttaatttgcatgtgtttgtagtttccaaagtttctcttgttattgatttctagcttcataccactgtgatcagaaacaatacttaatatgatttcaatttttttgaatttgttaagacatattttgtggcctaccatatggtctattctggagagcattctatatgtactagagaagaatgcgtacactggtgctactggataaaaagttctatatttatctattaggtccattttgtctaaagcacagatcaattccagtattttcttattaatttctttctggttgatctttccatctttgaaagaggggtattgaagtctcctactattattgttttggtatctattttttaccttcatgttgattaattaatatttgctttatatgtttaggtgttccaatgttgagtgcacatgtatttacaattgctatgacttcttaatgacttgagctctttaataatgactttctttgtctcttgtgaaaatttttgtgttaagttttttctatctagaataagtattgtccctgtcctctgttggtattatttgcaaggaatatcttctttcatcccctttgagggatgaaagcctatgtgcattcttaaggtgaaagtgtgtctcttgtgtgcagcatatagtgagagcttattttttttttctctttgcaacttttattttaggttcgggggtacatgtgcagttttattacatgagtaaattgcatgtcactggggtttggagcacaaatgaattcgtcactaaagtagtgagcatagtacctgataggtagtttttcagtcttcaccctcctcccatcctccaccctcaagtataccctggtttctattgttcccttctttgtgtccatgtgtactcagtgtttagctcccacttataactgagaacatgtggtatttggttttctgttcctacattaatttgcttaggataatggccttcatctgcatccagattgctgcaaaggacgtagtttcattgtttgtttatttttgaggcagtcttgctctgtcacctaggctggtgtagtggcaccatctcatttcacaaacctctgcctcctgggttcaagcaattctcctgcctcagcttccccagtagatgggattacaggtgcccaccaccaagcctggctaatgtttgtatttttagtagagacagggtttcaccatgttggccagggtggtcttgaacttctgacatcaggtgatctgctcaccttggcctctcaaagtgctgggattacaggtgtgagccactgtgcctggccaatttcattcttttttatggctgcatagtattccatggtgtatatataccacattttctttatccaattcaccactgatgggtatctagattggttccataactttgctattgtgaatagtagagctttgtttttttaatccattcagccactctgtgttttaattgaacaatttaatctgtttacattcagagtaattattgatatgccaaggacttactactgccattttgttaaatgttttctgattgctttatagcttatttgttctttttttcctctcctgtttatctttgtgatttagtgatttcctttagtgctaaatttgtattcttttatctttatcatttgtatatctgctttagtattctgctttgtggttaccacaaggcttacataaaatgtcttatagttataatagactattttatactaataacaacttaaattcagttgcatacaaaaactgtagaattctaccctcccaccagcgtgtgtgtgtgtgtatagtatatatagtatatatacacacatatatatgtatatgtacatatattcatatttcttttttacctttctgagttgggtgcaaccagttaatattttttgatgtcacaattttcatctttttagcttacatatttcttaatactttattgtagctatcattatttttgactgttttgaattttaacttccatcctagagttatatatacctatgttttgcagagtgccatagtagtatcagggtattctggatttgaatatatatctacctttatcagtggatttttttactttttatgttttcatgttagtaatgttctttttttctacttgaaaaactcccttaagcatttcttatagagcagctctagtggtgaagaattccctaaacttttgcttgtctgggaaagagtttatttcttctttttttctgaaggacagctttgctgtgtatagtatttttggctggcaggtttctgtgcatcagcactttgaatatatcatcccattctctcctggcctgcaggatttctactgagaaatctgctgatagtctaatggagattcccttatatgtaacatgatatttttatcttgctgcttttaatatcatttgtctttggtttttgacagtaattataaagggacctctttgggttaactctgtttggagattgttgagcttcatatatttggatgttcatctgtcttccaaggcttgaaacatgttatttttcatttatcaaaaacttatattcactaattttaaagttttacattttttaaaaaagttatctatattgtccttttattttttgaacatattaatcacaatttagaatcatttttgttaattccattacacttcttggttctatttatgttccctgtttttttccttggtttttatggttttcagtaaattcttacctttcaatatatttatttatttttggtttaatgccagacattgcgtattcaaaattttagagatgactcgaggctctggatgttgtgtgattttcctccagataatatattctttgttttgggcagtgagatagacaagggtaaaggttaggagaaattcacctaaattcagttaggatgaagtaatttaaatctaggtttcagtcctttggagaactgacctttttcaaattaccatttattttagatacatgaaaaccattttctatttattttaatacagtaaataaattctctgttaccagcaccttctaggaacatttttcttgttcttagttgggtgagagagctagatcttaacctatagttagggaggaaagattgtctcattggctcttccctgtccaccagaaacagcaatgctaacttatatgccattacttcttggcagtatattcaagaattgggttctttgagaaggactaacttacatgcccattacttcttggcagtatattcaagagttgggttccttgggagaaaagaggaggactagaaggttgctgaaatgttaacatgagacatcttactttttttgttgtctctgactcaattttgcaagataatgtgaagagggtgtgaatgactttctaggtctactcagtcaagtcacttttgctaccacctttgaaatgctgatgtacctatacatataactactaatactgatactgatctgagggagaccccttactgcagggttggtggccttgctgagtggaaaacctcattctctattcctaatagaacagcttgaacttttggaatggagtagaggcccttgaagggaggactatagctataaagcttgtgaagatatttgacaacatgcttaatatgatacatttaactggttggaaaggataagcttttttgtgttcacagcaagcaaagatgattataatccaattttagttttaaaattgggacttgtgctgaaacaccatagtcttgtgggcagagttgattgcttgtagaagacttctattagcaatcacttatactcggtgatcctcagaaaactgtttcttaaattatttttttcccttgattaatattttaatatggatacaaacaccaagaggccttctttgcagtgtcatttatctcttacaaatctcattttcctttcagatattatcagacgcgacacagcctcttgaaagtcagaatttttctcctgtggtgagagatgtaagttatgagcagaatattttttcataaatgtatttttttactaccatttttgctctcttctctctgtttctctctctctccatctttgtcattctgtcttttctctatcagtcatgacaccttgtttgtaggactaaaatgtggatactaaagaaatgtcttgtgtcatatacatttaggcagtgattacatccaacggtacattacctgataaatataaatatatccagacactccgggagagcaggtaagtattgttcaaatcctcttgttttatacatttattcttatcttggaattcatgagtattttttaatgcttcaatgggagttcttaatattaggtttgtacactatataaattttgataatgtttgaaaatgtaggaaatccaaactaagtggctggctgaaaccactgtgactgtttcccacttaacagtagtatagcgtcattttggctttctaatagcctgtgtagacgtatgggtaagcaagcaaaaatccaaaaggattgtccttcaggtagcttcttgtttttctctagcttttgatacatgatcatgcctcggttattttctcaaaggaaaatgagcacactagcctttctgtggtgctttcagtttgggttttagttgaggatttggactctcttttgtttagtataatgttcagggaagtgaattcgtggttacaaattaagagagttgtgaataaacaattgatgaaagtaagaggtagttttctccagggattcttattagggggaaggtgccaaaaagtaatgtagcaaatctactcaagagaatagttactttagaaaggtctttagctaaggcccttgtttccttaattgattttactacttaaagtttgaaaacattgattttatttttttggaatattctggtctcctccagccaattgcccttgatctgtgtttatatctactctgctggtcttttcacattaaaaaaggaacccatgtactgaagtatcaggagaaaccagagttggaaaaaaaaataacacaactgttttatcttctttttcagtggttctactacaagcagtaaacaaagagatctgcccagatcaagcgtactatagttaggcttttccatatgtgctggtttgattatagttactttttaatgtgtcagtgaaatatactggcataagacactaagtatttactttttttctttgtgaataagagaagctagctctataaccctgtaggagagtttcttacaagtaaaacttcacatctgtttgtttttcatagctctatgttttggttctcatcatccgatggtcttttacactactgaaactagtgacatgtctaacatgactggtcaagatgatatcttctctcttatgtgtcatatttcatgaagcagcaggtttacttggctccgattgctagaacttgctgccctctttcctgatggaaacttgtgccaagatactgataacaaaggttgcattttgaaaggaaatttactttatattgaaccctgccttattcttctcttcttttcatcacttagcacgtgattattaacctggagtctggggtttgggggtactgatgggctggacagtattagaatctaggagtgagtttctgttttgagcatttggtatttggaatgttttaattatacaaactatagaaaatattgttagacagaaaatataataaccgaataatagtggttctctctggaaggtgaaatgggagatggtagtagttaaatgagacctcttcacatcagtaatatgtgtgttgggatacagaaggacaggaattttttatacttatatttatttttatttgttaaaaattaatgaaaagttttttgggttaatgttcaagagacattatggcctaaagattctaaataacagagcctacttcagtccctggcactaggccatttctatggtttcatgttgataacagggaatttgattcaaaaaatatttaatgagtaaattccatatttaagacaaaatgtgctgagcattaatgggtttgtaaagatgggtaagtcttagatgctgccttggaggtcatagtttgtgggaaagacagaaagtcatctgcctggaaggtattaatataaattccagcagaatagactgttcaagaattaaaataatgcctaacactagtcttcagcttctacatttaacccatggcatgtgtgactcaaagtcatggttttgctattttcaagtgactctaaagatatatgacaaaacaatttctaatatggataataatattctcttcctcaaattgttgtcttctgggattgttgtgaggaataaataaggtttccaaggtaaaaccccttgtatgattttgaaccacatcttctggaaggataaaatgtgagaccaagttactcaatcacaagtaagccaagaaaatgtccagcatctgcacctcactagtaatactgggaattcatgaaatacatctatacgtctgtagatatatatctcagagatgtcaggtctattgttcttcattgtacaaaatgtaaaataattaaagaaacatgttgttatagggaactcattcatgttgaaattcttattttaagtaatgtgaagagaaaatgttgcttcattatttaggttttagtatgatttgtggaatgcaagattctgttatttaaaaaaaaatttttaattagccccttcccttttttttttttttttttttttttaaatagggaacgtgctcagtcactttcaatgggcaccaggagaaaggttttggttcttggatctggctacatatctgagcctgtattagaatatttatcaagagatggcaatatagaaataacagtaggtaaataagccctaatttttgaagaagaaaaatgtattttatagcgtgttgacttctacattttagaagactactttgtttctgaaatttgaaaatttatttttggagatgttaaaatatattttactctgacatatctaaaaatgtgacttttaattactgaaataggatctgacatgaagaatcaaattgaacagttaggcaagaaatataatattaatcctgttagcatggacatttgtaaacaagaagagaagctgggcttcttggtggcaaaacaggatcttgtcatcaggtcagtatctggcaagaaatgagtttcatatttttgttgttatttcttttctcatttttctttctctttcacttctcctctcttcatactcctttggagtaaagtttttaattgaagtatgggtccagaaggtgcacaaatcataagtatatagctcaatgaattttcacaaagtgaacacattctgtgatgattatttatgtcaggaaacaagagtatcagtaattccccagaagccctcctggtgcctctttctattcacttaccccatcctctaagggtaaacactatcctggcttctgacatttagtttctgcctgctttcagacttcatataattggaatcattcaatatatcctcatttgtggcttccttcttccattcaatgttgtgattacaagagtcatttgtgctattgtctgaaatgataatttattcattctcattgctgtatagtatttcattatgtgaatataccattattatttatttgactactatagatatttaggttgtttctggattttggctattataagtcacactgctatacacatttcggtatttgtcttttagtgaccatttcactaaaacagtaattatacatttctgttgggtatgtacttagaagtgggattgcaggtcataaggtattaatgttttatatacttaagtgtctattttatcctttggagttataaagcttaaatggaataagtagattatttagccttatgaccattttaagtctcatcatagcaacagatcctgaaatagagtattactaacaaaaatgcagatctaacatctagacacagaattacatgagattcactttataactaaatttggttttgatatttgagtacatatgtacacaaagaaaggaacaacagacatcagggcctgcttaagggtaaaaggtgggaggagggtgaggatcgaaaaactacccattgggtactatgcttattacctagataatgaaataatctgtacaccatacccccataacatacaatttacctgtataacaaacctgcacatgtacccctgaacctaaaataaaagtaataaataaataaataaataaatttggttttgaaatgtcaaagtgatgaaaatagtcattcattcctagaattttcccaagatgagaagaagaaagttatctgttccctcatagtgattacctagtcactttcacagatcagattcaaattgtatctttggaggaagttctgtgatgttctgctttaaaaaaaaaaaagcaggcttggaaaacatataatgaaaggtagttctgtaggctggcatttttacatttctcttgatatctttaaatacaggctaactttatgcacttaaaatgttactcaatttatataaatgttatgagcgttaatttttatttagaaaaaattgactaatgaccaactgaacctctctgatattaccatgaaaagaaattctgattcaaaatagaatgatctagccttctgagtagaacaataatttattttgccacacagaggtatctaaatatatcatttttaattaaaagtaaaataacatgataaaatccatttggaacagaatctgaaataaaatgtgacaagataatggacaaagacatcaatctcttaatatgtccaaattttaaaatttagcaaactagtggccttcaataggatttagcattagcaaaaactgttttatatttaatgttttctcatttggtgtattttctatgctgaataatggtaatgccattaaacaatgttatcctataacattccttaaattttttttttcttttttttgagacagtctcattttgtcacccaggctggaatgcagtggcacaatcttggctcactgcaaccttcacctcccagactcaagtgatcctcccacctcaaccacccaagtagctgggactacaggcacttgccactatgcctagctaaagtttgtatttttagtagcaatgggatatcaccatgttgctcaggctggtcttgaactcctcaagcaatctacttgcctcagcctctcaaagtgtcaggattacaggcataagccaccatgcctggctaattgttttattttttaagttggggaaagaatgttttgatataaccacagtgatagggactacaaaaaaatgagtaaagaatatgtttcacaaaaaatgtttagtaatatgtacaattctcatagttataatttttcaaatttagtttttaatttaatacaaagtagtaattgatttcaagtttctttgaaaattccacatgtattttgtgtgtatttttcacaagtgacattttcgaccttttggagtaattgggttggagtcaacttaaataatttaagagtatgtcccattgagtgcttactgtatcatattcgagacgaattatgaactctaatcccttcgtctaaaaaagtaccacaggcaaagtcctatcattctacttctcagcctattataaggccaaagttttaaaatattattagatcttagtaaaattttgattgagcaagtttaatatggagaatgtgatttaaaaatttctgtttaacctttaagatgatgacattgcacattttctattaatatttggttctaatagttttgcgagtattttgctcagtggtttccaaaagtctactaaatttgttagcagtaattatttttggtttaaggaaacaaaaacacagagaccaggagcttttgatactgataaaagacatacacaattttattctgcattataataaatttaaatagcaatactctattctgttgacctgaggaggaataaatgcctgaaaagtggttttattgtttttttttttttatgaagtagagaaaatctgaactatgcagaatgaatttgacaagtacaatgatatagtgtaatattgtttctgttggtttttatttttttttgttggtactgtagacagctttaccaaataggcattaaaaaacaaattaaatattatcatgtaattaacagagtattttattattcctagaatagaatatggatatgctcatttttataacactcatatttatgaaaatttaatttcttttgattatgcagtcccagagccaaattattataccacagccaaatttgttgaaaaccgaatcaaagttgagctttgagtacagcatcatccattgttcaccatgtggatttttagccattttgatagcatttacactgatcaacaccgttaaagcctgtctcctccgtggacccttcctggagtctactcaataatgtaatacattagctgtagaaatatagggatgaatggagcaaattccattaaatgttataaatatataactggatgcatgcagaggaaggatatctggctttggggaaatctgggaaagctttgtggaggaaaataactctttagaaatttcttaaagaggcaggtataatttggattggggttttctgtcgtgggctttccactaaaatagggctgctgttctttttaccttcacatcttccaaatggctatcaggaagtgtcaaactaggtcaagatggcccatgtattacattattgagtagactccaagaagggcccaaggaggagataggctttcatggtgttgatcggtgtaaaggtatcagaatggctaaaaatccacagggtgaacaacagatgatgctgtactcaaagctcaactttgattgggttttcaacaattttggctttgggattataatttggtgctgggtctgcataatcacaagatatcaaattttcataaatatgagtgttatataaatgagtatatccatagtctattgtaggaataataaaataccctgtcaattacatgacaatatttaagttgttttttactgactatttggtaaagctgtctgcagtaccaccaaaaaaataaaaaccaacataaacaatatcatactacatcattgtacttgtcaaattaactctgcatacttcagattttctctagttcattaaaaaaacaaaataaacaataaaaccacttctcaggcctttattcctcaacaggtaaatagaatagagtattgctatttagatgtgataacagggtgaatttattctgtggcccacagggagtcattgatcacgtctttccaagggaatatttatttattttactttcaacttttattttacaatcaggggttacatgtgcacatttattacaaaggtatattgtgtgattctgaggtctggagtatcaatgaatctgtcaccaagtagcaagcatagtacccagtaggttgtttttcagcccctgctccacttcttatctcttccctcttgtagtcctcagtgtctattgtttccatctttaattccatgtgtacccagtgttcagctcccacttacttgtaagtgagagcctgcagtatttggttttctgtttctgtgttaggtcgcataagataatagtttccagttgcatccatgttgctgcaaagaacactatttcattcttttttatggctatgtagtattcaatggtgtatatgtactacattttttaatccaattcacggtttttgtttttttatttttgtttcattttgttttgttttattgagacagggtctcactctattgcccaggctagtgtgcagtgctgtgatctcaactcactgcagccttgacctcctgagttcaagtgatcctcccacttcagtctctggagtagctgggactacaggcatgcgccaacatgctgggccaatttttgtatttgttgtagagatgatgtttcaccatcttgtccaagctggtctcgactcctgggcccaagcgactcacctgtctttggcctcccaaagttctgggattacaggcatgaggcacagcacctggccccaatcccactattgatgggcatgtcaattgattccatgtctttgttattgcgaatagtgctgtgatgaatatgtgagtgcctgtgtctttttggtagaataatctattttcctttggatagataaccagtaatgcgattgctaagtcgaatgatagttcaacctttagctctttgagaaatctctgaactatcctctgtagcagctgaactaatttacattcccaccaacagtgtataagtgttcccttttctgagagaataatttaatcagaacagtaccttaagatgcagctttaatacagttcaagtaccttagaaagtagagttgaaaaagggagtttctgattagtttcttcctgttttcaagcttgttgccttatgtattgcaccctcttgtggccaaggcctgcatcacaaacaaagttaacatggtcactgcaagctacatcacaccagcactaaaagaattggaaaagaggtaagttttaatcattttaaacaaactgagtggatccatggcattgtttttattttatatccaagttttcctttttcctctttccacctgctgcagtgtggaagatgctggcatcacaatcattggtgaattgggattggaccctggtctggatcacatgttagcaatggaaacaatagataaagccaaggaagtgggagccacggtgagcccaatttgtacccaaaggcaacattaaaatgtctaaattcttgtgtacagaatcaaagtatgagttataatatagcaacacctttaatcaaagttaaagattttttttttttgagactgagttttgctcttgttgcccaggctggagtgcaacggcacaatctcggctcactgcaacctccatctcccaggttcaagtaatactcctgcttcagcctcccaagtagctgggattacaggcacgcaccaccatgcccggctaattttgtatttttagtagagatggggtttctccatgttggtcaggccagtctcaaactcctgacctcaggtcatctgcccccctcagcctcccagaaattttaaataatatcttataactgcaaaaatatccatacaggaaaacaatgtccaagtcttaggtagcaaccaatttttggagaaacagatgctactataaaattttggcttaatacaaattttagactaccttgaagattctgtaataagatttgacttttaaaagtaagaagaatctttgggtatcagcaatctgttgatatgaaatatcttttatgaaatagtacctttggcgaggtactattgggtgtaaatgggatgattcagattgttttaggttttttgaatgtgtgtgtatatatatgtattaaaattatatatatgtaaatatataatttgaatacaaaagttagtaggaactgactaagcagaattgaactaagagttggatttttaactgaacaaaatgttagtcactatttttacagtttttttaaaaaaacatatatataaaattaactgttatttaatcaaatggtacatgctttgaagatgtatttttgaatgccttagtaactttaagaatgaaaaactgtagcaagtttactccattcatatcattgtcttcaattaattacttagacattctctaagttgtgaattcttccttatttattgattctttaatttatagattgaatcatatatttcctactgtggtgggcttccagcccctgaacattcaaacaatccattgagatataaatttagctggagtccagtgggagttttgatgaatgtaatgcagtctgccacctatctgctcgatggaaaggtgaggaatcttatccaagcatagtgatatcaacctgcaataacagcctgagacagtctaaatactccctgatcccaaaccctttaataatggtgatgctgcctgtctccattatctcctcctgtgttgtttcccagtgtatattttctgttctatttagcacggtatccttgctgttcctagcaaagcctgtgcctattctttgccctaggccttcttgcaagttgtttctgctattcaaaaattcacttctctatatatgtaaacactggttatctcttgatttatttcagcatgacaaacattttctgagtacctactatgtgctggactttgggcaagttacccaagactgtcacagagaatgaaattcatgacctcagaaagctcacagctggactggggatggggctcttggaatggagggtttggtgtgctggggaaagaaactataatttcttccccagctgccatgtaacagagcataagaactctccaagtttaatattattttcattattttacaataagaaagctatggctcagagagcatgtccaaggtcatttggctgagattcaaacccaggtctaactaactcttaaggctttttttctccttttgccagattgcctaatggctggaaagacagacatgggaataagtaattataatttaatagggaaatttccaaagtatgttaaagtggctcagagctacagatgggagaacagtttaattggccttggtgaaggggttgggggaaggaggatggggaaaagaaaaactagtggagaagaggatgtgcttggccttgaaggatatgagggtgccagctaggcagggatgttagcactcatggtatgacagttttataaggggaaaaatcaggtttagggagttaagataacagctacacttaaagagatggcaagtgacagagccagtatccaaatcatgtctgtctgacttcatagtttatgctcatagtttggagaaaaatgaaacgagactcagagtgcctctttgaattgtgtgttgtaggcacctcattcaccttgccctagtcctggccctgcagggagtgatgagtaatatgatcacatagatgtgtcttaaaaagattattttagcatcaatattttacattgtcaaggtatttgtgaatcgaaatgtatttcaggagtagagagaaagaatttcttaagcaacaaggtgaatgctgttattagccatggtcttttgtgcctgtgatatagagcataactcatacttggcttggaattacaaggggagtttaaaaagtaccagcgcctcatccccaaagattctgatttgcctgggtttgaggattttttaaatcttcctagtgattctaatatgcagctgaatttgagaacaactgatttaatgatcagagaatcacagctgcacttaaaatagtactaaacaagcaggacactttagcttgatgccggaatgatgtgtgacatgtttcaggataataaacaggatcaatgagttctggtctaattcctggcatccctcataacatagccaaagaccaggcagttgaaaagatctaccggatgtcagtttcctgatgtgtaatgtgagcactgatgttccatccaattctttaattctatgaatcatgaaggtttggaccagaatcttagtctgatgttttaccaagcacacatccttttacatgctacagcctatctcctattgcattccaacagctccttttaccttctgcaatcttaagagactggcctatgtttggtataaattaacatgtgaaggcaggacaaattcttgcctcagttgagtaaaacacttatttttaggaggccttataaatgctaaagatggtaacaacgaaagggtcagcagagtgcaaccatgtaaaagaaagagcaaggaacttggtatctgtgggcctggctttgtgctctagctctgccattactggttgtgagctttgggttccttgtctgaagataaagacaatcatgccagccttccagtgttcttagtcagtgaggtcacatagattgcaatctgcctaacagcagattgtcagccatgtaataaatctagcaaatattagctacataaggaaattagtggttgtgagagtttaagacaaacaagaggttattttcaggtagcagaagttgtatttgttgaattcgaagttctcctctttcatcctaaatgtcttttgtaaaaatgagtgctttttgcatctctctgacactggaggataccaagagaaaatatgacacttttctttgctaaaatgatacttttttcaaggtcccacctgcgtttcctcttgtggatgaagctttttgtgaccactccagcctctgttcactattattttgcctgaactgaatcaaagaatcagacactttgtttgtctgtgtcttaagaattttctaggtgctgcattattcagtcagtaattcaagtgtatcagtctgtttctcaagccagattgttgaacaacttaaaacctctcttgtattcttgcccttcactccctctgccatcaccaccctccactgtggagcgggtcttaagtaattcacagaatcataatgggaaaccaagtcacctctctgacttctgcttgaatttgaagacttaaacttttccagaaattggtcagtggcctatgaatttgaatgtgtctcattgtcttacatctttgtagtaatttctagccacttgtactatttctaacttctaatttgattcctaacaattctgattttcttccacatattcggagacagtgatttcatcttctcttagtttcattcccctcacagtggtggtgtacttttttacattgttggcatttgggagtgatatggcagagctttgccatcatcttctgcagtgtttcccagtctaacctggcaaaacatgtagtctaagtgagaagtgtgcttgctctgcaaacatcgctttaagataagttttctagccctctatccattgcctgccaccaccttgactgcagcacccaaccctttggaaatcaagagctaccactatcttcctttgcctgtaccaattctttatgcaactttcagtgcatcccaggtggcataatccagaatgctgtactttgtcaatatccctttcaaaatactgatccagaaataagcatgattaaggggacagtgtagttgaaggaacacagacattgcagtcaaatagaactgggttcagatttgcctttgggtgactcagcctcagttttcacctctgaaaaatgagttgtttcatggattaaagataatgcccagtacaaggcactgggcacatagtaggccctgaaaaaatgattgtatatttccttattagtttagtgatcaagcttactgtgattatttgaatatatatgatatatttattcaaatataacagtgattctctctacttgggagatttgtatttgtgtttgtgtgtatgtgtgaaagagttggacctagagaagcttgcaacagcagtgaataatgagcctgagcattgtcagagcagaaagaaatgtatattctctgcaaaacagacaaatatgtttctgttgtcattcaaattattaataaaataataaacaagaaagaacttattgttgaactacattatataccactagaaatacaactacgaatgaatatcaaatgacaatgattacccagtaatttttgggtcaggttgcttagccacctgccattctcactaaatgaatcattgccattcagctgacatttctccatcttctttgtaaggatttcatgatagacttatgaacttgctgctttatttatgattggtaagctagaatattgtggtcattattgaatttttctttcagaaattttcctgactttcaaacaagcaaacaaaacttaagaattgtcatgggttatagaaatgagatgggtgtgaagaccctaaaagtcaacgtttgcttttttgtttttaagagatggtgtcttgctgtgtcgctcaggctattctcaaactcctgggctcaggccatcctcctgcctcagcctcccgagtgaagtagctgggattacaggtgggcaccatcatgcccaacatttgctttcttagattttttcaaaaatttagaattctattaaataaactggttttaaagaagtagttcagaaatcatcccccctttggaaatgtttactctgcagtctagtagggttcacaagaaaattaaatggaaggcattcctcacatccttcattcagattttcacttttgaaattttaaatggtgcttggcactctccaaagtgagtctctgaactgaaaagactccagtggtcctaacattgcagtcagcatagcccttttctttctggggcttcatccacagataaatgagtttccattttctctctcatacaaaggcacgttcagaacccatctactaataaggcatgagaatcattaagtttagccaaaggcctcttcaggcaaatcctgtgaatccaccttaaagtacattggcagctccctggtacagtgttattttgtgcagctttagagaaactggtcagaaattgtttccagtggtacaataaggataaaatttagttccaccagcagcaacaaccagaattctcagacctaattttatcactccagaatgctttcctttatctcaacaataataataagccaataaactagaagaaaatttagtcttttaattaaaattagagacaaagaaatccttcattgcattttgagtatgtgtgatgggggtaggggtatgggaaagagaagtactgagatatcaaaagatttttatttcatcactcatgttattcccttcacaatttgtgcgatcttcaatgcatagatagtaactgctcaataaatttagctgtatgaatgaaatgtatgtgactgatgcctaatgtggcatttagaaggctcatgaaagtaaaagaaaagaatgagaatattgattgttgtttgagcatagaagccatttaaataattatctgttgcttttagcttatttaagtttattttttaaacatttttatataaaataattttgtttattttttaaacattttttatttttaaatatttagaaaataaattttttaaaaattaaaatgtttattttaaattttttttctaaatgtttatattttttaaaaaggcaaactctagtgggaaataatttagtgccattttatccaggggaaggaaaaaaattgaaaataagaccttatgtttagaaaaactaaatgttttaaagtcaaattttaaagcttgagagaagaattgctttgtgaattatgatattagtagaacagcttaagtataggatgatgattatggtggtttcattatttttattttttttttcaaatttctgttgctttctttgttcgtataatgcagataagccctaaaagaaaggaacagctgtaaccccatgtgtgccttctcaatgttcctcaggttgtgaatgttgcaggaggcatctcctttcttgatgccgttacgtccatggatttttttccaggattaaatttggaaggctatcctaacagagacagtacgaaatatgctgagatttatggcatttcttctgctcacactttgttgcgggggacactgagatataaggtaagcagctggactttagattcaacatgttgccagttgtgatgtaagtgatgtgtacacttaatttttcttagtagtcaaaataattaagatgataaaccaagttggagtctattttttcaggaatcttatgattgattgaatattttaggagactcacctcaaaataaaaagataattaattgactcattaggagttggggacaggcagggcttcaggcttagtgagagttggagatcaagtgacttccccaggacctgacttctcgtaacgtctcagctcttctttccagtattcacaggagtttcaagtgctgtgcgatggcagagtggtggcagtgctcccagcctctcaggatcaaattcagtgggaataaaaaatgataccgttctaatagccaaaaatacgatttttgtctcattagctccctaaggtcagtgggataaaatgtgcccatctactttggcctatgttatatgtcctagaactggagatggagtcaatgtcattaaaaaaacatggactggggcgggaggaaacggacgggatgaaaggggcttccccagagcaaatccaaaatgtttttaaaccacaaaatggggagtacattctaggagcattaatgcaaatgtccactacacatttattgatcacaaaaaaagcagttacaagtgtgacctaatcaagacaactggtgtaatagttttctagcataatatcacaaattgggtagcttgaaacaataggggtttattctctcaccattcaggaggccagaagtctgaaatcaagatgtgtgtgagtagggttggtgccttttggaggcttgactttgagacacatccatccaatttgtgcatcagtttcatatcaccttcttctgtgtgtctcactgtgttgcatcctcttcttattagggcactagtcattggacttagggttcaccctaaatccaggttgatttcatattgaggtccttaattaattctatctgcaaacattcaagtgaggccatattctgaggctgtggatgcccatgaatttggcgacagggccctaaactcaacctaatgtaaccagcatacctaaaatacatgtatttctcaggatcagagacctgactgcaaatctgtctgtcttgtgagatttcctggctaaggccagagaggggatttgtctcatgtgtgttatctagtattctctaattatctttttatctggaagatgtaaggcacatggacctgtcagtgatgacccttcaatcttgtgcctgcttttctgtcgttctcttatctttttttgctggctgttccttttgcctggaacttttcctccaggtatttgtatcatcctccttcgggctttattcttttcttctctgcagccctgcccctggtaaccatggttttattctctatctctgcatcttttactttcgtaaaaaaaaattctgcatgtaagtgagatcatgcaatattttactttttgtgtctcacttattccacttagcataatgtcttccaggttcacctgcctatctgttcttaaaaagaaaacaaccactatatttcttactcttgagagcttctgttacatttatgggcacttagcatttacttctcagcattttaagttataattttatgtatatttttcttatctcccaaaagagattgtaagctcctagagagcaggtgcttgtactccatgaatatgattttttcattgcccaggaatcaaatgagacatcagtgataatgtcctaccgagatctaggagctatggctcttctaccagcctttaggcacagtagaaattctagtctgaacttatgcatactatccccagctctggcagatttcttgatctattttcttcaatttgttcaaagaatatgagtaaaaacttttttttttggccagcagttaggatatatagctttcagtatagtcttttgacaaacatatagatccaggaatgaaggattttaatagatttaaacattgaataaaggtctcaacaaggtgtgcgagtgggagccatccccatggattccttgaggatggaagatatcacagtgttcgtaatcatgagggccaccatcctgggcaatgccagcaatccattggtaaccctaaaattctttccagtattggagatgagataccatcattcaaatatgacagagaggccttgaccactctccttctctgcctgcctgtcacctgcttgtccttctagacgcaggttaatagcaccttcctgtggtatcctccctaactccttccccttgaaccacccagccccatctatgtgagttccccttcttttgtgctcccacagctatgttttcctctaacctagcacctattaatataatactgtactacaatttagtcatcagtggtgatgcatttttgcttaccatggactaggcattatttgcttgacttatctgacccttctgttgactgcagtttctgcatggatgagactgcaattttttcatctctgtggcctcagtccctagcacaagtctgacatatggcagcagtttgctaaatgcttattgatgaatgaatacataaaataaattcatttctaggggtcagcataaaaatatatttctgagattaataaatagctcagaaatcagtattttagatgttggataaagggatctctagcacctatcaggtcccagaagcgagctaaacaagctagtgatattacttttctgtttctcctctttgaagtatatttgcatttgaattagagcaaatgagaattctttttttagtaacctttgaacagtttgaacaatataacctttagaaatgggaaaattgctagttggccagacattcagtagtgaaatatctgacgttacaggaactattttatattttaaaacctctgttgctccctagaattatttccctctctctcttttcttctctctctctctgcttgagcagtaattgcagataagataaatttcttatcttagccttctcaccctgcttcccatttgcacactggggaataaaccattatggtctttcttcacttcatctattattcagaatctatttatccagccatttgttctcttttagggattggaatgctgaataatggattggaatgttgaataatgttgaataccttcctatggtatcctccctaactccttccccttgaaccacccagccccatctatgtgagttccccttcttttgtgctcccacagctatgttttcctctaacctagcacctattaatataatactctactacagtttagtcgtcagtggtgacgcatttttgttcaccatggactaggcattatttgtttgacttatctgccccgaagtgggcattgggatgaagaatatgcacacaaataaaatataatataacataatataaagatacaatttccctgctttcaataccttcagtacagcagctgaatagacaattaattatgagctgcattaattgccatgataaaggtatatacaaaatgctacaagaagggagtgcccacttgtgagtgctaggggactcacaagaggtgctaaactgggtctgaagggaggagaaaacatttcataaatgaagaagtgtggccacaacattccagttacaggagcagcatgtacagaggccctggtttctggaagaacacagcacatttggggaatagtgaaagtggagatctgtgagttaggctagatgagcatgttgtgatgggttttatataatataccaaggacctcaggtttgctttgtagacaatgaaggacatatgaggaatttttatcagagtgatgatatcatcatatcttagaaaaatctctgattcctacctctttttctgctctccatatatcttgggggtggtggctctaattagtagattggattatctgcttttgaggaggatattcttacaagttcctccttccattataagaaagagctcaactgactgttctgttaaataatactctgctgagctgacagcatataacacataagaaaatttgcccttatgctgcccgaaaattccattcacatgttacccagaaacagaagtaaagacctaagttttgcaaggtcttttatgtaaaccagtagaattgttatgacactattgatctcagtttgagcttgaaattggttgtatctatatcctcaatacttttttggtgggttgtggggaggggtttctagtaaagacaggaaaacctgctaggcaaattctagaagagctgtagtactcatatcatcaatataaacagttttctaaattagttacaaagatatatccctcttttcaaaaatattttaagttttttcattggaagagaaaaattttatatactgcttattaattcttgtttcagggatatatgaaagctttgaatggatttgtaaaattaggtcttataaacagagaagcgcttcctgcctttagacctgaggccaaccctctcacctgggtgagtgactccgaattacgttctatctgctccttttcagaaataccttctgaaatggtcgggggtgaactctgctttggatctgcaaaagatcctattttttagcttatgagcaggaagataaatcctaactccaaagccccctttcccttcactgttgtggaagaatggagagggtgtggggctgacacatatcaggcctgtttcccgcagcaacttataccatttgactgctccttgagctgtgataagagatgcctttgctctctgcattaagctctgagctattccagttgtctaggtggtgacccagtgatgggagtcacttggacttcctgaaagtgagaatccaggagagttactagtgtattacagaactggatttccaacctccaaatcttgtcagtactctcatttggttacttataaccatgagtgatttgattttcagttaccgagcttatgcgtctatctcttgttgtaccaaaaccttaaggcctcagagttggaatgaactttggaggcttggaactgaatgcgaagtagagcttctactgactgctcagaattaactcattaccagctggttgttcaacttaaactagtgtgcctgtattccagtagaaatgccagaaatgtatcacaaggagaaaaggaccaatgttgctaattccattacatctcaaaaaaaagttttggtattcttgctagctagtttctttaaaaaattatttttgaattctttatgggtacatagtaggtgtatatatttatggaggacatgatatattttgaaataggcatgcaatgtataataaccccatctgggtaaatggggtatctaccacctcaagcattcatcatttctttgtgttagccagttgcttttacattcattttttttgtgtgcatttgtgaacctttccaatcttaagttttacctttcataaacttacttgttgggtaggagtgaacaagtatctgcagagagaacctggggttgaggcagctggtttgggaaggttggtgcagacattcttagggatggtcttttttgtaaaggaccagattataaaggttttagactttatgggccacacggtctctgtcagtaccactcagttctcccatagacgatacataaaccaatgagcatgactgtgtttcaataaaacttttatttaataaaacagacaatgagccaaatttggtctgtgaaccagtttgttgactcctgatctagagcatagcttttcctttgggaacctagaaaaagtcttgttttgaaagtagaaaaaactatggctactgtttaagtattcttcctttgagattttattacctgcaaggactttctcatctcttgaacctacaaaagaacctgggttgctactggcaaccactgcagtaagccttttccactttatcctggtcaaaatggactgctggagggtagggggagaatggataaaaacatagtgatagagaggatatacaaagacagggaatgtgctggtaagaactgcaccatctgttttgtgttgagttgatcttaaaacccatttttattggtgaaatatcgtttggaatagcagctatttattgatgtgtaggtatgcagatgtgtgtatgtgtgtgtatgtgttacaatttcctatttccaattatttttaattttaaaatttgaggtgtatttgaagttcagtgttttagtaggataatttttcagtcaattaaaaaaaaatttgttagggacttagaaatattgtatttctttttctaaccagaaacaactcctctgtgacctagttgggatttcaccctcctctgagcatgatgtgttgaaggaagctgttcttaagaaactaggaggagacaatacccagttggaggctgctgaatggtaggcacccaccactcaacttagagcaaaatatactggatcaatgattgctaatttctactcagaaaaagttaaatattttacatttgtctttgattaattcgttgctctaatgtgggtagagagattaccatgtgccatgttcatgtgggcataaagagtagataaagagaggagctcaatggcaattagaatttgagaaatgcttatctcgaaacactttaccactcagttcccaagcatagtggggtatttttgctttcctgtaggagatttggagtagtcatagaatattacatgaaaacattctttcatatgaccgctgtggcatcctgctggtatacctaatgttggagaactaacctataaattaatctcataaagatctttgtaagaacatgggattacaatccatcatctctgccctgctccagggagcagtgcctgggtgagtctggagaagattctgaaggttaaaacattgctattgttattgttattttacattttaggagaatcctacaacttgagttaccaccttgtaattctgccctttcaagattaaattcaaggagctcccttttgcttcccaaatgagaacatgtataggacttgaactaatgcagccatctcctgtgcttttcaggttgggcttacttggggatgaacaagttcctcaggcagagtccattctggatgccctctccaagcatttggtcatgaagctttcctatggtaggccatgaagtattagctaaatatacctaaagaagaatcataagatagagtcagtgcagataatcgtattggagggttggccccaagcagcaggaatgggaagatgtgatatgaagaaaagggcaattggactatttttccagagggaggaatttgagaattattaggtaaaaataaacttagttattatgggaaaggaaaaaatagttttactatttatttatttagttagttagttagttattttttgagacggtgtctcacgctatcaccaggctggagtgcagtggctccatctcggctcactgtacctctgcctcccaggttcaagagattctcctgcctcagcctcccgagtagctgggactacagttgcctgccaccatgcccagctaatttttgtatttttagtagagacaaggtttcaccatgttggccaggatggtcttcatctcctgacctcgtgatccgcccaccttagcctcccaaagtgctgggattacaggcatgagccaccgcgcccgacattactttttttttttaatgcattaggcaaatgccaaagaagcatttttggagcacactaaaaggcctctatagcagcgcttttgtgtctgtaaacaggatatggtgacatgaatccggataaaggaatgagatcagagttctaatttccttgaggttttcactttgactttgggctaaaaagctgtgtaaataattgtctagtgatcatatataaaaggagggaaaacttttaagaccagaagggcaggaggagatgacagacaatatttgagttgtaatgatagtggtatttttacttatctttatttgttttaaggtcctgaagaaaaagatatgattgtgatgagagacagctttggaatcagacatccttctggacatttagaacataaaacgattgatcttgtggcttatggggacatcaatggcttttcagccatggctaaaaccgtggggttacccaccgccatggcagccaaaatgttgcttgatggtaagtctgttcatttgaggtgtaagcctgtttctgtttcacctccaagccatttggtgcctggacatcaagtaattgctcttgagcgtaacacaggcatttactaaaagtgaaccatcttagaagatccaagcgcggactgcctgggaggtccccttttgatgagtaaagcatgtgggctttggagatagacctgacttccgcttccgctcagttccaaggtggctgagtgatgggctgcaagttactcagcacctttgagcaagagtttcctcatatttaaagtgggtattgccatccctgtttcagagttgttgtgacactaactgatataatacatgaagctgctacctcatagaggggtccccagggaatggtaacttccttcctttctttctttggtttttgccatgttgggagtgataactctaaaatagaaaaagatattcactccagattctcaagaatataggttaagttttattagtcttcatatctcagcatattattagtactcaataaatattttagactaaatgaataaaaatttcattcctccactctccctagaagatcacatagacattcaataagcattgcctctgtttgaccctcaagaaagaatgaggggagtagaaaatatcaaagcatataaatgtgaaataatgaaaaaaaaatgtatagtagcatatcagctaatgtcaaattaaatgaccaacttagttattgaggaatcaatgaagaagtaagtgataaaagttggggtcattgtggcctctagtgagagtgcccaggttctgtgtttcagtcatactaacaggtgcctgtgcccatataatcaaacctgttacaactcattctgttggtgtcttgccttttttaaaaaaaaatgacattagcaagttaagctgaccagtcagatttactttacttcttactttactttgttctctgattgtcctggcagaaattaatgcagttaagtgtacaagttctgacaatatccctaggtccggctgtgctttcaatctaaaatcaactccttttccttacaaagtgttgaataagtaataatttactaagggacgtttttaagcagctacttaaaaaatgcctaggcctccaagaattcattgactctgagtttgcttctttgtggagactcttgatggggaaattaacctctgacacaacctttgaaatggtaattacacatttattttcttccaggtgaaattggagccaaaggcctaatggggcccttttcaaaggagatctatggaccaatattggagcgaattaaagcagaaggcattatatatactacacagagtacaattaaaccataattgggaattatattttgtttttttcttcccaggcaatacacctctgaacatgtgtgtgataaatgggtttgctaatgtgctgttttaaagtataaagcataatatgttttggttaacacaatgtactttttgaactataaatctttattttaatatggaaatgtttggaacaggagatgcaagccactaacagagaactttaataattctaccctgtattttataaatacgtatgtgaaagtgatgatcatgctatttgttaatttattgtgccactttttttcttttgttttttttttgttgttgttgttttttgagatggagtctcgctctgtcacccaggctggagtgcagtggcgcgatctcggctcactgcaagctccgcctcctgggttcacgccattctcctgcctcagcctcccaagtagctgggactacaggcacccgccaccacgcccggctaaattttttgtatttttcagtagagacggggtttcaccatgttagccaggatgtccacttttttcttaaatgaacatattgtcttataaccagcaaatgaagttttacttttttagtcagaaaaaatttcgtcataaagctatatttttatatttttaaaattgatacagttagataccttcagattacctaagttaagtttttgccaaaatgctattttatgccctttttgtaaagttatgatttattttgtaaatatagttccttatagttttctcttgagtgtttggacatgagaatagataagattaaattacaatttacaagatcacttcattaggcacaggaaaagtatttaacaaaattcaacaccctttcacaataaaaaataaaaaataaaaaaccctcaacaaactaggaatagaaggaaacttcctcaacctgataaagggcatctatgaagaacccatggttagcatcatacttaatggtgaaggactggattatttccccctgagatcagaaacaagacaaggatgtctgctttggccatttctattcaacttgtactggacagtcaagaaagggcaattaggcaagaaaataaaataaaagtcatctaggttggaaaggaagaagtataactaaatagagatgggagacgatatggtggttttgtgcatagtaatccaagggaaaactatagaaaatccaaaggcatgatttattaataaataaattcagcaaagttacaggatacaggattaaatatgcagaaattggttatctttcaatatagtatcaatgaataaatcaaaaatgatgttaagaaaacaatttaattttcaacaatggtcaagcttggtggcttgtgcctgtaatcccaacactttgggagtctgaggcagaaagactgctagaggccaggagaccagcctgggaaacagtgagatcccatctctacaaaaacaaacaaacaaaaaacaacaagcaaacaaaaaacagctggatgtgatgacactcatctgtcgtctcagctactcagaggctgaggtgtaaggatggcttgagccccaaagttcgaagctgcaatgagctataactgggtcactgcaatccagcctggatgacagagttagaccttgactcaaaaaaaacaaaaaagaaaagaaaaaaattgacagcatcagaaagagtaaaataccaatataattgagggtccagaaatattgatcacatttatcatcaactgacttttgaaaatagtgctaagacaattcaatgggggcaaaatagtctttttcattaaatgatgcttggaaacatggatatccacatgcaaaataatgaaattggactcctacctcatattgcatataaaaattaactcaaagttgacctaagatctaaatgtaagagctaatattgtaagtatcttcaaagaaaacatggcgtaaatctttctgactttgaattaggcaatgatttcttaaataaatgacactaaaaacacaagcaacaaaagaagaaaataggtaaattaaaattaaaaccatttgtgtttccaaggacaatatcaagaaagcaaaaatacaacccacagaatgagataattttttttaaaatcatgtattagttaagggaggaatttgtatttagaatatgtaaagaactgttacaataataattaaaaggcatataattcaatttttaaaaatcaacaagagatctgaattaacagttctccaaagaagatacacaaaaggccaataagcacatgaaaaggttctcaacatcactagccatcagggaaatgcaaatgagatctacaatgagatatcacttaacactcactggaatagatatataatcaaagtgataatatcaagtgttggtgaggatgcagagaaattggaaccctcatgcactgttggtggatacccacgtgatataaaaacaaatgtctacacaaaaacttgtgcacagatgttctttgcagcattatttataatagcccaaaattgaaacagccctaatttctatcaactaacaagtgaataaacaaaacgtggcatatccatacaaggaatattattcagcaataaaaaaagagatgaagtactgatatgcaccacaacatggaaaaaccttgaaaacatatcaagtgaagaatgccagacatgaaaggccacatattatatgatttcatttatatgaaatgtccagaacaggcgaatatatatagagagaaggcagattagtggttgcttaggctgaagggttggggaggacaaaggggtgactgctaattagtatggggtttcttttttggggtgatgtaaacgttgtaaagttgattgtggtaatggtggcacaactctgaatatactaaaaaccactgaattgtatactttgaatgtttgaactgtgtggtatgtagattttatctcaagttgttataaaaaattaaattacaaa

SEQ ID NO: 36 - DNA, 5825 bp, Homo sapiens
  source 1..5825: mol_type = other DNA
agtttcttctaggaattcggagcctcccctgcagcgactcggaagattcgaggcggcgggggacaagtcggcgccccagagcggacgagtcaccaggtgtcaagatgctgcaagtacataggactggactgggcaggctgggggtcagcctctccaagggtcttcaccacaaagctgtgttggccgtccggagggaggatgtgaacgcctgggagagaagggccccgctagctcccaagcacatcaaaggcatcaccaatctgggatacaaggtcttgatacagccttcgaatcggcgggccattcatgataaggactatgtcaaagctggtggcattcttcaggaggatatttctgaagcttgtctaattttaggagttaaaagacctccagaggaaaaattaatgtccaggaagacttatgcatttttctcccacacaataaaagctcaggaggccaatatgggcttgttggatgagattctaaaacaggaaattcgccttattgattatgagaaaatggtggatcatagaggagtacgggtagtggcatttggacagtgggctggtgtggcaggaatgatcaacattttacatggaatgggtttaaggctccttgctttgggacatcacacaccttttatgcacattggcatggctcataactacaggaatagcagtcaggctgtgcaagctgtccgtgatgctggctatgaaatatctttgggtttgatgcctaagtcaataggacccttaacatttgtgttcacaggaactggtaatgtttctaagggagcccaagcaatctttaatgagctaccttgtgaatatgtggagccccatgaattaaaagaagtttcccaaactggagacctcagaaaagtgtatgggacggtgttaagtcgtcatcatcatcttgtcaggaaaacagatgctgtgtatgatcctgcagagtatgacaaacatccggagcgctacataagtcgttttaatactgatattgcaccctatacaacttgcttaattaatggaatctactgggaacaaaacactcctcgcctcctaacccgccaagatgctcagagtctcctggctccgggcaagttctcacctgctggtgtggaaggctgccctgcattaccacacaaactcgtggcaatatgtgacatttcagctgacacaggagggtctatagagtttatgactgagtgtacaacaatagagcatcccttttgcatgtatgatgcagaccagcatattattcatgacagtgttgaaggctcggggatcctgatgtgttccattgacaatttgccggcacagctcccaattgaagctacagaatgctttggagacatgctttacccttatgttgaagaaatgatattatcagacgcgacacagcctcttgaaagtcagaatttttctcctgtggtgagagatgcagtgattacatccaacggtacattacctgataaatataaatatatccagacactccgggagagcagggaacgtgctcagtcactttcaatgggcaccaggagaaaggttttggttcttggatctggctacatatctgagcctgtattagaatatttatcaagagatggcaatatagaaataacagtaggatctgacatgaagaatcaaattgaacagttaggcaagaaatataatattaatcctgttagcatggacatttgtaaacaagaagagaagctgggcttcttggtggcaaaacaggatcttgtcatcagcttgttgccttatgtattgcaccctcttgtggccaaggcctgcatcacaaacaaagttaacatggtcactgcaagctacatcacaccagcactaaaagaattggaaaagagtgtggaagatgctggcatcacaatcattggtgaattgggattggaccctggtctggatcacatgttagcaatggaaacaatagataaagccaaggaagtgggagccacgattgaatcatatatttcctactgtggtgggcttccagcccctgaacattcaaacaatccattgagatataaatttagctggagtccagtgggagttttgatgaatgtaatgcagtctgccacctatctgctcgatggaaaggttgtgaatgttgcaggaggcatctcctttcttgatgccgttacgtccatggatttttttccaggattaaatttggaaggctatcctaacagagacagtacgaaatatgctgagatttatggcatttcttctgctcacactttgttgcgggggacactgagatataagggatatatgaaagctttgaatggatttgtaaaattaggtcttataaacagagaagcgcttcctgcctttagacctgaggccaaccctctcacctggaaacaactcctctgtgacctagttgggatttcaccctcctctgagcatgatgtgttgaaggaagctgttcttaagaaactaggaggagacaatacccagttggaggctgctgaatggttgggcttacttggggatgaacaagttcctcaggcagagtccattctggatgccctctccaagcatttggtcatgaagctttcctatggtcctgaagaaaaagatatgattgtgatgagagacagctttggaatcagacatccttctggacatttagaacataaaacgattgatcttgtggcttatggggacatcaatggcttttcagccatggctaaaaccgtggggttacccaccgccatggcagccaaaatgttgcttgatggtgaaattggagccaaaggcctaatggggcccttttcaaaggagatctatggaccaatattggagcgaattaaagcagaaggcattatatatactacacagagtacaattaaaccataattgggaattatattttgtttttttcttcccaggcaatacacctctgaacatgtgtgtgataaatgggtttgctaatgtgctgttttaaagtataaagcataatatgttttggttaacacaatgtactttttgaactataaatctttattttaatatggaaatgtttggaacaggagatgcaagccactaacagagaactttaataattctaccctgtattttataaatacgtatgtgaaagtgatgatcatgctatttgttaatttattgtgccactttttttcttttgttttttttttgttgttgttgttttttgagatggagtctcgctctgtcacccaggctggagtgcagtggcgcgatctcggctcactgcaagctccgcctcctgggttcacgccattctcctgcctcagcctcccaagtagctgggactacaggcacccgccaccacgcccggctaaattttttgtatttttcagtagagacggggtttcaccatgttagccaggatgtccacttttttcttaaatgaacatattgtcttataaccagcaaatgaagttttacttttttagtcagaaaaaatttcgtcataaagctatatttttatatttttaaaattgatacagttagataccttcagattacctaagttaagtttttgccaaaatgctattttatgccctttttgtaaagttatgatttattttgtaaatatagttccttatagttttctcttgagtgtttggacatgagaatagataagattaaattacaatttacaagatcacttcattaggcacaggaaaagtatttaacaaaattcaacaccctttcacaataaaaaataaaaaataaaaaaccctcaacaaactaggaatagaaggaaacttcctcaacctgataaagggcatctatgaagaacccatggttagcatcatacttaatggtgaaggactggattatttccccctgagatcagaaacaagacaaggatgtctgctttggccatttctattcaacttgtactggacagtcaagaaagggcaattaggcaagaaaataaaataaaagtcatctaggttggaaaggaagaagtataactaaatagagatgggagacgatatggtggttttgtgcatagtaatccaagggaaaactatagaaaatccaaaggcatgatttattaataaataaattcagcaaagttacaggatacaggattaaatatgcagaaattggttatctttcaatatagtatcaatgaataaatcaaaaatgatgttaagaaaacaatttaattttcaacaatggtcaagcttggtggcttgtgcctgtaatcccaacactttgggagtctgaggcagaaagactgctagaggccaggagaccagcctgggaaacagtgagatcccatctctacaaaaacaaacaaacaaaaaacaacaagcaaacaaaaaacagctggatgtgatgacactcatctgtcgtctcagctactcagaggctgaggtgtaaggatggcttgagccccaaagttcgaagctgcaatgagctataactgggtcactgcaatccagcctggatgacagagttagaccttgactcaaaaaaaacaaaaaagaaaagaaaaaaattgacagcatcagaaagagtaaaataccaatataattgagggtccagaaatattgatcacatttatcatcaactgacttttgaaaatagtgctaagacaattcaatgggggcaaaatagtctttttcattaaatgatgcttggaaacatggatatccacatgcaaaataatgaaattggactcctacctcatattgcatataaaaattaactcaaagttgacctaagatctaaatgtaagagctaatattgtaagtatcttcaaagaaaacatggcgtaaatctttctgactttgaattaggcaatgatttcttaaataaatgacactaaaaacacaagcaacaaaagaagaaaataggtaaattaaaattaaaaccatttgtgtttccaaggacaatatcaagaaagcaaaaatacaacccacagaatgagataattttttttaaaatcatgtattagttaagggaggaatttgtatttagaatatgtaaagaactgttacaataataattaaaaggcatataattcaatttttaaaaatcaacaagagatctgaattaacagttctccaaagaagatacacaaaaggccaataagcacatgaaaaggttctcaacatcactagccatcagggaaatgcaaatgagatctacaatgagatatcacttaacactcactggaatagatatataatcaaagtgataatatcaagtgttggtgaggatgcagagaaattggaaccctcatgcactgttggtggatacccacgtgatataaaaacaaatgtctacacaaaaacttgtgcacagatgttctttgcagcattatttataatagcccaaaattgaaacagccctaatttctatcaactaacaagtgaataaacaaaacgtggcatatccatacaaggaatattattcagcaataaaaaaagagatgaagtactgatatgcaccacaacatggaaaaaccttgaaaacatatcaagtgaagaatgccagacatgaaaggccacatattatatgatttcatttatatgaaatgtccagaacaggcgaatatatatagagagaaggcagattagtggttgcttaggctgaagggttggggaggacaaaggggtgactgctaattagtatggggtttcttttttggggtgatgtaaacgttgtaaagttgattgtggtaatggtggcacaactctgaatatactaaaaaccactgaattgtatactttgaatgtttgaactgtgtggtatgtagattttatctcaagttgttataaaaaattaaattacaaa

SEQ ID NO: 37 - protein, 926 aa, Homo sapiens
MLQVHRTGLGRLGVSLSKGLHHKAVLAVRREDVNAWERRAPLAPKHIKGITNLGYKVLIQPSNRRAIHDKDYVKAGGILQEDISEACLILGVKRPPEEKLMSRKTYAFFSHTIKAQEANMGLLDEILKQEIRLIDYEKMVDHRGVRVVAFGQWAGVAGMINILHGMGLRLLALGHHTPFMHIGMAHNYRNSSQAVQAVRDAGYEISLGLMPKSIGPLTFVFTGTGNVSKGAQAIFNELPCEYVEPHELKEVSQTGDLRKVYGTVLSRHHHLVRKTDAVYDPAEYDKHPERYISRFNTDIAPYTTCLINGIYWEQNTPRLLTRQDAQSLLAPGKFSPAGVEGCPALPHKLVAICDISADTGGSIEFMTECTTIEHPFCMYDADQHIIHDSVEGSGILMCSIDNLPAQLPIEATECFGDMLYPYVEEMILSDATQPLESQNFSPVVRDAVITSNGTLPDKYKYIQTLRESRERAQSLSMGTRRKVLVLGSGYISEPVLEYLSRDGNIEITVGSDMKNQIEQLGKKYNINPVSMDICKQEEKLGFLVAKQDLVISLLPYVLHPLVAKACITNKVNMVTASYITPALKELEKSVEDAGITIIGELGLDPGLDHMLAMETIDKAKEVGATIESYISYCGGLPAPEHSNNPLRYKFSWSPVGVLMNVMQSATYLLDGKVVNVAGGISFLDAVTSMDFFPGLNLEGYPNRDSTKYAEIYGISSAHTLLRGTLRYKGYMKALNGFVKLGLINREALPAFRPEANPLTWKQLLCDLVGISPSSEHDVLKEAVLKKLGGDNTQLEAAEWLGLLGDEQVPQAESILDALSKHLVMKLSYGPEEKDMIVMRDSFGIRHPSGHLEHKTIDLVAYGDINGFSAMAKTVGLPTAMAAKMLLDGEIGAKGLMGPFSKEIYGPILERIKAEGIIYTTQSTIKP

SEQ ID NO: 38 - DNA, 60814 bp, Mus musculus
  source 1..60814: mol_type = other DNA
gattgattgattgattgattgattgattgatttttgcctaattgccctggcctaaccgtgcaatacaggttgagtcattggggtaaatgacaacatacttgtgctctcaggtgaaaaccattcattcagtgttttttccattaagtatgatacaaattttttcataattttcttctacaagggcattcctttccatctggttttgctatacgttttgtggtgattattttgtattatttagtttggtttctatcatggaagggtattaaagattttcaaggactgaatttattgtatcaactccattttccaaaaactcaagagaaatgattttcaaaacaaaaaatttactaaataaatcataactttacaaaaggcatgagaaattacgtttacaattgtttttagatatgtcagtctaaaaggtatgtagcagactcaatttgaaaaatacaaaaatataactatgtgaagttgttttttttttttctgggaaaggaaatagtacaactgcacttgctgataatatgttctcggggaaaaggaagcacaataaattaacaaactatgatcacctttacatacacacagaaagtacacaggagacttattaaagggttctgtgatgtctggattcttctactccaaaagctttactaaagtagagatttgcatttcaaaaataccattgccatgaacttactcaaaagacatcatgcttcatgctttagcacatcagcaagcatgtttgtcatacccaaggttaacaggtgtgatgcttcggaaaatacattcccatttatagcttgattgtactctgtgtgttaaatacaatgccttctgctttaatccgctccaatattggcccatagatctcctttgtaaagggccccattaggcctttggcttcaatttcacctaaaagaaaacaaattttgattcttgatttaaaggtgatgttaaaggcctatttctatggcaagagtcccaccccagggccaaacagtactgtattgaaaagcaatgctgttcaatctagatgtgaccataaaaggagggcagggagaactaaaacctgacagacatccttcaaagaaagaaaacctgacagacatcctcaacttagacccagagaggctaagtaacaaggaggttgacagtaccaccagaatgcaaaatcaactgagcattgctcacagggactcagagagactgaagtagcagtcgaagaacctgcataggtctgtgataggtcttctgcctattcgttatggttatgtagcttggtgctcttgtgagactcctaagagtgagaataggggagttccttgagaccccctttcctcctcctgggttggttcaaccagccttaatatgagagtttgtgtctagtcttatcacaccttgttgttccacgttcaggtgatatccctgggaggcctgctctcttttgaagggaaaaggaagagtggagagaggaagtcaggggtggagcgaggggaaacagcagttgggatgtaatgtatgagagaagaaagaagaagcagagaaaagaaaagggaaaatggttggctttaaagttttcaacgttgctaatggtgtaacacagtaagagtagattatacttagaatctgggctgtgtccaagctagtgatatgcgttatgacgctgtgctatggtgccaggaaatagcagggagtcatagaccccaatcaaccatgaggccaataaagaagaaggtgatactacacaggggcctgtgttgctggactgtgtttcccagtccttcagctctatttaatgcatcttcccttaacacacctttaaacttacactaactattcataataaaaccccactgtaagtcacaaagcatattgcaaatattttttcattatagataagatgttaacacatgagaattaattaaccacagattataattctgatgagctacaagtaaacaccctcggctaattcagaacgactctaaatatacaaataaaaacagaagtaagggagtcacccttcccagcagcccctctgcatggtagcaactttctatgctgtttcgctgaaaggttgcaacaagcctgtgcttggaatgacacaacccacgttcagtttgaggacaccttggcccacaggctttaagctacgtctattccaccattctgaggtcttgtgactgagttggatgaaaatttggtctcataccaggacctatgctcttctctcaccagtgtggcccccagccaggtattctccaagatggctcatttccattatttgcaggcactgacatttgaaggtgaagaggagccaggctcagccctgatgcgaatggaacttaccatcaagcaacattttggctgccatggctgtcggcaaccccacagttttagccattgccgagaagccgttgaagtctccataaaccacgagatcaatggtcttattttctaaatgtccagatggatgtctgatgccaaagctgtccctcatcacaatcatatccttttcttcagggccttaaaataaatacagatgtcttaaagcatcatcatagctcagctataattgcacttcaccttccctgcccttccggtttccccacccaactacatatgctcattagataactatgatgcttttctcccagagacccagtgaaagcctcaagggaatgggaacttggactgtatccctttctcagaatcgaaaagtctgcttggtgtgacaagctattctggcagacagacatttgctccccaaatattgttttggtatttatttgatacattaaatataaaattctttttccccctcccagtagttcccaaattcattatttcctaagaaattttctatcattgcctcttctcacacttctccttcctactgcatgactgctgtgcttgaaactgattctctggccagttcctatgattcctcctcagcagtgtttagagaaaccccacgacctaccataggagagttttgagaccaagtgcttggagaacgcgtccacaatggactccgcctgaggaacttgttcatcccccagtaagcccaacctgaaaagcacaagggtcgctcttagtgcacaacctatgcatgttctgcagtagcttcagggtcctggacttcctgagacactgctccttagcgcagggcggagatgatatgttacaagttcatttatgattaatttacattaatttacagaatagttgtcatatagtaagtataccatagtcacataagtgaatagtctgcagctacttgatccagaaacagagtgacaaaatgctttaaaataaacattatccaggtcataatcaccatcagggttcttcactatctactttttcacccaatataaacacaataaattataatctatcaggccaactaatgagcattaatcagtaataaacagctaattattcaaagacaaatacaaagcatgcagtctgtcatggccttttattggccagaaatgactgtcattagtcattggttcaatatgttttgatctaaatggaactattggtgggtgcttaccattctgcagcttccagctgggtattgtcccctcccagctttgtgaagacaacttccttaagcttctcgcatggggaggagcgggaaattccaactaggtcacataggagttgtttctgattgaaaaataaatatgaatagtctaagtacatatatagtaaaacattagttatctgagaaataaaccaacctagacattgggcattaagtacacttcaaaggtttattacaaataattgaaatagaaaattaagacataaagtgtgtgtgtatgtgtgtgtgtgtgtgtgtgtgcgtgcatgaatgtcttcacatcacaaatgcatgtgattccagaacaacaacaacaacaacaaaaaaaaacctcaataaagacttaatgctaaaaaggagagatactaatacatttcctggtcatgtattttccccaacacttttttccattcatgccccaccccaaattcctctacccctgcaccctgtagcaatcaatgtgaagcaggttaaactacaaggggtttagtgctgtggttgccagtggcaaccggagttcttttgtagcttcaatagatgagaaagatctcgaagggaattaaatttcaaaggaacacttaaaagagtggccaaggagttttcttatttctaaacaaaatttcccccccagggtccccaaaggaagctatggtctagatcagaagtcaaccagttggttcatggactcagtgtggttcatttcaccggaagttgtacctatagtgcttatgcactatctataccagtactagattattctgcagagactgctaggcccatgcagtctaaaatgtttacaatctgatgctgtgcagcaagcttgtctcttgtgttctggattctaacagcatcctgggatgacccaggaagagtacactgcttccaaggacttaggtatgagagagaaatgcctaaaactcagccacataaatggcaccatgagaggactgacaagattcataccagcatcctcttaaatatcacactcaacaggtccaaatgacatgtcaccacctacacaactggaaaatcggagtccaaagcaaagagtgaaggcatgacttccccattgttcagggaatgatcctggggaataagtgaatacaggaacaggccaggtaccttagcccattgctttctccattcatccaatgcactaaagtgggaggagatagtcaccagaactcatcaacatacagcagctagagaagggatgccttggctctgtgcaaagcagagcttgagacctagccattttaaaaggcgtgtgtgaagcagacagcacagaattacggaaatattcacccaggtgagagggttggcctcaggtcggagggcagggtatgcttctctgttgatgagtcccaacttcacaaatccattcaaggctttggaatatccctgaagcaagaattagtaagcagcaaattaatattttcctttgaagacaaatgaattttacagcccctgcccccatttttttcttgaagaggagatctgcctttacaactaattaactttttggagggtttttaatctttataacttatttggtaataaccttcagtggtgaaatgggatcaatattaaccattgtcatggtaattcaagtaactggtttatgaaaaagccttttttttttcctcagtactatattgtgtggtatgttctggcaacataagggctaatttcctggtgaattatataatgcagtttaacagaacattgtttaatagaacagtcagatttaatctttattacaacgttttgttttcaaatgctaaggatcacatacagggcctcatgcatgctaggaaagtactctctctaggtactctaggatagctcctagttctatcaattttacttctttgtgaagggaggaacaataatccacacccccaaagcagagaacccaccctacagtgaagacctgggtacagaagaaaagaagggcatcacgttagggctctgatacctggttttctcattttgaacatacttctaacatctcacccatgatcactctaagtccctattttatggttaggaaacatttcagatttgtttttgagaagtccatctaaccctttcatctccttgagcctcagtccatgtgttttaaatgatttgaacctcatcctaagctctcaagacatataacagaagaatgacactgacctcagaaataaactacaactaacaagacaaaaatgcatgcttttagctattagagaaacacgcagtgtgtcactcaatccagatttgcaccttattaagccataacaggaagatggcagctgcctgctactatgtggaacctgagaccatggtaactcttacaagaccaagaaactaaactatacctaccactctctcctcctacctgctcccaatccttaaagactaaatacaataaattcgctattttgagttgggtctcctactactactactactactactactaatactaataataatactaataataataataataataacaaatttaacagtgaactgggagagggagcatcatagctgcacagggcttatgagaataggcagcacttgggcgttcaccccaaaacaagacagttgtcattccatccctccacagctcataaaacattgcagagagcaaaaggaacataaaagctggaggacagggagaaggaaagagactccctaggggatagtccgaatcctatgaccacacaaatagccctgattaaaccaaatgggtcattacacacaatcaagtcataaatcttggaaagggataggaagggaaagggatgggtgggtgggggttagggaaaataagagggttgtagcaaggagtattaaagacagagagtagcaaccagagaaagaatgtaaccaaaataaattatataaaagtatgaacttgtcaagcaactaacttaataaagatttcaaaaaaaaaaaaaagactctaacttggcttctcattttaactactagaaaaaataagcatatatactgctcatatcaaaactagaaatttgcttaaaatatagcacttaccttatatctcagtgttccccttaataatgtatgggcagatgaaattccataaatctcagcatattttatgctatctctgttaggatagccttccaagtttagacctgggaagtaatccatgggtgtgacagagttgagaaaagagactccccctgtcacattcacaacctgaggaagattgaaaaaataactgaggttcacaacagtttgtttcctctgggtttatctgcattaactatacaaaagaaactaacagaatagagcttaggaaatattaataaaactacagttgatacttagcatgccattagccaaactgttcctcttggaataccttgccaggctccctgaccccaaacttctaaatcttaatatactttattttctaaacaaaagtcttgttttcaattggctcttgctcctactaaaaatagtatcattttgtatgcattaggctggtttgttggttggttggttagttcattggttggttggctggttggctggttggttggttggctggttggctggttggctggttggctggttggctggttggctggttggctggttggctggttggctggttggctggttggctggttggctggttggctggttgactggttggctggttggctggttggctggttggctggttggctggttggctggttcgctggttcgctggttggctggttggctggttggctggttcgctggttcgctggttcgatggttagtgggttgttttgctttttgtaatgtaatcacatcaagtatgcattcagtggcttgtatgctctgataacaatggcaaaactttttcctttggtcttacatctcttgtgcttttcagtcacccaagtattattttctgactagctaacatgtatatgtgccagacatcacacaagttgtagagagaataaggaaaacagtgaatgagatcacttcacatattctgcacattcttattcattgggagaaataaaaccatttgctgaatgtttagcaatcacctgtatctgcataatacttaaagcataagctggagggtgctgaaaaggcagcagaaggaagacctccattcaaaacagacacaatgccaagactatacaacaaagctcacatacaacttactgctgtactgcaggtcagaagttaagccttgattccccagagcctcctacccctccccagctcacaaagtgccatgaacactgttctgtattttaactttaaagtattacatttcctcttaagagcttgaagtctgtggctgtccttgagataatgaaaaacattctggaatgataaacttagggctgaggccgctgtctaactggactttgtcctggtggaaacaatttgaccctgcttctaaagctgcacaaaccaagactggaagaggcagtggccagggttcagagggatttttcccaagggaaagtgggctaaatggcattactgctcagactccatctgtacatgagctcacctgtaccaaggtgtgagagagaaaattgaaactaatttatacacaacctaggccccagaaagaaagggctgcagcctacccaggatgggtgaggtaagatctttttagttgcaaagtcacttgcagagtacatctagagaaccttagaaagaatccaaagaatgtgaagaatgcttcctatctcatttgctcagagccccctttgtagactgtattgaacttcttctaataggattctagttttaaaagcaacctaataaagaagccacaaggtgggtctccgcatccatcttgcttttataacttaaggcaattctaaagtcccgcttctttaattgaagatcaggacaattttttaaaagtaaatatcatggcaaggattgtagtaaaaaactataatataatcccagtgttggaatgctgggacaggagaattgctaaaagtttgaggcaagcctaaactatgttctaagactctacataataggaaagagagagagagagagagagagagagagagagagagagagagagagagagagagagagagagagagagagagagagagggagggagggagggagggaggaaggaaggaaggaaggaaggaaggaaggaaggaaggaaggaaggaaggaaggaagacctaaaatcatcaagattcatttagtacttgtaaataaaattaggtaactcatactgactgctagtgatttgtttagaaaaagcatcagtgtgaggataagaaaccaagagttagacagattccctgaaagcagacagacagggagaggacccatgaccaggtaatacagatttcaaatatccaagacagctaacttcttcctatccaggactttggcttcttcattaccctcctgatctgagacaaaatccagaaaacttaaacccttgtggactgttctgcccctattgccccttccctcatgaagagactgaatttggaacttccatggtcccacacacacccactttgctgaggatttttctcagtgcattgctgtgtgcatggttactcaccactaataaaagcctttctacaactggctgattctgtctgccttgtttgagatttttcttttctactacattctacactacagagttttccttttaatcctttaagtagcaaagaaaaatgaaccaatcgaagaccgcttcacagtaacactcctcaacatatgatagcagcaatgtccagtgtcctaggatcaaaaataagccttgtccagttaattattctattgagaacttcaaaatgcatctatccattttgtagaggacatgtattttcctctgctcagacaatgcttgcatctcagacccactcctggcaaatgcatctctcatgacatgacatgttgtggtggaaggcttctctgaggccaattctttttcgtacatgcacccaaccaatgcgaattataggcatctattttagaacaagcactcaaatcctcaaaatacgtgatgaaccagcctgcctttatcttgggcttgaaaggcatcttataataaagacaaactaggttattcctgtttatgactaaacttctgtttctcaaggactgggctttgtcacacatatcacctttaactacaaatgatgctgtacttcctgttccaggaaatgacaactatgcccttgtttcaaagaattattataactatcttgaacagtagtttgtttcaaaagcttgttatgaccagttgtcagttatgactaccctgttatgatcaccttgcgatcatgcctttgtttcaggaggtcatcatgactaacttaacttgttatgcttatgtcctgctcctataaccctatcaccaatttttcccaccaagtccctcatttggaaaacccctccccttgagctatgaaaaccttgtctttctcttatctaatgctgacttcttaaacctgccttagggggaggcagctctagcctgtgaatatcaatttttaaaaaacaagcttgctttaattaatcaattaaaaagcattaattaatttggtatggtgatttgattcagtggtctctttcctttctcctgtctttgggattaatgtaagcttcatcattaaaccaataagaaatttatcatggtaaaggtacaggttgttatttattaggaaccctcagtctttcaggtgccttgtgtgcttttcatccatattaatcacacgttttctcataggtgtaaacaggctgtgagtggcacagaatcagattcaagcctatctaactaacacactatctgtgttctttctactgtatcacatgagcctcttggttttgcttatttcttgatcagtattttcaaaaaggattctgatatgagtgcatctactgaaagctatgctagagtcaccacagggagccattgctaacttgatttcttaaaaaggaggaggggcttcctttgcacatgtgtgaatttcccactttccaaggctgggaactgtaccaccatctacacagggatggacgtaggagaaggctagttactgctcccacacacaggggaggaaatcagatttgctagaagtcaaaacaatatcaatggctagaacttaccacaagaacacagcacagtgtgatctactctcactcatagaccctttctaattactagaaaagtttaaggattcaactgtaagtcagagacagagaggtcacctatcttcatcattacagttctgagttctgtaaggcccattctgtggctgaggggaacagtgacagaggagatggtggcagaggggagagaagggcaagactcagagacatttttaagaagtttaacaataaagattggggaacagatctatagactcaaatcactgaaatttctcagacacagacaatccaagtatctaagtgtcagacactctgattgagttcagacaaaagaatagtgaacaaaggctgaagtggccaggaaactctacaagaggaaatctgaacaagaccttgaaaaagtatcattttagcagaaataagaaaactatcctgttttctcttcaaatcctccaatgtcagaaaaaaaaaaaagtaagaatgcactccattttagaaattgagttcagggtggaagaggggagcagggaggtggatttcaaatttaacaaccacaagttctgcagccagaaacaacttgtttctcctgggctctctggagcactaagttctttctaccgctaatatttagtcaagactcagggaatgccaataagcctgctgttaggcagattacaacctacatggcttcattaattcacatgacaggactgctgcagaggtttccaggcctagatctaggaatagttagaacagagaagaaaatgcccaagccatgtgctcatacagcttgggtatggctctcatctcatccctttatcagaggaaataaaggcgacattgacatccttcctcatcctgatgcatgcccctaagagtctctgctggcttagtcaggtttgacctgctgtgatccgctggtcactaactcatttgttctcatgttaggctgctccttaaaatcacttggtgagatttagaaattctccaaagtcaagcaacttaaaaatttcaggactcagttttaggcattcattctcttcaaactttcactataatttaatgtgcagtcacgtctgtcactcatgctgatctctcagacctgagtaaccatagctgacaggaacattcatcttgcttcttagtaaatcctttcaaaaacaaactctgtgattgctaactatactcaccctgtggctctaaactccttaactcccagatcatggtaattttataacctttgacaaatctttcctcattcctctctcccttcatattatctggaaaaggaatttgcaagtggtctgaccagttacgaactctcctaacccaaaatgtgatctgactctagatggctttggaggagctagtgacatgtttagtaaggaaacttaccaccaagcttgaggacctgagtttgaacctgcatagccgaagagaaccagttcctgctagtcttctttacctctcgatatgcgccatgacttgcacaggcatggatgcacatgcccataggtttcacacgcaataaaaaagttaacgtaaaagcaattttactgccattgaagttggttgaaaccagggttgagcctcagatgatggccttagccatcctgtctatgccttagtctcattattaaaaaaataataatgaaaataatactaataatactgttagattgcataggcagaagaaatattagtttcattttctgatgcattaaacattccatgtggagggccacttgtatccagccctgaattctctgaactcaggaatgtcagtcagctatctttggtgcttattccaaagtctaattcagggtgtgtcatgctgaaatcacttggaagataatgtaaatatactcaatatgtgtcatactgaagtcacttgaaatataaccactatttacatatataaataagataagaagtccattttcagaaggagccaagaaggaagaatgaacacaggattggttgaaaaattaaaggtgactcattaaattgaatagaaaaatatacactaggaaatatcagtggagacaagggagggcaggtactaatacttgtcagtaagagctgggtgttgacagaaacttacctgattggaagagagtccacacattggatgactgcttctccagagctttctctgagatagaactgttcatctaaggggaggacaccaaccctttgcacatgtgtgttcatgtgcatggccgtgatcagacaacactgatattggggggaagtggttggagtccctccccagcctgaaacctgtttgctcagagcatggggtggagcttgccgccctttcattctgccactcccactgccactgcctgccaccttgctgttccagagtcaaacacgtggtcacctgcaactttactccaggatgatttggtgggaaagggccccttcccctgcttcacaaccgagtatcagaaatagtaaaatcgaactttgatcagactggttgtcttggtttcatctttatcgcgccgcctagccccccctcttctcttgcagattccaagatgcctttccagactagaacccagacatgtgagccgctggccggccacaacagatctggcgcccacgggggactggaaaatggcagaggacatttgaaagaggcactggagcatttgaagaagtttggagctccatgaaagaagaaaataattaaaggaaattcgtaccagagaaggtcggtatggactaagttgaaacagcgttaaacctgagcgctaattcacttaggttcagaagtgaagtttatcagggactaggaacataacagacagttagccgtagctcccagaagctacatttttaggcgtgagaaaagaaagggtttattaaaaggaaattaataatcaggcagatggccgcagttaaaaactgcattatgtgggaggaaaatatcccaaaaagcggagaaaaatttcagggtgccatgctttgttctctctgggccttacggcagaagtgctctgccctccttgtatctttgtctaatgtctggtgcaccagtccgttcacatgttcaattcatgtatgttcgtgtccagtctgtatgaatgaatgctctatgttttatgttggataataaagatggctaaaaactttatctgcaaagcagagagcggccacaagcttcaaccaggaaacaggcacacagcaagagggcccctgctggaaaaactgttcattataggaaaaaagagtctgataaaaagctgattaatggttttttcctggagagttctctgcaatcgtgattaatgtgtttggcaaatgataatgtgctttttattctatgattgtagctagaaaaattaatgttctctgattggtctaaatgtaactgcttcatttgtttttgtttttgtttttttttttattggtaatgtgctctacgtgttttcacaatcagttcataagttgttggttaaaattaataattattacattggtaacaagtggttagccttaaaattgataactcaagtttagagtctttccaacacatggcataaggcaggccaagaggctgggtctctaaggatatttctagttgagataataattaatgtggttcctatcctagaaagtaaaattaaagataagatttaaaacaatgcctctttattaaagcattaaagcttacatgtattcataggtattaattggcagccaaactttgtaatatgatagtaatacttctaatattaattctataagtaattaattgttttaaaattgggttttaccttcccaaagttaatgttgcaaaaagaaacttaataattctttacagatctagccagatgctgttttaatgaagttcacattcatagtttataaagagtcaatcagattgtgaaatttatcaaggcattacaatctggtttgcctactttttatatagttattatagatccaaaagtttctttttctttctcctttgcatcttaatgattgtaaaggttttgcttctagtgagtttgcaataactaaaatattttgtctctaacatggctaaatattaatctacattatgtaaaaaaaatttatcatttctgcttctatacaagaagccaagagttttaatctttcaatgtatattgtttcctaagtggaaaatgttttattagctaatgcctctcaagagaagtttactttaaatccttgctctcacccaaaagattcagagacaatatctttttattacttagggttttagtttactacagaaagttttgcaaaaaataaagatagcttttacaattgttactaattataatcaatggtaattaaatattagctgtgcccaaaacaattctttggcaagagaaaacattattgtaaagtcattttctcatcctcccagccaatcgctggcccacgtggacccagctagctactggctgctgtggcacggggccttgagacacacagtttcccagagctatagtggtgtgagaggcctccataaggtgggcagccttacaacgaaggagagggccacgttcacctttgccccatgagcctccacatgtcccgaccagactagccatatcgttctcactaagacccagagtaagttttctggcctacaataaattgggctgccttcagcctcttctgcctgagagaaaggccaagggcagatggccaaggttgaagtggttttcacctcaaaacacagtttcaactgtggttctaataaagaattgtatattcgtatatttttagaaaacctataacaaaaaaatgtgtcttaaaaacctgactagtgtatgttccttgttcaatacagcaatttatttggttattaccaaatattaataactgacctattacataccatcattttaaatatattaacaatcaatatcccaaagataagttaaaaattggttttatgcatgcttttatatctttataaattcatgcatgcatacatcccaattattgtgtttaataataacccctatatgagacataagtaaatgtgaattagatcacatgcttagtctcttaagtttccccctgtttcaacacaaataatctagctgtgtgctatagatgttgttttaaaatgctgagcaatcaaaccttgatttgtatattaatagtcaatgttatatctcaaagctcacaattgcttaagattgttcatccctcagatgctattaattctcaaatttgcaattgcttatgcatatacaacacatagaaaaaatgctgtcctcttaagaaggcaatctttagacatttattaaattgttctaattgcctagacattttaaaacaatgccaggctagatttatatctcacacagtctataggtcatacatataagatagattggatatataaccttgttctttgtatattcataacagttatggtttaagataatagtttagtattcttgaaaattgtgcatgtataattctttccttctagtgtcctcagttactacttgttcccacataatagtgttaattctttaggacttatacttaataatttgctacaaataaatgctcttatttctagttaataaataaataaattatgcacatgtgactcttaataactttttaagctttctagttacaaccgctccttaagagaaacgattgaaagtgcaattagtcactgccctttacagtcaagtatttaaaatgttttgtcaacaagttattaaatcgaaggattagatattgtaaaactttaaaactttaacttcttaaaagacaaagaagggggaaattatatccccgtacatactatttagtacatttccatgcacatatttaaatcatacctttattttcaaattttgaaatttagaggtcaaagtaaattctttatatttatattctttataaatgctttatagtatcttaaaaggatctacttattggcttacagattgatcctaaacagctaccttattagggaagggaaaaaacaggttctctccacagaacgctacagaagcatgatgtttaattcgtgtgacaagctggcaggtaagttgactcagtgccctgactgaagtgactaggaaactaaattgggttcatgttttagatccattcttgcttgccatttttccagtttagactagcttcaagccttttaactttatggcaaaagaacatcagagactgtatattctgacttagtaagattagtcatttaataaagtcataatgatttttctcttttcttcagtgtggccagtcattctaactcaatcttagactggtctaatatttagtccaatattagaaattcctattgtaaataactaaaattcttagttacctaacaaagttaattctgaacacagcccccacttccagaaaatctctggcctttttactaattctaaaaccagcccagagactgagcttgagccttgctatttgcaactgaataaagtaccaggactttcccaaaagaagctttgtgatgttactcttcactgtctaagatttttgtctttcaagcaggtcaatcaacaccttcagcctgactgcagcggttgtgcatccccgcccccaagagcatgcaggtggcagctgctattttcattctaaagacattccagcacgtattctggctttcagtctgatttgagatttaggtttgctactagggctagaaaggtgaaaatttcaaatactaaaagaaattggtttttattctgatagacgggcttagtctcttagctggtctctggttttccacccttgctgttattgcaaggtccccttagttcctcagactgtggaaataacagagatgaagagggtgaccttcagctcctaatatagcctaagagccacaaatagtggtgttactagtgacataattcttgtcgaggccttgctaaatctgaggttgacaattctcctttaggagctgcacagtactcagaactgtgcatactgattagtgatcatacaaatacagtatgagtaccatcttgggtgcagaaatttactgcaggggaaggtccagcttgaccatttctgagttccctgtgagatacacccggtttggatggaggttggtatctagttcgttacattcaagaatatctttctaaggttctgcatcccctccccaaaagataccaagagccacaagtgtgggtcatgacagcacccacgggaggaatcaggtcaatatccccccagccatggttaaagcccactcatctaaggatgagaaaatcatttgatcacctcagttaagcgtggccttattaagcttaattaatagaggagagagaggttggagaccataccattagaagggcaagcccttcacagcctcccaccctaaaaagccaattggccctgtactacggagccagtcgtcaccccctcctctctgttccccacctatcatccaagaatgtggaggaaaatccatgtactctccttcagtgttatcataataaagtgcctttcatattcgtacagtcagtccttgccagtgtcccaacgccctagaaggactgtgcagctgactacttggtattcagtaaaaatttcaactaggaagttacctattcagtaactaattaacatttaaaagacagagccaacacctcaggactagtctactagatgtttactaggacagaaaagacagtcctatccagatacagttttccataagaaaagttaagaaatcccactaagataagtttcttccttaggccctaagaattcaggcagaactatagggcataaataaattttatgcctcaacctagccttttcttttttatattaacaacagggaggagatgtaacctccccagcctgaaacctgtttgctcaggtttcagaagagagcatcggggcggagcttgccgccctaccattctgccactcccactgctgaggcctgctgctttgctgttccagagtcaaacacgtggtcacctgcaactttactccaggatgatttggtgggaatgggccccttcccctgcttcataacccagtgtcggaaatagtaaaatcgaatcttgatcagactggttgtcttggttccatctttatcacgccgcctagccccccacccccccttctcttgcagatttcaagatgcctttccagactagaacccagacatgtgagccgctggccggccgcaacagctttggccaccactgcaggtgtttatctctaaggctcccatgatgcacctatcttaccttcccattgagcagatagctagcaggctgcattatgttcatcaagactcccactggactccagctgaatttgtatcgcaagggattatctgaatgttcaggggccggaaggccaccacagtaggaaacataagactcaacctgcaaattgagaaataaatgaaatcagtaatattaagaagactaattattaagttattaattagggttaatgatatcaatctggtgaatatgataccattatttatttttttaagttattgaggcttttaaatatacatccttgaagacacttattaacacagattctatatacagatgcacacacatatatatgtatataaaaatatataaactctgctagagataatagctaaattttcattaaaaattcaactgttagagctgcagagataggtcagtacttaagagtgcttactgccctcgcagagaaccatgcttcattcccatgccaggcagcttacaacctcctgtaatcccagcctaattagaaaatccaatacccccttatagcctctgaggacatccacacatgtgtgtatatgtacatgcaaacatatacatacacataaatgaaaataaaacaaatcttttaaataaataaataaaaattcaactattgattcactttttctttgtcagttcttataagttttatatccaaaaaaaaaatatatatatatgtgaaaactccaaatcacttaatttgagtgaccatatatacatatacagttattctttgtcttatgtaattacttaattaaacaacagacaaaaatcccaaaggtttttttctaacttctaaatgcaaagtattatcaaagaatctgtctcaaaggcaaaatctttaagaaacaaaatgattacaataacatctatttctctagaactgttactaagatctaagttgttgtttgactcttcagatgtatctgtaggttctgtagtttttgtaattcctagccaaagtaagagctgtagtctagcaaatcactaatgagaagcaaaagacaatcctatatcccctgaggggatatctgctcaccgtggctcccagctctttggcagtatcaattgtttccattgccaacatgtgatcaagaccagggtctaatcccaactcaccaatgactgtaatgccagcatcatccacactgccacaggtagaaaagaagaaaatggaaaagtcagacacagaagaggaacgatcccgaggtggcaccttactggaggcggtggcttacctcttttccagttctttcatggctggtgtaatatagcttgcagtgaccatattaaccctgctttcgatgcaggctttggccaccacaggatgaagcacataaggcaacaagctgaaaacagagggagaagagattcagaagctttggttcccagtcctctctccccttccaggatgcttgaactataccacagctcagtcctaagaaaacgcccaaattaaagaattctctcagaaatgaccaaggacttctgaattacagagcagatttgtcctgctgtctaccatacagtgctcttcactaaccctttctgtagtttttctttttctttttgttacattcgctcattatgcttgtctatgtatgcatgtaacatatcatctgaatcacaaatatctcataattcagaaattctatttctagtttcctaacactaaaagcacaaggcaaacactgaagggtgtctagcagcatgttcataacagcccaaatcaaaacaacaaaattccatcaaagttcacacagtaaagaatacacagccggttctcataatcacaatatcaaatgaatgaaggcagtcacaaacaaggacatactgaataatccaattctacaaactttgaagacatgcaaaaagctaatctagatgttagaagaaagaacagtgcttctgggatagagtcaacaagtttacaaaggttaataagagggcttgtaggatattatcaacattgttccttgatgtggatagtggttagaaaatgtattaatgttgcagcaatttactgggcaacataactgtgattttttgattcaagagtgtgagtgtatgtgtgtttgtttgctcacccgtgagtgtgcaaataaacatacacatgcaagaggaagaatgtgagagagaaagatattgagagatagatggtgagacaaaaagacaagagacagagagaatattgaaacaaagagtcagagacaaaaaaaatttaccaattagcccagactggcctcaaactttcaattttcctctcttagcctcaagcaagtgctgaaaatacaggaaagtaccaccacatcacaccattctcatacttatttatattttcatacatatgttcttccaataaaatgtttatgagagaagacagagaaagaaaaagcaaggaggaaagagagacagggagaaaataagagaagattaaaacaaccagatgcagggctggagagatggctcagcatttaagagcactgactgctcttccagaggtcctgagttcaattcccagcaaccacatggtggctcacaaccatctgtaatgggatctgatgccctcttttggtgtgtcctgatgacagctacagtgtattcatatacataaaataaataaatcttaaaaaaaaaaagatgcacagagaactttttgttgcccactacctgatcacaagatcctgggattccaccagggattgcagctttgcctcttgcttgccaacagtcaaactgacagggttaatgttgtatttcttgctcagctgctgcatttggttggtcatgtcagagcctatttcaagaatacaaaaatccactttttagagatttcattccactggagacattttcatagctccacaacttttcaaaacttagaggtaaaaaatcatctaaggttcatattcaacaggcaatggaattagtttttattctttaaaaataaaaacaactcattacctagtgttatttctatattgttgtctcttgacagatattccaaaacaggcccagatacatagccagacccaaggaccaaaaccttcttcttggtactcattgaaaggaattgaatgcgttctctgtttgaaaaataaggggaaagccgataattaagtttatctcttttaaataacaaaatcgtgtgatctccaaacacaacagaaacttacatgctgaagcaacaaaatctccctgcacttagaatgacatgctatatccatttgttttagatcttgtataatagaccctggcctctgagatgctcaaggtctacagacttccatgaattctggagctagaatgaggacggggctacttttttctctgcttacttacaggcaaataacttggctcatactttgtcctcccagaagtatttactcaaaacgattttgcgtctggatgctggatacctcatttcctcctcgccacagtccaaggaaacaacaacctgaacaagggcttactgctacctgcatcaaaacctgttttgtcagatatctttaggctaatgtaagagattaagactagaaagtattttaattcttgacccatgtattatgttaaaggttgtactttgacaaacaatgacctccgtggtagtgacggcatcttgcttcaacaacaccctttattgtttacttcattatgttttgagtgtgggagtgattaacgaatggttttaacagagattctcagatgtcctcacgcatctataggcacagtctggtacgggaggtttcatagcaaactcggatggtggggtctttagggtattctcacactgcaatctgctaactttttcatttgtaataagtgaataaatactaatataaaaatgttcccatccatcttgacccagtaacacatgcacactaatgataggagatctccttcaactacaattcccatccatttaactgaaagaaccaccattacctgcttattagatattctttccagtagtaccttatataatttgtataaataaaacattattttgatgtaccaatataaaatctttaaaacactcctagattccaattcagtccatatcaacggtacccctaaccccaaattccagattaatagtcactgtgctaagcaatgaaaagaatgtaaaactaaagtaaattccctttttaggtaagatctccatgatcagattctggccataagaatacactagggaatgaaagaaggcagttccaggaatggcagcccatcagcccagctgccccaggagctctgatgcacacagactgaggaaatgacggactggttgacacatcattacactcacagtgaggaatcatccaggtaacaaggaaaaacagaattataaataacaggcaaatacgatgcttccctatgagaaactctcttacagaactataaaggcagtttgaatgttagaatatggaattcttttagaagtttagaattcttttttttaaatacatttttttaaagatttatttatttattatatgtgtatacactatagctgtctccagatactccaaaagagagcatcagatttttgttaccgatggttgtgagccaccatgtggttgctgggatttgaactcaagacctttggaagagcagtcggtgctcttaaccgctgagccatctcccctgccctagaattctttagaaagaatactcaagcatttaatgccgaaagattccgttgccacatttagttcagcagttctacacaagctaacacatattaagaaacacaatgcgcgcgcgcgtgcgtgcgtgcgtgcgtgcgtgcgtgcgtgtgtgtgtgtgtgtgtgtgtgtgtgtgtgtgatttaaaaaaaacttgattctggcacagctctttatttcttgcagcaagaactacttaaaaaaaaaaaaaggtaaggagggtgttctattgtgtcctgttttcccctggcttctctcagcaattcagcttgtatgttattttgcaaaatgtgaaaagaccaatggaagtacagtctgtgggggaaacaagacaatttcaagaaacatcagtgtttcctgactctgaacaacagagtcttttaaggaagcacagggctggcttgatggattcacaggctttttaggccacctttctcttctaccggctgccttcttcactgtctctgttcatttctgttcacgatcccactgtgctaaccatcatactgaacaaaccaaagtccaaaccttcaacaaaagcccaagcagaaagcaccacagaaagggttaatatgctgatttcctctgggggtggaaagatgaatttagaaatcactgtgtgcaaactgaagagggccgacagaactattcaaacatcaacactgctccactcttccactggcaggccagaccccagcgtattcgcactgccgtggggatgcaatgctgctcttcaaagaaaaagcacgctgttttcagctgttgttgcttgctttagatttgtaacatatcaaaatgtaaatgctatataagtctaatattaataactctccttgaaacctttaagcatatctatcaaatccaaggtaagaataaagtacaatatcagaatttttttttatcagtacatacctgctttctcggagtttctgtatatatttgtatttgtcagtcaatagaccattggacgtaatcactgcctagacgtgcatacacaagacagtcctttagtattcaacatttagtcctaggactatgaacacatgactgatagaagagacagaataactgaggtgtgggggagagagaggggagagaaaagaatgggaaggacagaaacagagagacgtcgtcaggaaagaatgaaaagagtacacatcacaaagagaataattgaagaggatggtagggaaaggttgcgttcacgtgaagagattctcataacttacgtctcgcaccactggggagaagttctggctttccagaggttgtgatgcatcggataacaactgtggagaaagagagcaggggagaggtcaacaaaaccgtgggcaaatggcacttatcaacagactaaaattctgcttaaagaaaacgttttctgaatattgctgcatataaatgaatgctgtaaaaatccatatagttccatgtgaagaaatataatgttgcaaggcaaggtctgatattaaaactaaaatttgattgtaatcacctttgcttgccgagaacacacaaaaactcttctttcataagcaaagtattatattaaatttctcaagtattagagccatagtcttccctctgagtgcttctgtcccatcccaaaacttcaagctgttccataagaagcaaacaagcacacaaacctcactgcagaccctttggtgatatttagttttatggggaacagaacacttcaaaactcaggaataatgagtagtatctataatctgtccattttctttgaagatagatcagatagatattgacagaagacacctcctctccctctctccctctctccctctctccctctccccctctccccctctccctctctccctccctccctctctccctccctccctctctccctccctccctctctccctctctccctccctccctctctccctctctccctccctccctccctccctccctccctctctccctctctccctctctacctctctccctctctccctctctccctaaaacctcaggcagagtttcttagaactggccttgaactcacagaactctacctacctctgtcttagagtgctaggattaaaagtatgagccatcacacctgacccatttcctaccacaactgaaaagccttctctttttctcaagatccagaaaccaacagagtatcagtcagaactgtcttagggtaaggtctagaacacttacccaaataaggaacaagtcataacactgaggtggtggaagtgggggggggtgttgggggaggaaggaggagatactaaaaacaatataacattgttttttagtgcatctgaagcagatagccaacaagaagttattgaatccaagggaaatgaagcttctttatcatccccaagcttgtgccctgtattaccacagaatctgtatgggcgttaagtcaaaagtaaaaaactaacaaaaatgtaccaataaaatgacttctaatgacattctgctagtcccacagaccaatgccttgctcagccaccatcagggaaagttcctcctacagcagatgggaacaaacacagagagacatcatgcagaaagtgagagaccttgcaatactcagccctaaatggaatagttccatcaaatccctctcagcaaggctcaggaaaccctgtggaagaggtaacagaaagagtataagagccagaggggatggaggacacaaagaggataatgtcctctgagtcaacaggaccaaagctcctatgagctcacagagactgacacagcatgcacagggccggctcaggtcggcaccagctccttggcatacatattctggctttcaatttagtggttttctgggactcaagtgtgttaatgagtgggtctctgtgacttctcttgggctctttttcttctgtttacttgtttcatccagttctgatgtgttagtttttgttttatcttcttatattttattccactatattctattattacccctgagcagcctgccccatccccatcaagagacacacagtgagtaaatcctgataagaagggaggtagagaggagggggaagaagtggagggaggaaaaatcataatcagaatagaaaatatgagaaaaaactattttcaatagaaggaaatatatgataaagaaaaaagtaagaatgagggaaaatagagtagcatgtatactttaaaatagttcctgttacagtatggaagaaagagaccccacagctctgttacagtatgtaagaaagagactccacagctctgttacagtatgtaagaaagagatccccacagctcgtgtgtctgaccgaacacttggtagcactgctttggaagatctcggagcctctgaagcagtgaagctgaagaatgtgggaccctgagggcaccccttgaggtctatgttttagctttgttttcatcctctctttacttctggatgctcccaaatacaatcaagcaccctcatgttcctgtcatcgccactgtcatcccttctgccatgtgtccaaataaaactttcttctcttagctgctttttgtcagatacttgatcacagcaatgagaaaagctatacaggaagttggtacaggcaagtggggtctttgctatgataaactggaccataggagtattagtccttaggtgcaagattattagaaaaatatgaaagagtgtaggctagagaagtcctcaaatatcacaagcaaagcttaatgggccattctgatgggaactggaaagaccaaaatgtcaatgagggaggcatattgtaaagtctgggacaatagagtttcagacacaaacaggacattgtcaggaactagaccagaagccattgtgttacattctttttaaaagtccagttgtttacgcccgtgtccaaaaacttaactgaagccaacttcagaagtgatagactagggactaggtgtctggaaagattgctcaacagttaggaacacttgttgcccatgcagaagacccaggtgtgtttcccagaactcgcataatggctcacagtgatctataagaaatgtatatacttacaattcaataaatattttaagaaattaagtagtgaactaatttgttcagtggagaaaaagttgaagacagtgcaaccattcagagtatggtatggctgtgtgtacacatcagagtattactgtgtttgcccattttactgtgagagagagcaaaaattcccagaactcacacaaattgagagaatggagtccacacacagacagcagcacatacacacacacaaccaaataaaaattataaattattctatagaacataccaattaaacaagataaaagtaaatgcagtagtagcaagacataagaactacattaaactcattcataaacacaaatgtaataatgtttttttggtttttttttgtttgtttttgttgttgttgttgttgtttttgagacagggtttctctgtatagccctggctgtcctggaactcactctgtagaccaggctggccttgaactcagaaatccgactgcctctgcctcccaagtgttgggattaaaagtgtgagccaccactccaggcataataattttaaacaagataaactgatgtgtgaactcaataagagtacaattaatagatatacatgaaaacacgtgtacatgcatctgcaaatactcacatactcaagatatattagaaaaactctttagagtaaaattgttagtcatatctccaaacagaaaaccacctaaatgtcaaccaatgttaaaataaacagatgatgatatagccattagcaacccgaacttggttacaaatagaagattaatacattgcagttaagttgagtttattctatgaatgtaaacatggctaatttgaaaatcagttgacagaaattatcatattagcaaatcaaaaatcatataatcttgatgaatgcaaaatataactaccattataatagttcaaaagtctttgatatgtaaaagtatgtgaaaatgttatagtaaatggtagattttctgatgaaactattctctaagggatagaagggaccaaagagtcagaggatggggagattggctgtaaaatgccaacttctaagaacaacacagatatgacaaccatggctatggtactggcactaggtccacacaagttggcccttcttggcactaagctatagagagggctgggctgggctgggctgggctgtagaatttattgttgatctactaatgaccaatagattctgacttgaggacaggcatggtatcaacttgtatgcccaccggtagtcagaccaagttctgacagtccccgtgcacgaccacacagacagctctgttaaactctgaagaacactaaacaaaagcgaagtaagtaaatactgaaaagaggcaggggcagggttgggagagataggtcagaggttggagaagataggagaaggtaacctgagtacatagtgtgtatgtataaaactttaaaagaatgaaacctattaacaaaagaggatttaaagaggcaaagattatgaactaaccagtaccccggagctcttgactctagctgcatatgtgtcaaaaaatagcctagtcggccatcactggaaagagaggcccattggacacgcaaacaggggaatgccagggccaaaaaaatgggaatgggtgggtagggaagtgggggggagggtatgggggacttttaggatagcattggaaatgtaattgaggaaaatatataataaaaaatattaaaaaaaaataaataaagaggcaaagatacaagttgcctgctactattattcaccataaaagcacaaatatctttcaaaggtaccaaaaccttcagggttagaggtcaggacagcagacatgaacgttggaaaagaggagtgattaggttactgggtgagaacaaagaagtgtgggggatgctagtgctgtctgacatttgacctatggctctcattgcaatgtgaccattttcacttttttttttttccttaaatggtaggagatttttttttttgagattataacatttctcccttcccatttctctctccaaaccctcatatattactttccccaatctctttcaaattcatagattcttttatcattgttactgcatgcatgtgtatgtatatatgtatacatatatattacagtctatttaatgctaatgctatttaatgctacttgtatgcatgtttcactcttaccgaacacatgccttcaatacttttaacactttatattttacataatatgaaagctttatatttatactgttttatttaataaaaattgtattctcgagtgttgtcatgatgagtcacactggctaactctcatgatgttttactgcaaattcatttaattgacccccccccacacacatacacacataccaatatgaaatgtactacactgaatgataatttttattaaagacaagtaggaggttagtggtatggccaagcaggttgccacctgctcttgtcaccaaggttatgttgttacctgcccaaaacttctacaagatcaggtcaagtacagatgggggaagggttcataagacctcattcccagccacctgctattggtggttgatggttgctcaaggagggacagtcatttttcttaggggtaagatgcccaggctttagtagatggcttctcattcaatacatataggcagtattaatagtcctcaggagttgctatttttttaatatatgaattaagaagggagatattttgtgggtttctgggcagagttggaggtggaaggagagggtagataaaataaaaatacactgtaaacagttttaaaagtagtgacatcatagcagttgcagtcaaatggaactaggaaaaaaataattctgagcatggcaacccagacccagaaagacaaacatggtacatgctcatttataaatggaatttaactgttacatggaggataatcatgttgctgtccacagacctagagaagctaaataacaaggaggtctcaggagtggatgcttggatctccctgggaaagggaaacagaatagatttttgtgggtggatggggatggaaacaggagggatcgggtggaggaaggagggacagagagcctagattgccaggatccagaagctggagaactcagagacttggggtcgaaccaaacaagactgagcaaaataaaatagaatggaaaaaggtaatgaaatgattcctaatgatattctgctgtactcataggatcagtgcccagcccattcttcagctaaaggcttcttcccctggctgataatggatgcagagaccctcagccaaacattaggtggggctcgtgaaccccactgaagacaggagaggaaggatcatcataaccagaggggtcaaggacaccaggagcacagggcccacagaaccaactaagaaggactcataggggccggcatgggtcttcactaggtcctctgcatacatgctttgattctttacattggggttttcataagaatcctaatgatgctcctattggaggtatctctcttttgtcttcttatgggacttttccctcctactgggttgcctcatccagccttgatttgagggtttgtacctagtcttactgcatcttgttatgctgaagtcagttgatatccctgggaagcctgctcttttctgaagggaaatggagaaggagtagatcttggggagaggagatgggggtgggccactgggaggaatagggtgaggggaagctgaggtcacaatgaattgtatgggagcagaataaataaataaataaaaattgaaaaagaaatattagtagtcacatccattcacatgactacttgatgttcaacatttaacagaatataagttgggctctaatacacaattttcacatggacaaggaggacttaaagtcaggaatttgccagcacttatggctactcttaatactctgctggagctagggtaactcaatccactttccagcatcaggaagtttcacgttagatttaggagactaagctcttaacaactatccctcctttaaatacgaggtgtaaaccctaggaaaaaagttcaggcaataagcaactgcaggaagggtttagacacagagcgacagcaggcttggagagtagcatcagaacagaggagcatccacaggagtagccgagtttggtgctgctttgacgatgagccttgggtgacgttctaatttcattctgtcagtgtcataaaccatagtgaccaaaggaacttactggaggaaagagtttatcttggcttactagtctatcacgtagggaagtctggccaagaacttgaagaaatcatggaggaatgctacttgctgtcttccctgctcaagttgcactagcttagaaatacagcccaggacctcctgcctaagcagcgatgctgacaaccctggggcccttctgtatcaagtgataatcaaggcagcctcctccagaatccacaaaccagactgatgtatacaattcctcaggtgatacagtccctcaaagcattggttcctgtcaggggactctaggatgatcaattgacaaaattaatcaggataatcaatagaagttcctgaaatcaactattaataagttttaaatgtattcccttctgagtagcagatataattgtgtgttttcctgctgcatcttgacaacgcatccatactacgcatgctccccacacacgagtaccattagaaggcatctcagatctcagctgtggtggtggttcagtagcttcatgttgagtcccccgggccatgtgactttcagctcactttatcacacagatgtggttattaagttaccaagagccagaaaagtatgcaacacgcaaggtattttgagactgagaagccatattgacataaattttataagatatatcttactgaaccacattttgcaattaaactttgtcataagtatgcatgtatagagaagatatatgtgtaggttctaatactatgtatagtttcagacattaactggggagtcccagaatgagtccccggcagataagagaaactaacataaagatcacaaaatggctttcaaaactgtgttctatgtgataaagaaaaatacattcataacaagaaaagcagggagctgagccatggtgacacacaacttaaatcacagcattggggaggcagagacagatggatctttgaggtagaggccatcctggtctacagagcaacttgcagaacagccaaggctacacagagaaactctatctcaaaaaaaccagtattgatagtgaagccagaagcctccaaccggaccagtgacttattgcagtgaacatctgcaagcagataagtgtggagaagaaggtatgctatgacagtaaacacactgatactctacagcttccacaattagatctgtttttcttttgtgggggacattgcaagctaagaaggtgggaatgaagggacagggaagtggatgggattgggttgcatgatgtgaagtgtacaaagaatcaacacaaagttaaataataataaaaaaataaaaaacaaaaaaacaataaaataagttgatgatgatggtgataatggtggtggtgatgatgaaattatggagattctcatcagagaaaaagaaaactaccaagaaacaaataattactattctgaagttaaaaagtatacagatatgtttgccaccaggataaaacctactatggtatttacagacctgaagcattcttctgaattctgaaggttcctatactctcacacacatatacatacatacatacatacatacatacatacatacataaatacatccttacatctatccatagatagatagatagatagatagatagatgcatacattcaattaataatgataataaatattgtttcaaaaataaatctagtcatcagagaagcaatgtcaaggaaagtgaatataaaacagagctgggacatttgttattctttttccctctagatacacatgcaagcactgagacatttacatctctagaatgttaaatcttaggaaaaccattaagattgtatactaaaagcagcaaattttacagatggggacctgagtgatcttgggttctacaaatctagctgacgcccactctcacttgccattgctgctctcttaattccactacctatcctcagagccaactccagacacactgcagactcagcctgcattggcacatgatagcaggcaagaaatatctccccaagttgtcctctgacctccacaggtacatcacagtgtaagcacactgctgtatgcacacatacacatccaaacagatcaacaaacaaataaaacatgttcccatggtattttctaatgtgtgcatcctccaatttcttctcagtaagcctttgtttactcagagaagacaacgtcagagcagaagttttgattattctcccactgttcactatggtcccatttcacagtcaaagagaaatggagaacaaggacacaaacaagagctgctattcataaatttggaatctctgccagaggctgacggctgttagcgacaaaaacataacacatgctaagtcagaacccaagataaacactggtggaagaaagagtttctctcgtaaacaggatggaatctagattacacagcatttcagaagagtcacaggagacaaaaaccacctcagcagaagccatcagtaaacacagcactagcaactcacatctagtgatattttgtatatttccagttgtcctgatctattaattgtatcacagtaaaaatgtcctcaatataaaatgatctagatagcaagggaattgatgataaagcaattttacctgcccccctcacagtgttgtaaacacaataaaatgtatataatgagataaatgtaattgacagtgagcatgaagtcagagacatcgtccccaggagacaagtaacagatcatgtaaaataaaaatggacctataagtacatttggggaagggctggtttttcattttacaacagatttcttagatacagaagaccagagcgttcataataatatactcattcataataatatgtatatatgcatttataataatgtatataataatataatattaatagctgtacttaaagtctcagagttgctgtggagcctaccggtccccttaccatttcttcaacataagggtaaagcatgtctccaaagtattccgtagcctcgattggaagctgtgctggcaaattgtcaatggaacacatcaggatcccggagccttcaacactaagagaggcaacatcctttattcaaatgcacaaaatatgacttgaaatttttcccagactctactccattccaacatttgtatcactgctattaatatatttatagtcttgagaaaaaggcataacactataaacaagactgcttttcaaacaaaactttttttttccaacttggcagcctatctcccatttaactataatgtatacttctttaagcatccaaatttcaatgtaattttcactacttttaaactgtgtttaagcatatacctccaagctttcacaccttcatacagaagggaaatattgagtgatccaaactagaacattaaggtaaagaatcatcaactttgacaaacccacctgtcgtgaataatttgctggtctgcgtcatacatgcaaaagggccgttctatggtggtgcactcagtcataaaatctatggaccctcccgtgtctgctgaaatgtcacatattgccacaagtctgaaaataacatgaacactctgatgagagggtagaattctcaggcctttgggaagactaacaatgatgaaaaataaagtgtgcttgaaacaaattgcatatatttcccaggaacttagaatcttcgttctacagttcaatctttgggacagtgtgttgacctaagacagagggttctttagtttccatgagaatcatgaagggaaaaaaggggaggaaaggaatctgaaagaaaggggtggggaaaatcccttccctacagaaaactttgatagcagacagaataaggtgacctagttcctcatctgtaaataagtattgtagacagtacagctgcctctcactctgtcacacctccacatcctcagattcaactgagacctgaaagcacttgaggaaaatgttgcatctatagtcagcatacagacaatatatcccttctcattattagttctctccttaccaagcagtagttctgcactgagatgcttgtcctcctgccccccacctctcaccacccccaccctgctccttttttgaaaggttttgctgagcccagacttagtcattcgttagaccattatagaacaattactaagcttggggcagtactgctttatcttaaaacaaaacaaaacaaaacaaaacgaacaaacaaaaatcacactgttaacaaaccttcccacttgtttttgtttctaacaagcccttcaatgaaacttgcaaggacaaaattattaatacattgtgacccatactttcgaagtcaaatgaagtttgctgttgagatagcaacttttctgccaaaaacgtgttcccattcctcattttctgaatatgtgatgcatagagaagcatcactgtgaactgagctcatccagcgatgtgtccacctgtacatcccagaattatcgactcttatgtaaatatagaggaggttccgcagacaaagccccttgctttctttgctgagggagaaaatgctttgaggattattccaagttctctccttacttgctaccagtaataaaatttgcttcccactcactgcagggttttgcttatcagctttgagctcatcaaatgaaccaaccattgtgttgtgttagcagtgcgcttacattatctaatctcctgttacattgttgttcctcctggtccactgacatttgcttcagagcagcaaacaacccaactctgcctctctgaagtttagcttagcggaatccctggtgcataaggtgtttaaaaatatttagtaatcaactcactcaatagccaagaaccatttaaatgtctattttataccaagtacttactaaggtcaagaccgaaagggccctatctttcaggagtataaatttcaagaaaatagagtgtgagtgttttcaaaacaaagtaataaactaaaactagagaggaaggagattaatggtgtagagatttctgaagaggagggggctttggaaccagaaataagtgagaagtcaatctcaaaaggcgatgggtaactgcaagatgcttctaagagacccaggcagaccatctctaaaatgagctttctggcatatacaaacattgtggtctttgatccctaataatagtgctgcagctaaaaaaaaacaaaacaaacaaacaaacaaacaacaaaaaaaccttcatttgttgatgatgtacaagcccttacttgtgtggtaattcaggacatccttcaacaggaacaactgaggacttcactgggaccaggagactctgagcatcctgacgtgtgagcaggcgaggggtgttttgctcccagtagattccattaattaaacaagttgtatagggcgcaatctgtcaaacaaagttccatgttcaaccaaacaggatgtagcccatgttaataccgtgtcctgagaggacactgtctcacagctaggagtaaagggcaagctaagccatcagtgtcacatctacagaacatttccaggtcgcctgccctcttctgaggaaagggtaacaatcacccacctttcactgtagaattactccatgataggatataaatataataaatacatcaaggttttattctgtaatatattgcacagctggggggggggagaacatactcaattctctgagcactattgctcttatctgccaaatgagagtaatgatcttcaaaagaaccctttgaggatgaatcagataacctctcaggggaactgcttgcacatacttacacatacatgtacatgcacatgtggtccgcacctcaccatcgctcttggtacacatctgatctaatcactaatacttaaggttcaagataaaattaggggcagagaatcacttaggtggctcgtccttacggacactatctcctattctcagtatgccttattgcctggaggtctttttctagcgggatggtcagcccagaagttgtatacatacaagtaacgctgaaaagactgagcatgttgtagttattattttaggcagagagagagagagaaggagaacgtggaaaaggccatgcatttgagagggatcaaggcatagtccttgcacagggttggaaggaggaaagtggggactaaacagcatatgggaagccaaaaggaagcagtgaatatatagatttcaaactgttttcaacttaaattgtttgcatagatttttgtttgtttgtttgtttgtttgttttgttccttatgcgcctcaaggcaatcttacacagaagggaaaagctttctttgtagcacactatccatgctaaaatagctaattttcataccagaacctacaagtgctacattgtgaagacacatttaaaattagttaaggcatttcttttcataatgtgtgtatggactttgagttagtatttgctttttagtttgttggcttattttgaggacagctgttacatgactaattgtgtttcttagaaaataaaattgctgtttgcccaccagagggattggtttgtaggtcatattttggacataaagaagatagtcccgtgcttgtatagctggaccatgtctcatcctgcagtcagtcacaagcatttggagatctctggctgctatggttgggatgttgagtgaccatgaagacccagtcttggcactacttaagtgaagtaagatcttgaaagggtagactctggtgcaaagtcttcaaatcactgggctatgcctttaagtggttattagaatttctcctctatctctcttctctcttctgatctggggggcttcctccaaaccactgccagccccaacccttagcagatgcaaaagcagcatgactgtctggtctttgatttaaatcttttccagaactgtgtgtttcaataaatcttttctttttataaaattactttgggtattttgttataaacatgcaacactgactcattaaacacaatgattgatgctgagagaaggaatcgttactcttactatgctgggaaatattgaggcactttggaatggattatctggcagaagctggacatggaaggacaggctagaagaacatctcgaaacaagctggagtatcactcaggggacaaatacttgcctgtcatacacaaaggcctagcttccatccctggtaccataaacaaagttataagaagtctgtaaccagtcataggggtagcttgtgcctatgctgccagaactcaggagagagagccagaaaatgtgagggtttgaggataggagggaatgcaagtgaagtctgaacaaacctaagccacagaaaggggcttgtctcaaaaaacaaataggttggggagcatggtgcggggcaggggtggggggccagggtataggggactttcgggatagcatttgaaatgcaaatgaagaaaataattgaaaaaacagaaaatgaaaacaaaggacaaataataaaaggatgaggagaaagaaagaaagagagagagagagagagagagagagagaaagaaagaaagaaagaaagaaagaaagaaagaaagaaagaaagaaagaaagaaagaaagaaagaaaaggggagggagggaggaaggaaggaaggaaggaaggaagcaatgaatgacatggggatgggagggagaaagagggaatgaaagaaaagaaggatgagaggaaagggaagaggaaaaaatgtaaaatgtctgaagtagaacatgtcaaaagaatcagaggagccagacaactgtctcctagagttaatgtctacagtgtagtccaaatgagtggtgcacgatggagccatgggaacggagacaaagaagggattccagaaaagtaaggcaaccggaaatgcacaacctctcctggaagaaacatccggcagcatgcaatcctaggcaaggaaggcttcagactcctgcgtctagcctatgacaacagtcacatgatccacagagggaaagcttccccagccagggtgaaggtgcacaaaacgtggtgtgtactgtacccaggatgcttgtcttggagctttagcagttgaagtctgtctgaatgggtttgggttttaccttggtccagtgactcctttcttatttctgctcctgccttttggaatggaatgtataccatgaagctaacaggctgctgtggcttagaaatatccagctgggtttttattttacatgcctttacagccaaagatgttattcttgggttctaagtgaattttaagaaatttctgaaatttgagttaatcgatgagttaagacattggaactgtcggggatgaaacacacattatgttaaatgtgaggaaataagttttattgtgtccaggacacaatactgtggttgaaataacagttgtaccttgaacttatgaaaaggttatgtgcatggtatttagagagtagaaatggaacccaaccatgctactaagaggtgacacctttggaaatgattaaattcgatagagatagtagaatatccatgatgaatcctcatggtttaattgacagagggagagagactacacacagagagatccacatgccctcctgtcatgatgtgtcacaccaatgggaacccttggaaaagtctgaactatgctttgttcttcaggcttccaaaatggtaagctaaatgatctcttttctttcccacatcttggtcttttatcttaaagccaactgagatgctaggtgaagttgtggttacaatattttggccttgggaaaaaagataaacttttgaagatgttgctcagtggtagagaatttgttgagatgtgagaaacactagattcttttcaaattacaaacaaacagttttaaaaaggttaggacacatgtacgttaagacaaagccttagataaagtgcttttcttttcaacagtgataaccagtgtaagctaacataaaaacaaggctgtctaggaattggtttagaagaaatgagtacacgcattctttaacagagagaccatgaagcaatgaacatccaacagaagccactacagccactaaccaaagaccacctcaaacccagccagagccaactactaaaaccaacgcatgagaaccaaaggactcccagtcagtctacccgaggacctcaagaatcaaggaaccaaggtacacactcacaatttccctgtcagaaaacagcctaactatggtgtgtcacagtacccaaatgaaaaagcaactagatagatactcacatctgtattaaaacgacttgtgtagcgctcaggatatttctcgtattctacgggatcgtacacaccatctgttttcctgacaagatgatgatggcgacttaacaccgtcccatacactttcctgaggtcttacaaagaaagacgaaattagtaaagtaagtgacaaatgataacgatttatggtaataaaagactaacacaaagactttttaaaacggtgaaagagataaggtcttccctctccccttacctccagtttttgaaacttctcttaattcgtggggctccacatactcacaaggtagctcattaaagacttcctgggctccctgtaaacatgcataagtaaatcaaacctcagtagggacggcgccaacacaatttcctgcaaggactcacagttttgcagttccacttgccctgtcttataaccaggatcacaacacagaagcactgatctcacaggccagctctggtttcagttgcattagaatggactgccatttaactaattgttcaggatagcaatgtttaaaaatgtggatgtaatacatgtctatttactttttgatttgctatagaggtcattttaagggagagaatggtccacgatagaaggaaaaagatcagatgtgtgctttacaacttaacacgaacagagacattctgtttaggagacatcttccctacagacttattattcaattacaagtttctccagggtgactacaggtgcagcctcaaaaccattccagaagaaaacattaatttcagccttagtcccatacactttagttactaggaaggagggaggatgggaagatggacaggttaagagacgattcaaattctaatacagtggttctcagccctttaatacagctcttcatgttgtggtgactccaactataaaattatttttgtttctacttcataactgtaattctgagttatgaaggttatgaatcgcaatgtaagtatgtgtgttttccagcctctgtaaatagtgggtggatctcataaagaggtcgcaaccttcaggttgagaactggtgttctaatagatgaaatcaactaaaagcaaagaaaatacctgaaccgttactgttctgtgctacaagaataacgtacaacaaaaatatcccaggggctggagagagagctcagtgattaagaatacggactgctcatctagaggacccaggttcagttctcaggacccatatggcacttcacaactgtccataactccagctctgggggatctgacactctcataccagtgcagataaagtcaaattaaataaaattatttaaaaagacagcccatattgaatggtcactcattctacttgaaatgcccatgacatgttcagcatttcggattgcttgccagaaaggataggtggagttaccttggatacattgccagtccctgtgaagacaaacgttaagggtcctattgactttggcattaaacccagagatatttcatagccagcatcacggacagcctgcacggcctggctgctgttcctgtagttgtgagccatgccaagatgctgcaagcaagcatacacagttcacgtcagtccactagctcaaatatgtttacatactgaagacatgatgtttccaacaaataagcaaataatctctcaccataaaaggcgtatgatgtcccaaagcaaggagccttaagcccattccatgtaaaatattaatcattcctgtcacagagccagaacaataagaagaccattaagtggagttgattaacaatgcacaaaataaactctgttcacaaccatttatcagaagaaacgataccttctttttccctctttgtccatttaaaccctgcttagtatcctaaggaagcagagaccataattacctcagattcataagggacctcagaacatcctgcccgttctctctactttctatcaaatccctcacccacctaggtcctcatctgttttttctcatattagccccgagagaagaaaatccctcttctcatagaaggttaacctctcctgtctgcatatcacaacctgacctggcctttcatagcaagggattcactcttgaatggcctgcctcatagtttaaacacactcccatctcccaagccaaaaccaagcaagcaaatacacaaagaagccacttgggcctaggggtcctaaacgatactcagtagcttacgtcacaaaacttgatggactttagattctgacttacttgtcagaattgtttggcaatgtagactctcctggattgacactccattcccctcagtatccctgacttgatggcatccttctttcctccttccctttctttgtgtcttactatatagcacaggctacccttgaactccctatgaagctcaggccatcctcaatttccagagcttcctgtcttactctcaagtactaggattgtgagcagatacagccatatctgcatccagaatcaatcttactttctttaagtgtctctctgttatgtatattatcaccttcaggattgttttgggggctccattctcttaacagcttcattttcccttagctaatctcatcacatcctaaatgtaagtgttttcttggtatggatgatccccacattcatacttccagtatgagtttttcttttgatttttaggatgaagtcattttttttaataccttaagtgggatgtcctcagacatctaaaactcagcatgcttatagcaaagatttcattctctaattctccttatttataatcttccatgcctttcccaacaccttggtcacacatacaattctacccaagtttcttttatctttttgttgaagtcaacttttaaataaattgtttatgtgcaataaaatgcaacttgaagtgcatagtttattaactattaacaaattgccacacacatgtgtcacagacttgagatgggaactgttgctgttacctgtatagattagattctttggttctatgtaaatgaagttataagcacagactctatggtctgtctcatttcatctagcatagttttacacttacatcactaattttgttcctttttagtgctgctgtttcccattttatggctatatcacaatctatctatctatttccatttgcattgtttccagagtactaaaaatttctgtaaaaaaatctttttgtggtgcagacacacaaatgttacatgacacacattcgatttgtgtatgtgtttagcttaagtggaatgacacctgggataataacaataccctccccaagaatcacagattgtctgacaagtatcccagtacccaaacaaaataggctattgccactgcccttggttgcatcccagaacttgaaggtaaaaccctattcctgaacacaccatacacttgagatacatgacttcaaggaatcaggatggaactgatctggaagcctccccccccccccccggaggactagctctgatagactgtcatgcaagattccaagggaaaaaagcaatcataggcatttatcagaagaaatgatacatttttttctctttgtccattttaaacctgcttagtgccctaaggaatcactaaggtatgtaggaagctaggtagagtcctacctagctgtaaagtctacaggctacaacaatgactggtccagcaatgcattcccaatggtgcaattgtgctcttttatattaaggataactaacagatgtctaattgtacttaagacctgctcagtaggagaggacccatgtacatcaatataaaactagccatggctggaaggcttatagacttttgactttttagagaccttactattgccattttctaaaatcaatataattcctgcactgtaactatttatacttatcctcactgataggtgtagctttcacccttcatcaaagcttcttttgtcagtagataaagattataatggagatctgcagttggtcaaaaggcagagaataagtgaccatgggtgcccagccttgacctgatacatctatagtataaccctacacctaagactacaggaaaactgtgcaagaaatgcagaaagattgtaagagccacaggagtaggtcatctgctcctaaatagagttttctagacatggcaggaaagctgtagttatgaagtgttgataatatggctacctaaacaagtcctgcatagtgataagaccagttgatatgccaaggcgtgtgagagaaatttcataaggcctagccctagatgaagaacttaagggagccaatgaatgctgacaaaatgagagcccatttcctgcaggaatgagctccctcataggttatccaatgccaagtggtcagccatgaacacatgtccatacaagcaacactgaatgaactcagtagggtgtgtgtatgtatatgacaatagtaatttaagaagaggtcatgaatttcagagggagtgtgaggaatataggaagggcttagaaggaaagctgaaaatgatgtaaatacagtactcatgagtgaaattcacaaaaaatttaaaaatgtgaattaaagtaaaattcttcttgagtttgtcaagctcattagccttagtgctttcttggtctttcaatatttgcagaataggttgcaatgtcctttcttatatatttttccctctagatagttccatcccctttggtaattactctatctagataatttttcaattttattgatttacccatgcctgcttcccctcccccttacttgcttaaataggtttaatcatccactgagtcctagctccagatgctatctcaaggacaatcctgacagctgtttccagctccatgctcttcctctagaaagtagcacttacttgtgtgttacctcaccctcatgattgttttaaataataaaagccatatcccattcatagacagtctcttgggtgagaatgaacactcacctgggtacgtctcaaaaggccttggatgggtcaacaaggctgacccatcacttggttaaagtgaccttctttcccccagattttctacctacagtgtctcattaccaaacaaagctatgttctccaacaggaccatcgagaaagctacaagctcttcctaggtattaaccttttgtcttattcttctttcttttctttgtggaaagaaacactgaaaagagagaatatacatggactctggagacacacttagcaagcttcctagagtcttgccaaacctacctgccacacccgcccactgtccgaaagccactatccgagaccctctgtgatccaccattttctcataatcaatgagccggatttcctgaagaaagggaaagaagaaactgttacaaccaggtaaatttagctcagttggacaagccagccacatataaagaaggaagaattttatattttttcctctgagaaaatgcatgttgatttacaaacccattaccagcaggtgtgtcattaaatgctttgaaaatgcttggtcacaaaccagctgtaatgagaggtaatggaataagctctgtgttacattccttgattgaggcaaggaaataactgagacaagggtttcctgtctgttcctcaagctgccccaattgatttgaaacacacttatgtctccatttatcttcaatcttttgatcccaaagaaaacacatcttccctaaggtacatcccagtgtgtgagtaaatcctggctcaccctaagaccttccttaccagtcttaactattacctcttccttcctccctgcctccctccctcccttcatctgtctttgtcttgctgtctttgtctctctgtctttgtctgtcagtctgtctttctttttaaagattgatctattttatgtatatgagtacactgtcgatgtcttcaaacacaccagaagagggtatctgatcccattacagatggtcctgagccaccatgtggttgctaggaattgaactcaggacctctggaagagcagccagtgctcttaaccactgtaccatctctctagcccactattgcctctttcttgcatagttgcttcttggtcattttaaataccttatttaccttaaaaattgtaacaatacagcccaacactttctctctttccctctctctttggcattttatctcctctttctttctcttctctttctgtctttacatttcttttcttcatctttgccaaacacaaattctttctctttcttcaaggaccttccctctctcaggaaagccccactaaggttgtcttaaaagctatagtgcaattacacgatatttttagcagtcatttatctctttgaagatagtaggagttaagggtcttaacttacagactggctgtttagcatgggcaaggtcttgggttgcagccccagtactgggggaaaggtatacaacttaaaaccaagtttacatctgtaatggcatttaatgtatctccagctcagaatgacttgttagatagctgccccaaagccttcccactgagtctatttttattaattttctaatgaggaccaaaagttcatataacagaaagtctctgaagttctccttaaagactttatttgcttctggtgaaatgtttttgcagcattgttttcattaattgctttgaatctgctcttcagaaacaaattaaccactacccaggatgagtggttttagaattaataggaacatatcctgttgttccgtatataatccatttccttcgttgcttatactgtcagtaaacgcaatcctctattctattctcagaatatcatatactatcatataatataatatcaaagattctgatatccttactcctggccttgagcctgagctatgctctgtatcttcactcaatctccatactcatccaatgacctggctgtagacacctcctcatctctgccagtcacaagaagctgttctcagctaccatgtccgacataccaccatttctgaaaactgtcaaaacgtagatatctgacaagctaagatgctcaactcccaacattcccacccatgcctgttgtcatgtgagcactttataaatgactgtctatgaggctcctttttagttgttctgatgccataggactgtgtaccctcagttaagttactgcacctctatgagacctgtctgaaaaacatggtgttaaattgcatgatctctgaggcaatttccagctctataatcctacatgatggtatctctgtacttgactttataaattagtaactgttttgtgggggacacatttatcctctacttcagaatatatagaagaaagagatgcattgcatctctaaactccaaaacctgctcggcagaggatacctctccatcaggcaccagccataggccatgtctcctactgattgacaggacctactgcttaatgccatcactactcctgaacactgatcacagttacctgtttcagaacctcatccaacaggttcatattggcttcctgagcttttattgtgtgggaaaagaaggcgtacgtcttcttggacatcagtttttcctctggaggcctttttactcccaaaatcagacaggcttctgtgatgtcttcctgaagaatgccaccagccctgacatactcctggggacgcaatttggagcaaagattactcccttagacttcagtactcatgctctctccaagcaaaagtacaaacagccttgggaaggatagagtttggtaaatgagaacaaacaacactacagctacttttaacgtttatgcttcctcaaaaaagataaatctatctggcgtggcgtgtatagaaaagagatttagaaattctgctattcactcacttttaagagcagattaaactctgatcacaaatggaggcaggaggagcagattttcaaggtcaatattggctccctaacaagttccaggatagtctgagaacccttctaagaacaagacaaaaacggtgaaagtttcttctatatgaatagtatgctgaactatctcatgtcccatggtgatttgttcccttttgtattgagagattacacatacatccacactgatttctttcattgcttttggttcagttaacatctatcattgttctaacaggaagatccatgctaggaataacacagtgaaagacaggcaaggtgcccttcaaggagtttataagatgccaggaacattcaaaagaactacagtcagaagaatccgaggaacactctaggcgagtggctattaagacatagacagtcaagttcatcaaaaatgtcgggtgggttgtgagaggtggaaatacagactacaccagcatcatcaagaggtcagaaggcaggggcacacatcagcaaggagaggtcagaaggcaggcacacatcagccagaagaggtcagaaggcagtggcacacatcagcaagaagaggtcagaagacaggcacacatcagcaagaagaggtcagaaggcagtggcacacatcagcaagaagaggtcagaagtcagaggcacacatcagccagaagaggtcagaagtcaggggggcacacatcagcaagaagaggtcagcagtcaggggcacacatcagcaagaagaggtcagaaggcagtggcacacatcagcaagaagaggtcagaaggcagtggcacacatcagcaagaagaggtcagaagacaggcacatatcagcaagaagaggtcagaaggcagtggcacacatcagcaagaagaggttaaaagacaggcacacatcagcaagaagaggtcagaaggcagggccagacatacatagacaagaaatgctgtgcacatagcacagtgtggaaaatgccacctcagctaatatgagatctaccagataatgaataacaatattagttgactgttaccaattcattagtgagtgttaggatggattctctgtataagccatctcacctaattctcatataattcccgtgaggtagaactaccccagttttataaaaactactgtgtagcctactggcaggtgggtggcacatgccttaaatcccagcactgttgtattcacaggtattttaaaagttgaaagtagtcaccttgtcatggatggcccgtctgttggaaggctgtatcaagaccttgtagcccagtttggtgatgcccttgatgtgcttgggagccagaggcgccctgcgctcccaggcgttcacgtcctccctccggagtgccatcactggcttgtggtggaggcctctggagaggcaggctctaagcctggccagtctcggcctctgcgctctcagcatcttcaaatgtggtagctgtaaagggaaggttgaacagtagcacggaaaagggtggggagctcacagatcgattctggtctccttagaaatctgttgcaaaatagaaccaacccgtttgatagacaagaacaggataaatctctgaccgtaaatgtgcctttaaccaaccaaattaaagtgtctaaaactggccaagcataagttcttaaatgtttttagcacaaaagtttaaacacactagtctactacacaaagacaaagagataaccgatatcattttttttacaaatgggtctctattttgatggtgtaaagcttgaaattcctataatggtatacaagagtcatcaaatgctcgttgtgaagtgtttccatatgcttcctaaataggagtcattctgtgcactctgacccctagtggccgcaaatgggttttgttttgttttgttttttaaccctccaccatagttgtatcatcagaaataaaagggaagactgtataaccttaataccacatgatcaacaaagcttggtggagcaagcctgtaatctcagtactgaagagactgaagcaaagagggctccatgagttcaaagccaatcagggctatacaatcagaccctgtctcaaaaaagggaaaacaaacactaacaataataaaataataataataaacctctagcccagaaacttagaatacccgatatacaattggcaaaacacatgaaactcaagaagagggaagaccaaagtgtggacacttccctatccttagaattgggaacaaaacacccatggaaggagttacagagacaaagtttggagctgagatgaaaggatggaccattcagagactgccctacccggggatccatcccataatcagccaccaaacgtagacactattgcatataccagaaagattttgaagaaaggactctgataatagctgtctcttgtgaggctatgccagtgcctggcaaacacagaagtggatgctcacagtcagctattggatggaacacagggcccccaatggaggagctagagaaagtacccaaggaactaaaggggtctgcaaccctataggtggaacaacaatatgaactaaccagtacccccagagctcgtgtctctagctgcatatgtatcagaagatggcctagtcctccatcattgggaagagaggcccattggtattgcaaactttatatgccccagttcaggggaacaccagggccaagaagtgggagtgggtgggtaggggagcagggcaggggagggtatagaggactttcaggatagcatttgaaatgtaaatgaagaaaatactaataaaaaaattggaaaaaataataataaacctcacagtcatttttccatatctttactgtactgaatttaccaaaagttctgtttcctcagctactgtaagttaataaaaccaaccagtcaattacatggcggccttcaccacagcagcgtgccattaaagaagagctgcttaagcctctgcctcagactctggtctctatctcatagatccattagggttcctctccatatgcagccctcagcccttcatacagaagccctgagagtctcccagttctctcactgctgtttaagtatgcaagatctcactcatttaggcagtcagaacgaacatatttacaaaatgaatattctgtgtcctgtacaagtaagttttatatagattcccactccacacacacaaatacctaggtctaaaagcatttatcttttttggaagtttgtatacaccttgaagccgcatattgctgatgatagttttctatagctttcagatgcaattatttttctatgtagggttggcagcaaattatggagattatctctcaaacacacgaacacacacacacacaccctacaggtgattttatacttctagaaatttcctactcattacagatagcacacaataaaacttttatgaaacacatttctgttacttcaaatttcaagttctaaggaacacctttctcaaagattaagagaatgaccaaacactcacaaatacttgctatctataatagagcacttcacaataagaccagaggagaaagcaagaaggaagaagtccaggaagaaatagctacacacagcagaaaccacacagcaagagaccagtccaggctttggctggggctcttgctggaggccaggaatttcagaagagactgtttacagaagatgaaaactgagaggaagtacctgaacaatctgtggtgacccaactggattgaccagccccttgctttccccatgcgtcaccagagtctctgctgtgagaccttctacagctcccagagtagcagctagaaagtactccattgcctgggcctgttctgactacatagaaacagatccctctccatcactctaagctcccacctgtaagtgtataatgaacttctgtcttaaaagaagttatcttctaagtcgggtatggtggcaaatgccttttatccaaccactcaagaggcaaaggcagggagatctctgtgagttcaagactagcctgctacagccagggcttgttacacagagaaacactgtctcaaaacaaaacaaaacaaaacaaaacaaaacaaaacaaaaacaacaacgaaactcttctttttttttttttttcaaaaagcatgtccttaagactttatttttttttccattttttattaggtatttagctcatttacatttccaatgctataccaaaagtcccccatatccacccacccccactcccctgcccacccactccccctttttggccctggtgttcccctgtactggggcatataaagtttgcaagtccagaaactcttcttaatatgcatttcattctttgattttaatggctcaggcagctttaatggccaataagctcattaaagcttagaaagacacagctgaacattttacacaccatacaggtgaaaaaccaggtgcttaacatttcagcaaagcatttctaactctgcatgcattttttggtttatttatataattttgtttttcaaaggaactaaattaaactcaggcccaaatgtgtaatagttactatcccagccaaagactgaacatttttttacttaaatgccaattccaaagacagtgtttcctttggacatgaacgcacgtgagcacacacacacacacacacacacacacacacacacacacacacactgcatttcttgaatttgtattatgaacttcccttcttggccctatatgaaggcacgtgaacctgttcctattacataataccccttcttgcaagcttgattattaaccaaagtcctgttcccaagctcggtgatatcctttccttctctagagttggtgtggtggtctaggagactgcaggagatacacataccatacagcctaaggcagtggttctcaaccttccttatgttgtaacccttgaatatattcttcgtgttgtagagacccccaaccataatattatttttgttgctacatcgtaactataatgctactgttaggaatcataatgttaatatctgtgttttcagatgttcttaggtgacccctgagaaagagtcatttgatactcccccacacacacaaatggttgtgactcacgggttaagaacccctcatctaaagaaactagaagacaaaatcagagactgaatcttaggaaactacattcaatgtttataaggaattctaaaagcattcaattcctgtattttatattttacaacaaacaaaacaatagttgccattgattattgtaaggctagcactatgctgcaccctttgcagatactagctcatttccttccggcaaaaatctcaagtagatagtgtcattattaccactcacggatgaagaataggaccccagagactaacttttgactgggaacagaattactaggacaaagcttaaaacctctggttttagtctgctttgctaaaggccagagactgtactcccagaggagatttgacacacggctaatctcaccagcgctggcttcaaaaagaaatctagtgtccactgtccactttagtaaattatcaattctcccctcctgacccctattcctctgtcaactgaaaatcttacccgagttcctgccaaggtaaattctagacctctcggtgtttgggaacaaaaagagctaagattttaggcggtattactggtcacttataagttggtgaactttgaaacttggtcacagcacagagctttccagtcacttaaggtcatcaagaaataggaaagtggttgaggatagagctcaaagaaagaacacgtgcctagcatgaaaacaggtcgcatccccaacaccatgaaaccagaaggaaagaagtagagcatcatgggtattccagagcaacactgacaggaagaggtatttacccgccgtggccttgagagatctttgggatttcctggcccagaatcttgtgatctttgcgataatattaggaagtaaatgtcatgtttattttccggcaaggtggaatgttttcggcttatccattgactctagtaagatcagctgattttacagaatactgataagagcagccgagtaagaaggacgtgaggacaggagatgggaaaggtttaggtagactcgcagaaaggccaacatttcaaatacaaaaatgtaagtttaaaagtattctctgtgcctttatgaaaaaaaaaaaatagctctacggcatatttctactcacataggatttgtcaggaacttgaaatgcctctgacagtcaccagagtcccaccctgagttgcaaagacggagaacgctctgctctgtagcaacagagctcgctctgtaacagcacctccagcctgctctttttcaggttctcacacttgatcgtgcagcaggtaatttactaaatgagccatctccccaggcccttcacggagctctgtgattcttctaccaaacatcaaaatttcctctcggttcctccaaccctttctatcgcatctactcaagcaaaatgctttgtaaacttgggaatcattgaaccaatgtgcacgcacagaatatgaacacctaagcattcccttaacatttgcccacttcccctgcgacatgaataaggttaggaatgcgcattcattattgaagaaattaagaccaaatgttccgactctggtctccatactcaggcctgtcttagggaccatttgttttaaatctttaagataatccctcgccccataactcagtgagtgaaactactaaatcacatgtagatgtttgtcaatgccaagttatttgcagcaatcagatatacacctgcaataaatattaagggttaacagacatcagatgaaaaaaaaagttaatatataagaaatacctagacaaatggatggacctggagggcatcatcctgagtgaggtaacccaatcacaaaggaactcacacaatatgtactcactgataagtggatattagcccagaaacttagaatacccaagatataagatacaatttgctaaacacatgaaactcaagaagaacgaagaccaaagtgaggacactttgccccttcttagaattgggaacaaaacacccatggaaggagttacagagacacagtttggagctgagacgaaaggatggaccatctagagactgccatatctggggatccatcccataatcagcctccaaatgctgacaccattgcatacactaacaagattttgctgaaaggacccagatatagctgtctcttgtgagactatgccggggcctagcaaacacagaagtggatgctcacagtcagctattggatggatcacagggcccccaatggaggagctagagaaagtaaccaaggagctaaagggatctgcaaccctataggtggaacaacaatatgaactacccagtactccccagagctcgtgtctctagctgcatatgtatcagaagatggccttgtcggccatcagtggaaagaggcccattggttgtgcaaactttatctgcctctgtacaggggaacactagtgccaagaagtgggagtgggtgggtgggggagtgggtgggggagtgtgttggggacttttgggatagcattggaaatgtaaatgaaataaatgcgtaataaaaattacaaattatttaatttgtctttttagttcattcaaaaatgtaatgtcttttaaaatatacttggaattttaagatttattttatttagtgtacttgtgtgccggcacaggtgtgtgtgtgtgtgtgtgtgtgtgtgtgtgtgtgtgtgtttctgtgtctgtgctggtagagcctctgaagccagaagaggatatccagactcttggactgcacttccgggtatttatgggagctggataccaaactctggttctttgcaaaaccagcaagtgcttttcttctctgggccacaaatattagtattttaaaacaaatacacattggtatatttccatataccatatcttcctagtgcacgtaagctgacatagtggctccatagctatgcttcttagcctgttctccagaccatagtgttcctgggaatttcaaaagagttccagtgttgacaaagcatctgacaatggcttagtctcagctaagccaaatccctcccaactcaaggcttattggaacgaagcctggaattgctaaggcactggccccagatttttcacttcccttttcgccagtacatttttcaaatgcttccatgtggaacctattataacctagccaaatggttaaaatcaaattacattttttttttaaagcacctgactgcgatggtcagtcttcactatcatcttgactgaatttagagtcacccaggagacacacctgtggcatgtctgtaaggcagctccagagacatttaacagaggaaggaagaccctccctgaacagggatggtaccatcccacagggtgggatctgggactgaataaaaggaaagcatcagcatccctctcttgctaaggaccacataggctcagcaacctcgtactccacgtctcatgccttcccttcaggaaaaattacatccccttttgtaatgtgagccaagataaatcttttccaccttaaattgcttctattgggttgtttgcttgtttgtttgcttgcttttgccacaggaacaaaagtaatcctatggagagttttagggagctggattccaaactctagttctctgtaaaatccggcttttcatctccctagccacacatatcagtattttaaaacaaaatacacactggcatattttcatatgccatgtcttcctagtgcatgcaaactcacaggtctcttagtgttaatttatctggtctgagaatattcacattgtctcacagtctctcagctgctgccatgcagtgataagagaaatctgactctcaaatcctggtttgatggcacacgcctttaattctagtacatgagatggaggcagagacatgtggatggatctctgtgagttcaaggatagactgtccttcgtgagttccagaagagccagagctacatagtaagaccctgactcaaaaaaataaaataaataaaacaatcaatcaaacaaacaaaaagctgactctcatagctaatggatttagactcagtgaaatagaaatcaataatctacagtagaatacaaaatgtctatctgctacattcatgtctccttgcaaaatttatagtccaggtaggaaatatgattattcctttctgtattccaagtgaatggcacatcagaaaacctcaacaattttaaaaaagaataaatctatttcaccaatatcactaactgatcactaattgagaaaatgccttatagctggatctcatggaggcatttcctcaaaaggggctcctttctttgtgattaactccagctttcgtcaagttgacacacaaaaccagccagtacaatatatttatctggattcaaacagtgcatgttacatgcatgcatccaaaaaacacatgctacccattaataaactagagttctatgattttgtgagccagttaaattttttaaaattacgtatgtaaaagaaatgacaaaatttaaatagacatcataaaagcctaggcagtaaaggatgcatgatggatacattaagaaccagattgtcaactcatcttccctacccctgcatacagagaaagaggtggggggagaaaagagggagaggaagggggagagagagaataaataaatctttgaacataatatcttttaaaagaaaaggaaaggtccctctagccatattatcctgattccattaagctcaggagagagtgggcagtgtagctacacgtgcactgggtgttttagaacataaatcagccctttaaatccagccaaagtacactaaacaggaaagggaagcactaagagtctcacctgagtctgcagcagtggatcattaaccacactaaccagaagccaaagtttccaacaggagacagatgagaggagtttggcccccgacctggtcctagcacctgactttactcattttgtgcatcatctacacaatccgactaactctccaggctgttcgagccttcatcccactagtgagctgagccagtctcacatgctgtagacaataaatgtgaaacgtttcccatcctgctgtttcccttaatgtaagaataaccacacccctagtagaagacactacctatcctttgccgccctatgataaattatgcttctcagggagaaagaaacaaaaaaattacaaagcctgtcttgaatgccactctttggacacggtgaacatgtaaaattcattaccgttttaactcttttatttcaacaagtttaagacgtgtcttcacattttagcatccctgaagtaagaatgcatcttaaattgaagacagcttacattcaagaaatgcaggaattgtgataagacgtgtggtttttctagaagattggggaaataccgcccataaataataaactcccttgtcttcctaagtcactttgcagagtgacaaccagattgcacagccagcaagttctgagccctaggtccactcaccttcatgggagggaggagtggatagcaaaatacaggaattcatgaccacatacacacacacacacacaccccacacacacacacacacacacaccaggccatctgtgttttgaataaagaactggaaggactgtataaatcattggtcattcagtgtgttgtaccgagcctcaccacagctacaccttgtccagattctcacaattcatgacccataatatccaagaaatgacagtctctacagatttaggaactatttcatttattcattcattctttctgtctctctgtgtgtctctgtctctctctgtctctgtctctctctctctgtccctccctctccaccccccccaccccattcaaccatgcagagttggttacaaagtgtgtccacagagctctctccttgagcacagccgcccagagtatcgagagttttggacacaacttgtttaactttttcatgcccatctcaaacggattgcagacagacccagccaaccaggtctctctcagtcacagttactagccctaaacctgcagctaggacagggaaagtttggtatcagaattccacccagccagggggactaggaggagatcagaaagaatcttgctctcttcatcaaccactacaggcaaagaagctttcagtccaagaataaataagttattcactctgcaactttaattttaacactgacccgtattgtcttcctcagcaacagaccacaaccaccagagcccacctgccaaaaagtgcatgcccctccccctttctattcagaagccgtagagctgagcctgaagcccttcctcccccccagcaaagctgggcagcgatgcctaaagcttgtgtcacctccctagttctgtcacagtgagcttcgaggtcgccccgcggctttggggcgggaggccgatttctcgactaacagctggagtcccgcggctgagctgggagctccaagtgcatcctgcaggcagaggtcgcgcctgcatccgagtcgccctcccggcccccttctcaccttgtaggttatggatcagtccccagccacccagagctcttgaatctggaccctacagactgatttccctgcaaaaaaaattgactaggggggcgggggcccagaa

SEQ ID NO: 39 - DNA, 3701 bp, Mus musculus
  source 1..3701: mol_type = other DNA
ttctgggcccccgcccccctagtcaattttttttgcagggaaatcagtctgtagggtccagattcaagagctctgggtggctggggactgatccataacctacaagctaccacatttgaagatgctgagagcgcagaggccgagactggccaggcttagagcctgcctctccagaggcctccaccacaagccagtgatggcactccggagggaggacgtgaacgcctgggagcgcagggcgcctctggctcccaagcacatcaagggcatcaccaaactgggctacaaggtcttgatacagccttccaacagacgggccatccatgacaaggagtatgtcagggctggtggcattcttcaggaagacatcacagaagcctgtctgattttgggagtaaaaaggcctccagaggaaaaactgatgtccaagaagacgtacgccttcttttcccacacaataaaagctcaggaagccaatatgaacctgttggatgaggttctgaaacaggaaatccggctcattgattatgagaaaatggtggatcacagagggtctcggatagtggctttcggacagtgggcgggtgtggcaggaatgattaatattttacatggaatgggcttaaggctccttgctttgggacatcatacgccttttatgcatcttggcatggctcacaactacaggaacagcagccaggccgtgcaggctgtccgtgatgctggctatgaaatatctctgggtttaatgccaaagtcaataggacccttaacgtttgtcttcacagggactggcaatgtatccaagggagcccaggaagtctttaatgagctaccttgtgagtatgtggagccccacgaattaagagaagtttcaaaaactggagacctcaggaaagtgtatgggacggtgttaagtcgccatcatcatcttgtcaggaaaacagatggtgtgtacgatcccgtagaatacgagaaatatcctgagcgctacacaagtcgttttaatacagatattgcgccctatacaacttgtttaattaatggaatctactgggagcaaaacacccctcgcctgctcacacgtcaggatgctcagagtctcctggtcccagtgaagtcctcagttgttcctgttgaaggatgtcctgaattaccacacaaacttgtggcaatatgtgacatttcagcagacacgggagggtccatagattttatgactgagtgcaccaccatagaacggcccttttgcatgtatgacgcagaccagcaaattattcacgacagtgttgaaggctccgggatcctgatgtgttccattgacaatttgccagcacagcttccaatcgaggctacggaatactttggagacatgctttacccttatgttgaagaaatgttgttatccgatgcatcacaacctctggaaagccagaacttctccccagtggtgcgagacgcagtgattacgtccaatggtctattgactgacaaatacaaatatatacagaaactccgagaaagcagagaacgcattcaattcctttcaatgagtaccaagaagaaggttttggtccttgggtctggctatgtatctgggcctgttttggaatatctgtcaagagacaacaatatagaaataacactaggctctgacatgaccaaccaaatgcagcagctgagcaagaaatacaacattaaccctgtcagtttgactgttggcaagcaagaggcaaagctgcaatccctggtggaatcccaggatcttgtgatcagcttgttgccttatgtgcttcatcctgtggtggccaaagcctgcatcgaaagcagggttaatatggtcactgcaagctatattacaccagccatgaaagaactggaaaagagtgtggatgatgctggcattacagtcattggtgagttgggattagaccctggtcttgatcacatgttggcaatggaaacaattgatactgccaaagagctgggagccacggttgagtcttatgtttcctactgtggtggccttccggcccctgaacattcagataatcccttgcgatacaaattcagctggagtccagtgggagtcttgatgaacataatgcagcctgctagctatctgctcaatgggaaggttgtgaatgtgacagggggagtctcttttctcaactctgtcacacccatggattacttcccaggtctaaacttggaaggctatcctaacagagatagcataaaatatgctgagatttatggaatttcatctgcccatacattattaaggggaacactgagatataagggatattccaaagccttgaatggatttgtgaagttgggactcatcaacagagaagcataccctgccctccgacctgaggccaaccctctcacctggaaacaactcctatgtgacctagttggaatttcccgctcctccccatgcgagaagcttaaggaagttgtcttcacaaagctgggaggggacaatacccagctggaagctgcagaatggttgggcttactgggggatgaacaagttcctcaggcggagtccattgtggacgcgttctccaagcacttggtctcaaaactctcctatggccctgaagaaaaggatatgattgtgatgagggacagctttggcatcagacatccatctggacatttagaaaataagaccattgatctcgtggtttatggagacttcaacggcttctcggcaatggctaaaactgtggggttgccgacagccatggcagccaaaatgttgcttgatggtgaaattgaagccaaaggcctaatggggccctttacaaaggagatctatgggccaatattggagcggattaaagcagaaggcattgtatttaacacacagagtacaatcaagctataaatgggaatgtattttccgaagcatcacacctgttaaccttgggtatgacaaacatgcttgctgatgtgctaaagcatgaagcatgatgtcttttgagtaagttcatggcaatggtatttttgaaatgcaaatctctactttagtaaagcttttggagtagaagaatccagacatcacagaaccctttaataagtctcctgtgtactttctgtgtgtatgtaaaggtgatcatagtttgttaatttattgtgcttccttttccccgagaacatattatcagcaagtgcagttgtactatttcctttcccagaaaaaaaaaaaacaacttcacatagttatatttttgtatttttcaaattgagtctgctacataccttttagactgacatatctaaaaacaattgtaaacgtaatttctcatgccttttgtaaagttatgatttatttagtaaattttttgttttgaaaatcatttctcttgagtttttggaaaatggagttgatacaataaattcagtccttgaaaatctttaatacccttccatgatagaaaccaaactaaataatacaaaataatcaccacaaaacgtatagcaaaaccagatggaaaggaatgcccttgtagaagaaaattatgaaaaaatttgtatcatacttaatggaaaaaacactgaatgaatggttttcacctgagagcacaagtatgttgtcatttaccccaatgactcaacctgtattgcacggttaggccagggcaattaggcaaaaatcaatcaatcaatcaatcaatcaatcaatc

SEQ ID NO: 40 - protein, 926 aa, Mus musculus
MLRAQRPRLARLRACLSRGLHHKPVMALRREDVNAWERRAPLAPKHIKGITKLGYKVLIQPSNRRAIHDKEYVRAGGILQEDITEACLILGVKRPPEEKLMSKKTYAFFSHTIKAQEANMNLLDEVLKQEIRLIDYEKMVDHRGSRIVAFGQWAGVAGMINILHGMGLRLLALGHHTPFMHLGMAHNYRNSSQAVQAVRDAGYEISLGLMPKSIGPLTFVFTGTGNVSKGAQEVFNELPCEYVEPHELREVSKTGDLRKVYGTVLSRHHHLVRKTDGVYDPVEYEKYPERYTSRFNTDIAPYTTCLINGIYWEQNTPRLLTRQDAQSLLVPVKSSVVPVEGCPELPHKLVAICDISADTGGSIDFMTECTTIERPFCMYDADQQIIHDSVEGSGILMCSIDNLPAQLPIEATEYFGDMLYPYVEEMLLSDASQPLESQNFSPVVRDAVITSNGLLTDKYKYIQKLRESRERIQFLSMSTKKKVLVLGSGYVSGPVLEYLSRDNNIEITLGSDMTNQMQQLSKKYNINPVSLTVGKQEAKLQSLVESQDLVISLLPYVLHPVVAKACIESRVNMVTASYITPAMKELEKSVDDAGITVIGELGLDPGLDHMLAMETIDTAKELGATVESYVSYCGGLPAPEHSDNPLRYKFSWSPVGVLMNIMQPASYLLNGKVVNVTGGVSFLNSVTPMDYFPGLNLEGYPNRDSIKYAEIYGISSAHTLLRGTLRYKGYSKALNGFVKLGLINREAYPALRPEANPLTWKQLLCDLVGISRSSPCEKLKEVVFTKLGGDNTQLEAAEWLGLLGDEQVPQAESIVDAFSKHLVSKLSYGPEEKDMIVMRDSFGIRHPSGHLENKTIDLVVYGDFNGFSAMAKTVGLPTAMAAKMLLDGEIEAKGLMGPFTKEIYGPILERIKAEGIVFNTQSTIKL